(19) United States
(12) Patent Application Publication
(10) Pub. No.: US 2018/0369129 A1
(43) Pub. Date: Dec. 27, 2018

(54) MICROBIOME-COMPATIBLE COSMETICS

(71) Applicant: AOBIOME LLC, Cambridge, MA (US)

(72) Inventors: Larry Weiss, San Francisco, CA (US); David R. Whitlock, Cambridge, MA (US)

(21) Appl. No.: 15/741,417

(22) PCT Filed: Jul. 1, 2016
(86) PCT No.: PCT/US2016/040723

§ 371 Date: Jan. 2, 2018

Related U.S. Application Data
(60) Provisional application No. 62/188,343, filed on Jul. 2, 2015.
(60) Provisional application No. 62/189,105, filed on Jul. 6, 2015.

(51) Int. Cl. A61K 8/9789; A61Q 5/02; A61Q 19/10; A61K 8/44; A61K 8/60; A61K 8/34; A61K 8/64; A61K 8/73

(52) U.S. Cl. CPC A61K 8/9789; A61Q 5/02; A61Q 19/10; A61K 8/44; A61K 2800/87; A61K 8/345; A61K 8/645; A61K 8/731; A61K 2800/30; A61K 8/602

(57) ABSTRACT

The systems and methods of the disclosure provide, inter alia, cosmetic products, e.g., finished cosmetic products that may be considered to be “biome-friendly” or “biome-compatible.” The systems and methods of the disclosure may provide for use of cosmetic products, e.g., finished cosmetic products, that may be used in combination with bacteria, e.g., non-pathogenic bacteria, e.g., ammonia oxidizing bacteria, which may be used in the form of a preparation or composition to be applied to a subject.

### CROSS-REFERENCE TO RELATED APPLICATIONS

This application claims the benefit of priority to U.S. Provisional Patent Application Ser. No. 62/188,343 filed Jul. 2, 2015, and U.S. Provisional Patent Application Ser. No. 62/189,105 filed Jul. 6, 2015, the entire disclosure of each of which is hereby incorporated herein by reference in its entirety for all purposes.

### BACKGROUND

Beneficial bacteria, e.g., non-pathogenic bacteria, can be used to suppress the growth of pathogenic bacteria. Bacteria and other microorganisms are ubiquitous in the environment and are naturally present on subjects, for example, human and animal subjects. Bacteria are a normal part of the environment of all living things. In the gut, these bacteria are not pathogenic under normal conditions, and in fact improve health by rendering the normal intestinal contents less hospitable for disease causing organisms. Disease prevention is accomplished in a number of ways: nutrients are consumed, leaving less for pathogens; conditions are produced, such as pH and oxygen tension, which are not hospitable for pathogens; compounds are produced that are toxic to pathogens; pathogens are consumed as food by these microorganisms; less physical space remains available for pathogens; and specific binding sites are occupied leaving fewer binding sites available for pathogens. The presence of desirable bacteria is seen as useful in preventing disease states.

There is a need in the art to provide products, e.g., cosmetic products, that maintain or sustain at least some level of beneficial bacteria on or in subjects. This may be accomplished so that beneficial bacteria may regulate or suppress the growth of non-autotrophic bacteria, e.g., pathogenic bacteria, and/or maintain a suitable microbiome on a subject to benefit from health improvements that the non-pathogenic bacteria may provide.

### SUMMARY

This disclosure provides, inter alia, a finished cosmetic product. The finished cosmetic product may comprise a cosmetic product, e.g., shampoo, e.g., body cleanser, disposed in an end-use container, wherein the finished cosmetic product has one or more of the following properties:

a) said cosmetic product, or said finished cosmetic product, is substantially free of a preservative, e.g., a paraben;

b) said end-use container is configured to reduce retrograde flow;

c) said cosmetic product, or finished cosmetic product, is irradiated; or

d) said cosmetic product consists essentially of the composition of one of (i) or (ii):
(i) water, cocamidopropyl betaine, rosa damascena flower water, decyl glucoside, pyrus malus (apple) fruit extract, glycerin, hydrolyzed adansonia digitata (baobab) seed protein, hydroxypropylcellulose;
(ii) water, cocamidopropyl betaine, rosa damascena flower water, decyl glucoside, pyrus malus (apple) fruit extract, glycerin, hydrolyzed quinoa, hydroxypropylcellulose, and citric acid.

In some embodiments, the finished cosmetic product may have 1, 2, 3, 4, or all of the properties of a, b, c, and wherein the cosmetic product comprises a shampoo. The finished cosmetic product may have 1, 2, 3, 4, or all of the properties of a, b, c, and d(i). The finished cosmetic product may have 1, 2, 3, 4, or all of the properties of a, b, c, and wherein the cosmetic product comprises a cleanser, e.g., body cleanser. The finished cosmetic product may have 1, 2, 3, 4, or all of the properties of a, b, c, and d(ii).

In embodiments the finished cosmetic product may have the property of a. The finished cosmetic product may have the property of b. The finished cosmetic product may have the property of c. The finished cosmetic product may have the property of d(i). The finished cosmetic product may have the property of d(ii).

In embodiments, the finished cosmetic product may have the properties of a and b. The finished cosmetic product may have the properties of a and c. The finished cosmetic product may have the properties of a and d(i). The finished cosmetic product may have the properties of a and d(ii). The finished cosmetic product may have the properties of b and c. The finished cosmetic product may have the properties of b and d(i). The finished cosmetic product may have the properties of b and d(ii). The finished cosmetic product may have the properties of c and d(i). The finished cosmetic product may have the properties of c and d(ii).

In embodiments, the finished cosmetic product may have the properties of a, b, and c. The finished cosmetic product may have the properties of a, b, and d(i). The finished cosmetic product may have the properties of a, b, and d(ii). The finished cosmetic product may have the properties of b, c, and d(i). The finished cosmetic product may have the properties of b, c, and d (ii).

In embodiments, the cosmetic product, or finished cosmetic product is an irradiated finished cosmetic product. The cosmetic product, or finished cosmetic product may be gamma-irradiated finished cosmetic product.

In embodiments the finished cosmetic product or cosmetic product may be substantially free of a preservative listed in Annex VI, e.g., substantially free of at least 1, 2, 5, 10, 15, 20, 30, 40, 50, or all of the preservatives listed in Annex VI. The finished cosmetic product or cosmetic product may have less than about 500 ppb of a preservative, e.g., less than 500 ppb. The cosmetic product or cosmetic product may have less than about 500 ppb of a preservative listed in Annex VI, e.g., less than 500 ppb of a preservative listed in Annex VI. The finished cosmetic product or cosmetic product may not include a preservative. The finished cosmetic product or cosmetic product may not include a preservative disclosed in Annex VI.

In embodiments, the cosmetic product or finished cosmetic product, if exposed to challenge with a microbe, e.g., a bacterium or fungus, will support growth of said microbe, e.g., as determined by U.S.P. 51, Antimicrobial Effectiveness Testing. The cosmetic product or finished cosmetic product, in the absence of a treatment, e.g., sterilization treatment or the addition of a preservative, supports microbe growth, e.g., bacterial or fungal growth, e.g., as measured by U.S.P. 51, Antimicrobial Effectiveness Testing. The cosmetic product or said finished cosmetic product may be free of preservatives, e.g., preservative free.

This disclosure provides, inter alia, a cosmetic e.g., shampoo, e.g., body cleanser, disposed in an end-use container, wherein a finished cosmetic product comprises: water, cocamidopropyl betaine, rosa damascena flower water, decyl glucoside, pyrus malus (apple) fruit extract, glycerin, hydrolyzed adansonia digitata (baobab) seed protein, hydroxypropylcellulose.

This disclosure provides, inter alia, a cosmetic product, e.g., shampoo, e.g., body cleanser, disposed in an end-use container, wherein a finished cosmetic product comprises: water, cocamidopropyl betaine, rosa damascena flower water, decyl glucoside, pyrus malus (apple) fruit extract, glycerin, hydrolyzed quinoa, hydroxypropylcellulose, and citric acid.

This disclosure provides, inter alia, a cosmetic product, e.g., shampoo, e.g., body cleanser, disposed in an end-use container, wherein a finished cosmetic product consisting essentially of:

water, cocamidopropyl betaine, rosa damascena flower water, decyl glucoside, pyrus malus (apple) fruit extract, glycerin, hydrolyzed adansonia digitata (baobab) seed protein, hydroxypropylcellulose.

This disclosure provides, inter alia, a cosmetic product, e.g., shampoo, e.g., body cleanser, disposed in an end-use container, wherein a finished cosmetic product consisting essentially of:

water, cocamidopropyl betaine, rosa damascena flower water, decyl glucoside, pyrus malus (apple) fruit extract, glycerin, hydrolyzed quinoa, hydroxypropylcellulose, and citric acid.

This disclosure provides, inter alia, a cosmetic product cosmetic product, e.g., shampoo, e.g., body cleanser, disposed in an end-use container, wherein a finished cosmetic product consisting of:

water, cocamidopropyl betaine, rosa damascena flower water, decyl glucoside, pyrus malus (apple) fruit extract, glycerin, hydrolyzed adansonia digitata (baobab) seed protein, hydroxypropylcellulose.

This disclosure provides, inter alia, a cosmetic product cosmetic product, e.g., shampoo, e.g., body cleanser, disposed in an end-use container, wherein a finished cosmetic product consisting of:

water, cocamidopropyl betaine, rosa damascena flower water, decyl glucoside, pyrus malus (apple) fruit extract, glycerin, hydrolyzed quinoa, hydroxypropylcellulose, and citric acid.

In embodiments, the cosmetic product, or said finished cosmetic product, comprises a component added to provide one or more of the following: a fragrance, a color, viscosity, foam forming and foam stability, adhesion, moisture retention, moisture binding, pH stabilization, cleansing, thickening, softening, conditioning, e.g., hair or skin conditioning, lipid layer enhancing, barrier-forming, or film-forming.

In embodiments, the cosmetic product, or said finished cosmetic product, comprises one or more of an antioxidant, fatty substance/oil, thickener, softener, emulsifier, light-screening agent, foam forming and foam stability, antifoaming agent, moisturizer, fragrance, surfactant, filler, sequestering agent, polymers, acidifying or basifying agent, dyes, colorant, pigment, pearlizer, opacifier, organic or inorganic particle, viscosity modifier, cleanser, adherent, moisture binder, pH stabilizer, conditioner, de-tangler, biobased surfactant cleanser, lipid layer enhancer, skin conditioner, and natural hair nutrient such as botanicals, fruit extracts, sugar derivatives and/or amino acids, hydrolyzed proteins, or vitamins.

In embodiments, the cosmetic product or said finished cosmetic product is one of the following:

a baby product, e.g., a baby shampoo, a baby lotion, a baby oil, a baby powder, a baby cream; a bath preparation, e.g., a bath oil, a tablet, a salt, a bubble bath, a bath capsule; an eye makeup preparation, e.g., an eyebrow pencil, an eyeliner, an eye shadow, an eye lotion, an eye makeup remover, a mascara; a fragrance preparation, e.g., a colognes, a toilet water, a perfume, a powder (dusting and talcum), a sachet; hair preparations, e.g., hair conditioners, hair sprays, hair straighteners, permanent waves, rinses, shampoos, tonics, dressings, hair grooming aids, wave sets; hair coloring preparations, e.g., hair dyes and colors, hair tints, coloring hair rinses, coloring hair shampoos, hair lighteners with color, hair bleaches; makeup preparations, e.g., face powders, foundations, leg and body paints, lipstick, makeup bases, rouges, makeup fixatives; manicuring preparations, e.g., basecoats and undercoats, cuticle softeners, nail creams and lotions, nail extenders, nail polish and enamel, nail polish and enamel removers; oral hygiene products, e.g., dentrifices, mouthwashes and breath fresheners; bath soaps, e.g., foaming body cleansers, and detergents, deodorants, douches, feminine hygiene deodorants; shaving preparations, e.g., aftershave lotions, beard softeners, talcum, preshave lotions, shaving cream, shaving soap; skin care preparations, e.g., cleansing, depilatories, face and neck, body and hand, foot powders and sprays, moisturizing, night preparations, paste masks, skin fresheners; and suntan preparations, e.g., gels, creams, and liquids, and indoor tanning preparations.

In embodiments, the finished cosmetic product is a shampoo. The shampoo may comprise water, cocamidopropyl betaine, rosa damascena flower water, decyl glucoside, pyrus malus (apple) fruit extract, glycerin, hydrolyzed adansonia digitata (baobab) seed protein, hydroxypropylcellulose. The shampoo may consist essentially of water, cocamidopropyl betaine, rosa damascena flower water, decyl glucoside, pyrus malus (apple) fruit extract, glycerin, hydrolyzed adansonia digitata (baobab) seed protein, hydroxypropylcellulose. The shampoo may consist of water, cocamidopropyl betaine, rosa damascena flower water, decyl glucoside, pyrus malus (apple) fruit extract, glycerin, hydrolyzed adansonia digitata (baobab) seed protein, hydroxypropylcellulose.

In embodiments, the finished cosmetic product is a foaming body cleanser. The foaming body cleanser may comprise water, cocamidopropyl betaine, rosa damascena flower water, decyl glucoside, pyrus malus (apple) fruit extract, glycerin, hydrolyzed quinoa, hydroxypropylcellulose, and citric acid. The foaming body cleanser may consist essentially of water, cocamidopropyl betaine, rosa damascena flower water, decyl glucoside, pyrus malus (apple) fruit extract, glycerin, hydrolyzed quinoa, hydroxypropylcellulose, and citric acid. The foaming body cleanser may consist of water, cocamidopropyl betaine, rosa damascena flower water, decyl glucoside, pyrus malus (apple) fruit extract, glycerin, hydrolyzed quinoa, hydroxypropylcellulose, and citric acid.

In embodiments, the finished cosmetic product may be a conditioner. The conditioner may comprise hydroxypropyl cellulose, cationic guar, coconut oil, and a fragrance. The conditioner may consist essentially of hydroxypropyl cellulose, cationic guar, coconut oil, and a fragrance. The conditioner may consist of hydroxypropyl cellulose, cationic guar, coconut oil, and a fragrance.

In embodiments, the finished cosmetic product, or cosmetic product may be treated by sterilization. The sterilization may comprise irradiation. The sterilization may comprise heating. The finished cosmetic product, or cosmetic product may be sterile, e.g., as determined by sterility assurance level testing. The finished cosmetic product or cosmetic product may have at least 10, 20, 30, 40, 50, 60, 70, 80, 90, 95, 99, or 99.9% of the bacteria, are dead or incapable of cell division. The finished cosmetic product may be provided that has all bacteria, spores, mold and fungal species dead or incapable or cell division.

The finished cosmetic product or cosmetic product may have at least 10, 20, 30, 40, 50, 60, 70, 80, 90, 95, 99, or 99.9% of the bacteria, have radiation induced DNA damage sufficient to inhibit cell division. The finished cosmetic product may be provided that has all bacteria, spores, mold and fungal species having radiation induced DNA damage sufficient to inhibit cell division.

In embodiments, the end-use container is unopened, e.g., the factory seal has not been broken.

In embodiments, the cosmetic product, or said finished cosmetic product, is irradiated, e.g., with ionizing radiation, e.g., with gamma rays, e.g., with x-rays, e.g., from an isotope, e.g., cobalt 60, or with ultraviolet, e.g., ultraviolet C (UVC). The irradiation may be sufficient to provide a sterile product. The sterile product may be characterized in that it is substantially free from microorganisms, e.g., bacteria, e.g., fungi, capable of growth, e.g., consistent with U.S.P. Chapter 1211, as determined by U.S.P. 71 Sterility Testing Methods and Standards.

The finished cosmetic product may be provided wherein the sterile product is characterized in that it is substantially free from microorganisms, e.g., bacteria, e.g., fungi, e.g., spores, capable of growth, e.g., as determined by standard culture methods as described in U.S.P. 71.

When challenged for microorganisms capable of growth, said cosmetic product or finished cosmetic product shows no growth, e.g., when said microorganisms are measured by U.S.P. 71 Sterility Testing Methods and Standards.

When challenged for microorganisms capable of growth, said cosmetic product may show no growth, e.g., when said microorganisms are measured by standard culture methods ad described in U.S.P. 71.

In embodiments, the cosmetic product, or said finished cosmetic product, comprises an exogenously added additive selected from an oxidant, e.g., a naturally occurring oxidant, a free radical scavenger, or a free radical quencher. The cosmetic product, or said finished cosmetic product, contains a plurality of components, and is irradiated after mixture of the plurality of components. The finished cosmetic product may be irradiated after said cosmetic product is disposed in the end-use container. The finished cosmetic product may be irradiated after closure of the end-use container. The finished cosmetic product may be irradiated after sealing of the end-use container. The finished cosmetic product may be irradiated prior to closure of the end-use container.

In embodiments, the finished cosmetic product, or cosmetic product may comprise an indicator that indicates if said cosmetic product, or said finished cosmetic product, has been irradiated.

In embodiments, the cosmetic product, or finished cosmetic product, is heated, e.g., by microwave oven or autoclave. The heating may be sufficient to provide a sterile product. The sterile product may be characterized in that it is substantially free from microorganisms, e.g., bacteria, e.g., fungi capable of growth, e.g., as determined by U.S.P. 71 Sterility Testing Methods and Standards.

The finished cosmetic product may be provided wherein the sterile product is characterized in that it is substantially free from microorganisms, e.g., bacteria, e.g., fungi capable of growth, e.g., as determined by standard culture methods as described in U.S.P. 71.

When the finished cosmetic product or cosmetic product is challenged for microorganisms capable of growth, said cosmetic product shows no growth, e.g., when said microorganisms are measured by when said microorganisms are measured by U.S.P. 71 Sterility Testing Methods and Standards.

When the finished cosmetic product is challenged for microorganisms capable of growth, said cosmetic product shows no growth, e. g., when said microorganisms are measured by standard culture methods as described in U.S.P. 71.

In embodiments, the cosmetic product, or said finished cosmetic product, contains a plurality of components, and is heated after mixture of the plurality of components. The finished cosmetic product or cosmetic product may be heated after said cosmetic product is disposed in the end-use container. The finished cosmetic product or cosmetic product may be heated after closure of the end-use container. The finished cosmetic product or cosmetic product may be heated after sealing of the end-use container. The finished cosmetic product or cosmetic product may be heated prior to closure of the end-use container. The finished cosmetic product or cosmetic product may comprise an indicator that indicates if said cosmetic product, or said finished cosmetic product, has been heated. The cosmetic product, or finished cosmetic product may be heated at or above 121 degrees Celsius for at least 15 minutes during or after formulation, e.g., after mixing, or filling, e.g., after disposing.

The cosmetic product, or finished cosmetic product may not be heated above 140 degrees F. during or after formulation, e g., mixing, or filling, e.g., after disposing.

In embodiments, the end-use container is configured to inhibit retrograde flow, e.g., backflow, e.g., reverse flow, e.g., rearward movement, of material into said end-use container. The end-use container may be configured to inhibit retrograde flow, e.g., backflow, e.g., reverse flow, e.g., rearward movement, of material, e.g., said cosmetic product, into said end-use container. The end-use container may be configured to inhibit retrograde flow, e.g., backflow, e.g., reverse flow, e.g., rearward movement, of material, e.g., a contaminant, into said end-use container. The contaminant may be atmospheric, e.g., an aerosol, or a liquid, e.g., water, or solid, or a gas. The end-use container may comprise a reservoir in which said cosmetic product is disposed, and a dispenser through which said cosmetic product from said reservoir can be dispensed, wherein said dispenser inhibits retrograde flow of material into said reservoir. The end-use container may comprise a reservoir in which said cosmetic product is disposed, and a dispenser through which said cosmetic product from said reservoir can be dispensed, wherein said dispenser inhibits retrograde flow of dispensed cosmetic product, or atmospheric aerosols, into said reservoir. The end-use container may comprise an anti-retrograde flow dispenser comprising a first pressure activated valve disposed in said dispenser and proximal to said reservoir and a second pressure activated valve disposed in said dispenser and distal to said reservoir, wherein the activation pressure of said first valve is higher than the activation pressure of said second valve. The end-use container may comprise an anti-retrograde mechanism configured to prevent movement of the cosmetic product in a direction opposite the operational direction associated with dispensing the finished cosmetic product.

In embodiments, the amount of cosmetic product in said end-use container may be sufficient for no more than X applications, wherein X is between about 1 and about 180. The amount of cosmetic product in said finished cosmetic product may be selected such that the finished cosmetic product is sufficient for no more than X applications, wherein X is about 1 and about 180. The amount of cosmetic product in said finished cosmetic product may be selected such that the finished cosmetic product is sufficient for no more than about 28 days of use. The amount of cosmetic product in said finished cosmetic product may be equal to or less than an amount sufficient for 10, 20, 30, 40 or 50 uses or applications. The amount of cosmetic product in said finished cosmetic product may be selected such that the finished cosmetic product is sufficient for not more than X days of Y/day use, wherein X is about two weeks to about 6 weeks and Y is about zero per day use to about six times per day use.

In embodiments, the finished cosmetic product may have a deterioration-based expiration date. The finished cosmetic product may have an indication of deterioration-based expiration date. The finished cosmetic product may have a biome-compatible-based expiration date. The finished cosmetic product may have an indication of a biome-compatible-based expiration date. The finished cosmetic product may comprise an indication of expiration, or lifetime, e.g., recommended lifetime. The finished product may comprise an indication of expiration, or lifetime, e.g., recommended lifetime, after a preselected period of time, e.g., expressed in days or weeks. The finished cosmetic product may comprise an indication of expiration, or lifetime, e.g., recommended lifetime, after the preselected period of time, e.g., expressed in days, that is less than X days from the date of one of manufacturing, filling, sealing, shipping, releasing into commerce, or selling, wherein X is about 5-7 days, about 5-10 days, about 7-14 days, about 14-21 days, about 21-28 days, about 28-35 days, about 35-42 days, about 42-49 days, about 49-56 days, about 56-63 days, about 63-70 days, about 70-77 days, about 75-100 days, about 100-150 days, about 150-200 days, about 200-300 days, about 300-400 days, about 400-750 days. The finished cosmetic product may comprise an indication of expiration, or lifetime, e.g., recommended lifetime, after the preselected period of time, e.g., expressed in days, that is less than X days from the date of one of manufacturing, filling, sealing, shipping, releasing into commerce, or selling, wherein X is about 28 days, e.g., 28 days, or, e.g., about 180 days, e.g., 180 days. The finished cosmetic product may comprise an indication of expiration, or lifetime, e.g., recommended lifetime, expressed as a preselected period of time, e.g., days from the opening or unsealing of said finished cosmetic product. The finished product may comprise an indication of expiration, or lifetime, e.g., recommended lifetime, expressed as a preselected period of time, e.g., days from the opening or unsealing of said finished cosmetic product that is less than X days from the date of opening or unsealing, wherein X is about 5-7 days, about 5-10 days, about 7-14 days, about 14-21 days, about 21-28 days, about 28-35 days, about 35-42 days, about 42-49 days, about 49-56 days, about 56-63 days, about 63-70 days, about 70-77 days, about 75-100 days, about 100-150 days, about 150-200 days, about 200-300 days, about 300-400 days, about 400-750 days. The finished cosmetic product may comprise an indication of expiration, or lifetime, e.g., recommended lifetime, expressed as a preselected period of time, e.g., days from the opening or unsealing of said finished cosmetic product that is less than X days from the date of opening or unsealing, wherein X is about 28 days, e.g., 28 days. The indication of expiration, or lifetime, e.g., recommended lifetime, may be expressed as a preselected number of uses or applications. The indication of expiration, or lifetime, e.g., recommended lifetime, may be expressed as a preselected number of uses or applications, wherein the preselected number is between about 5-7, about 5-10, about 7-14, about 14-21, about 21-28, about 28-35, about 35-42, about 42-49, about 49-56, about 56-63, about 63-70, about 70-77, about 75-100, about 100-150, about 150-200, about 200-300, about 300-400, about 400-750 days.

In embodiments, the finished cosmetic product may have an expiration date, or lifetime, e.g., recommended lifetime. The expiration date, or lifetime, e.g., recommended lifetime, may be expressed:

a) in units of time, e.g., days, from a preselected event, e.g., unsealing of said finished cosmetic product or the first use of said finished cosmetic product; or

b) as the number of uses or applications.

The finished cosmetic product may have an expiration date, or lifetime, e.g., recommended lifetime, expressed as a. The finished cosmetic product may have an expiration date, or lifetime, e.g., recommended lifetime, expressed as b. The finished cosmetic product may have an expiration date, or lifetime, e.g., recommended lifetime, expressed as a and b. The finished cosmetic product may have an expiration date, or lifetime, e.g., recommended lifetime, expressed as a orb.

In embodiments, the end-use container may comprise a polymer, e.g., polyethylene terephthalate (PET), high density polyethylene (HDPE), polypropylene, polycarbonate, polytetrafluoroethylene (Teflon®), polyviylidene fluoride (PVDF), or a cellulosic. The end-use container may comprise glass. The finished cosmetic product may comprise a sensor, e.g., an oxygen sensor, that indicates a presence of viable bacterial. The end-use container may allow passage of at least about 5, 10, 20, 30, 40, 50, 60, 70, 80, 90, 95, 99, or 100 percent of transmission of ionizing radiation, e.g., with gamma rays, e.g., with x-rays, e.g., from an isotope, e.g., cobalt 60, or with ultraviolet, e.g., ultraviolet C (UVC) through the end-use container.

This disclosure provides, inter alia, a method of distributing a finished cosmetic product, comprising:

a) supplying (or causing a designee to supply) an end-user with a first unit of a finished cosmetic product;

b) providing (or causing a designee to provide) said end-user, or an entity designated by said end user, e.g., a second end-user, with:
i) a subsequent unit of said finished cosmetic product, or a unit of a second finished cosmetic product; or
ii) a notification that said first unit has reached the end of its recommended life;

c) optionally, providing (or causing a designee to provide) said end-user or an entity designated by said end user, e.g., a second end-user, with information regarding disposal, e.g., recycling, said first unit of finished cosmetic product, e.g., a finished cosmetic product on which the recommended life has elapsed.

thereby distributing a finished cosmetic product.

In embodiments, the method may comprise

a) supplying an end-user with a first unit of a finished cosmetic product;

b) providing said end-user, with:
i) a subsequent unit of said finished cosmetic product; or
ii) a notification that said first unit has reached the end of its recommended life; and

c) providing said end-user, with information regarding disposal, e.g., recycling, said first unit of finished cosmetic product, e.g., a finished cosmetic product on which the recommended life has elapsed.

thereby distributing a finished cosmetic product.

In embodiments, the method may comprise the finished cosmetic product comprising a finished cosmetic product as disclosed throughout this disclosure. The first unit of finished cosmetic product may comprise a cosmetic product described herein. The first unit may comprise a cosmetic product that is free, or substantially free, of bacteria or fungi. The first unit may comprise a finished cosmetic product comprising a cosmetic product that is free, or substantially free, of preservative.

In embodiments the first unit of finished cosmetic product comprises a cosmetic product selected from:

a baby product, e.g., a baby shampoo, a baby lotion, a baby oil, a baby powder, a baby cream; a bath preparation, e.g., a bath oil, a tablet, a salt, a bubble bath, a bath capsule; an eye makeup preparation, e.g., an eyebrow pencil, an eyeliner, an eye shadow, an eye lotion, an eye makeup remover, a mascara; a fragrance preparation, e.g., a colognes, a toilet water, a perfume, a powder (dusting and talcum), a sachet; hair preparations, e.g., hair conditioners, hair sprays, hair straighteners, permanent waves, rinses, shampoos, tonics, dressings, hair grooming aids, wave sets; hair coloring preparations, e.g., hair dyes and colors, hair tints, coloring hair rinses, coloring hair shampoos, hair lighteners with color, hair bleaches; makeup preparations, e.g., face powders, foundations, leg and body paints, lipstick, makeup bases, rouges, makeup fixatives; manicuring preparations, e.g., basecoats and undercoats, cuticle softeners, nail creams and lotions, nail extenders, nail polish and enamel, nail polish and enamel removers; oral hygiene products, e.g., dentrifices, mouthwashes and breath fresheners; bath soaps, e.g., foaming body cleansers, and detergents, deodorants, douches, feminine hygiene deodorants; shaving preparations, e.g., aftershave lotions, beard softeners, talcum, preshave lotions, shaving cream, shaving soap; skin care preparations, e.g., cleansing, depilatories, face and neck, body and hand, foot powders and sprays, moisturizing, night preparations, paste masks, skin fresheners; and suntan preparations, e.g., gels, creams, and liquids, and indoor tanning preparations.

In embodiments, the first unit of finished cosmetic product comprises a shampoo.

In embodiments, the first unit of finished cosmetic product comprises a foaming body cleanser.

In embodiments, the first unit of finished cosmetic product comprises a conditioner.

In embodiments the method may comprise wherein said end-user is supplied with, e.g., by sale, or gifting, said first unit of a finished cosmetic product from an internet-based outlet. The end-user may be supplied with, e.g., by sale, or gifting, said first unit of a finished cosmetic product from a non-internet-based outlet, e.g., a store. Providing said end-user may comprise providing said end-user with i. Providing said end-user may comprise providing said end-user with ii. Providing said end-user may comprise providing said end-user with i and ii. The provision may be made within a preselected number of days, e.g., 1 day to about 28 days, e.g., 3-7 days, prior to the end of the recommended life of the first unit of a finished cosmetic product. The provision may be made within a preselected number of days, e.g., 1 day to about 28 days, e.g., 21-25 days, after supply of said first unit of a finished cosmetic product. The provision may be made within a preselected number of days, e.g., 1 day to about 28 days, prior to or subsequent to a first expected use of the finished cosmetic product. The provision may be made within a preselected number of days, e.g., 1-28 days, e.g., 3-7 days, of an expiration date.

In embodiments the subsequent unit of finished cosmetic product may comprise a cosmetic product described herein. The subsequent unit of finished cosmetic product may comprise a cosmetic product that is free, or substantially free, of bacteria or fungi. The subsequent unit of finished cosmetic product may comprise a cosmetic product that is free, or substantially free, of preservative.

In embodiments, the subsequent unit of finished cosmetic product comprises a cosmetic product selected from one of the following:

a baby product, e.g., a baby shampoo, a baby lotion, a baby oil, a baby powder, a baby cream; a bath preparation, e.g., a bath oil, a tablet, a salt, a bubble bath, a bath capsule; an eye makeup preparation, e.g., an eyebrow pencil, an eyeliner, an eye shadow, an eye lotion, an eye makeup remover, a mascara; a fragrance preparation, e.g., a colognes, a toilet water, a perfume, a powder (dusting and talcum), a sachet; hair preparations, e.g., hair conditioners, hair sprays, hair straighteners, permanent waves, rinses, shampoos, tonics, dressings, hair grooming aids, wave sets; hair coloring preparations, e.g., hair dyes and colors, hair tints, coloring hair rinses, coloring hair shampoos, hair lighteners with color, hair bleaches; makeup preparations, e.g., face powders, foundations, leg and body paints, lipstick, makeup bases, rouges, makeup fixatives; manicuring preparations, e.g., basecoats and undercoats, cuticle softeners, nail creams and lotions, nail extenders, nail polish and enamel, nail polish and enamel removers; oral hygiene products, e.g., dentrifices, mouthwashes and breath fresheners; bath soaps, e.g., foaming body cleansers, and detergents, deodorants, douches, feminine hygiene deodorants; shaving preparations, e.g., aftershave lotions, beard softeners, talcum, preshave lotions, shaving cream, shaving soap; skin care preparations, e.g., cleansing, depilatories, face and neck, body and hand, foot powders and sprays, moisturizing, night preparations, paste masks, skin fresheners; and suntan preparations, e.g., gels, creams, and liquids, and indoor tanning preparations.

In embodiments, the subsequent unit of finished cosmetic product may comprise a shampoo.

In embodiments, the subsequent unit of finished cosmetic product may comprise a foaming body cleanser.

In embodiments, the subsequent unit of finished cosmetic product may comprise a conditioner.

In embodiments the subsequent unit of said finished cosmetic product, or a unit of a second finished cosmetic product, may be delivered, e.g., by mail or a commercial delivery entity, to said end-user. The subsequent unit of said finished cosmetic product, or a unit of a second finished cosmetic product, may be delivered, e.g., by mail or a commercial delivery entity, to an entity, e.g., a second end user, designated by said end-user. The subsequent unit of said finished cosmetic product, or a unit of a second finished cosmetic product, may be delivered, e.g., by mail or a commercial delivery entity, to a location designated by said end-user. The notification, may be delivered, e.g., by mail or a commercial delivery entity, or electronically, e.g., by internet or telephone, e.g., by telephone call, e.g., by recorded message, or text message to said end-user. The notification, may be delivered, e.g., by mail or a commercial delivery entity, or electronically, e.g., by internet or telephone, e.g., by telephone call, e.g., by recorded message, or text message, to an entity, e.g., a second end user, designated by said end-user. The notification, may be delivered, e.g., by mail or a commercial delivery entity, or electronically, e.g., by internet or telephone, to a location designated by said end-user.

In embodiments the method may comprise c) providing (or causing a designee to provide) said end-user or an entity designated by said end user, e.g., a second end-user, with information regarding disposal, e.g., recycling, of said first unit of finished cosmetic product, e.g., a finished cosmetic product on which the recommended life has elapsed. The information may comprise the name or location, e.g., address, of an entity (a collection entity) which will accept said first unit of finished cosmetic product, e.g., after its recommended life. The method may comprise providing said end-user, or designee, with a container configured to receive said first unit of finished cosmetic product, e.g., after its recommended life. The container may be provided with the first unit of finished cosmetic product. The container may be provided with the notification. The container may comprise a mailing label addressed to said collection entity. The collection entity may be a recycler.

In embodiments, the method may comprise d) providing (or causing a designee to provide) said end-user or an entity designated by said end user, e.g., a second end-user, with information regarding disposal, e.g., recycling, of said subsequent unit of finished cosmetic product, e.g., a finished cosmetic product, or cosmetic product, on which the recommended lifetime has elapsed. The information may comprise the name or location, e.g., address, of an entity (a collection entity) which will accept said subsequent unit of finished cosmetic product, e.g., after its recommended life. The method may comprise providing said end-user, or designee, with a container configured to receive said subsequent unit of finished cosmetic product, e.g., after its recommended life. The container may be provided with the subsequent unit of finished cosmetic product. The container may be provided with the notification. The container may comprise a mailing label addressed to said collection entity. The collection entity may be a recycler.

This disclosure provides, inter alia, a method of obtaining a finished cosmetic product, comprising:

a) receiving a first unit of a finished cosmetic product;

b) receiving:
i) a subsequent unit of said finished cosmetic product, or a unit of a second finished cosmetic product; or
ii) a notification that said first unit has reached the end of its recommended life;

c) optionally, receiving information regarding disposal, e.g., recycling, said first unit of finished cosmetic product, e.g., a finished cosmetic product on which the recommended life has elapsed.

thereby obtaining a finished cosmetic product.

In embodiments, the method may comprise

obtaining, e.g., manufacturing, a finished cosmetic product of any of the above claims, said finished cosmetic product comprising a cosmetic product, e.g., shampoo, e.g., body wash, disposed in an end use container;

communicating to an end user of said finished product, one or more of the following:
the finished cosmetic product, or cosmetic product, is biome-friendly, e.g., biome-compatible;
the finished cosmetic product, or cosmetic product, should not be used after an indicated expiration date, or lifetime, e.g., recommended lifetime, based on, e.g., deterioration, e.g., biome-compatibility,
   wherein said indicated expiration date or lifetime, e.g., recommended lifetime is expressed:
(a) in units of time, e.g., days, from a preselected event, e.g, unsealing of said finished cosmetic product or a first use of said finished cosmetic product; or
(b) as the number of uses or applications;
the finished cosmetic product, or cosmetic product, should not be used after X applications, for example, wherein X is between about 1 and about 180; and
the finished cosmetic product, or cosmetic product, should not be used after X days of use, e.g., X days of Y/day use, wherein X is between about (7 days) one week and about 42 days (6 weeks), and Y is between about zero uses per day and about ten uses per day. For example X days may be about 7-10, 10-13, 14-17, 18-21, 22-25, 26-29, 30-33, 34-37, 38-42 days; and Y may be about 0-1, 2-4, 5-7, 8-10 uses per day.

In embodiments, the method may comprise shipping said finished cosmetic product.

This disclosure provides, inter alia, a method of making a finished cosmetic product, comprising disposing a cosmetic product in an end-use container to form a filled end-use container; and treating said filled end-use container to kill or inactivate bacteria, thereby providing a finished cosmetic product.

This disclosure provides, inter alia, a method of making a biome-friendly cosmetic product comprising selecting a first component, e.g., a surfactant, from a list of biome-friendly components, selecting a second component, e.g., a humectant, from a list of biome-friendly components, and providing a mixture of said first and second component, thereby making a biome-friendly cosmetic product.

In embodiments, the first, second, or first and second components of the method are selected from Table 3. The method may comprise determining the biome-friendliness expiration date or recommend life of said finished cosmetic product. Determination may comprise evaluating the viability of an AOB or of the ability of a AOB to produce nitrite after contact with the mixture of the first and second component.

This disclosure provides, inter alia, a method of making a finished cosmetic product comprising providing a first and second component, wherein each has been shows to be biome-friendly, combining said first and second component to form a mixture, determining if said mixture is biome-friendly, thereby making a finished cosmetic product.

In embodiments, determining may comprise evaluating the viability of an AOB or of the ability of a AOB to produce nitrite after contact with the mixture of the first and second component.

This disclosure provides, inter alia, a method of manufacturing (evaluating) a cosmetic product or finished cosmetic product comprising:
providing a product having at least 2 components selected from Table 3;
acquiring an evaluation of whether the cosmetic product is safe for bacteria, e.g., beneficial bacteria, e.g., AOB, on the skin of the user,
thereby manufacturing the cosmetic product or finished cosmetic product.

In embodiments, the evaluation may comprise evaluating the viability of an AOB or of the ability of a AOB to produce nitrite after contact with the product.

This disclosure provides, inter alia, a method of evaluating a cosmetic product or a finished cosmetic product, comprising:
contacting an aliquot of cosmetic product with a test organism, e.g., an ammonia oxidizing bacteria; and
evaluating an effect of the cosmetic product on the test organism,
   wherein evaluating comprises evaluating the effect of the cosmetic product on an ability of the test organism, e.g., ammonia oxidizing bacteria, to produce nitrite.

In embodiments, evaluating may comprise determining if the ability of the ammonia oxidizing bacteria to produce nitrite meets a preselected criteria. Evaluating the effect of the cosmetic product on the ability of the test organism provides for the finished cosmetic product to be identified as “tested and confirmed to have the live test organisms.” Evaluating the effect of the cosmetic product on the ability of ammonia oxidizing bacteria to produce nitrite provides for the finished cosmetic product to be identified as “tested and confirmed to have live ammonia oxidizing bacteria.”

This disclosure provides, inter alia, A method of selecting an ammonia oxidizing bacteria-friendly excipient comprising:
obtaining an ammonia oxidizing bacteria (AOB) cell suspension, e.g., from a continuous culture system;
harvesting AOB cells from the cell suspension;
washing the AOB cells in a storage solution, e.g., a storage solution comprising 50 mM Na₂HPO₄— 2 mM MgCl₂, pH 7.6;
suspending the AOB cells in the storage solution at a final optical density (OD₆₀₀) of 5.0 (˜10¹⁰ cells/ml);
storing the AOB cells at about 4° C.;
diluting the AOB cells to a final optical density (OD₆₀₀) of 0.5 (˜10⁹ cells/ml) in 10 ml AOB medium supplemented with ammonium (NH₄⁺), e.g., 50 mM ammonium, containing an excipient at a pre-determined final concentration;
incubating at 30° C. for a first pre-determined time period to provide an incubated culture;
collecting an aliquot of the incubated culture; and
measuring a concentration of nitrite in a supernatant of the incubated culture.

In embodiments, the supernatant may be provided by centrifuging the aliquot of the incubated culture to provide the supernatant and a bacterial pellet. The method may comprise identifying the excipient as an ammonia oxidizing bacteria-friendly ingredient based on the concentration of nitrite in the supernatant of the incubated culture. The method may further comprise washing the bacterial pellet in the AOB medium, suspending the bacterial pellet in the AOB medium supplemented with NH₄⁺, incubating the bacterial pellet in the AOB medium supplemented with NH₄⁺, recovering AOB cells at a second pre-determined time periods to provide recovered AOB cell samples, and measuring the recovered AOB cell sample for at least one of an OD₆₀₀ value and nitrite accumulation.

In embodiments, the method may comprise identifying the excipient as an ammonia oxidizing bacteria-friendly ingredient based on at least one of the OD₆₀₀ value and nitrite accumulation in the recovered AOB cell sample. Harvesting AOB cells from the cell suspension may comprise centrifuging the cell suspension. The pre-determined final concentration of excipient is between about 0% and about 100%. The first pre-determined time period is at least one of about 1 minute, about 10 minutes, about 60 minutes, about 2 hours, about 4 hours, about 6 hours, about 12 hours, and about 24 hours. The second pre-determined time period is at least one of about 1 minute, about 10 minutes, about 60 minutes, about 2 hours, about 4 hours, about 6 hours, about 12 hours, about 24 hours, about 36 hours, about 48 hours, about 72 hours, about 96 hours.

This disclosure provides, inter alia, method of selecting an ammonia oxidizing bacteria-friendly excipient comprising obtaining a sample of ammonia oxidizing bacteria (AOB), contacting the AOB with an excipient for a first pre-determined time period to provide an incubated culture, collecting an aliquot of the incubated culture. And measuring a concentration of nitrite in a supernatant of the incubated culture.

In embodiments, identifying the excipient as an ammonia oxidizing bacteria-friendly excipient based on the concentration of nitrite in the supernatant of the incubated culture.

In embodiments, the method may comprise contacting the AOB from the incubated culture with NH₄⁺, and measuring the recovered AOB cell sample for at least one of an OD₆₀₀ value and nitrite accumulation after a second pre-determined time period. The method may further comprise identifying the excipient as an ammonia oxidizing bacteria-friendly ingredient based on at least one of the OD₆₀₀ value and nitrite concentration in the recovered AOB cell sample.

The OD₆₀₀ value in the recovered AOB cell sample may allow identification of an ammonia-oxidizing bacteria-friendly excipient, if the OD₆₀₀ is greater than or equal to about 0.01.

This disclosure provides, inter alia, a method of producing a biome-friendly composition comprising acquiring knowledge that a compound is friendly to ammonia oxidizing bacteria; and combining the compound with ammonia oxidizing bacteria to provide a cosmetic product.

This disclosure provides, inter alia, method of maintaining ammonia oxidizing bacteria (AOB) on a subject comprising applying a cosmetic product or a finished cosmetic product as described throughout the disclosure. The method may comprise applying the preparation comprising AOB to the subject prior to applying the cosmetic product or the finished cosmetic product. The method may comprise applying the preparation comprising AOB to the subject subsequent to applying the cosmetic product or the finished cosmetic product. The method may comprise applying the preparation comprising AOB to the subject prior to applying the cosmetic product or the finished cosmetic product, wherein the preparation comprising AOB is applied between about one of the following ranges: about 1-5, 5-10, 10-20, 20-30. 30-40, 40-50, 50-60 minutes, 2-5, 5-10, 10-15, 15-20, 20-25 hours, 2-5, 5-10, 10-15, days, 3-4, 5-10 weeks prior to applying the cosmetic product or the finished cosmetic product. The method may comprise applying the preparation comprising AOB to the subject subsequent to applying the cosmetic product or the finished cosmetic product, wherein the preparation comprising AOB is applied between about one of the following ranges: about 1-5, 5-10, 10-20, 20-30. 30-40, 40-50, 50-60 minutes, 2-5, 5-10, 10-15, 15-20, 20-25 hours, 2-5, 5-10, 10-15, days, 3-4, 5-10 weeks subsequent to applying the cosmetic product or the finished cosmetic product. The method may comprise not applying a non biome-friendly cosmetic product or finished cosmetic product prior to or subsequent. At least one of the preparation of AOB and the cosmetic product, e.g., finished cosmetic product is applied to a pre-defined area of the subject. The pre-defined area of the subject is at least one of a portion of a head, e.g., a face, cheek, chin, eyelid, lip, nose, scalp, hair, forehead; neck; underarm; arm; hand; leg; foot; chest; abdomen region; buttocks; genital area; and back. The AOB may be any one or more AOB descried herein. The AOB may be N. eutropha D23.

Throughout this disclosure, natural rose hydrosol may be referred to as rosa damascena flower water; and rosa damascena flower water may be referred to as natural rose hydrosol, i.e., the two terms are interchangeable, as used throughout the disclosure

### BRIEF DESCRIPTION OF FIGURES

FIG. 1A shows the nitrite production after incubation with Foaming Soap Rose scented and Unscented and recovery of N. eutropha D23, after 1 minute incubation. The nitrite concentration is plotted relative to time.

FIG. 1B shows the nitrite production after incubation with Foaming Soap Rose scented and Unscented and recovery of N. eutropha D23, after 10 minute incubation. The nitrite concentration is plotted relative to time.

FIG. 1C shows the nitrite production after incubation with Foaming Soap Rose scented and Unscented and recovery of N. eutropha D23, after 60 minute incubation. The nitrite concentration is plotted relative to time.

FIG. 2A shows the nitrite production after incubation with various concentrations of Body Wash and recovery of N. eutropha D23, after 1 minute incubation. The nitrite concentration is plotted relative to time.

FIG. 2B shows the nitrite production after incubation with various concentrations of Body Wash and recovery of N. eutropha D23, after 10 minute incubation. The nitrite concentration is plotted relative to time.

FIG. 2C shows the nitrite production after incubation with various concentrations of Body Wash and recovery of N. eutropha D23, after 60 minute incubation. The nitrite concentration is plotted relative to time.

FIG. 3A shows the nitrite production after incubation with various concentrations of Shampoo and recovery of N. eutropha D23, after 1 minute incubation. The nitrite concentration is plotted relative to time.

FIG. 3B shows the nitrite production after incubation with various concentrations of Shampoo and recovery of N. eutropha D23, after 10 minute incubation. The nitrite concentration is plotted relative to time.

FIG. 3C shows the nitrite production after incubation with various concentrations of Shampoo and recovery of N. eutropha D23, after 60 minute incubation. The nitrite concentration is plotted relative to time.

FIG. 4A shows the nitrite production after incubation with Conditioner and recovery of N. eutropha D23, after 1 minute incubation. The nitrite concentration is plotted relative to time.

FIG. 4B shows the nitrite production after incubation with Conditioner and recovery of N. eutropha D23, after 10 minute incubation. The nitrite concentration is plotted relative to time.

FIG. 4C shows the nitrite production after incubation with Conditioner and recovery of N. eutropha D23, after 60 minute incubation. The nitrite concentration is plotted relative to time.

FIG. 5 is a table of a study of surfactants and their compatibility with N. eutropha D23.

### DETAILED DESCRIPTION

The systems and methods of the disclosure provide, inter alia, cosmetic products, e.g., finished cosmetic products that may be considered to be “biome-friendly” or “biome-compatible.” The systems and methods of the disclosure may provide for use of cosmetic products, e.g., finished cosmetic products, that may be used in combination with bacteria, e.g., non-pathogenic bacteria, e.g., ammonia oxidizing bacteria, which may be used in the form of a preparation or composition to be applied to a subject.

Systems and methods of the disclosure may also provide for manufacturing of the cosmetic product, e.g., finished cosmetic product, quality control testing, and testing to determine the compatibility of a product, or one or more components of a product with bacteria, e.g., non-pathogenic bacteria, e.g., ammonia oxidizing bacteria, and or with a microbiome of a subject.

Systems and methods of the disclosure may provide for commercial release and distribution of the product, e.g., finished cosmetic product, into commerce, including methods of dispensing the product into commerce, refilling the product, and recycling the product.

### 1. Definitions

An ammonia oxidizing bacterium refers to a bacterium capable of oxidizing ammonia or ammonium to nitrite and, under certain conditions, nitric oxide. This may be accomplished at a rate. The rate, e.g., a pre-determined rate, may refer to the conversion of ammonium ions (NH₄⁺) (e.g., at about 200 mM) to nitrite (NO₂⁻) at a rate of at least 50, 75, 125, or 150 micromoles NO₂⁻ per minute, e.g., about 100-150, 75-175, 75-125, 100-125, 125-150, or 125-175 micromoles/minute, e.g., about 125 micromoles NO₂⁻ per minute. In embodiments, the rate, e.g., a pre-determined rate, may refer to the conversion of ammonium ions (NH₄⁺) (e.g., at about 200 mM) to nitrite (NO₂⁻) at a rate of at least 50, 75, 125, or 150 nanomoles NO₂⁻ per minute per ml, e.g., about 100-150, 75-175, 75-125, 100-125, 125-150, or 125-175 nanomoles/minute/ml, e.g., about 125 nanomoles NO₂⁻ per minute per ml for a continuous culture, for example having an OD of about 0.5.

Examples of ammonia oxidizing bacteria include Nitrosomonas eutropha strains, e.g., D23 and C91, and other bacteria in the genera Nitrosomonas, Nitrosococcus, Nitrosospira, Nitrosocystis, Nitrosolobus, and Nitrosovibrio. D23 Nitrosomonas eutropha strain refers to the strain, designated AOB D23-100, deposited with the American Tissue Culture Collection (ATCC) (10801 University Blvd., Manassas, Va., USA) on Apr. 8, 2014 having accession number PTA-121157. The nucleic acid sequence(s), e.g., genome sequence, of accession number PTA-121157 are hereby incorporated by reference in their entireties. In certain embodiments, the N. eutropha is a strain described in PCT Application No. PCT/US2015/025909, filed Apr. 15, 2015, herein incorporated by reference in its entirety.

Optimized Nitrosomonas eutropha (N. eutropha), as that term is used herein, refers to an N. eutropha having an optimized growth rate; an optimized NH₄⁺ oxidation rate; or optimized resistance to NH₄⁺. In an embodiment it differs from naturally occurring N. eutropha by at least one nucleotide, e.g., a nucleotide in a gene selected from ammonia monooxygenase, hydroxylamine oxidoreductase, cytochrome c554, and cytochrome c_{M}552. The difference can arise, e.g., through selection of spontaneously arising mutation, induced mutation, or directed genetic engineering, of the N. eutropha. In an embodiment it differs from a naturally occurring N. eutropha in that it has a constellation of alleles, not present together in nature. These differences may provide for one or more of a treatment or prevention of a skin disorder, a treatment or prevention of a disease or condition associated with low nitrite levels, a treatment or prevention of body odor, a treatment to supply nitric oxide to a subject, and a treatment to inhibit microbial growth.

As used herein, an “autotroph”, e.g., an autotrophic bacterium, is any organism capable of self-nourishment by using inorganic materials as a source of nutrients and using photosynthesis or chemosynthesis as a source of energy. Autotrophic bacteria may synthesize organic compounds from carbon dioxide and ATP derived from other sources, coxiation of ammonia to nitrite, oxidation of hydrogen sulfide, and oxidation of Fe2⁺ to Fe3⁺ Autotrophic bacteria of the present disclosure are incapable of causing infection.

As used herein, “axenic” refers to a composition comprising an organism that is substantially free of other organisms. For example, an axenic culture of ammonia oxidizing bacteria is a culture that is substantially free of organisms other than ammonia oxidizing bacteria. An axenic composition may comprise elements that are not organisms, e.g., it may comprise nutrients or excipients. Any embodiment, preparation, composition, or formulation of ammonia oxidizing bacteria discussed herein may comprise, consist essentially of, or consist of optionally axenic ammonia oxidizing bacteria.

In some embodiments, “substantially free” denotes undetectable by a method used to detect the item that is indicated as “substantially free.” For example, “substantially free of a preservative” denotes that it is undetectable by a method used to detect a preservative. “Substantially free of microorganisms” denotes undetectable by a method used to detect other organisms, e.g., plating the culture and examining colony morphology, or PCR for a conserved gene such as 16S RNA.

A test, such as a minimum inhibitory concentration (MIC) test, may be performed. The MIC is defined as the lowest concentration of an anti-microbial that will inhibit the visible growth of a microorganism after incubation for a pre-determined period of time, for example, overnight incubation, for example, 12 hour, or 24 hour incubation.

To “culture” refers to a process of placing an amount of a desired bacterium under conditions that promote its growth, i.e., promoting cell division. The conditions can involve a specified culture medium, a set temperature range, and/or an agitation rate. Bacteria can be cultured in a liquid culture or on plates, e.g., agar plates.

Complete N. europaea medium refers to the N. europaea growth medium described in Ensign et al., “In vitro activation of ammonia monooxygenase from Nitrosomonas europaea by copper.” J Bacteriol. 1993 April; 175(7):1971-80.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

As used herein, the term “optimized growth rate” refers to one or more of: a doubling time of less than about 4, 5, 6, 7, 8, 9, or 10 hours when cultured under batch conditions as described in PCT/US2015/025909, filed Apr. 15, 2015; a doubling time of less than about 16, 18, 20, 22, 24, or 26 hours, when grown under chemostat conditions as described in PCT/US2015/025909, filed Apr. 15, 2015; or growing from an OD600 of about 0.15 to at least about 0.3, 0.4, 0.5, 0.6, 0.7, or 0.8 over about 1 or 2 days. In an embodiment, optimized growth rate is one having a doubling time that it is at least 10, 20, 30, 40, or 50% shorter than that of a naturally occurring N. eutropha.

As used herein, “optimized NH₄⁺ oxidation rate” refers to a rate of at least about 50, 75, 125, or 150 micromoles per minute of converting NH₃ or NH₄⁺ into NO₂⁻. For instance, the rate may be at least about 50, 75, 125, or 150 micromoles per minute of converting NH₄⁺ (e.g., at about 200 mM) to NO₂⁻. In an embodiment, an optimized NH₄⁺ oxidation rate is one in which NH₃ or NH₄⁺ is converted into NO₂⁻, at least 10, 20, 30, 40, or 50% more rapidly than is seen with a naturally occurring N. eutropha. In embodiments, the rate of at least about 50, 75, 125, or 150 nanomoles per minute per ml of converting NH₃ or NH₄⁺ into NO₂⁻. For instance, the rate may be at least about 50, 75, 125, or 150 nanomoles per minute per ml of converting NH₄⁺ (e.g., at about 200 mM) to NO₂⁻ for a continuous culture having an OD of about 0.5.

As used herein, “optimized resistance to NH₄⁺” refers to an ability to grow in conditions of greater than 50, 75, 100, 125, 150, 175, 200, 225, 250, 275, or 300 mM NH₃ or NH₄⁺ for at least about 24 or 48 hours. In an embodiment, an optimized resistance to NH₄⁺ refers to the ability to grow at least 10, 20, 30, 40, or 50% more rapidly, or at least 10, 20, 30, 40, or 50% longer, in the presence of a selected concentration of NH₃ or NH₄⁺ than can a naturally occurring N. eutropha.

Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated. In some embodiments, the delivery of one treatment is still occurring when the delivery of the second begins, so that there is overlap. This is sometimes referred to herein as “simultaneous” or “concomitant” or “concurrent delivery”. In other embodiments, the delivery of one treatment ends before the delivery of the other treatment begins. This is sometimes referred to herein as “successive” or “sequential delivery” or “consecutive delivery.” In embodiments of either case, the treatment is more effective because of combined administration. For example, the second treatment is a more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive (i.e., synergistic). The delivery can be such that an effect of the first treatment delivered is still detectable when the second is delivered.

Administered “in combination,” as used herein, means that two (or more) different preparations, e.g., cosmetic product, e.g., finished cosmetic product, e.g., ammonia oxidizing containing product, are delivered to the subject over a given period of time. The two or more different preparations may be delivered simultaneously, or successively.

A “natural product” is or may comprise a product that may be at least partially derived from nature. It may be anything or comprise anything produced by a living organism, and may include organisms themselves. Natural products may include or comprise an entire organism, and part of an organism (e.g., a leaf of a plant), an extract from an organism, an organic compound from an organism, a purified organic compound from an organism. Natural products may be or comprise organic substances found and cells, including primary metabolites (amino acids, carbohydrates, and nucleic acids) and secondary metabolites (organic compounds found in a limited range of species, e.g., polyketides, fatty acids, terpenoids, steroids, phenylpropanoids, alkaloids, specialized amino acids and peptides, specialized carbohydrates). Natural products may be or comprise polymeric organic materials such as cellulose, lignin, and proteins.

Natural products may be or comprise products for commercial purposes, and may refer to cosmetics, dietary supplements, and foods produced from natural sources. Natural products may have pharmacological or biological activity that may be of therapeutic benefit, e.g., in treating disease or conditions. Natural products may be included in traditional medicines, treatments for cosmetological purposes, and spa treatments. A natural product referred to herein may comprise any one or more of the components described as a natural product to be incorporated into a preparation or formulation comprising one or more other components, e.g., excipients. The preparation or formulation referred to as a natural product may comprise a natural product defined herein and one or more additional components or ingredients. Any of the compositions, preparations, or formulations discussed throughout this disclosure may be or comprise one or more natural products.

As used herein, “presence” or “level” may refer to a qualitative or quantitative amount of a component, e.g., any one or more of an ammonia oxidizing bacteria, ammonia, ammonium ions, urea, nitrite, or nitric oxide. The presence or level may include a zero value or a lack of presence of a component.

The terms “polypeptide”, “peptide” and “protein” (if single chain) are used interchangeably herein to refer to amino acid polymers. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

As used herein, a “subject” may include an animal, a mammal, a human, a non-human animal, a livestock animal, or a companion animal. The term “subject” is intended to include human and non-human animals, for example, vertebrates, large animals, and primates. In certain embodiments, the subject is a mammalian subject, and in particular embodiments, the subject is a human subject. Although applications with humans are clearly foreseen, veterinary applications, for example, with non-human animals, are also envisaged herein. The term “non-human animals” of the disclosure includes all vertebrates, for example, non-mammals (such as birds, for example, chickens; amphibians; reptiles) and mammals, such as non-human primates, domesticated, and agriculturally useful animals, for example, sheep, dog, cat, cow, pig, rat, among others.

As used herein, the term “surfactant”, includes anionic, cationic, non-ionic, and amphoteric compounds that may lower the surface tension, or interfacial tension, between two liquids or between a liquid and a solid. Surfactants may act as detergents, wetting agents, emulsifiers, foaming agents, and dispersants. Surfactants may be referred to as excipients. Surfactants may include one or more of the following, alone, or in combination with those listed, or other surfactants or surfactant-like compounds, and other compounds described throughout this disclosure: cocamidopropyl betaine (ColaTeric COAB), polyethylene sorbitol ester (e.g., Tween 80 and Tween 20), ethoxylated lauryl alcohol (RhodaSurf 6 NAT), sodium laureth sulfate/lauryl glucoside/cocamidopropyl betaine (Plantapon 611 L UP), sodium laureth sulfate (e.g., RhodaPex ESB 70 NAT), alkyl polyglucoside (e.g., Plantaren 2000 N UP), sodium laureth sulfate (Plantaren 200), Dr. Bronner's Castile soap, Dr. Bronner's baby soap, Lauramine oxide (ColaLux Lo), sodium dodecyl sulfate (SDS), polysulfonate alkyl polyglucoside (PolySufanate 160 P), sodium lauryl sulfate (Stepanol-WA Extra K) and combinations thereof. Dr. Bronner's Castile soap and baby soap comprises water, organic coconut oil, potassium hydroxide, organic olive oil, organic fair deal hemp oil, organic jojoba oil, citric acid, and tocopherol.

Surfactants may include Sodium Laurylglucosides Hydroxypropylsulfonate (Suga®nate 160NC), lauramidopropyl betaine (Cola®Teric LMB); Cocamidopropyl hydroxysultaine (Cola®Teric CBS); disodium cocoamphodiacetate (Cola®Teric CDCX-LV); sodium laurylglucosides hydroxypropyl phosphate (Suga®Fax D12).

Surfactants may include sodium lauroyl methyl isethionate (Iselux® LQ-CLR-SB); sodium methyl cocoyl taurate (Pureact WS Conc.); Aqua (and) Sodium Lauroyl Methyl Isethionate (and) Cocamidopropyl Betaine (and) Sodium Cocoyl Isethionate (and) Sodium Methyl Oleoyl Taurate (Iselux®SFS-SB).

As used herein, “transgenic” means comprising one or more exogenous portions of DNA. The exogenous DNA is derived from another organism, e.g., another bacterium, a bacteriophage, an animal, or a plant.

“Finished cosmetic product” as that term is used herein, refers to a product that is ready and/or suitable for release into commerce. For example, a finished cosmetic product may be a product, e.g., a cosmetic product, that contains one or more components, and has been prepared and packages for use by an end-user, e.g., a consumer. In an embodiment a finished cosmetic product is disposed in the end-use container in which it will be used by the end-user. In an embodiment, a finished cosmetic product may be one or more of manufactured, mixed, disposed in an end-use container, sterilized, tested, and sealed, in any particular order.

A “unit” of a finished cosmetic product refers to a single entity of finished cosmetic product that may form an individual or complete component for use by end-user or for sale. In some embodiments a unit may be a single entity but may form an individual component of a larger or more complex whole. In an embodiment, a unit may be an individual end-use container that contains a cosmetic product, for sale or for use by an end-user.

“End-user”, as that term is used herein, refers to a person who will use a finished cosmetic product, e.g., by applying the finished cosmetic product to himself or herself or which applies, or provides, the finished cosmetic product, to a subject, e.g., another person, or an animal, e.g., a companion animal.

“Recommended life”, as that term is used herein, refers to a suggested period of time that may be provided by a manufacturer of the finished cosmetic product. The recommended life may be based on testing performed that establishes that use of the product during the period of time provides for no noticeable adverse effects on the user.

“End-use container”, as that term is used herein, refers to a vessel that houses a cosmetic product, e.g., finished cosmetic product. The end-use container may allow for delivery of the finished cosmetic product from the vessel to the outside environment. In certain embodiments, the end-use container may prevent or reduce retrograde flow of the contents of the container. The end-use container may be configured to provide one-way flow and/or zero-dead volume.

The end-use container may be comprised of any suitable material that is compatible with the contents of the container and the external environment. For example, the end-use container may be made of glass, aluminum, or one or more polymers, for example, a high density polyethylene polymer.

The end-use container may comprise a delivery system, e.g., a system or mechanism that may allow contents from the container to be disposed outside of the container. For example the delivery system may comprise a dispenser, for example, utilizing a pump.

“Microbiome” refers to a population, e.g, one or more microorganisms that live on a surface of a subject, e.g., in the gut, mouth, skin, and/or elsewhere in a subject. The population may have one or more beneficial functions and/or benefits, relevant to supporting the life of a subject.

“Preservative”, as that term is used herein, refers to a compound that kills or inhibits the growth of a microbe, e.g., a bacterium or fungus. Exemplary preservatives include those listed in Annex VI at the end of the Detailed Description, herein. Preservatives referred to herein may be referred to as anti-bacterial preservatives. Preservatives referred to herein may not be referring to anti-oxidant preservatives.

As used herein, “Sterility Assurance Level” is the probability of a single unit, for example, a single cosmetic product, e.g., single finished cosmetic product being non-sterile after it has been subjected to sterilization. Sterility Assurance Level (SAL) may be about 10⁻⁵ or as 10⁻⁶, which is means a 1 in 100,000 chance (for 10⁻⁵) or a 1 in 1,000,000 chance (for 10⁻⁶) of a non-sterile unit. SAL may also describe the killing efficacy of a sterilization process. A very effective sterilization process has a very low SAL, for example, 10⁻⁵, or 10⁻⁶.

As used herein, “treatment of a disease or condition” refers to reducing the severity or frequency of at least one symptom of that disease or condition, compared to a similar but untreated patient. Treatment can also refer to halting, slowing, or reversing the progression of a disease or condition, compared to a similar but untreated patient. Treatment may comprise addressing the root cause of the disease and/or one or more symptoms.

As used herein a “therapeutically effective amount” refers to a dose sufficient to prevent advancement, or to cause regression of a disease or condition, or which is capable of relieving a symptom of a disease or condition, or which is capable of achieving a desired result. A therapeutically effective dose can be measured, for example, as a number of bacteria or number of viable bacteria (e.g., in CFUs) or a mass of bacteria (e.g., in milligrams, grams, or kilograms), or a volume of bacteria (e.g., in mm³).

As used herein, the term “viability” refers to an autotrophic bacteria's, e.g., ammonia oxidizing bacteria's, ability to oxidize ammonia, ammonium, or urea to nitrite at a pre-determined rate. In some embodiments, the rate refers to the conversion of ammonium ions (NH₄⁺)(e.g., at about 200 mM) to nitrite (NO₂⁻) at a rate of at least 50, 75, 125, or 150 micromoles NO₂⁻ per minute, e.g., about 100-150, 75-175, 75-125, 100-125, 125-150, or 125-175 micromoles/minute, e.g., about 125 micromoles NO₂⁻ per minute.

“Activation,” as used herein, is used relative to autotrophic bacteria, e.g., ammonia oxidizing bacteria. Activation refers to any action that may place the ammonia oxidizing bacteria in a potentially more active state, e.g., a growth state. Activation may relate to stimulation of autotrophic bacteria, e.g., ammonia oxidizing bacteria, to assist in some way in the conversion of at least one of ammonia, ammonium ions, and urea into nitrite, nitric oxide, or nitric oxide precursors. Activation may relate to helping establish a bacterial colony, e.g., to allow for the autotrophic bacteria, e.g., ammonia oxidizing bacteria, to compete with other existing bacteria. Activation may relate to providing an environment that may favor sustainability and/or growth of autotrophic bacteria, e.g., ammonia oxidizing bacteria. Activation may relate to accelerating availability of the autotrophic bacteria, e.g., ammonia oxidizing bacteria to an environment or a surface. “Activation” may provide for ammonia oxidizing bacteria to be in an “activated” or “growth state.” “Activation” may take place with the use of an activator. The ammonia oxidizing bacteria may come into contact with the activator to provide an ammonia oxidizing bacteria in an “activated” or “growth” state. This may occur within or outside of a container, e.g., end-use container, delivery device, or delivery system, e.g., within a first chamber, a second chamber, a mixing chamber, a third or additional chamber, or combinations thereof. The activator may be at least one of ammonia, ammonium ions, or urea. The activator may be an ammonium salt, e.g., ammonium chloride or ammonium sulfate. The concentration of the activator, e.g., ammonium salt, e.g., ammonium chloride or ammonium sulfate may be in a range of about 10 micromolar to about 100 millimolar. In certain aspects the concentration of the activator, e.g., ammonium salt, e.g., ammonium chloride or ammonium sulfate may be in a range of about 0.5 mM to about 50 mM. The activator may be in a solution, suspension, a powder, e.g., crystalline form, a media, a buffer, or disposed in or provide as a suitable carrier for maintaining the activator. The activator may be present within the container, e.g., end-use container, or may be present separately from the container, e.g., in another container. The ammonia oxidizing bacteria may be in any suitable form for maintaining the AOB in a desired state, e.g., a storage state, e.g., an aqueous suspension, gel, or powder form. The at least one of ammonia, ammonium ions, or urea may be in a medium or a buffer to promote growth of ammonia oxidizing bacteria, e.g., an AOB media or a growth media. A time-release, or controlled release activator, e.g, urea may be used as an activator.

“Actuation,” as used herein, means that some action is being taken, e.g., a process is being started or something is being put into motion. In some embodiments, actuation may refer to the breaking of a barrier of a container, e.g., end-use container, mixing of the contents of the container, or the initiation of movement of one or more contents of a container, e.g., delivery of one or more contents of the container to outside of the container, e.g., to a surface or an environment. In some embodiments, actuation of the barrier may comprise one or more materials degrading over time that will allow contact of contents of the first chamber and the second chamber, e.g., a controlled release of contents of the first chamber, or a controlled release of contents from the second chamber, or both.

Actuation may also mean some action that allows delivery of contents of the container, e.g., end-use container to outside of the container, e.g., to a surface or an environment. In an embodiment, if actuation of an end-use container containing a finished cosmetic product occurs, a seal of a container may be broken in order to deliver the finished cosmetic product outside of the container. In an embodiment, if actuation of an end-use container containing a finished cosmetic product occurs, a container may be opened, e.g., a valve may be opened, or pump may be triggered in order to deliver the finished cosmetic product outside of the container.

A “barrier,” as used herein, may mean any structure or configuration that may serve to obstruct passage or to maintain separation of the contents of the container, e.g, a finished cosmetic product, e.g., between a first chamber and a second chamber of a container. The barrier may be in the form of a valve, e.g., a check valve, filtering material, film, wax, lipid, polymer, or controlled release material, e.g., slow release material. The barrier may be a material or a structure that upon actuation of a container, it may allow passage of contents from the container to the outside of the container. The barrier may be a material or a structure that upon actuation of the container, it may allow passage of contents from a first chamber into a second chamber, passage of contents from a second chamber into a first chamber, or both. The barrier may be disrupted upon actuation, e.g., through piercing, puncturing, stabbing, perforating, penetrating, splitting, twisting, opening or tearing the barrier. The barrier may be in a form of a valve, e.g., a check valve, a flexible or inflexible material that may not degrade upon contact with one or more contents of the container, or a flexible or inflexible material that may degrade upon contact with one or more contents of the container, a filter material. The barrier may be made of any material suitable for its purpose, e.g., a material that may serve to obstruct passage or to maintain separation, e.g., a polymeric material or metal material.

The barrier may mean any structure that may provide for sealing of the container, e.g., sealing of the container so as to not allow the contents of the container, e.g, a finished cosmetic product, to be exposed to the environment outside of the container, and to not allow anything in the environment outside of the container to enter the interior of the container, e.g, the finished cosmetic product.

In some embodiments, the states most relevant to the present disclosure are the state of growth, e.g., maximal growth, characterized by a pH of at least about 7.6, ammonia, trace minerals, oxygen and carbon dioxide. Another state may be characterized by a pH of about 7.4 or less and characterized by an absence of carbon dioxide. Under low carbon dioxide conditions, ammonia oxidizing bacteria, e.g., Nitrosomonas, continues to oxidize ammonia into nitrite and generates ATP, but lacking carbon dioxide, e.g., lacking sufficient carbon dioxide, to fix and generate protein, it instead generates polyphosphate, which it uses as an energy storage medium. This may allow the ammonia oxidizing bacteria to remain in a “storage state” for a period of time, e.g., a pre-determined period of time, for example, at least 1, 2, 3, 4, 5, 6, 7, days, 1, 2, 3, 4 weeks, 1, 2, 3, 4, 5, 6, 7, 8, 9, 10, 11, 12 months, 1, 2, 3, 4, or 5 years. In some embodiments, the ammonia oxidizing bacteria may remain in a storage state for at least about 6 months to about 1 year.

As used herein, “growth state” refers to autotrophic bacteria, e.g., ammonia oxidizing bacteria, in a state or in an environment, e.g., a media, e.g., a culture media, e.g., a growth media, that may have a pH of at least about 7.6. Levels of at least one of ammonia, ammonium ions, and urea may be between about 1 micromolar and 1000 millimolar. Levels of trace materials are between about 0.01 micromolar iron and 200 micromolar iron. Levels of oxygen are between about 5% and 100% oxygen saturation (e.g., of media). Levels of carbon dioxide are between about 20 ppm and 10% saturation (e.g., of media). In certain aspects, levels of at least one of ammonia, ammonium ions, and urea may be between about 10 micromolar and 100 millimolar. Levels of trace materials are between about 0.1 micromolar iron and 20 micromolar iron. Levels of oxygen are between about 5% and 100% oxygen saturation. Levels of carbon dioxide are between about 200 ppm and 5% saturation (e.g., of media).

As used herein, “polyphosphate loading state” refers to autotrophic bacteria, e.g., ammonia oxidizing bacteria, in a state or in an environment, e.g., a media, e.g., a culture media, e.g., a growth media, that may have a pH of about 7.4, or less. Levels of at least one of ammonia, ammonium ions, and urea are between about 1 micromolar and 2000 millimolar. Levels of trace materials are between 0.01 micromolar iron and 200 micromolar iron. Levels of oxygen are between about 0% and 100% O₂ saturation (e.g., of media). Levels of carbon dioxide are between/less than about zero and 400 ppm, and phosphate levels greater than about 1 micromolar. In certain aspects, levels of at least one of ammonia, ammonium ions, and urea are between about 10 micromolar and 200 millimolar. Levels of trace materials are between 0.1 micromolar iron and 20 micromolar iron. Levels of oxygen are between about 5% and 100% O₂ saturation. Levels of carbon dioxide are between/less than about zero and 200 ppm, and phosphate levels greater than about 10 micromolar.

A purpose of the polyphosphate loading state may be to provide AOB with sufficient ammonia, ammonium ions, and/or urea, and O₂ such that ATP can be produced, but to deny them CO₂ and carbonate such that they are unable to use that ATP to fix CO₂ and instead use that ATP to generate polyphosphate which may be stored by the bacteria.

As used herein, the term “storage state” refers to autotrophic bacteria, e.g., ammonia oxidizing bacteria, in a state or in an environment, e.g., a media, e.g., a culture media, e.g., a growth media, having a pH of about 7.4 or less (in some embodiments, the pH may be 7.6 or less). Levels of at least one of ammonia, ammonium ions, and urea are between about 1 and 1000 micromolar. Levels of trace materials are between about 0.1 and 100 micromolar. Levels of oxygen are between about 0 and 100% saturation (e.g., of media). Levels of carbon dioxide are between about 0 and 800 ppm. In certain aspects, levels of at least one of ammonia, ammonium ions, and urea are between about 10 and 100 micromolar. Levels of trace materials are between about 1 and 10 micromolar. Levels of oxygen are between about 0 and 100% saturation (e.g., of media). Levels of carbon dioxide are between about 0 and 400 ppm.

Ammonia oxidizing bacteria (AOB) are produced according to some embodiments of the present disclosure by generating AOB biomass during a growth state, then exposing the AOB to a polyphosphate loading state and then removing the media and resuspending the AOB in a buffer, e.g., a storage buffer (i.e., the storage state).

“Growth media” or “AOB media,” as referred to herein comprises the following components of Table 1 or Table 2:

TABLE 1

| | Weight/Volume | | Final Concentration | |
|---|---|---|---|---|
| | (in ~1.5 L) | | (in ~1.5 L) | |
| (NH₄)₂SO₄ (MW 132.14) | 4.95 | g | 50 mM NH₄⁺ | |
| KH₂PO₄ (MW 136.1) | 0.616 | g | 3.0 | mM |
| 1M MgSO₄ 7H₂O | 1.137 | ml | 0.76 | mM |
| 1M CaCl₂ 2H₂O | 0.3 | ml | 0.2 | mM |
| 30 mM FeCl₃/50 mM EDTA | 0.5 | ml | 10 μM/16.7 μM | |
| 50 mM CuSO₄ 5H₂O | 30 | μl | 1.0 | μM |

Add 1400 ml ddH₂O to flask. Autoclave (or filter-sterilize). Store at room temperature.

After autoclaving add:

| | | | |
|---|---|---|---|
| Phosphate Buffer (pH 8) | 100 | ml | 32 mM KH₂PO₄/ |
| | | | 2.7 mM NaH₂PO₄•H₂O |
| 5% Na₂CO₃ | 12 | ml | 0.04% |

(expected pH is about 8.0)
Store at room temperature

TABLE 2

| | Batch medium | | Feeding solution | |
|---|---|---|---|---|
| | Weight/Volume (1 L) | | Weight/Volume (1 L) | |
| | (Final concentration) | | (Final concentration) | |
| (NH₄)₂SO₄ (MW 132.14) | 3.3 | g | 13.2 | g |
| | (50 mM NH₄⁺) | | (200 mM NH₄⁺) | |
| KH₂PO₄ (MW 136.1) | 1.23 | g | 0.41 | g |
| | (9.0 | mM) | (3.0 | mM) |
| 1M MgSO₄ | 0.758 | ml | 0.758 | ml |
| | (0.76 | mM) | (0.76 | mM) |
| 1M CaCl₂ | 0.2 | ml | 0.2 | ml |
| | (0.2 | mM) | (0.2 | mM) |
| 30 mM FeCl₃/50 | 0.333 | ml | 0.333 | ml |
| mM EDTA | (10 μM/16.7 μM) | | (10 μM/16.7 μM) | |
| 50 mM CuSO₄ | 20 | μl | 20 | μl |
| | (1.0 | μM) | (1.0 | μM) |
| ddH₂O | 1000 | ml | 1000 | ml |

Autoclave each solution and store at room temperature.

“Deterioration-based expiration date,” as used in here, is a date after which a product, e.g. a cosmetic product, e.g, a finished product, is expected to degrade in some way that would make it unsuitable for its intended purpose. In some instances it may be a date after which a product, e.g., a cosmetic product, e.g., a finished cosmetic product has degraded. An indication that the product has degraded may be provided by way of an indicator located on or in the cosmetic product or cosmetic product packaging, e.g, end-use container that provides for some sign that the product has degraded. For example, this may be accomplished by way of a color indicator.

“Biome-compatible-based expiration date,” as used here, is a date after which a product, e.g. a cosmetic product, e.g, a finished product, is expected to become contaminated in some way that would make it unsuitable for its intended purpose. In some instances it may be a date after which a product, e.g., a cosmetic product, e.g., a finished cosmetic product has become contaminated. An indication that the product has become contaminated may be provided by way of an indicator located on or in the cosmetic product or cosmetic product packaging, e.g, end-use container that provides for some sign that the product has been contaminated. For example, this may be accomplished by way of a color indicator. A contaminant may refer to something that may make the product unsuitable for its intended use, and may include any item that is not in the finished cosmetic product at the time of sealing the product and/or subsequent to sterilization of the product.

“Biome-friendly” refers to something, e.g, a product, e.g., a cosmetic product, e.g., a finished cosmetic product that may allow for minimal disruption of a microbiome of a subject. For example, biome-friendly refers to a product that may be applied to a subject that may allow the microbiome at the point of application to be maintained, minimally disrupted, and/or able to return to the microbiome after a period of time after application of the product. In embodiments, biome-friendly may refer to ammonia oxidizing bacteria-friendly, in that the product may allow for minimal disruption of the ammonia oxidizing bacteria of a subject.

In embodiments, “biome-friendly” may be referred to as “biome-compatible.”

“Recycler” refers to any entity or person that may accept an item to undergo reuse or renewal of that item in the same or different form. The recycler may accept an item that may then be subjected to or suitable for further use or activity. The recycler may pass the item through a system for further treatment or use.

Throughout this disclosure, formulation may refer to a composition or preparation.

TABLE 3
Potential components of a biome-friendly product.

| Component | Category |
|---|---|
| Decyl glucoside | Surfactant/Cleanser |
| cocamidopropyl betaine (ColaTeric COAB) | Surfactant/Cleanser |
| polyethylene sorbitol ester (e.g., Tween 80) | Surfactant/Cleanser |
| ethoxylated lauryl alcohol (RhodaSurf 6 NAT) | Surfactant/Cleanser |
| sodium laureth sulfate/lauryl glucoside/ | Surfactant/Cleanser |
| cocamidopropyl betaine (Plantapon 611 L UP) | |
| sodium laureth sulfate (e.g., RhodaPex ESB 70 | Surfactant/Cleanser |
| NAT) | |
| alkyl polyglucoside (e.g., Plantaren 2000 N UP) | Surfactant/Cleanser |
| sodium laureth sulfate (Plantaren 200) | Surfactant/Cleanser |
| Dr. Bronner's Castile soap | Surfactant/Cleanser |
| Dr. Bronner's baby soap | Surfactant/Cleanser |
| Lauramine oxide (ColaLux Lo) | Surfactant/Cleanser |
| sodium dodecyl sulfate (SDS) | Surfactant/Cleanser |
| polysulfonate alkyl polyglucoside (PolySufanate | Surfactant/Cleanser |
| 160 P) | |
| sodium lauryl sulfate (Stepanol-WA Extra K) | Surfactant/Cleanser |
| Sodium Laurylglucosides Hydroxypropylsulfonate | Surfactant/Cleanser |
| (Suga ®nate 160NC) | (bio-based) |
| lauramidopropyl betaine (Cola ®Teric LMB) | Surfactant/Cleanser |
| Cocamidopropyl hydroxysultaine (Cola ®Teric | Surfactant/Cleanser |
| CBS) | |
| disodium cocoamphodiacetate (Cola ®Teric | Surfactant/Cleanser |
| CDCX-LV) | |
| sodium laurylglucosides hydroxypropyl phosphate | Surfactant/Cleanser |
| (Suga ®Fax D12). | |
| sodium lauroyl methyl isethionate (Iselux ® | Surfactant/Cleanser |
| LQ-CLR-SB) | |
| sodium methyl cocoyl taurate (Pureact WS Conc.) | Surfactant/Cleanser |
| Aqua (and) Sodium Lauroyl Methyl Isethionate | Surfactant/Cleanser |
| (and) Cocamidopropyl Betaine (and) | |
| Sodium Cocoyl Isethionate (and) | |
| Sodium Methyl Oleoyl Taurate (Iselux ®SFS-SB) | |
| Coco glucoside (Plantacare 818) | Surfactant/Cleanser |
| | (bio-based) |
| Sodium cocoyl-glycinate | Surfactant/Cleanser |
| | (bio-based) |
| Caprylic/Capric triglyceride (Myritol) | Conditioner |
| Cationic guar (N-Hance) | Conditioning/de- |
| | tangling |
| Coconut oil | Conditioning |
| Apple saccharides, e.g., pyrus malus (apple) fruit | Humectant |
| extract e.g., pyrus malus (apple) fruit extract and | |
| glycerin (Botanimoist AMS); | |
| Hydrolyzed quinoa | Moisture binding |
| Hydrolyzed adansonia digitata (Baobab) Seed | Softener |
| Protein | |
| Hydroxyethylcellulose (Natrosol) | Viscosity modifier |
| Hydroxypropylcellulose (Klucel MCS) | Viscosity modifier/ |
| | film former |
| Hydrolyzed soy protein (Soy Tein NPNF) | |
| Natural Rose Hydrosol | Fragrance |
| Natural Rose Water | Fragrance |
| Damascena flower water | Fragrance |
| Coco-glucoside and glyceryl oleate (Lamisoft ® | Lipid layer enhancer |
| PO 65) | |
| Polysorbate 80 | Emulsifier |
| Citric acid | pH stabilizer |
| Squalene | Moisturizer |
| Water (i.e. deionized water) | |

### 2. Cosmetic Products

Cosmetic products, e.g., finished cosmetic products, are provided that may be biome-friendly or biome-compatible. The cosmetic products may comprise one or more components that may be biome-friendly.

The cosmetic products of the present disclosure may be, or include, or be disposed in any one or more of a baby product, e.g., a baby shampoo, a baby lotion, a baby oil, a baby powder, a baby cream; a bath preparation, e.g., a bath oil, a tablet, a salt, a bubble bath, a bath capsule; an eye makeup preparation, e.g., an eyebrow pencil, an eyeliner, an eye shadow, an eye lotion, an eye makeup remover, a mascara; a fragrance preparation, e.g., a colognes, a toilet water, a perfume, a powder (dusting and talcum), a sachet; hair preparations, e.g., hair conditioners, hair sprays, hair straighteners, permanent waves, rinses, shampoos, tonics, dressings, hair grooming aids, wave sets; hair coloring preparations, e.g., hair dyes and colors, hair tints, coloring hair rinses, coloring hair shampoos, hair lighteners with color, hair bleaches; makeup preparations, e.g., face powders, foundations, leg and body paints, lipstick, makeup bases, rouges, makeup fixatives; manicuring preparations, e.g., basecoats and undercoats, cuticle softeners, nail creams and lotions, nail extenders, nail polish and enamel, nail polish and enamel removers; oral hygiene products, e.g., dentrifices, mouthwashes and breath fresheners; bath soaps, e.g., foaming body cleansers, and detergents, deodorants, douches, feminine hygiene deodorants; shaving preparations, e.g., aftershave lotions, beard softeners, talcum, preshave lotions, shaving cream, shaving soap; skin care preparations, e.g., cleansing, depilatories, face and neck, body and hand, foot powders and sprays, moisturizing, night preparations, paste masks, skin fresheners; and suntan preparations, e.g., gels, creams, and liquids, and indoor tanning preparations.

A finished cosmetic product may be provide comprising a cosmetic product, e.g, a shampoo, a body cleanser, a conditioner. The finished cosmetic product may be disposed in an end-use container.

The finished cosmetic product may have one or more properties: (a) The cosmetic product, or finished cosmetic product may be substantially free of a preservative, e.g., a paraben. (b) The end-use container may be configured to reduce retrograde flow. (c) The cosmetic product, or finished cosmetic product may be sterilized. The sterilization may include irradiation, e.g., gamma irradiation, or heat sterilization. (d) one of the following compositions of (i) and (ii): (i) The cosmetic product may comprise, consist essentially of, or consist of a viscosity modifier, one cleanser/surfactant, two cleansers/surfactants, a humectant, a skin conditioner, and a fragrance.

The cosmetic product or finished cosmetic product may comprise, consist essentially or consist of the following composition(s):

| | | Preferred | |
|---|---|---|---|
| | Preferred | Concentration | |
| Component | Concentration | Range | Description |
| Klucel MCS | 1.5% | 1.0-2.0% | Viscosity |
| (Hydroxypropyl | | | Modifier |
| Cellulose) | | | |
| Cocamidopropyl | 20.0% | 15.0%-30.0% | Cleanser/ |
| Betaine | | | Surfactant |
| Decyl Glucoside | 7.5% | 0.0-10.0% | Cleanser/ |
| | | | Surfactant |
| Botanimoist AMG | 3.0% | 0.0-4.0% | Humectant |
| (Glycerine + Apple | | | |
| extract | | | |
| Hydrolyzed Baobab | 2.0% | 0.0-4.0% | Skin |
| Protein | | | Conditioner |
| Natural Rose | 10.0% | 0.0-15.0% | Fragrance |
| Hydrosol | | | |

This product may be used as a cosmetic product, e.g., for a shampoo, e.g., for a body wash. The product includes water to make 100%. In some embodiments, the cosmetic product, e.g., shampoo, may or may not contain citric acid, and the citric acid may be needed in cases where pH stabilization is required or desired.

(ii) The cosmetic product or finished cosmetic product may comprise, consist essentially or consist of the following composition(s):

| | | Preferred | |
|---|---|---|---|
| | Preferred | Concentration | |
| Component | Concentration | Range | Description |
| Klucel MCS | 0.25% | 0.10-0.50% | Viscosity |
| (Hydroxypropyl | | | modifier |
| Cellulose) | | | |
| Cocamidopropyl | 8.00% | 5.0-12.0% | Cleanser/ |
| betaine | | | Surfactant |
| Decyl Glucoside | 4.0% | 0.0-6.0% | Cleanser/ |
| | | | Surfactant |
| Botanimoist AMG | 2.0% | 0.0%-4.0% | Humectant |
| (Glycerine + Apple | | | |
| extract) | | | |
| Hydrolyzed Quinoa | 1.0% | 0.0-3.0% | Skin |
| Protein conditioner | | | Conditioner |
| Natural Rose | 7.5% | 0.0%-10.0% | Fragrance |
| Hydrosol | | | |
| Citric Acid | Added as needed, | As needed, | pH |
| | usually in | usually in | stabilizer |
| | very small | very small | |
| | amounts, for pH | amounts, for pH | |
| | stabilization | stabilization | |

This product may be used as a cosmetic product, e.g., for a cleanser, e.g., for a body, hands, or face. The product includes water to make 100%. In some embodiments, the cosmetic product, e.g., cleanser, may or may not contain citric acid, and the citric acid may be needed in cases where pH stabilization is required or desired.

Other hydrolyzed protein may be used, and may include, but is not limited to rice, soy baobab, and oat. Other fragrance alternatives may be contemplated.

The finished cosmetic product may have one or more, or all the properties described herein.

Other products are contemplated, including hair and/or skin conditioners that may comprise, consist essentially of, or consist of the following

| | | Preferred | |
|---|---|---|---|
| | Preferred | Concentration | |
| Component | Concentration | Range | Description |
| Hydroxypropyl | 1.5% | 0.50-2.50% | Viscosity |
| cellulose | | | modifier/film |
| (Klucel MCS) | | | former |
| Cationic guar | 0.5% | 0.10-1.5% | Conditioning and |
| (N-Hance) | | | detangling |
| Coconut oil | 1.0% | 0.0-3.0% | Conditioning |
| Fragrance | As desired | As desired | |

The product includes water to make 100%. In some embodiments, the cosmetic product, e.g., conditioner may or may not contain citric acid, and may be needed in cases where pH stabilization is required or desired.

In accordance with one or more non-limiting embodiments, a cosmetic product or finished cosmetic product, for example, a biome-friendly shampoo, may comprise, consist essentially or consist of the following composition(s):

| Component | Sample Ingredient(s) | % range (example %) | |
|---|---|---|---|
| Surfactant/ | Sodium laurylglucosides | 50-70% | (64.3%) |
| cleanser(s) | hydroxypropylsulfonate | | |
| | Lauramidopropyl betaine | 20-40% | (28.8%) |
| Fragrance | Rosa Damascena Flower Water | 3-10% | (4.6%) |
| Softener | Hydrolyzed Adansonia Digitata | 1-5% | (1.85%) |
| | Seed Protein | | |
| Moisturizer | Squalene | 0.1-2% | (0.45%) |

In accordance with one or more non-limiting embodiments, a cosmetic product or finished cosmetic product, for example, a biome-friendly face and/or body cleanser, may comprise, consist essentially or consist of the following composition(s):

| Component | Sample Ingredient(s) | % range (example %) | |
|---|---|---|---|
| | Deionized Water | 70-80% | (76.25%) |
| Surfactant/ | Lauramidopropyl betaine | 5-12% | (8%) |
| cleanser(s) | | | |
| | Decyl Glucoside | 3-7% | (4%) |
| Fragrance | Rosa Damascena Flower Water | 4-10% | (7.5%) |
| Humectant | Pyrus Malus Fruit Extract in | 2-5% | (3%) |
| | glycerin | | |
| Moisture | Hydrolyzed Quinoa | 0.5-2% | (1%) |
| binder | | | |
| Viscosity | Hydroxypropylcellulose | 0.1-1% | (0.25%) |
| modifier/ | | | |
| film former | | | |

In accordance with various disclosed and non-limiting embodiments, the amount of each individual component in the product may vary by, for example, 1%, 5%, or 10%, while still maintaining similar properties of the product.

The cosmetic product may be preservative free, e.g., it does not include a preservative. Preservatives may be identified as the compounds listed in Annex VI at the end of the Detailed Description, herein. At most, the finished cosmetic product may have less than 500 ppb of a preservative, e.g, one or more of those listed in Annex VI. In embodiments, the finished cosmetic product may have less than 100 ppb of a preservative, e.g., one or more of those listed in Annex VI. A brief list of materials that could be used as “natural” preservatives include: Neem Oil, Lemon juice or oil, Bee Propolis, Rosemary Extract, Grapefruit Seed Extract, Citric acid, Alpha tocopherol (also known as vitamin E), Potassium Sorbate, Phenoxyethanol, Salicylic Acid, Sodium Benzoate, Sorbic Acid, Plant Essential Oils (Thyme, Oregano, Lemongrass, Lavender, Rosemary, and others), Lactic Acid.

At most, the finished cosmetic product may have between about 1 ppm and 10 ppb of preservative, e.g., one or more of those listed in Annex VI; for example, between about 10 ppb and 50 ppb, for example, between about 50 ppb and 100 ppb, e.g., less than 1000, 900, 800, 700, 600, 500, 400, 300, 200, 100, 50, 40, 10, 5, 1 ppb of one or more preservatives, e.g., one or more preservatives listed in Annex VI.

In some embodiments, the preservative-free property of the cosmetic product, e.g., finished cosmetic product may be evidenced through testing disclosed herein. For example, upon exposure to one or more microorganisms, e.g, microbe, e.g., a bacterium or fungus, e.g., growth of the one or more microorganisms will be supported. For example, the cosmetic product or finished cosmetic product, if exposed to challenge with a microbe, e.g., a bacterium or fungus, will support growth of said microbe, e.g., as determined by U.S.P. 51, Antimicrobial Effectiveness Testing (USP31-NF26 Page 67), herein incorporated by reference in its entirety. The cosmetic product or finished cosmetic product, in the absence of a treatment, e.g., sterilization treatment or the addition of a preservative, supports microbe growth, e.g., bacterial or fungal growth, e.g., as measured by U.S.P. 51, Antimicrobial Effectiveness Testing.

In some embodiments, the preservative-free property of the cosmetic product, e.g., finished cosmetic product may be evidenced by, in the absence of a treatment, e.g., sterilization treatment or the addition of a preservative, supports one or more microorganisms, e.g, microbe, e.g., a bacterium or fungus.

In certain embodiments, the cosmetic product may be disposed in an end-use container, and the finished cosmetic product may comprise, consist essentially of, or consist of water, cocamidopropyl betaine, rosa damascena flower water, decyl glucoside, pyrus malus (apple) fruit extract, glycerin, hydrolyzed adansonia digitata (baobab) seed protein, hydroxypropylcellulose. The cosmetic product may be a shampoo.

In certain embodiments, the cosmetic product may be disposed in an end-use container, and the finished cosmetic product may comprise, consist essentially of, or consist of water, cocamidopropyl betaine, rosa damascena flower water, decyl glucoside, pyrus malus (apple) fruit extract, glycerin, hydrolyzed quinoa, hydroxypropylcellulose, and citric acid. The cosmetic product may be a cleanser.

In certain embodiments, the cosmetic product may be disposed in an end-use container, and the finished cosmetic product may comprise, consist essentially of or consist of hydroxyethyl cellulose, myritol 313 C8-10 triglycerides, coco-glucoside and glyceryl oleate, polysorbate-80, and natural rose water. The cosmetic product may be a conditioner.

The cosmetic product or finished cosmetic product may comprise a component added to provide one or more of the following: a fragrance, a color, viscosity, foam forming or foam stability, adhesion, moisture retention, moisture binding, pH stabilization, cleansing, thickening, softening, conditioning, e.g., hair or skin conditioning, lipid layer enhancing, barrier-forming, or film-forming.

The finished cosmetic product or the cosmetic product, may comprise one or more of an antioxidant, fatty substance/oil, thickener, softener, emulsifier, light-screening agent, foam forming and foam stability, antifoaming agent, moisturizer, fragrance, surfactant, filler, sequestering agent, polymers, acidifying or basifying agent, dyes, colorant, pigment, pearlizer, opacifier, organic or inorganic particle, viscosity modifier, cleanser, adherent, moisture binder, pH stabilizer, conditioner, de-tangler, biobased surfactant cleanser, lipid layer enhancer, skin conditioner, and natural hair nutrient such as botanicals, fruit extracts, sugar derivatives and/or amino acids, hydrolyzed proteins, or vitamins.

### 3. Containers, e.g., End-Use Container, Delivery Devices

Containers and/or delivery devices, e.g., containers, e.g., delivery devices, e.g., end-use containers are provided as a housing for the cosmetic product, e.g., a finished cosmetic product. In some embodiments, the container, or delivery device may also serve the purpose of delivering the cosmetic product, e.g., finished cosmetic product, e.g., to a surface or a subject.

The container and/or delivery device may be configured to store and/or deliver any cosmetic product disclosed herein. The cosmetic product may be delivered to a site, an environment, or a surface, e.g., of a subject, with or without additional components. In certain embodiments, other components may be delivered simultaneously or consecutively, e.g., at least partially before or at least partially after, the delivery of the cosmetic product commences. In certain embodiments, the container or delivery device may comprise or be referred to as a delivery system. In some embodiments, the delivery of one component is still occurring when the delivery of the second begins, so that there is overlap. This is sometimes referred to herein as “simultaneous” or “concomitant” or “concurrent delivery”. In other embodiments, the delivery of one component ends before the delivery of the other treatment begins. This is sometimes referred to herein as “successive” or “sequential delivery” or “consecutive delivery.”

A barrier may be provided as part of or within the container to prevent fluid communication between the interior of the container and the exterior environment. The barrier may be in the form of a valve, e.g., check valve, filtering material, film, wax, lipid, polymer, control release material, e.g., a gel, and other materials that may either provide a permanent or temporary barrier between the interior of the container and the exterior environment.

Upon actuation of the container, the barrier may be disrupted to allow disposal of the cosmetic product from the container to the exterior environment, or a site, an environment, or a surface, e.g., of a subject, to contact the cosmetic product with a site, an environment, or a surface, e.g., of a subject.

The container may comprise a delivery system. The delivery system may be an applicator or be configured to deliver the contents of the cosmetic product.

The delivery system may be configured to deliver a cosmetic product to a surface of a subject, e.g., to a skin surface. The cosmetic product may be in the form of a particle, or a plurality of particles having a particle size to enhance delivery or enhance positioning or contact with a desired target site. The cosmetic product may be in the form of a liquid, solid, in a suspension or in a solution.

In certain embodiments, the delivery system may comprise a pump to deliver the contents of the interior of the container to a target site, e.g., an environment, e.g., a surface of a subject, e.g., skin of a subject.

In some embodiments, the container may be a single-use container. The container may or may not be pre-loaded (e.g., loaded by a manufacturer or user) with contents, e.g., cosmetic product, and may be used once by a user, e.g., a consumer or medical professional to deliver the contents of the container to a target site, e.g., an environment, e.g., a surface of a subject, e.g., skin of a subject.

In other embodiments, the container may be a multiple-use container in which the container may or may not be pre-loaded (e.g., loaded by a manufacturer or user) with contents, e.g., cosmetic product, and may be used once by a user, e.g., a consumer or medical professional to deliver the contents of the container to a target site, e.g., an environment, e.g., a surface of a subject, e.g., skin of a subject. The container may be re-loaded (e.g., loaded by a manufacturer or user) with contents e.g., ammonia oxidizing bacteria, and ammonia, ammonium ions and urea, and may be used again by a same or different user, e.g., a consumer or medical professional to deliver the contents of the container to a target site, e.g., an environment, e.g., a surface of a subject, e.g., skin of a subject.

Pre-loading or re-loading of the contents, e.g., cosmetic product may comprise a sterilization process to ensure that the contents of the container are sterilized.

In some embodiments, the container may be in the form of a syringe, bottle, ampule, applicator, pouch, e.g., spout pouch, e.g., with screw top. A pump may be attached to the bottle in order to dispense the contents from the container. The container may provide for an aerosol spray or mist. The container may be a squeezable container to allow dispensing of the contents through an opening that is covered by a closure. The container may have a screw type closure, a non-spill closure, a snap cap closure, or a snap flap closure. The container may have an over cap that resides over the dispensing area in which dispensing of the contents occurs. The closure may be fully removable or partially removable, e.g., fully removable form the body of the container, or partially removable and attached by a hinge. The container may be a single use package, e.g., a laminated packet, e.g., that may be torn open to dispense the contents, and disposed after use.

The container, e.g., end-use container may be configured to inhibit retrograde flow, e.g., backflow, e.g., reverse flow, e.g., rearward movement, of material, e.g., the cosmetic product, into said end-use container. The container, e.g, end use container may be configured to inhibit retrograde flow, e.g., backflow, e.g., reverse flow, e.g., rearward movement, of material, e.g., a contaminant, into said end-use container. The contaminant may be is atmospheric, e.g., an aerosol, or a liquid, e.g., water, or solid, or a gas.

The end-use container may comprise a reservoir in which said cosmetic product is disposed, and a dispenser through which said cosmetic product from said reservoir can be dispensed, wherein said dispenser inhibits retrograde flow of material into said reservoir.

The end-use container may comprise a reservoir in which said cosmetic product is disposed, and a dispenser through which said cosmetic product from said reservoir can be dispensed, wherein said dispenser inhibits retrograde flow of dispensed cosmetic product, or atmospheric aerosols, into said reservoir.

The end-use container may be an anti-retrograde flow dispenser comprising a first pressure activated valve disposed in said dispenser and proximal to said reservoir and a second pressure activated valve disposed in said dispenser and distal to said reservoir, wherein the activation pressure of said first valve is higher than the activation pressure of said second valve.

The end-use container may comprise an anti-retrograde mechanism configured to prevent movement of the cosmetic product in a direction opposite the operational direction associated with dispensing the finished cosmetic product.

In some embodiments, the container may be substantially free of organisms, e.g., microorganisms. In embodiments the container may be free of other organisms. The container may be sterilized to provide for a container substantially free or free of organisms, e.g., microorganisms.

The container may be disposed in a powder, cosmetic, cream, stick, aerosol, salve, wipe, or bandage. The container may be provided as a powder, cosmetic, cream, stick, aerosol, salve, wipe, or bandage.

In some embodiments, the container may comprise an indicator component. The indicator component may a color marker that may develop a color upon the contact of a microorganisms to the interior of the container.

The container may be constructed of any material suitable for housing the contents, e.g., a cosmetic product, e.g., a finished cosmetic product disclosed herein. For example the container may be constructed and arranged to be at least partially resistant to at least one of gaseous exchange, water, and light. For example, the container may be constructed of a glass or polymeric material.

The end-use container may be composed of or comprise a polymer, e.g., polyethylene terephthalate (PET), high density polyethylene (HDPE), polypropylene, polycarbonate, polytetrafluoroethylene (Teflon®), polyviylidene fluoride (PVDF), or a cellulosic. The end-use container may be composed of or comprise glass. A sensor, e.g., an oxygen sensor, may be included in the end-use container that may indicate a presence of viable bacterial. The end-use container may allow for passage of at least about 5, 10, 20, 30, 40, 50, 60, 70, 80, 90, 95, 99, or 100 percent of transmission of ionizing radiation, e.g., with gamma rays, e.g., with x-rays, e.g., from an isotope, e.g., cobalt 60, or with ultraviolet, e.g., ultraviolet C (UVC) through the end-use container.

In some embodiments, one or more other organisms, such as ammonia oxidizing bacteria may be included in the container. An organism of the genus selected from the group consisting of Lactobacillus, Streptococcus, Bifidobacter, and combinations thereof, may be provided in the container. An activator for the ammonia oxidizing bacteria may be provided in the container.

The containers described herein may be adapted to deliver one or more cosmetic products. The containers described herein may be adapted to deliver one or more therapeutic products.

The weight of the container, delivery system, or delivery device, including or not including the contents of the container may be less than about 50, 100, 200, 300, 400, 500, 600, 700, 800, 900, 1000, 1500, or 2000 grams.

### 4. Dispensing the Cosmetic Product/Methods of Delivering the Cosmetic Product

The product may be dispensed in discrete volumes or amounts from the container, e.g., end-use container. The container may dispense the same volume or approximately same volume for each actuation of the container. The product may be dispensed, for example in a discrete volume of between about 0.1 ml and about 5 ml. The discrete volume can be, for example about 0.1 ml, 0.2 ml, 0.25 ml, 0.3 ml, 0.4 ml, 0.5 ml, 0.6 ml, 0.7 ml, 0.75 ml, 0.8 ml, 0.9 ml, 1.0 ml, 1.1 ml, 1.2 ml, 1.4 ml, 1.5 ml, 1.6 ml, 1.8 ml, 2.0 ml, 2.25 ml, 2.5, ml, 2.75 ml, 3 ml, 3.25 ml, 3.5 ml, 3.75 ml, 4.0 ml, 5.0 ml, 6.0 ml, 7.0 ml, 8.0 ml, 9.0 ml, or 10 ml.

The container may dispense the same amount or approximately same amount for each actuation of the container. The product may be dispensed, for example in a discrete amount of between about 0.1 grams (g) and about 5 g. The discrete amount can be, for example about 0.1 g 0.2 g, 0.25 g, 0.3 g, 0.4 g, 0.5 g, 0.6 g, 0.7 g, 0.75 g, 0.8 g, 0.9 g, 1.0 g, 1.1 g, 1.2 g, 1.4 g, 1.5 g, 1.6 g, 1.8 g, 2.0 g, 2.25 g, 2.5, g, 2.75 g, 3 g, 3.25 g, 3.5 g, 3.75 g, 4.0 g, 5.0 g, 6.0 g, 7.0 g, 8.0 g, 9.0 g, or 10 g.

Containers may be configured to dispense a first volume or amount for a first actuation, and a second volume or amount for a second actuation.

The total volume of cosmetic product in the container may be between about 0.1 and about 100 fluid ounces, about 0.2 and about 50 fluid ounces, about 0.5 and about 25 fluid ounces, about 1.0 and about 10 fluid ounces, about 2.0 and about 7 fluid ounces, about 3 and about 5 fluid ounces. In some embodiments, the volume may be about 3.4 fluid ounces.

The container may be constructed to contain between about 0.1 and about 100 fluid ounces, about 0.2 and about 50 fluid ounces, about 0.5 and about 25 fluid ounces, about 1.0 and about 10 fluid ounces, about 2.0 and about 7 fluid ounces, or about 3 and about 5 fluid ounces. In some embodiments, the container may be constructed to contain about 3.4 fluid ounces. The container may be a one-chamber container, or any other container disclosed herein.

### 5. Use of the Product

The product, e.g., cosmetic product, e.g., finished cosmetic product, may be used one time (1×) per day, twice (2×) per day, three times (3×) per day, 4× per day, 5× per day, 6× per day, 7× per day, 8× per day, or more. The product may be used 1× per week, 2× per week, 3× per week, 4× per week, 5× per week, 6× per week, or 7× per week.

The amount of cosmetic product in the end-use container may be sufficient for no more than a pre-determined amount of applications, for example, X applications, wherein X is between about 1 and about 60, for example, between about 1-3, 4-6, 7-9, 10-13, 14-17, 18-21, 22-25, 26-29, 30-33, 34-37, 38-41, 42-45, 46-49, 50-53, 54-57, 58-60. The amount of cosmetic product in the end-use container may be sufficient for no more than a pre-determined amount of applications, for example, X applications, wherein X is between about 1 and about 180, for example, between about 1-60, 61-120, 121-180. The amount of cosmetic product in the end-use container may be sufficient for no more than a pre-determined amount of applications, for example, X applications, wherein X is between about 1 and about 750, for example, between about 1-100, 101-200, 201-300, 301-400, 401-500, 501-600, 601-750.

The amount of cosmetic product in the finished cosmetic product may be selected such that the finished cosmetic product is sufficient for no more than a pre-determined amount of applications, for example, X applications, wherein X is between about 1 and 180. In embodiments, X may be between about 1 and about 180, for example, between about 1-60, 61-120, 121-180. The amount of cosmetic product in the end-use container may be sufficient for no more than a pre-determined amount of applications, for example, X applications, wherein X is between about 1 and about 750, for example, between about 1-100, 101-200, 201-300, 301-400, 401-500, 501-600, 601-750.

For example, for a shampoo, the number of applications may be about 15-40, for example, 1 per day for about 4 weeks. For a cleanser, the number of applications may be about several per day, for about 4 weeks, for example about 15-90 applications.

The amount of cosmetic product in the finished cosmetic product may be selected such that the finished cosmetic product may be used for no more than about a pre-determined number of uses, for example, between about 500-750 uses, 200-500 uses, 100-200 uses, 50-100 uses, 40-50 uses, 30-40 uses, 20-30 uses, 10-20 uses, 5-10 uses, or 1-5 uses.

The amount of cosmetic product in the finished cosmetic product may be selected such that the finished cosmetic may be used for no more than about a pre-determined number of days, for example, between about 500-750 days, 300-500 days, 100-300 days, 50-100 days, 40-50 days, 30-40 days, 20-30 days, 10-20 days, 5-10 days, or 1-5 days. The pre-determined number of applications, uses, or days may be about 28.

The amount of cosmetic product in the finished cosmetic product may be selected such that the finished cosmetic product is sufficient for not more than X days of Y/day use, wherein X is between about one day and about 42 days (6 weeks), and Y is between about zero uses per day and about ten uses per day. For example X days may be about 1-6, 7-10, 10-13, 14-17, 18-21, 22-25, 26-29, 30-33, 34-37, 38-42 days; and Y may be about 0-1, 2-4, 5-7, 8-10 uses per day.

The finished cosmetic product may have an expiration date. This may be a date upon which, after such date, the finished cosmetic product should not be used, e.g. it should be disposed of. The expiration date may be a deterioration-based expiration date. This date may be a date after which a product, e.g. a cosmetic product, e.g, a finished product, is expected to degrade or become contaminated in some way that would make it unsuitable for its intended purpose. In some instances it may be a date after which a product, e.g., a cosmetic product, e.g., a finished cosmetic product has degraded or become contaminated. An indication that the product has degraded or become contaminated may be provided by way of a symbol or one or more written words on the end-use container. In embodiments, an indicator located on or in the cosmetic product or cosmetic product packaging, e.g, end-use container that provides for some sign that the product has degraded or become contaminated. For example, this may be accomplished by way of a color indicator.

The expiration date may be a biome-compatible-based expiration date. This date may be a date after which a product, e.g. a cosmetic product, e.g, a finished product, is expected to become contaminated in some way that would make it unsuitable for its intended purpose. In some instances it may be a date after which a product, e.g., a cosmetic product, e.g., a finished cosmetic product has become contaminated. An indication that the product has become contaminated may be provided by way of a symbol or one or more written words on the end-use container. In embodiments, an indicator located on or in the cosmetic product or cosmetic product packaging, e.g, end-use container that provides for some sign that the product has been contaminated. For example, this may be accomplished by way of a color indicator. A contaminant may refer to something that may make the product unsuitable for its intended use, and may include any item that is not in the finished cosmetic product at the time of sealing the product and/or subsequent to sterilization of the product.

The finished cosmetic product may have an indication of expiration, or lifetime. This indication of expiration may be a recommended lifetime of the product, and it may be a preselected period of time. The preselected period of time may be expressed as a unit of time, for example, expressed in days, weeks, or months.

The finished cosmetic product may comprise an indication of expiration, or lifetime, e.g., recommended lifetime, after the preselected period of time, e.g., expressed in days, that is less than X days from the date of one of the following: manufacturing, filling, sealing, sterilization, shipping, releasing into commerce, or selling. In embodiments, X may be about 5-7 days, about 5-10 days, about 7-14 days, about 14-21 days, about 21-28 days, about 28-35 days, about 35-42 days, about 42-49 days, about 49-56 days, about 56-63 days, about 63-70 days, about 70-77 days, about 75-100 days, about 100-150 days, about 150-200 days, about 200-300 days, about 300-400 days, about 400-750 days. In certain embodiments, X may be about 28 days, e.g., 28 days (4 weeks).

In embodiments, X may be related to the opening or unsealing of the finished cosmetic product, or the first use of the finished cosmetic product. about 5-7 days, about 5-10 days, about 7-14 days, about 14-21 days, about 21-28 days, about 28-35 days, about 35-42 days, about 42-49 days, about 49-56 days, about 56-63 days, about 63-70 days, about 70-77 days, about 75-100 days, about 100-150 days, about 150-200 days, about 200-300 days, about 300-400 days, about 400-750 days. In certain embodiments, X may be about 28 days, e.g., 28 days.

In embodiments, the indication of expiration, or lifetime, e.g., recommended lifetime, is expressed as a preselected number of uses or applications. The indication may be expressed as a preselected number between about 5-7, about 5-10, about 7-14, about 14-21, about 21-28, about 28-35, about 35-42, about 42-49, about 49-56, about 56-63, about 63-70, about 70-77, about 75-100, about 100-150, about 150-200, about 200-300, about 300-400, about 400-750 days.

In embodiments, the finished cosmetic product may have an expiration date, or lifetime, e.g., recommended lifetime that is expressed:

a) in units of time, e.g., days, from a preselected event, e.g., unsealing of said finished cosmetic product or the first use of said finished cosmetic product; and/or

b) as the number of uses or applications.

The product may be used in conjunction, e.g., in combination, with a bacteria-containing product, e.g., an ammonia oxidizing bacteria (AOB) containing product, e.g., an N. eutropha containing product, e.g., an N. eutropha D23 containing product.

Methods may be provided that allow for maintenance of bacteria, e.g., AOB, e.g. N. eutropha, e.g., an N. eutropha D23, using one or more cosmetic products, e.g, finished cosmetic products disclosed herein.

Methods may comprise application of bacteria, e.g., AOB, e.g. N. eutropha, e.g., an N. eutropha D23, prior to or subsequent to use of one or more cosmetic products or cosmetic products, e.g, finished cosmetic products disclosed herein.

The methods may comprise waiting a period of time, e.g., a pre-determined period of time before applying the cosmetic product. The period of time may comprise a period of time in which a non-biome-friendly cosmetic product is not used.

Use of the cosmetic product, e.g., finished cosmetic product may occur on a regular basis, e.g., every day, every week, every month, or in one of the following ranges, every 1-2 days, every 2-5 days, every 5-10 days, every 10-15 days, every 15 to 30 days.

In embodiments, no non-biome friendly cosmetic product may be applied to the subject in the period between the last application of the cosmetic product and the next application of the cosmetic product and/or the bacteria, e.g., ammonia oxidizing bacteria.

### 6. Sterilization of Product

Sterilization may be used to prepare the product, e.g., cosmetic product, e.g., finished cosmetic product for use, e.g., for sale, or for consumer use. The product may be sterilized at one or more steps in the manufacturing process.

Sterilization may be performed by irradiation or by heat. A finished cosmetic product may be provided that comprises a sterilized, e.g., irradiated, e.g., heat sterilized, cosmetic product disposed in a container. The container may be preferably a bacteria proof container, or a container that may be sealed from the outside environment.

Sterilization, e.g., heat sterilization or irradiation, may be performed on single components of the cosmetic product, a mixture of two or more components of the cosmetic product, or all of the components of the product in a mixture, to provide the cosmetic product, e.g., finished cosmetic product. Sterilization may be performed at any step during the manufacturing process. For example, sterilization may be performed prior to disposing in a container, e.g., prior to being disposed in a container for commercial release. Sterilization may be performed after disposing the cosmetic product, or one or more components of the cosmetic product in a container, e.g., in a container for commercial release. Sterilization may be performed after disposing the cosmetic product in a container, e.g., in a container for commercial release prior to or after sealing the container for commercial release.

Determination that a finished cosmetic product or cosmetic product is sterile may be measured by as described below. The finished cosmetic product may be determined to be sterile, when at least 10, 20, 30, 40, 50, 60, 70, 80, 90, 95, 99, or 99.9% of all of the bacteria, mold, fungus, or viruses are dead or incapable of cell division. The finished cosmetic product may be determined to be sterile, when all bacteria, mold, fungus, or viruses are dead or incapable of cell division. The finished cosmetic product may be determined to be sterile, when the Sterility Assurance Level (SAL) of less than about 10⁻¹, 10⁻², 10⁻³, 10⁻⁴, 10⁻⁵, 10⁻⁶, 10⁻⁷, 10⁻⁸, 10⁻⁹ is achieved. The finished cosmetic product may be determined to be sterile, when radiation induced DNA damage is sufficient to inhibit cell division. In embodiments, sterile may be considered an absolute state in which everything, e.g., all bacteria, mold, fungus, and viruses, are dead, e.g., dead in view of the limits to a given testing methodology, e.g., Sterility Assurance Level. The sterility testing may be performed as outlined in the U.S. Pharmacopeia at USP31-NF26, page 670 <1211> (Pharmacopeial Forum: Volume No. 30(5), page 1729) “Sterilization and Sterility Assurance of Compendial Articles” and USP31-NF26, page 85 <71> “Sterility Tests”, each of which is incorporated herein by reference in their entireties. The finished cosmetic product may be considered sterile, when the end-use container is unopened, e.g., the factory seal has not been broken.

Irradiation, e.g., gamma irradiation, may be performed on single components of the cosmetic product, a mixture of two or more components of the cosmetic product, or all of the components of the product in a mixture, providing the cosmetic product. Irradiation may be performed at any step during the manufacturing process. For example, irradiation may be performed prior to disposing in a container, e.g., prior to being disposed in a container for commercial release. Irradiation may be performed after disposing the cosmetic product, or one or more components of the cosmetic product in a container, e.g., in a container for commercial release. Irradiation may be performed after disposing the cosmetic product in a container, e.g., in a container for commercial release prior to or after sealing the container for commercial release.

The cosmetic product, or finished cosmetic product, may be irradiated, e.g., with ionizing radiation, e.g., with gamma rays, e.g., with x-rays, e.g., from an isotope, e.g., cobalt 60, or with ultraviolet, e.g., ultraviolet C (UVC). The cosmetic product, or finished cosmetic product may be irradiated in order to sufficiently provide a sterile product. In embodiments, as defined in Chapter 1211 of Sterilization and Sterility Assurance Compendial Articles of the U.S. Pharmacopeia (referenced in the preceeding paragraph and incorporated herein by reference in its entirety), “within the strictest definition of sterility, a specimen would be deemed sterile only when there is complete absence of viable microorganisms from it.” The sterile product may be characterized in that it is free, e.g., substantially free, from microorganisms, e.g., bacteria, e.g., fungi, capable of growth, e.g., as determined by U.S.P. 71 Sterility Testing Methods and Standards (referenced in the preceeding paragraph and incorporated herein by reference in its entirety). For example, when challenged for microorganisms capable of growth, said cosmetic product shows no growth on appropriate culture media, e.g., when said microorganisms are measured by U.S.P. 71 Sterility Testing Methods and Standards.

The cosmetic product, or finished cosmetic product, may comprise an exogenously added additive selected from an oxidant, e.g., a naturally occurring oxidant, a free radical scavenger, or a free radical quencher. Free radical scavengers or oxidants may be added to the cosmetic product as part of the sterilization procedure. The free radical scavengers or oxidants may include one or more of the following tocopherols, tocotrienols, ascorbic acid, polyphenols, isoflavones, coenzyme Q10, and other similar compounds. The free radical scavengers or oxidants may be selected from the group consisting of lipid soluble or water soluble free radical scavengers, or combinations thereof.

The cosmetic product, or said finished cosmetic product, may contain a plurality of components, and may be irradiated after mixture of the plurality of components. In embodiments, the finished cosmetic product is irradiated after said cosmetic product is disposed in the end-use container. In embodiments, the finished cosmetic product may be irradiated after closure of the end-use container. In embodiments, the finished cosmetic product may be irradiated after sealing of the end-use container. In embodiments, the finished cosmetic product is irradiated prior to closure of the end-use container. In embodiments, the finished cosmetic product, or the end-use container comprises an indicator that may indicate if said cosmetic product, or said finished cosmetic product, has been irradiated.

In embodiments, the cosmetic product may arrive to a processing facility. The product may be received by lot and product code, which allows for run generation, scheduling, processing, certification, and release of product for shipment. The product may be loaded into a carrier according to suitable conditions and configurations. Dosimeters may be placed in, around, or outside of the carrier. The product may then be exposed to irradiation, for example cobalt 60, for example, cobalt 60 source rack. Dosimeters may be analyzed after irradiation of the product is complete, to confirm that the required dose has been delivered. The dosimeters may be provided to validate the process. If the processing history is acceptable, and/or according to specifications, the product may be released and shipped for use or further distribution.

In embodiments, the irradiation may be performed as a batch process. In other embodiments, the irradiation may be performed as a continuous process. A single unit, or multiple units, e.g., packaged boxes of units may be irradiated in either a batch process or a continuous process.

In embodiments, the radiation absorbed dose is provided as kGy. For example, the radiation absorbed in order to provide sterilization may be between about 10 kGy to about 25 kGy. In embodiments, the radiation absorbed may be between about 15 kGy to about 25 kGy.

In embodiments the Sterility Assurance Level (SAL) of the finished cosmetic product may be less than about 10⁻¹, 10⁻², 10⁻³, 10⁻⁴, 10⁻⁵, 10⁻⁶, 10⁻⁷, 10⁻⁸, 10⁻⁹, achieved through the conditions described herein. In embodiments, a SAL of 10⁻³ or greater may be achieved.

The time of exposure is provided depending on the density of the product being irradiated. For example, the time of exposure for a single unit of product, may be less than the time of exposure of multiple units of product, due to differences in density.

In embodiments, heat sterilization may be utilized. The cosmetic product or finished cosmetic product may be heated, e.g., by microwave oven or autoclave. The heating may be sufficient to provide a sterile product. The sterile product may be characterized in that it is substantially free from microorganisms, e.g., bacteria, e.g., fungi capable of growth, consistent with U.S.P. Chapter 1211, as determined by U.S.P. 71 Sterility Testing Methods and Standards, each of which is referenced above and incorporated herein in their entireties. The cosmetic product or finished cosmetic product may be determined to be sterile, when challenged for microorganisms capable of growth, said cosmetic product shows no growth e.g., when said microorganisms are measured by a testing methodology described herein.

In embodiments, the cosmetic product, or finished cosmetic product may contain a plurality of components and is heated after mixture of the plurality of components. In embodiments the cosmetic product or finished cosmetic product is heated after the cosmetic product is disposed in the end-use container. In embodiments, the finished cosmetic product is heated after closure of the end-use container. In embodiments, the finished cosmetic product is heated after sealing the end-use container. In embodiments, the finished cosmetic product is heated prior to closure of the end-use container. In embodiments, the finished cosmetic product or the end-use container comprises an indicator that indicates if said cosmetic product, or said finished cosmetic product, has been heated. In embodiments, the cosmetic product, or finished cosmetic product is heated at or above 121 degrees Celsius for at least 15 minutes during or after formulation, e.g., after mixing, or filling, e.g., after disposing, or after sealing the end-use container. The cosmetic product, or finished cosmetic product may not be heated above 140 degrees F. during or after formulation, e g., mixing, or filling, e.g., after disposing, or after sealing the end-use container. The may be due to the end-use container and/or the contents of the end-use container, e.g., the cosmetic product undergoing degradation above a temperature of 140 degrees F.

### 7. Methods of Manufacturing a Cosmetic Product, e.g., Finished Cosmetic Product

Methods of making a finished cosmetic product are provide that may comprise disposing a cosmetic product in an end-use container to form a filled end-use container. The filled end-use container may then be treated to kill or inactivate bacteria, which may then provide the finished cosmetic product. The killing or inactivation of bacteria may be accomplished through a sterilization technique described herein.

Other methods may be provided, that allow for manufacturing or making of a biome-friendly cosmetic product. The method may comprise selecting a first component, e.g., a cleanser or surfactant, from a list of biome-friendly components; selecting a second component, e.g., a viscosity modifier, from a list of biome-friendly components; and providing a mixture of the first and second components (or more), thereby making a biome-friendly cosmetic product.

The method may further comprise selecting a third component, e.g., a humectants, from a list of biome-friendly components. The method may further comprise selecting a fourth component, e.g., a fragrance, from a list of biome-friendly components. Additional components that may be selected include, e.g., fifth, sixth, and seventh components, e.g., conditioner, lipid layer enhancers, and emulsifiers.

Once the mixture has been obtained, treatment of the mixture, e.g., sterilization may be accomplished, before or after sealing the cosmetic product in an end-use container, in order to

Methods may also be provided where a first and second component is provided, wherein each has been show to be biome-friendly. The method may comprise combining the first and second component to form a mixture. Determination if the mixture is biome-friendly may be accomplished through the methods disclosed herein, thereby making a finished cosmetic product.

Further methods may be provided of manufacturing, e.g., evaluating a cosmetic product or a finished cosmetic product. The method may comprise providing a product having at least 2 components selected from Table 3. The method may comprise acquiring an evaluation of whether the cosmetic product is safe for bacteria, e.g., beneficial bacteria, e.g., ammonia oxidizing bacteria on the skin of the user, thereby manufacturing the cosmetic product or finished cosmetic product. The evaluation may comprise evaluating the viability of an ammonia oxidizing bacteria, as disclosed herein, or of the ability of the ammonia oxidizing bacteria to produce nitrite after contact with the cosmetic product, or one or more components of the cosmetic product.

### 8. Testing Cosmetic Product, e.g., a Finished Cosmetic Product, or a Component Thereof

A test may be performed in order to confirm that the product, e.g., cosmetic product, e.g., finished cosmetic product, or a component thereof is free of microorganisms, e.g., bacteria, e.g. fungus.

A method of evaluating a product, e.g., a cosmetic product, or a finished cosmetic product, or a component thereof, may comprise contacting a portion, e.g., an aliquot of the product with a test organism, e.g., ammonia oxidizing bacteria, and evaluating the effect of the cosmetic product on the test organism. Evaluating may comprise evaluating the effect of the cosmetic product on the ability of the test organism, e.g., ammonia oxidizing bacteria, to produce nitrite.

Evaluating the product may comprise determining if the ability of the ammonia oxidizing bacteria to produce nitrite meets a preselected criteria, e.g., at least has the ability to recover so as to produce nitrite over a given period of time as shown in the Figures of the Examples disclosed herein.

Evaluating the product may provide for the finished cosmetic product to be identified as “tested and confirmed as biome-friendly” or “tested and confirmed as bio-compatible.”

Evaluating the product may provide for the finished cosmetic product to be identified as “tested and confirmed to have the live test organisms.” Evaluating may provide for the finished cosmetic product to be identified as “tested and confirmed to have live ammonia oxidizing bacteria.”

Methods may be provided to select an ammonia oxidizing bacteria-friendly ingredient. The method may comprise obtaining an ammonia oxidizing bacteria (AOB) cell suspension, e.g., from a continuous culture system. The AOB cells may be harvested from the cell suspension, and the cells may be washed in a storage solution. Harvesting the AOB cells from the cell suspension may comprise centrifuging the cell suspension. The storage solution may comprise 50 mM Na₂HPO₄— 2 mM MgCl₂, pH 7.6. The AOB cells may be suspended in the storage solution at a final optical density (OD₆₀₀) of 5.0 (˜10¹⁰ cells/ml), and the cells may be stored at about 4° C. The AOB cells may be diluted to a final optical density (OD₆₀₀) of 0.5 (˜10⁹ cells/ml) in 10 ml of AOB medium supplemented with ammonium (NH₄⁺), for example about 50 mM ammonium containing a product, e.g., a cosmetic product, or component, e.g., excipient, such as those listed in Table 3, or any other excipient desired, at a pre-determined final concentration, and incubating for a first pre-determined time period to provide an incubated culture. The incubation may occur at about 30° C. An aliquot of the incubated culture may be collected, and the concentration of nitrite in the supernatant of the incubated culture may be measured. The supernatant may be obtained through centrifugation of the culture to provide the supernatant and the bacterial pellet.

The AOB medium disclosed herein may be supplemented with ammonium (NH₄⁺), for example about 1, 5, 10, 20, 30, 40, 50, 60, 70, 80, 90, 100, 120, 140, 160, 180, 200, 250, 300, 350, 400, 450, 500 mM ammonium, or higher concentrations.

Based on the nitrite measurement, either one measurement or more than one measurement, over a period of time, product or the component, e.g., excipient, may be identified as an ammonia oxidizing bacteria-friendly ingredient based on the concentration of nitrite in the supernatant of the incubated culture.

The method may further comprise washing the bacterial pellet in the AOB medium, suspending the bacterial in the AOB medium supplemented with NH₄⁺. The method may further comprise incubating the bacterial pellet in the AOB medium supplemented with NH₄⁺, and recovering AOB cells at a second pre-determined time period to provide recovered AOB cell samples. The recovered AOB cell sample may be measured for at least one of an OD₆₀₀ value and nitrite accumulation.

Based on at least one of the OD₆₀₀ value and nitrite accumulation in the recovered AOB cell sample, the product or component, e.g, excipient may be identified as an ammonia oxidizing bacteria-friendly ingredient.

In embodiments, a method of selecting an ammonia oxidizing bacteria-friendly ingredient is provided comprising obtaining a sample of ammonia oxidizing bacteria (AOB), and contacting the with a product or component, e.g., an excipient for a first pre-determined time period to provide an incubated culture. The method may further comprise collecting an aliquot of the incubated culture, and measuring a concentration of nitrite in a supernatant of the incubated culture. The method may further comprise identifying the product or component, e.g., excipient as an ammonia oxidizing bacteria-friendly ingredient based on the concentration of nitrite in the supernatant of the incubated culture.

The method may further comprise contacting the AOB from the incubated culture with NH₄⁺, and measuring the recovered AOB cell sample for at least one of an OD₆₀₀ value and nitrite accumulation after a second pre-determined time period. The method may further comprise identifying the product or component, e.g., excipient as an ammonia oxidizing bacteria-friendly ingredient based on at least one of the OD₆₀₀ value and nitrite accumulation in the recovered AOB cell sample.

The pre-determined final concentration of product or component, e.g., excipient may be between about 0% and about 100%. The first pre-determined time period may be at least one of about 1 minute, about 10 minutes, about 60 minutes, about 2 hours, about 4 hours, about 6 hours, about 12 hours, and about 24 hours. The second pre-determined time period is at least one of about 1 minute, about 10 minutes, about 60 minutes, about 2 hours, about 4 hours, about 6 hours, about 12 hours, about 24 hours, about 36 hours, about 48 hours, about 72 hours, about 96 hours.

Depending on the ingredient, excipient, or composition tested, the concentration of nitrite measured may allow for identification of a biome-friendly ingredient, excipient, or composition, e.g., an ammonia oxidizing bacteria-friendly ingredient, excipient, or composition. In certain embodiments, nitrite production measured may be at or above a certain value of nitrite production in order to qualify as a biome-friendly ingredient, excipient, or composition. This nitrite production concentration may be measured at a certain period of time after contacting the AOB with the incubated culture with NH₄⁺. The period of time may be at 1 minute, 5, 10, 20, 30, 40, 50, 60, 120, minutes. The period of time may be at 3, 4, 5, 10, 15, 20, 24, 36, 48, 72, or 96 hours, or more.

The nitrite production may be at or above a certain value, e.g., at or above 10 micromolar, 20, 50, 100, 200, 300, 400, 500, 600, 700, 800, 900, 1000, 1500, 2000, 3000 micromolar nitrite concentration, or more, in order for the ingredient, excipient, or composition to be identified as a biome-friendly ingredient.

In embodiments, a nitrite production of greater than 1000 micromolar, measured at the end of a 48 hour period would be indicative of a biome-friendly ingredient, excipient, or composition. In other embodiments, nitrite production of greater than 100 micromolar, measured at the end of a 48 hour period would be indicative of a biome-friendly ingredient, excipient, or composition. In other embodiments, nitrite production of greater than 10 micromolar, measured at the end of a 48 hour period would be indicative of a biome-friendly ingredient, excipient, or composition.

Methods of producing a biome-friendly composition may comprise acquiring knowledge that a compound, or more than one compound is friendly to ammonia oxidizing bacteria, and packaging the compound, or more than one compound in an end-use container, to provide a product, e.g., a cosmetic product, e.g., a finished cosmetic product.

Methods of producing a biome-friendly composition may comprise acquiring knowledge that a compound is friendly to ammonia oxidizing bacteria, and combining the compound with ammonia oxidizing bacteria to provide a cosmetic product.

### 9. Method of Distributing the Product

Methods described herein provide for methods of distributing biome-friendly finished cosmetic products. In some embodiments, a finished cosmetic product has a predetermined useful life, after which the danger of bacterial contamination is unacceptable. Methods of the disclosure provide an end-user or subscriber with periodic notice that a product is near or at the end of its recommended useful life, or provide a new unit of the finished cosmetic product. Optionally, the method includes a “recycling” function.

A method of distributing a finished cosmetic product may comprise supplying, or causing a designee to supply, an end-user with a first unit of a finished cosmetic product. The method may further comprise providing, or causing a designee to provide the end-user or an entity designated by the end-user, for example, a second end-user with one or both of the following: i) a subsequent unit of said finished cosmetic product, or a unit of a second finished cosmetic product; and/or ii) a notification that said first unit has reached the end of its recommended life. The method may further comprise, optionally, providing, or causing a designee to provide, the end-user or an entity designated by the end-user, e.g, a second end-user, with information regarding disposal, e.g., recycling, the first unit of finished cosmetic product, e.g., a finished cosmetic product on which the recommended life has elapsed. This may allow distribution of a finished cosmetic product.

The method may further comprise supplying an end-user with a first unit of finished cosmetic product, and providing the end-user with a subsequent unit of the finished cosmetic product, or a notification that the first unit has reached the end of its recommended life. The method may further comprise providing the end-user, with information regarding disposal, e.g., recycling, said first unit of finished cosmetic product, e.g., a finished cosmetic product on which the recommended life has elapsed, thereby distributing a finished cosmetic product.

The first unit of the finished cosmetic product may comprise a cosmetic product as described herein. The first unit of finished cosmetic product may comprise a cosmetic product that is free, or substantially free, of bacteria or fungi. The first unit of finished cosmetic product may comprise a cosmetic product that is free, or substantially free, of preservative.

The first unit of finished cosmetic product may comprise a cosmetic product selected from, or include, or be disposed in any one or more of a baby product, e.g., a baby shampoo, a baby lotion, a baby oil, a baby powder, a baby cream; a bath preparation, e.g., a bath oil, a tablet, a salt, a bubble bath, a bath capsule; an eye makeup preparation, e.g., an eyebrow pencil, an eyeliner, an eye shadow, an eye lotion, an eye makeup remover, a mascara; a fragrance preparation, e.g., a colognes, a toilet water, a perfume, a powder (dusting and talcum), a sachet; hair preparations, e.g., hair conditioners, hair sprays, hair straighteners, permanent waves, rinses, shampoos, tonics, dressings, hair grooming aids, wave sets; hair coloring preparations, e.g., hair dyes and colors, hair tints, coloring hair rinses, coloring hair shampoos, hair lighteners with color, hair bleaches; makeup preparations, e.g., face powders, foundations, leg and body paints, lipstick, makeup bases, rouges, makeup fixatives; manicuring preparations, e.g., basecoats and undercoats, cuticle softeners, nail creams and lotions, nail extenders, nail polish and enamel, nail polish and enamel removers; oral hygiene products, e.g., dentrifices, mouthwashes and breath fresheners; bath soaps, e.g., foaming body cleansers, and detergents, deodorants, douches, feminine hygiene deodorants; shaving preparations, e.g., aftershave lotions, beard softeners, talcum, preshave lotions, shaving cream, shaving soap; skin care preparations, e.g., cleansing, depilatories, face and neck, body and hand, foot powders and sprays, moisturizing, night preparations, paste masks, skin fresheners; and suntan preparations, e.g., gels, creams, and liquids, and indoor tanning preparations.

In embodiments, the first unit of finished cosmetic product may comprise a shampoo. In embodiments, the first unit of finished cosmetic product may comprise a foaming body cleanser. In embodiments, the first unit of finished cosmetic product, may comprise a conditioner.

The methods may provide for supplying the end-user with, e.g., by sale, or gifting, the first unit of a finished cosmetic product from an internet-based outlet. The methods may provide for supplying the end-user with, e.g., by sale, or gifting, said first unit of a finished cosmetic product from a non-internet-based outlet, e.g., a store.

Provision of a second end-user with one or both of the following: i) a subsequent unit of said finished cosmetic product, or a unit of a second finished cosmetic product; and/or ii) a notification that said first unit has reached the end of its recommended life may be made within a preselected number of days, for example, after supply of the first unit of a finished cosmetic product, or prior to the end of the recommended life of the first unit of a finished cosmetic product or prior to the first expected use of the product, or when the first unit has reached a reduced level of activity as determined, for example by a biosensor.

The preselected number of days may be calculated by e-mail, an electronic application, e.g., a smartphone application, or a Bluetooth weight sensor built into a bottle “dock”.

The preselected number of days may be about 7 days, 14, days, 21 days, 28 days, 35 days, or 42 days, or between about 5-7 days, 8-11 days, 12-15 days, 16-19 days, 20-23 days, 24-27 days, 28-31 days, 32-35 days, 36-39 days, 40-43 days.

The second unit, or subsequent unit of finished cosmetic product may comprise a cosmetic product described herein. The subsequent unit of finished cosmetic product may comprise a cosmetic product that may be free, or substantially free, of bacteria or fungi. The subsequent unit of finished cosmetic product may comprise a cosmetic product that is free, or substantially free, of preservative.

The subsequent unit of finished cosmetic product may comprise a cosmetic product selected from, or include, or be disposed in any one or more a baby product, e.g., a baby shampoo, a baby lotion, a baby oil, a baby powder, a baby cream; a bath preparation, e.g., a bath oil, a tablet, a salt, a bubble bath, a bath capsule; an eye makeup preparation, e.g., an eyebrow pencil, an eyeliner, an eye shadow, an eye lotion, an eye makeup remover, a mascara; a fragrance preparation, e.g., a colognes, a toilet water, a perfume, a powder (dusting and talcum), a sachet; hair preparations, e.g., hair conditioners, hair sprays, hair straighteners, permanent waves, rinses, shampoos, tonics, dressings, hair grooming aids, wave sets; hair coloring preparations, e.g., hair dyes and colors, hair tints, coloring hair rinses, coloring hair shampoos, hair lighteners with color, hair bleaches; makeup preparations, e.g., face powders, foundations, leg and body paints, lipstick, makeup bases, rouges, makeup fixatives; manicuring preparations, e.g., basecoats and undercoats, cuticle softeners, nail creams and lotions, nail extenders, nail polish and enamel, nail polish and enamel removers; oral hygiene products, e.g., dentrifices, mouthwashes and breath fresheners; bath soaps, e.g., foaming body cleansers, and detergents, deodorants, douches, feminine hygiene deodorants; shaving preparations, e.g., aftershave lotions, beard softeners, talcum, preshave lotions, shaving cream, shaving soap; skin care preparations, e.g., cleansing, depilatories, face and neck, body and hand, foot powders and sprays, moisturizing, night preparations, paste masks, skin fresheners; and suntan preparations, e.g., gels, creams, and liquids, and indoor tanning preparations.

In embodiments, the subsequent unit of finished cosmetic product may comprise a shampoo. In embodiments, the subsequent unit of finished cosmetic product may comprise a foaming body cleanser. In embodiments, the subsequent unit of finished cosmetic product may comprise a conditioner.

The subsequent unit of the finished cosmetic product, or the second finished cosmetic product may be delivered by any suitable means to provide the end-user with the product. For example, the product may be delivered by mail, or by a commercial delivery entity, to said end-user.

The subsequent unit of said finished cosmetic product, or a unit of a second finished cosmetic product, may be delivered, e.g., by mail or a commercial delivery entity, to an entity, e.g., a second end user, designated by said end-user.

The subsequent unit of said finished cosmetic product, or a unit of a second finished cosmetic product, may be delivered, e.g., by mail or a commercial delivery entity, to a location designated by said end-user.

The notification may be delivered, e.g., by mail or a commercial delivery entity, or electronically, e.g., by internet or telephone, e.g., by telephone call, e.g., by recorded message, or text message to said end-user. The notification, may be delivered, e.g., by mail or a commercial delivery entity, or electronically, e.g., by internet or telephone, e.g., by telephone call, e.g., by recorded message, or text message, to an entity, e.g., a second end user, designated by said end-user.

The notification, may be delivered, e.g., by mail or a commercial delivery entity, or electronically, e.g., by internet or telephone, e.g., by recorded message, or text message, to a location designated by said end-user.

Options for recycling the finished cosmetic product may be provide. The method may comprise providing (or causing a designee to provide) the end-user or an entity designated by said end user, e.g., a second end-user, with information regarding disposal, e.g., recycling, of said first unit of finished cosmetic product, e.g., a finished cosmetic product on which the recommended life has elapsed.

The information may comprise the name or location, e.g., address, of an entity (a collection entity) which will accept said first unit of finished cosmetic product, e.g., after its recommended life.

The method may further comprise providing the end-user, or designee, with a container configured to receive the first unit of finished cosmetic product, e.g., after its recommended life. The container configured to receive the first unit of finished cosmetic product may be provided with the first unit of finished cosmetic product. This container may be provided with the notification. The container may comprise a mailing label addressed to the collection agency, e.g., recycler.

Methods may also comprise providing (or causing a designee to provide) the end-user or an entity designated by said end user, e.g., a second end-user with information, as described above with regard to the first unit, regarding disposal, e.g., recycling, of said subsequent unit of finished cosmetic product, e.g., a finished cosmetic product, or cosmetic product, on which the recommended lifetime has elapsed.

Method of obtaining a finished cosmetic product may also be provided and may comprise receiving a first unit of finished cosmetic product. The method of obtaining may further comprise receiving: i) a subsequent unit of said finished cosmetic product, or a unit of a second finished cosmetic product; and/or ii) a notification that said first unit has reached the end of its recommended life. The method of obtaining may further comprise, optionally, receiving information regarding disposal, e.g., recycling, said first unit of finished cosmetic product, e.g., a finished cosmetic product on which the recommended life has elapsed, thereby obtaining a finished cosmetic product.

Further methods of distributing a finished cosmetic product are provided. The methods may comprise obtaining, e.g., manufacturing, a finished cosmetic product of any of the above claims, said finished cosmetic product comprising a cosmetic product, e.g., make-up, e.g., eyeliner, disposed in an end use container. The method may comprise communicating to an end user of said finished product, one or more of the following: the finished cosmetic product, or cosmetic product, is biome-friendly, e.g., biome-compatible; the finished cosmetic product, or cosmetic product, should not be used after an indicated expiration date, or lifetime, e.g., recommended lifetime, based on, e.g., deterioration, e.g., biome-compatibility.

The indicated expiration date or lifetime, e.g., recommended lifetime may be expressed as described herein. For example, the expiration date may be expressed: (a) in units of time, e.g., days, from a preselected event, e.g, unsealing of said finished cosmetic product or a first use of said finished cosmetic product; and/or (b) as the number of uses or applications; and/or the finished cosmetic product, or cosmetic product, should not be used after X applications; and/or the finished cosmetic product, or cosmetic product, should not be used after X days of use, e.g., X days of Y/day use, wherein X is between about (7 days) one week and about 42 days (6 weeks), and Y is between about zero uses per day and about ten uses per day. For example X days may be about 7-10, 10-13, 14-17, 18-21, 22-25, 26-29, 30-33, 34-37, 38-42 days; and Y may be about 0-1, 2-4, 5-7, 8-10 uses per day. The method may further comprise shipping said finished cosmetic product.

### 10. Use of Product in Conjunction with Ammonia Oxidizing Bacteria

The products disclosed herein may be used in conjunction with application of a non-pathogenic bacteria, e.g., ammonia oxidizing bacteria to the subject, e.g., user of non-pathogenic bacteria, e.g., ammonia oxidizing bacteria.

Methods of maintaining ammonia oxidizing bacteria (AOB) on a subject may comprise applying a cosmetic product or a finished cosmetic product as described throughout this disclosure. The method may further comprise applying a preparation comprising AOB to the subject prior to applying the cosmetic product or the finished cosmetic product. The method may comprise applying a preparation comprising AOB to the subject subsequent to applying the cosmetic product or the finished cosmetic product.

The method may further comprise applying the preparation comprising AOB to the subject prior to applying the cosmetic product or the finished cosmetic product, wherein the preparation comprising AOB is applied between about one of the following ranges: about 1-5, 5-10, 10-20, 20-30. 30-40, 40-50, 50-60 minutes, 2-5, 5-10, 10-15, 15-20, 20-25 hours, 2-5, 5-10, 10-15, days, 3-4, 5-10 weeks prior to applying the cosmetic product or the finished cosmetic product.

The method may further comprise applying the preparation comprising AOB to the subject subsequent to applying the cosmetic product or the finished cosmetic product, wherein the preparation comprising AOB is applied between about one of the following ranges: about 1-5, 5-10, 10-20, 20-30. 30-40, 40-50, 50-60 minutes, 2-5, 5-10, 10-15, 15-20, 20-25 hours, 2-5, 5-10, 10-15, days, 3-4, 5-10 weeks subsequent to applying the cosmetic product or the finished cosmetic product.

The method may comprise not applying a non biome-friendly cosmetic product or finished cosmetic product prior to or subsequent.

In the method and system embodiments of this disclosure, at least one of the preparation of AOB and the cosmetic product, e.g., finished cosmetic product is applied to a pre-defined area of the subject. The pre-defined area of the subject may be at least one of a portion of a head, e.g., a face, cheek, chin, eyelid, lip, nose, scalp, hair, forehead; neck; underarm; arm; hand; leg; foot; chest; abdomen region; buttocks; genital area; and back.

The ammonia oxidizing bacteria referred to herein is described in the following sections.

### 11. Ammonia Oxidizing Bacteria (AOBs)

Autotrophic ammonia oxidizing bacteria, which may be referred to herein as AOBs or AOB, are obligate autotrophic bacteria as noted by Alan B. Hooper and A. Krummel at al. Alan B. Hooper, Biochemical Basis of Obligate Autotrophy in Nitrosomonas europaea, Journal of Bacteriology, February 1969, p. 776-779. Antje Krummel et al., Effect of Organic Matter on Growth and Cell Yield of Ammonia-Oxidizing Bacteria, Arch Microbiol (1982) 133: 50-54. These bacteria derive all metabolic energy only from the oxidation of ammonia to nitrite with nitric oxide (NO) as an intermediate product in their respiration chain and derive virtually all carbon by fixing carbon dioxide. They are incapable of utilizing carbon sources other than a few simple molecules.

Ammonia oxidizing bacteria (AOB) are widely found in the environment, and in the presence of ammonia, oxygen and trace metals will fix carbon dioxide and proliferate. AOB may be slow growing and toxic levels of ammonia may kill fish and other organisms before AOB can proliferate and reduce ammonia to non-toxic levels. Slow growth of AOB also may delay the health benefits of the NO and nitrite the AOB produce when applied to the skin.

Supplementing the aquarium, skin, or process with sufficient viable AOB grown and stored for that purpose is desired. AOB do not form spores, so storage in the dry state with high viability is difficult, and storage in the wet state leaves them metabolically active.

Decay of nitrifying capacity during storage of AOB for wastewater treatment has been studied, as for example (Munz G, Lubello C, Oleszkiewicz J A. Modeling the decay of ammonium oxidizing bacteria. Water Res. 2011 January; 45(2): 557-64. Oi: 10.1016/j.watres.2010.09.022.)

Growth, prolonged storage, and restoration of activity of Nitrosomonas is discussed by Cassidy et al. (U.S. Pat. No. 5,314,542) where they disclose growing Nitrosomonas, removing toxic waste products, storing in sterile water of appropriate salinity for periods of time up to one year, and then reviving by adding buffer (CaCO₃) and 200 ppm, of ammonium, which reviving takes 72 hours.

The present disclosure provides that if AOB are kept under conditions of low carbon dioxide but with sufficient oxygen and ammonia, where they accumulate polyphosphate for a period of about one doubling time (˜10 hours), then they accumulate sufficient polyphosphate to greatly extends their storage viability, storage time and accelerate their revival both with and without addition of buffer and ammonia.

The presence of sufficient stored polyphosphate allows AOB the ATP resources to maintain metabolic activity even in the absence of ammonia and oxygen, and to survive insults that would otherwise be fatal.

As obligate autotrophs, AOB synthesize protein via the fixing of CO₂ using the energy and reducing equivalents generated by the oxidation of ammonia to nitrite. Growth requires ammonia, oxygen, minerals and carbon dioxide.

Nitrosomonas may exist in several metabolic states, according to “Polyphosphate and Orthophosphate Content of Nitrosomonas europaea as a Function of Growth” by K. R. Terry and A. B. Hooper, Journal of Bacteriology, July 1970, p. 199-206, Vol. 103, No. I.

The AOBs contemplated in this disclosure may comprise mutations relative to wild-type AOBs. These mutations may, e.g., occur spontaneously, be introduced by random mutagenesis, or be introduced by targeted mutagenesis. For instance, the AOBs may lack one or more genes or regulatory DNA sequences that wild-type AOBs typically comprises. The AOBs may also comprise point mutations, substitutions, insertions, deletions, and/or rearrangements relative to the sequenced strain or a wild-type strain. The AOBs may be a purified preparation of optimized AOBs.

In certain embodiments, the AOBs are transgenic. For instance, it may comprise one or more genes or regulatory DNA sequences that wild-type ammonia oxidizing bacteria lacks. More particularly, the ammonia oxidizing bacteria may comprise, for instance, a reporter gene, a selective marker, a gene encoding an enzyme, or a promoter (including an inducible or repressible promoter). In some embodiments the additional gene or regulatory DNA sequence is integrated into the bacterial chromosome; in some embodiments the additional gene or regulatory DNA sequence is situated on a plasmid.

In some preferred embodiments, the AOBs differ by at least one nucleotide from naturally occurring bacteria. For instance, the AOBs may differ from naturally occurring bacteria in a gene or protein that is part of a relevant pathway, e.g., an ammonia metabolism pathway, a urea metabolism pathway, or a pathway for producing nitric oxide or nitric oxide precursors. More particularly, the AOBs may comprise a mutation that elevates activity of the pathway, e.g., by increasing levels or activity of an element of that pathway.

The above-mentioned mutations can be introduced using any suitable technique. Numerous methods are known for introducing mutations into a given position. For instance, one could use site-directed mutagenesis, oligonucleotide-directed mutagenesis, or site-specific mutagenesis. Non-limiting examples of specific mutagenesis protocols are described in, e.g., Mutagenesis, pp. 13.1-13.105 (Sambrook and Russell, eds., Molecular Cloning A Laboratory Manual, Vol. 3, 3.sup.rd ed. 2001). In addition, non-limiting examples of well-characterized mutagenesis protocols available from commercial vendors include, without limitation, Altered Sites® II in vitro Mutagenesis Systems (Promega Corp., Madison, Wis.); Erase-a-Base® System (Promega, Madison, Wis.); GeneTailor™ Site-Directed Mutagenesis System (Invitrogen, Inc., Carlsbad, Calif.); QuikChange® II Site-Directed Mutagenesis Kits (Stratagene, La Jolla, Calif.); and Transformer™ Site-Directed Mutagenesis Kit (BD-Clontech, Mountain View, Calif.).

In some embodiments of the disclosure, the ammonia oxidizing bacteria may be in the form of a preparation. The ammonia oxidizing bacteria may be used in conjunction or in combination with the cosmetic products of the present disclosure. For example, AOBs or a preparation of AOBs may be in the form of a product that is separate from the cosmetic product, or may be provided together with the cosmetic product, e.g., in the same or different end-use container.

The AOB may be axenic. The preparation, e.g., formulation, e.g., composition of ammonia oxidizing bacteria may comprise, consist essentially of, or consist of axenic ammonia oxidizing bacteria. The ammonia oxidizing bacteria may be from a genus selected from the group consisting of Nitrosomonas, Nitrosococcus, Nitrosospira, Nitrosocystis, Nitrosolobus, Nitrosovibrio, and combinations thereof.

In some embodiments, the preparation of ammonia oxidizing bacteria may comprise a concentration or amount of ammonia oxidizing bacteria in order to at least partially treat a condition or disease. The preparation of ammonia oxidizing bacteria may comprise a concentration or amount of ammonia oxidizing bacteria in order to alter, e.g., reduce or increase, an amount, concentration or proportion of a bacterium, or genus of bacteria, on a surface, e.g., a skin surface. The bacteria may be non-pathogenic or pathogenic, or potentially pathogenic.

In some embodiments, the preparation of ammonia oxidizing bacteria may comprise between about 10⁸ to about 10¹⁴ CFU/L. The preparation may comprise at least 10⁸, 10⁹, 10¹⁰, 10¹¹, 2×10¹¹, 5×10¹¹, 10¹², 2×10¹², 5×10¹², 10¹³, 2×10¹³, 5×10¹³, or 10¹⁴; or about 10⁸-10⁹, 10⁹-10¹⁰, 10¹⁰40¹¹, 10¹¹-10¹², 10¹²-10¹³, or 10¹³-10¹⁴ CFU/L. In some embodiments, the preparation may comprise at least 10⁸, 10⁹, 10¹⁰, 10¹¹, 2×10¹¹, 5×10¹¹, 10¹², 2×10¹², 5×10¹², 10¹³, 2×10¹³, 5×10¹³, or 10¹⁴; or about 10⁸-10⁹, 10⁹40¹⁰, 10¹⁰40¹¹, 10¹¹-10¹², 10¹²-10¹³, or 10¹³-10¹⁴ CFU/ml.

In certain aspects, the preparation may comprise between about 1×10⁹ CFU to about 10×10⁹ CFU. In certain aspects, the preparation may comprise between about 1×10⁹ CFU/L to about 10×10⁹ CFU/L.

In some embodiments, the preparation of ammonia oxidizing bacteria may comprise between about 0.1 milligrams (mg) to about 1000 mg of ammonia oxidizing bacteria. In certain aspects, the preparation may comprise between about 50 mg and about 1000 mg of ammonia oxidizing bacteria. The preparation may comprise between about 0.1-0.5 mg, 0.2-0.7 mg, 0.5-1.0 mg, 0.5-2 mg, 0.5-5 mg, 2.5-5 mg, 2.5-7.0 mg, 5.0-10 mg, 7.5-15 mg, 10-15 mg, 15-20 mg, 15-25 mg, 20-30 mg, 25-50 mg, 25-75 mg, 50-75 mg, 50-100 mg, 75-100 mg, 100-200 mg, 200-300 mg, 300-400 mg, 400-500 mg, 500-600 mg, 600-700 mg, 700-800 mg, 800-900 mg, 900-1000 mg, 100-250 mg, 250-500 mg, 100-500 mg, 500-750 mg, 750-1000 mg, or 500-1000 mg.

In some embodiments, the preparation of ammonia oxidizing bacteria may comprise a mass ratio of ammonia oxidizing bacteria to an excipient, e.g., a pharmaceutically acceptable excipient or a cosmetically acceptable excipient in a range of about 0.1 grams per liter to about 1 gram per liter. The preparation may comprise a mass ratio of ammonia oxidizing bacteria to an excipient in a range of about 0.1-0.2, 0.2-0.3, 0.1-0.5, 0.2-0.7, 0.5-1.0, or 0.7-1.0 grams per liter.

In some embodiments, the preparation of ammonia oxidizing bacteria may comprise, consist essentially of, or consist of ammonia oxidizing bacteria in a buffer solution comprising, consisting essentially of, or consisting of disodium phosphate and magnesium chloride, for example, 50 mM Na₂HPO₄ and 2 mM MgCl₂.

The preparation may comprise a volume of between about 0.1 and about 100 fluid ounces, about 0.2 and about 50 fluid ounces, about 0.5 and about 25 fluid ounces, about 1.0 and about 10 fluid ounces, about 2.0 and about 7 fluid ounces, about 3 and about 5 fluid ounces. In some embodiments, the preparation may comprise a volume of about 3.4 fluid ounces.

The preparation may be provided in a container constructed to contain between about 0.1 and about 100 fluid ounces, about 0.2 and about 50 fluid ounces, about 0.5 and about 25 fluid ounces, about 1.0 and about 10 fluid ounces, about 2.0 and about 7 fluid ounces, about 3 and about 5 fluid ounces. In some embodiments, the preparation is a container constructed to contain about 3.4 fluid ounces. The container may be a one-chamber container, or any other container disclosed herein.

In some embodiments, the preparation of ammonia oxidizing bacteria may be in a growth state. A growth state may be provided by exposing ammonia oxidizing bacteria to an environment that may promote growth. The growth state may be a state, e.g., ammonia oxidizing bacteria in an environment that allows immediate availability of ammonia oxidizing bacteria to convert ammonium ions (NH₄⁺) to nitrite (NO₂⁻). The growth state may comprise providing ammonia oxidizing bacteria in an environment having a pH of greater than about 7.6. The growth state may also comprise providing ammonia oxidizing bacteria in an environment having ammonia, ammonium ions, and/or urea, trace minerals and sufficient oxygen and carbon dioxide, as described in Section 1.

In some embodiments, the preparation of ammonia oxidizing bacteria may be in a polyphosphate loading state, wherein the state or the environment, e.g., a media, e.g., a culture media, e.g., a growth media, may have a pH of less than about 7.4. Levels of at least one of ammonia, ammonium ions, and urea may be between about 10 micromolar and 200 millimolar. Levels of trace materials may be between 0.1 micromolar iron and 20 micromolar iron. Levels of oxygen may be between about 5% and 100% oxygen saturation. Levels of carbon dioxide may be between/less than about zero and 200 ppm, and phosphate levels greater than about 10 micromolar. The purpose of the polyphosphate loading state is to provide AOB with ammonia and oxygen such that ATP can be produced, but to deny them carbon dioxide and carbonate such that they are unable to use that ATP to fix carbon dioxide and instead use that ATP to generate polyphosphate which may be stored.

In some embodiments, the preparation of ammonia oxidizing bacteria may be in a storage state. A storage state may be defined as ammonia oxidizing bacteria in an environment in which they may be stored to be later revived. The storage state may be a state, e.g., ammonia oxidizing bacteria in an environment that allows availability of ammonia oxidizing bacteria after being revived, e.g., after being place in an environment promoting a growth state for a pre-determined period of time. The pre-determined period of time for revival may be less than 72 hours. For example, the pre-determined period of time may be less than about 75 hours, or less than about 72 hours. The pre-determined period of time may at least partially based on a period time of about 0.2-10 times, 0.3-5 times, 0.5-3 times, 0.5-1.5 times, or 0.5 to 1 times the doubling time for the ammonia oxidizing bacteria. The pre-determined period of time may be at least partially based on a period of time of about one doubling time for the ammonia oxidizing bacteria. The pre-determined period of time may be between about 8 hours and 12 hours. The pre-determined period of time may be about 10 hours. The pre-determined time may be less than about 75 hours, 72 hours, 70 hours, 68 hours, 65 hours, 60 hours, 55 hours, 50 hours, 45 hours, 40 hours, 35 hours, 30 hours, 25 hours, 20 hours, 15 hours, 10 hours, 5 hours, 4 hours, 3, hours, 2 hours, or 1 hour. The pre-determined period of time may be between about 5 minutes and 5 hours. The pre-determined period of time may be about 5-10 minutes, 10-15 minutes, 15-20 minutes, 20-25 minutes, 25-30 minutes, 30-45 minutes, 45-60 minutes, 60 minutes-1.5 hours, 1.5 hours-2 hours, 2 hours-2.5 hours, 2.5 hours-3 hours, 3 hours-3.5 hours, 3.5 hours-4 hours, 4 hours-4.5 hours, 4.5 hours-5 hours. In some embodiments, the pre-determined period of time may be about 2 hours.

The storage state may comprise providing ammonia oxidizing bacteria in an environment having a pH of less than about 7.4. The storage state may also comprise providing ammonia oxidizing bacteria in an environment having ammonia, ammonia ions, and/or urea, trace minerals, oxygen, and low concentrations of carbon dioxide, as described in Section 1.

Storage may also be accomplished by storing at 4° C. for up to several months. The storage buffer in some embodiments may comprise 50 mM Na₂HPO₄— 2 mM MgCl₂ (pH 7.6).

In some embodiments, ammonia oxidizing bacteria may be cyropreserved. A 1.25 ml of ammonia oxidizing bacteria mid-log culture may be added to a 2 ml cryotube and 0.75 ml of sterile 80% glycerol. Tubes may be shaken gently, and incubate at room temperature for 15 min to enable uptake of the cryoprotective agents by the cells. The tubes may be directly stored in a −80° C. freezer for freezing and storage.

For resuscitation of cultures, frozen stocks may be thawed on ice for 10-20 minutes, and then centrifuged at 8,000×g for 3 minutes at 4° C. The pellet may be washed by suspending it in 2 ml AOB medium followed by another centrifugation at 8,000×g for 3 minutes at 4° C. to reduce potential toxicity of the cryoprotective agents. The pellet may be resuspended in 2 ml of AOB medium, inoculated into 50 ml of AOB medium containing 50 mM NH₄⁺, and incubated in dark at 30° C. by shaking at 200 rpm.

In some embodiments, the preparation of ammonia oxidizing bacteria may comprise ammonia oxidizing bacteria in a storage state and/or ammonia oxidizing bacteria in a polyphosphate loading state, and/or ammonia oxidizing bacteria in a growth state.

In some embodiments, upon actuation of the container, delivery system or device, ammonia oxidizing bacteria in a storage state or a polyphosphate loading state may be mixed with an activator. The activator may be in a form to provide a pH of at least about 7.6. The activator may be in a form to provide an environment having ammonia, ammonium ions, and/or urea, trace minerals and sufficient oxygen and carbon dioxide. The activator may revive or at least partially revive the ammonia oxidizing bacteria in a storage state or a polyphosphate loading state to a growth state. The time that it takes to revive the ammonia oxidizing bacteria from a storage state (or a polyphosphate loading state) may be a pre-determined period of time. For example, the pre-determined period of time may be less than about 75 hours, or less than about 72 hours. The pre-determined period of time may at least partially based on a period time of about 0.2-10 times, 0.3-5 times, 0.5-3 times, 0.5-1.5 times, or 0.5 to 1 times the doubling time for the ammonia oxidizing bacteria. The pre-determined period of time may be at least partially based on a period of time of about one doubling time for the ammonia oxidizing bacteria. The pre-determined period of time may be between about 8 hours and 12 hours. The pre-determined period of time may be about 10 hours. The pre-determined time may be less than about 75 hours, 72 hours, 70 hours, 68 hours, 65 hours, 60 hours, 55 hours, 50 hours, 45 hours, 40 hours, 35 hours, 30 hours, 25 hours, 20 hours, 15 hours, 10 hours, 5 hours, 4 hours, 3, hours, 2 hours, or 1 hour.

In some embodiments, the container may comprise ammonia oxidizing bacteria in a growth state, and in at least one of a storage state and a polyphosphate loading state, so as to provide ammonia oxidizing bacteria immediately to an environment to begin converting at least one of ammonia, ammonium ions, and urea to nitrite, while allowing for revival of the ammonia oxidizing bacteria in at least one of the storage state and the polyphosphate loading state over a period of time. This may allow for a controlled release of the stored ammonia oxidizing bacteria over a period of time.

Without wishing to be bound by theory, by maintaining ammonia oxidizing bacteria under conditions or in an environment of low carbon dioxide, with sufficient oxygen and ammonia, they may accumulate polyphosphate for a pre-determined period, e.g., for a period of about one doubling time, e.g., for about 8-12 hours, e.g., for about 10 hours. The ammonia oxidizing bacteria may accumulate sufficient polyphosphate to extend their storage viability, storage time, and accelerate their revival. This may occur with or without the addition of buffer and ammonia.

The presence of sufficient stored polyphosphate may allow the ammonia oxidizing bacteria the ATP resources to maintain metabolic activity even in the absence of ammonia and oxygen, and to survive insults that would otherwise be fatal.

The process of oxidation of ammonia to generate ATP has two steps. The first step is the oxidation of ammonia to hydroxylamine by ammonia monoxoygenase (Amo), followed by the conversion of hydroxylamine to nitrite by hydroxylamine oxidoreductase (Hao). Electrons from the second step (conversion of hydroxylamine to nitrite) are used to power the first step (oxidation of ammonia to hydroxylamine).

If an ammonia oxidizing bacteria does not have hydroxylamine to generate electrons for Amo, then hydroxylamine is not available for Hao. For example, acetylene irreversibly inhibits the enzyme crucial for the first step in the oxidation of ammonia to nitrite, the oxidation of ammonia to hydroxylamine. Once AOB are exposed to acetylene, Amo is irreversibly inhibited and new enzyme must be synthesized before hydroxylamine can be generated. In a normal consortium biofilm habitat, AOB may share and receive hydroxylamine form other AOB (even different strains with different susceptibilities to inhibitors) and so the biofilm tends to be more resistant to inhibitors such as acetylene than an individual organism. AOB can use stored polyphosphate to synthesize new Amo, even in the absence of hydroxylamine.

Any embodiment, preparation, composition, or formulation of ammonia oxidizing bacteria discussed herein may comprise, consist essentially of, or consist of optionally axenic ammonia oxidizing bacteria.

### 12. Methods of Producing Ammonia Oxidizing Bacteria

Methods of culturing various ammonia oxidizing bacteria, e.g., Nitrosomonas species are known in the art. Ammonia oxidizing bacteria may be cultured, for example, using the media described in Table 1 or Table 2, above.

Ammonia oxidizing bacteria may be grown, for example, in a liquid culture or on plates. Suitable plates include 1.2% R2A agar, 1.2% agar, 1.2% agarose, and 1.2% agarose with 0.3 g/L pyruvate.

In some embodiments, ammonia oxidizing bacteria may be cultured in organic free media. One advantage of using organic free media is that it lacks substrate for heterotrophic bacteria to metabolize except for that produced by the autotrophic bacteria. Another advantage of using the as-grown culture is that substantial nitrite accumulates in the culture media, and this nitrite is also inhibitory of heterotrophic bacteria and so acts as a preservative during storage.

In some embodiments, an ammonia oxidizing bacteria with improved, e.g. optimized, properties is produced by an iterative process of propagation and selecting for desired properties. In some embodiments, the selection and propagation are carried out simultaneously. In some embodiments, the selection is carried out in a reaction medium (e.g., complete N. europaea medium) comprising 50 mM, 75 mM, 100 mM, 125 mM, 150 mM, 175 mM, 200 mM, 225 mM, 250 mM, 275 mM, or 300 mM NH₄⁺, e.g., at least 200 mM NH₄⁺. In some embodiments, the period of propagation and/or selection is at least 1, 2, 3, or 6 months. In embodiments, the period of propagation and/or selection is at least 1, 2, 4, 6, 8, or 10 years.

In some aspects, the ammonia oxidizing bacteria are manufactured on a commercial scale. In some embodiments, commercial scale refers to a liquid culturing method with a culture medium volume of at least 10,000, 20,000, 30,000, 50,000, or 100,000 liters (L). In some embodiments, the bacteria are produced in a bioreactor. The bioreactor may maintain the bacteria at a constant temperature, e.g., about 26-30 degrees Celsius using, for example a thermal jacket for insulation, a temperature sensor, and a heating or cooling element. The bioreactor may have an apparatus for stirring the culture to improve distribution of nutrients like ammonia, urea, oxygen, carbon dioxide, and various minerals. The bioreactor may also have an inlet tube for addition of new medium, and an outlet tube for collection of cells. The bioreactor may also have an aerator for distributing oxygen and/or carbon dioxide to the culture. The bioreactor may be, e.g., a batch reactor, a fed batch reactor, or a continuous reactor. In some embodiments, commercial scale production of ammonia oxidizing bacteria yields a batch of 1,000 to 100,000 L per day at about 10¹² CFU/liter. The commercial scale production may yield e.g., a batch of 1,000-5,000, 5,000-10,000, 10,000-50,000, or 50,000-100,000 L/day. The commercial scale production may yield e.g., a batch of 1,000-5,000, 5,000-10,000, 10,000-50,000, or 50,000-100,000 L per batch. In some embodiments, the yield is at a concentration of at least 10⁸, 10⁹, 10¹⁰, 10₁₁, 2×10¹¹, 5×10¹¹, or 10¹², or about 10¹⁰-10¹¹, 10¹¹-10¹², 10¹²-10¹³, or 10¹³-10¹⁴ CFU/L. In some embodiments, the yield is at a concentration of at least 10⁸, 10⁹, 10¹⁰, 10¹¹, 2×10¹¹, 5×10¹¹, or 10¹², or about 10¹⁰-10₁₁, 10¹¹-10¹², 10¹²-10¹³, or 10¹³-10¹⁴ CFU/ml.

In some embodiments, typically including commercial scale production, quality control (QC) testing steps are carried out. The general steps of QC may comprise, 1) culturing ammonia oxidizing bacteria, 2) performing a testing step on the culture or an aliquot thereof, and 3) obtaining a value from the testing step, and optionally: 4) comparing the obtained value to a reference value or range of acceptable values, and 5) if the obtained value meets the acceptable reference value or range, then classifying the culture as acceptable, and if the obtained value does not meet the acceptable reference value or range, then classifying the culture as unacceptable. If the culture is classified as acceptable, the culture may, e.g., be allowed to continue growing and/or may be harvested and added to a commercial product. If the culture is classified as unacceptable, the culture may, e.g., be safely disposed of or the defect may be remedied.

The testing step may comprise measuring the optical density (OD) of the culture. OD is measured in a spectrophotometer, and provides information on the amount of light transmitted through the sample as distinguished from light absorbed or scattered. In some embodiments, the OD600 (e.g., optical density of light with a wavelength of 600 nm) may be determined. This measurement typically indicates the concentration of cells in the medium, where a higher optical density corresponds to a higher cell density.

The testing step may comprise measuring the pH of the culture. The pH of an ammonia oxidizing bacteria culture indicates the rate of nitrogen oxidation, and can also indicate whether the culture comprises a contaminating organism. pH may be measured using, e.g., a pH-sensing device comprising a electrode (such as a hydrogen electrode, quinhydron-Electrode, antimony electrode, glass electrode), a pH-sensing device comprising a semiconductor, or a color indicator reagent such as pH paper.

In certain embodiments, producing the ammonia oxidizing bacteria comprises carrying out various quality control steps. For instance, one may test the medium in which the ammonia oxidizing bacteria is grown, e.g., to determine whether it has an appropriate pH, whether it has a sufficiently low level of waste products, and/or whether it has a sufficiently high level of nutrients. One may also test for the presence of contaminating organisms. A contaminating organism is typically an organism other than ammonia oxidizing bacteria, for instance an organism selected from Microbacterium sp., Alcaligenaceae bacterium, Caulobacter sp., Burkodelia multivorans, Escherichia coli, Klebsiella pneumoniae, and Staphylococcus aureus. One may test for contaminants by, e.g., extracting DNA, amplifying it, and sequencing a conserved gene such as 16S rRNA. One may also test for contaminants by plating culture on agar plates and observing colony morphology. Ammonia oxidizing bacteria typically forms red colonies, so non-red colonies are often indicative of contaminating organisms.

### 13. Compositions Comprising Ammonia Oxidizing Bacteria

The present disclosure provides, inter alia, compositions comprising ammonia oxidizing bacteria, e.g., a preparation of ammonia oxidizing bacteria, or a purified preparation of ammonia oxidizing bacteria. The compositions comprising ammonia oxidizing bacteria, e.g., a preparation of ammonia oxidizing bacteria, or a purified preparation of ammonia oxidizing bacteria may be provided in a cosmetic product or a therapeutic product. The compositions may comprise natural products comprising ammonia oxidizing bacteria.

In some aspects, the present disclosure provides compositions, e.g., preparations, with a defined number of species. For instance, this disclosure provides a composition having ammonia oxidizing bacteria, or more specifically having one genus of ammonia oxidizing bacteria, or more specifically, having one species of ammonia oxidizing e.g., N. eutropha, and one other type of organism, and no other types of organism. In other examples, the composition has ammonia oxidizing bacteria, or more specifically has one genus of ammonia oxidizing bacteria, or more specifically, having one species of ammonia oxidizing e.g., N. eutropha and 2, 3, 4, 5, 6, 7, 8, 9, or 10 other types of organism, and no other types of organism. Suitable ammonia-oxidizing bacteria for this purpose include those in the genera Nitrosomonas, Nitrosococcus, Nitrosospira, Nitrosocystis, Nitrosolobus, or Nitrosovibrio.

In some embodiments, one or more other organisms besides ammonia oxidizing bacteria may be included in the preparation of ammonia oxidizing bacteria. For example, an organism of the genus selected from the group consisting of Lactobacillus, Streptococcus, Bifidobacter, and combinations thereof, may be provided in the preparation of ammonia oxidizing bacteria. In some embodiments, the preparation may be substantially free of other organisms.

In some embodiments, the composition, e.g., preparation, comprising ammonia oxidizing bacteria provides conditions that support ammonia oxidizing bacteria viability. For instance, the composition may promote ammonia oxidizing bacteria growth and metabolism or may promote a dormant state (e.g., freezing) or storage state as described herein, from which viable ammonia oxidizing bacteria can be recovered. When the composition promotes growth or metabolism, it may contain water and/or nutrients that ammonia oxidizing bacteria consumes, e.g., as ammonium ions, ammonia, urea, oxygen, carbon dioxide, or trace minerals.

Preparations of ammonia oxidizing bacteria may comprise between about between about 10⁸ to about 10¹⁴ CFU/L. The preparation may comprise at least about 10⁸, 10⁹, 10¹⁰, 10¹¹, 2×10¹¹, 5×10¹¹, 10¹², 2×10¹², 5×10¹², 10¹³, 2×10¹³, 5×10¹³, or 10¹⁴; or about 10⁸-10⁹, 10⁹-10¹⁰, 10¹⁰-10¹¹, 10¹¹-10¹², 10¹²-10¹³, or 10¹³-10¹⁴ CFU/L.

Preparations of ammonia oxidizing bacteria may comprise between about between about 10⁸ to about 10¹⁴ CFU/ml. The preparation may comprise at least about 10⁸, 10⁹, 10¹⁰, 10¹¹, 2×10¹¹, 5×10¹¹, 10¹², 2×10¹², 5×10¹², 10¹³, 2×10¹³, 5×10¹³, or 10¹⁴; or about 10⁸-10⁹, 10⁹-10¹⁰, 10¹⁰-10¹¹, 10¹¹-10¹², 10¹²-10¹³, or 10¹³-10¹⁴ CFU/ml.

In some embodiments, the preparation of ammonia oxidizing bacteria may comprise between about 0.1 milligrams (mg) to about 100 mg of ammonia oxidizing bacteria. In certain aspects, the preparation may comprise between about 50 mg and about 1000 mg of ammonia oxidizing bacteria. The preparation may comprise between about 0.1-0.5 mg, 0.2-0.7 mg, 0.5-1.0 mg, 0.5-2 mg, 0.5-5 mg, 2.5-5 mg, 2.5-7.0 mg, 5.0-10 mg, 7.5-15 mg, 10-15 mg, 15-20 mg, 15-25 mg, 20-30 mg, 25-50 mg, 25-75 mg, 50-75 mg, 50-100 mg, 75-100 mg, 100-200 mg, 200-300 mg, 300-400 mg, 400-500 mg, 500-600 mg, 600-700 mg, 700-800 mg, 800-900 mg, 900-1000 mg, 100-250 mg, 250-500 mg, 100-500 mg, 500-750 mg, 750-1000 mg, or 500-1000 mg.

In some embodiments, the preparation of ammonia oxidizing bacteria my comprise a mass ratio of ammonia oxidizing bacteria to an excipient, e.g., a pharmaceutically acceptable excipient or a cosmetically acceptable excipient in a range of about 0.1 grams per liter to about 1 gram per liter. The preparation may comprise a mass ratio of ammonia oxidizing bacteria to an excipient in a range of about 0.1-0.2, 0.2-0.3, 0.1-0.5, 0.2-0.7, 0.5-1.0, or 0.7-1.0 grams per liter.

In some embodiments, the preparation of ammonia oxidizing bacteria may be ammonia oxidizing bacteria in a buffer solution comprising, consisting essentially of, or consisting of disodium phosphate and magnesium chloride, for example, 50 mM Na₂HPO₄ and 2 mM MgCl₂. The preparation may be provided in a buffer at a pre-determined volume of, for example, between about 0.1 and about 100 fluid ounces, about 0.2 and about 50 fluid ounces, about 0.5 and about 25 fluid ounces, about 1.0 and about 10 fluid ounces, about 2.0 and about 7 fluid ounces, about 3 and about 5 fluid ounces. In some embodiments, the preparation may be provided in a container. The preparation may be provided in a container constructed to contain about 3.4 fluid ounces, or any other volume disclosed herein. The preparation may be in a form that may be capable of being aerosolized, sprayed or misted, i.e., in the form of a mist.

The ammonia oxidizing bacteria may be combined with one or more excipients, e.g., one or more pharmaceutically acceptable excipients or cosmetically acceptable excipients. In some embodiments, “pharmaceutically acceptable excipient” refers to a pharmaceutically-acceptable material, composition, or vehicle, such as a liquid or solid filler, diluent, solvent, or encapsulating material. In some embodiments, each excipient is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

In some embodiments, a cosmetically acceptable excipient refers to a cosmetically acceptable material, composition, or vehicle, such as a liquid or solid filler, diluent, solvent, or encapsulating material. In some embodiments, each excipient is cosmetically acceptable in the sense of being compatible with the other ingredients of a cosmetic formulation, and suitable for use in contact with the tissue or organ of humans and animals without excessive toxicity, irritation, allergic response, immunogenicity, or other problems or complications, commensurate with a reasonable benefit/risk ratio.

The excipient, e.g., the pharmaceutically acceptable excipient or the cosmetically acceptable excipient may be provided in the containers and kits of the present disclosure, e.g., within a preparation of ammonia oxidizing bacteria, within an activator, or within one or more chambers, e.g., a first chamber, second chamber, or mixing chamber of the container.

While it is possible for the active ingredient, e.g., ammonia oxidizing bacteria, to be administered alone, in many embodiments it is present in a pharmaceutical formulation, preparation, or composition, or a cosmetic formulation, preparation, or composition. Accordingly, this disclosure provides a pharmaceutical formulation (preparation or composition) or a cosmetic formulation (preparation or composition) comprising ammonia oxidizing bacteria and a pharmaceutically acceptable excipient or a cosmetically acceptable excipient. Pharmaceutical compositions and cosmetic compositions may take the form of a formulations as described below.

The pharmaceutical and cosmetic formulations (e.g., preparations or compositions) described herein may include those suitable for oral (e.g., by way of, or for the purposes of depositing in the gastrointestinal tract), parenteral (including subcutaneous, intradermal, intramuscular, intravenous, and intraarticular), inhalation (including fine particle dusts or mists which may be generated by means of various types of metered doses, pressurized aerosols, nebulizers or insufflators, and including intranasally (nasal) or via the lungs (pulmonary)), rectal and topical (including dermal, transdermal, transmucosal, buccal, sublingual, and intraocular) administration, although the most suitable route may depend upon, for example, the condition and disorder of the recipient.

The formulations (e.g., preparations or compositions) may conveniently be presented in unit dosage form and may be prepared by any of the methods known in the art of pharmacy or cosmetology. Typically, methods include the step of bringing the active ingredient (e.g., ammonia oxidizing bacteria) into association with a pharmaceutical or a cosmetic carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations may be presented as discrete units such as capsules, cachets or tablets, each containing a predetermined amount of ammonia oxidizing bacteria; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste. Various pharmaceutically acceptable carriers and their formulation are described in standard formulation treatises, e.g., Remington's Pharmaceutical Sciences by E. W. Martin. See also Wang, Y. J. and Hanson, M. A., Journal of Parenteral Science and Technology, Technical Report No. 10, Supp. 42:2 S, 1988.

The ammonia oxidizing bacteria compositions, or preparations, can, for example, be administered in a form suitable for immediate release or controlled (extended) release. Suitable examples of sustained-release systems include suitable polymeric materials, for example semi-permeable polymer matrices in the form of shaped articles, e.g., films, or microcapsules; suitable hydrophobic materials, for example as an emulsion in an acceptable oil; or ion exchange resins. Controlled (sustained)-release systems may be administered orally; rectally; parenterally; intracistemally; intravaginally; intraperitoneally; topically, for example as a powder, ointment, gel, drop or transdermal patch; bucally; or as a spray.

Preparations for administration can be suitably formulated to give controlled release of ammonia oxidizing bacteria. For example, the formulations, preparations, or compositions may be in the form of particles comprising one or more of biodegradable polymers, polysaccharide jellifying and/or bioadhesive polymers, or amphiphilic polymers. These compositions exhibit certain biocompatibility features which allow a controlled release of an active substance. See U.S. Pat. No. 5,700,486. The preparation may comprise a controlled release material.

In certain instances in this disclosure sustained-release or control-release systems may be referred to as a barrier.

Exemplary compositions, e.g., as a preparation, may include suspensions which can contain, for example, microcrystalline cellulose for imparting bulk, alginic acid or sodium alginate as a suspending agent, methylcellulose as a viscosity enhancer, dicalcium phosphate, starch, magnesium stearate and/or lactose and/or other excipients, binders, extenders, disintegrants, diluents and lubricants, mannitol, lactose, sucrose and/or cyclodextrins. Also included in such formulations may be high molecular weight excipients such as celluloses (avicel) or polyethylene glycols (PEG). Such formulations can also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g. Carbopol 934). Lubricants, surfactants, glidants, flavors, coloring agents and stabilizers may also be added for ease of fabrication and use. The surfactant may be a zwitterionic surfactant, a non-ionic surfactant, or an anionic surfactant.

Surfactants may include one or more of the following, alone, or in combination with those listed, or other surfactants or surfactant-like compounds: cocamidopropyl betaine (ColaTeric COAB), polyethylene sorbitol ester (e.g., Tween 80), ethoxylated lauryl alcohol (RhodaSurf 6 NAT), sodium laureth sulfate/lauryl glucoside/cocamidopropyl betaine (Plantapon 611 L UP), sodium laureth sulfate (e.g., RhodaPex ESB 70 NAT), alkyl polyglucoside (e.g., Plantaren 2000 N UP), sodium laureth sulfate (Plantaren 200), Dr. Bronner's Castile soap, Dr.

Bronner's baby soap, Lauramine oxide (ColaLux Lo), sodium dodecyl sulfate (SDS), polysulfonate alkyl polyglucoside (PolySufanate 160 P), sodium lauryl sulfate (Stepanol-WA Extra K) and combinations thereof. Dr. Bronner's Castile soap and baby soap comprises water, organic coconut oil, potassium hydroxide, organic olive oil, organic fair deal hemp oil, organic jojoba oil, citric acid, and tocopherol. Castile soaps, e.g., Dr. Bronner's Castile soap, and many natural and baby soaps are comprised of water, organic or non-organic animal or vegetable oil, sodium or potassium hydroxide, organic olive oil, organic fair deal hemp oil, organic jojoba oil, citric acid, and tocopherol.

Surfactants may include Sodium Laurylglucosides Hydroxypropylsulfonate (Suga®nate 160NC), lauramidopropyl betaine (Cola®Teric LMB); Cocamidopropyl hydroxysultaine (Cola®Teric CBS); disodium cocoamphodiacetate (Cola®Teric CDCX-LV); sodium laurylglucosides hydroxypropyl phosphate (Suga®Fax D12).

Surfactants may include sodium lauroyl methyl isethionate (Iselux® LQ-CLR-SB); sodium methyl cocoyl taurate (Pureact WS Conc.); Aqua (and) Sodium Lauroyl Methyl Isethionate (and) Cocamidopropyl Betaine (and) Sodium Cocoyl Isethionate (and) Sodium Methyl Oleoyl Taurate (Iselux®SFS-SB)

In some embodiments, surfactants may be used with ammonia oxidizing bacteria in amounts that allow nitrite production to occur. In some embodiments, the preparation may have less than about 0.01% to about 10% of surfactant. In some embodiments, the concentration of surfactant used may be between about 0.0001% and about 10%. In some embodiments, the preparation may be substantially free of surfactant.

In some embodiments, the formulation, e.g., preparation, may include other components that may enhance effectiveness of ammonia oxidizing bacteria, or enhance a treatment or indication.

In some embodiments, a chelator may be included in the preparation. A chelator may be a compound that may bind with another compound, e.g., a metal. The chelator may provide assistance in removing an unwanted compound from an environment, or may act in a protective manner to reduce or eliminate contact of a particular compound with an environment, e.g., ammonia oxidizing bacteria, e.g. a preparation of ammonia oxidizing bacteria, e.g., an excipient.

Formulations (e.g., preparations) may also contain anti-oxidants, buffers, bacteriostats that prevent the growth of undesired bacteria, solutes, and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of a sterile liquid carrier, for example saline or water-for-injection, immediately prior to use. Extemporaneous solutions and suspensions may be prepared from powders, granules and tablets of the kind previously described. Exemplary compositions include solutions or suspensions which can contain, for example, suitable non-toxic, pharmaceutically acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor. An aqueous carrier may be, for example, an isotonic buffer solution at a pH of from about 3.0 to about 8.0, a pH of from about 3.5 to about 7.4, for example from 3.5 to 6.0, for example from 3.5 to about 5.0. Useful buffers include sodium citrate-citric acid and sodium phosphate-phosphoric acid, and sodium acetate/acetic acid buffers. The composition in some embodiments does not include oxidizing agents.

Excipients that can be included are, for instance, proteins, such as human serum albumin or plasma preparations. If desired, the pharmaceutical composition, e.g., a preparation, may also contain minor amounts of non-toxic auxiliary substances, such as wetting or emulsifying agents, surfactants, preservatives, and pH buffering agents and the like, for example sodium citrate, sodium acetate or sorbitan monolaurate. In some embodiments, excipients, e.g., a pharmaceutically acceptable excipient or a cosmetically acceptable excipient, may comprise an anti-adherent, binder, coat, disintegrant, filler, flavor, color, lubricant, glidant, sorbent, preservative, or sweetener. In some embodiments, the preparation may be substantially free of excipients.

Exemplary compositions for aerosol administration include solutions in saline, which can contain, for example, benzyl alcohol or other suitable preservatives, absorption promoters to enhance bioavailability, and/or other solubilizing or dispersing agents. Conveniently in compositions for aerosol administration the ammonia oxidizing bacteria may be delivered in the form of an aerosol spray presentation from a pressurized pack or a nebulizer, with the use of a suitable propellant, e.g., dichlorodifluoro-methane, trichlorofluoromethane, dichlorotetrafluoroethane, carbon dioxide, nitrogen, or other suitable gas. In the case of a pressurized aerosol the dosage unit can be determined by providing a valve to deliver a metered amount. Capsules and cartridges of e.g., gelatin can be formulated to contain a powder mix of the ammonia oxidizing bacteria and a suitable powder base, for example lactose or starch. In certain embodiments, ammonia oxidizing bacteria is administered as an aerosol from a metered dose valve, through an aerosol adapter also known as an actuator. Optionally, a stabilizer is also included, and/or porous particles for deep lung delivery are included (e.g., see U.S. Pat. No. 6,447,743). The composition or preparation may be in a form that may be capable of being aerosolized, sprayed or misted, i.e., in the form of a mist. The preparation of ammonia oxidizing bacteria may be ammonia oxidizing bacteria in a buffer solution comprising, consisting essentially of, or consisting of disodium phosphate and magnesium chloride, for example, 50 mM Na₂HPO₄ and 2 mM MgCl₂.

Formulations may be presented with carriers such as shea or cocoa butter, synthetic glyceride esters or polyethylene glycol. Such carriers are typically solid at ordinary temperatures, but liquefy and/or dissolve at body temperature to release the ammonia oxidizing bacteria.

Exemplary compositions for topical administration include a topical carrier such as Plastibase (mineral oil gelled with polyethylene). In some aspects, the composition, e.g., preparation, and/or excipient may be in the form of one or more of a liquid, a solid, or a gel. For example, liquid suspensions may include, but are not limited to, water, saline, phosphate-buffered saline, or an ammonia oxidizing storage buffer.

Gel formulations may include, but are not limited to agar, silica, polyacrylic acid (for example Carbopol®), carboxymethyl cellulose, starch, guar gum, alginate, clays, or chitosan. In some embodiments, the formulation, e.g., preparation, may be supplemented with an ammonia source including, but not limited to one or more of ammonia, ammonium ions, e.g., ammonium chloride or ammonium sulfate, and urea.

In some embodiments, an ammonia oxidizing bacteria composition, e.g., preparation, is formulated to improve NO penetration into the skin. A gel-forming material such as KY jelly or various hair gels would present a diffusion barrier to NO loss to ambient air, and so improve the skin's absorption of NO. The NO level in the skin will generally not greatly exceed 20 nM/L because that level activates GC and would cause local vasodilatation and oxidative destruction of excess NO.

It should be understood that in addition to the ingredients particularly mentioned above, the formulations, e.g., preparations, as described herein may include other agents conventional in the art having regard to the type of formulation in question.

The formulation, e.g., preparation, e.g., composition may be provided in a container, delivery system, or delivery device, having a weight, including or not including the contents of the container, that may be less than about 50, 100, 200, 300, 400, 500, 600, 700, 800, 900, 1000, 1500, or 2000 grams.

Suitable unit dosage formulations are those containing an effective dose, as hereinbefore recited, or an appropriate fraction thereof, of ammonia oxidizing bacteria.

A therapeutically effective amount of ammonia oxidizing bacteria may be administered as a single pulse dose, as a bolus dose, or as pulse doses administered over time. Thus, in pulse doses, a bolus administration of ammonia oxidizing bacteria is provided, followed by a time period wherein ammonia oxidizing bacteria is administered to the subject, followed by a second bolus administration. In specific, non-limiting examples, pulse doses are administered during the course of a day, during the course of a week, or during the course of a month.

In some embodiments, a preparation of ammonia oxidizing bacteria, e.g., a formulation, e.g., a composition, may be applied for a pre-determined number of days. This may be based, for example, at least in part, on the severity of the condition or disease, the response to the treatment, the dosage applied and the frequency of the dose. For example, the preparation may be applied for about 1-3, 3-5, 5-7, 7-9, 5-10, 10-14, 12-18, 12-21, 21-28, 28-35, 35-42, 42-49, 49-56, 46-63, 63-70, 70-77, 77-84, 84-91 days. In certain aspects, the preparation may be applied for about 16 days.

In some embodiments, a preparation of ammonia oxidizing bacteria, e.g., a formulation, e.g., a composition, may be applied a pre-determined number of times per day. This may be based, for example, at least in part, on the severity of the condition or disease, the response to the treatment, the dosage applied and the frequency of the dose. For example, the preparation may be applied 1, 2, 3, 4, 5, 6, 7, 8, 9, 10, 11, 12, 13, 14, 15, 16, 17, 18, 19, 20, 21, 22, 23, 24 times per day.

In some embodiments, the preparation may be applied one time per day. In other embodiments, the preparation may be applied two times per day. In some embodiments, the preparation may be applied a first pre-determined amount for a certain number of days, and a second pre-determined amount for a certain subsequent number of days. In some embodiments, the preparation may be applied for about 16 days.

In some embodiments, the ammonia oxidizing bacteria is administered for about 1-3, 3-5, 5-7, 7-9, 5-10, 10-14, 12-18, 12-21, 21-28, 28-35, 35-42, 42-49, 49-56, 46-63, 63-70, 70-77, 77-84, 84-91 days, e.g., for about 1 month, for about 2 months, for about 3 months. In some embodiments, the ammonia oxidizing bacteria is administered for an indefinite period of time, e.g, greater than one year, greater than 5 years, greater than 10 years, greater than 15 years, greater than 30 years, greater than 50 years, greater than 75 years.

Ammonia oxidizing bacteria may be associated with a variety of consumer and therapeutic products, and examples of such products are set out below. In some embodiments, the ammonia oxidizing bacteria associated with a product is admixed with the product, for example, spread evenly throughout the product, and in some embodiments, the ammonia oxidizing bacteria associated with a product is layered on the product.

In some embodiments, the ammonia oxidizing bacteria is associated with a powder. Powders are typically small particulate solids that are not attached to each other and that can flow freely when tilted. Exemplary powders for consumer use include talcum powder and some cosmetics (e.g., powder foundation, including pressed powders). Other powders may be contemplated for use in conjunction with ammonia oxidizing bacteria systems and methods of the present disclosure.

In some embodiments, the ammonia oxidizing bacteria is associated with a cosmetic. The cosmetic may be a substance for topical application intended to alter a person's appearance, e.g., a liquid foundation, a powder foundation, blush, or lipstick. The cosmetic may be any substance recited in the Food and Drug Administration regulations, e.g., under 21 C.F.R.§ 720.4.

The preparation, e.g., cosmetic, may be provided as or disposed in at least one of a baby product, e.g., a baby shampoo, a baby lotion, a baby oil, a baby powder, a baby cream; a bath preparation, e.g., a bath oil, a tablet, a salt, a bubble bath, a bath capsule; an eye makeup preparation, e.g., an eyebrow pencil, an eyeliner, an eye shadow, an eye lotion, an eye makeup remover, a mascara; a fragrance preparation, e.g., a colognes, a toilet water, a perfume, a powder (dusting and talcum), a sachet; hair preparations, e.g., hair conditioners, hair sprays, hair straighteners, permanent waves, rinses, shampoos, tonics, dressings, hair grooming aids, wave sets; hair coloring preparations, e.g., hair dyes and colors, hair tints, coloring hair rinses, coloring hair shampoos, hair lighteners with color, hair bleaches; makeup preparations, e.g., face powders, foundations, leg and body paints, lipstick, makeup bases, rouges, makeup fixatives; manicuring preparations, e.g., basecoats and undercoats, cuticle softeners, nail creams and lotions, nail extenders, nail polish and enamel, nail polish and enamel removers; oral hygiene products, e.g., dentrifices, mouthwashes and breath fresheners; bath soaps and detergents, deodorants, douches, feminine hygiene deodorants; shaving preparations, e.g., aftershave lotions, beard softeners, talcum, preshave lotions, shaving cream, shaving soap; skin care preparations, e.g., cleansing, depilatories, face and neck, body and hand, foot powders and sprays, moisturizing, night preparations, paste masks, skin fresheners; and suntan preparations, e.g., gels, creams, and liquids, and indoor tanning preparations.

In some embodiments, preparation, e.g., cosmetic, may be provided as or disposed in at least one of a baby product, e.g., a baby shampoo, a baby lotion, a baby oil, a baby powder, a baby cream; a bath preparation, e.g., a bath oil, a tablet, a salt, a bubble bath, a bath capsule; a powder (dusting and talcum), a sachet; hair preparations, e.g., hair conditioners, rinses, shampoos, tonics, face powders, cuticle softeners, nail creams and lotions, oral hygiene products, mouthwashes, bath soaps, douches, feminine hygiene deodorants; shaving preparations, e.g., aftershave lotions, skin care preparations, e.g., cleansing, face and neck, body and hand, foot powders and sprays, moisturizing, night preparations, paste masks, skin fresheners; and suntan preparations, e.g., gels, creams, and liquids.

Other components may be added to pharmaceutical formulations, e.g., preparations, or cosmetic preparations as selected by one skilled in the art of cosmetic formulation such as, for example, water, mineral oil, coloring agent, perfume, aloe, glycerin, sodium chloride, sodium bicarbonate, pH buffers, UV blocking agents, silicone oil, natural oils, vitamin E, herbal concentrates, lactic acid, citric acid, talc, clay, calcium carbonate, magnesium carbonate, zinc oxide, starch, urea, and erythorbic acid, or any other excipient known by one of skill in the art, including those disclosed herein.

In some embodiments, the preparation may be disposed in, or provided as, a powder, cosmetic, cream, stick, aerosol, salve, wipe, or bandage.

In some embodiments, ammonia oxidizing bacteria is associated with a cream. The cream may be a fluid comprising a thickening agent, and generally has a consistency that allows it to be spread evenly on the skin. Exemplary creams include moisturizing lotion, face cream, and body lotion.

In some embodiments, the ammonia oxidizing bacteria is associated with a stick. A stick is typically a solid that, when placed in contact with a surface, transfers some of the stick contents to the surface. Exemplary sticks include deodorant stick, lipstick, lip balm in stick form, and sunscreen applicator sticks.

In some embodiments, the ammonia oxidizing bacteria is associated with an aerosol. An aerosol is typically a colloid of fine solid particles or fine liquid droplets, in a gas such as air. Aerosols may be created by placing the ammonia oxidizing bacteria (and optionally carriers) in a vessel under pressure, and then opening a valve to release the contents. The container may be designed to only exert levels of pressure that are compatible with ammonia oxidizing bacteria viability. For instance, the high pressure may be exerted for only a short time, and/or the pressure may be low enough not to impair viability. Examples of consumer uses of aerosols include for sunscreen, deodorant, perfume, hairspray, and insect repellant.

In some embodiments, the ammonia oxidizing bacteria is associated with a salve. A salve may be a topically applied agent with a liquid or cream-like consistency, intended to protect the skin or promote healing. Examples of salves include burn ointments and skin moisturizers.

In some embodiments, the ammonia oxidizing bacteria is associated with a wipe. A wipe may be a flexible material suitable for topically applying a liquid or cream onto skin. The wipe may be, e.g., paper-based or cloth based. Exemplary wipes include tissues and wet wipes.

The compositions comprising ammonia oxidizing bacteria may also comprise one or more of a moisturizing agent, deodorizing agent, scent, colorant, insect repellant, cleansing agent, or UV-blocking agent.

For instance, the moisturizing agent may be an agent that reduces or prevents skin dryness. Exemplary moisturizing agents include humectants (e.g., urea, glycerin, alpha hydroxy acids and dimethicone) and emollients (e.g., lanolin, mineral oil and petrolatum). Moisturizing agents may be included, e.g., in ammonia oxidizing bacteria-containing creams, balms, lotions, or sunscreen.

A deodorizing agent may be an agent that reduces unwanted odors. A deodorizing agent may work by directly neutralizing odors, preventing perspiration, or preventing the growth of odor-producing bacteria. Exemplary deodorizing agents include aluminum ions (e.g., aluminum chloride or aluminum chlorohydrate), cyclomethicone, talc, baking soda, essential oils, mineral ions, hops, and witch hazel. Deodorizing agents are typically present in spray or stick deodorants, and can also be found in some soaps and clothing.

An insect repellant may be an agent that can be applied to surfaces (e.g., skin) that discourage insects and other arthropods from lighting on the surface. Insect repellants include DEET (N,N-diethyl-m-toluamide), p-menthane-3,8-diol (PMD), icaridin, nepetalactone, citronella oil, neem oil, bog myrtle, dimethyl carbate, Tricyclodecenyl allyl ether, and IR3535 (3-[N-Butyl-N-acetyl]-aminopropionic acid, ethyl ester).

A cleansing agent may be an agent that removes dirt or unwanted bacteria from a surface like skin. Exemplary cleansing agents include bar soaps, liquid soaps, and shampoos.

A UV-blocking agent may be an agent that can be applied to a surface to reduce the amount of ultraviolet light the surface receives. A UV-blocking agent may block UV-A and/or UV-B rays. A UV blocking agent can function by absorbing, reflecting, or scattering UV. Exemplary UV-blocking agents include absorbers, e.g., homosalate, octisalate (also called octyl salicylate), octinoxate (also called octyl methoxycinnamate or OMC), octocrylene, oxybenzone, and avobenzone, and reflectors (e.g., titanium dioxide and zinc oxide). UV-blocking agents are typically presents in sunscreens, and can also be found in skin creams and some cosmetics.

In some embodiments, ammonia oxidizing bacteria is associated with a conditioner. Conditioner generally refers to a substance with cream-like consistency that can be applied to hair to improve its appearance, strength, or manageability.

In some embodiments, ammonia oxidizing bacteria is associated with cloth. Cloth generally refers to a flexible material suitable to be made into clothing, e.g., having enough material strength to withstand everyday motion by a wearer. Cloth can be fibrous, woven, or knit; it can be made of a naturally occurring material or a synthetic material. Exemplary cloth materials include cotton, flax, wool, ramie, silk, denim, leather, nylon, polyester, and spandex, and blends thereof.

In some embodiments, ammonia oxidizing bacteria is associated with yarn. Yarn generally refers to a long, thin spun flexible material that is suitable for knitting or weaving. Yarn can be made of, e.g., wool, cotton, polyester, and blends thereof.

In some embodiments, ammonia oxidizing bacteria is associated with thread. Thread generally refers to a long, thin spun flexible material that is suitable for sewing. Thread generally has a thinner diameter than yarn. Thread can be made of, e.g., cotton, polyester, nylon, silk, and blends thereof.

Articles of clothing such as, for example, shoes, shoe inserts, pajamas, sneakers, belts, hats, shirts, underwear, athletic garments, helmets, towels, gloves, socks, bandages, and the like, may also be treated with ammonia oxidizing bacteria. Bedding, including sheets, pillows, pillow cases, and blankets may also be treated with ammonia oxidizing bacteria. In some embodiments, areas of skin that cannot be washed for a period of time may also be contacted with ammonia oxidizing bacteria. For example, skin enclosed in orthopedic casts which immobilize injured limbs during the healing process, and areas in proximity to injuries that must be kept dry for proper healing such as stitched wounds may benefit from contact with the ammonia oxidizing bacteria.

In some aspects, the present disclosure provides a wearable article comprising an ammonia oxidizing bacterium or ammonia oxidizing bacteria as described herein. A wearable article may be a light article that can be closely associated with a user's body, in a way that does not impede ambulation. Examples of wearable articles include a wristwatch, wristband, headband, hair elastic, hair nets, shower caps, hats, hairpieces, adhesive plastic films and patches, adhesive microneedle patches and arrays, and jewelry. The wearable article comprising ammonia oxidizing bacteria described herein may provide, e.g., at a concentration that provides one or more of a treatment or prevention of a skin disorder, a treatment or prevention of a disease or condition associated with low nitrite levels, a treatment or prevention of body odor, a treatment to supply nitric oxide to a subject, or a treatment to inhibit microbial growth.

In some embodiments, the ammonia oxidizing bacteria is associated with a product intended to contact the hair, for example, a brush, comb, shampoo, conditioner, headband, hair elastic, hair nets, shower caps, hats, and hairpieces. Nitric oxide formed on the hair, away from the skin surface, may be captured in a hat, scarf or face mask and directed into inhaled air.

Articles contacting the surface of a human subject, such as a diaper, may be associated with ammonia oxidizing bacteria. Because diapers are designed to hold and contain urine and feces produced by incontinent individuals, the urea in urine and feces can be hydrolyzed by skin and fecal bacteria to form free ammonia which is irritating and may cause diaper rash. Incorporation of bacteria that metabolize urea into nitrite or nitrate, such as ammonia oxidizing bacteria, may avoid the release of free ammonia and may release nitrite and ultimately NO which may aid in the maintenance of healthy skin for both children and incontinent adults. The release of nitric oxide in diapers may also have anti-microbial effects on disease causing organisms present in human feces. This effect may continue even after disposable diapers are disposed of as waste and may reduce the incidence of transmission of disease through contact with soiled disposable diapers.

In some embodiments, the product comprising ammonia oxidizing bacteria is packaged. The packaging may serve to compact the product or protect it from damage, dirt, or degradation. The packaging may comprise, e.g., plastic, paper, cardboard, or wood. In some embodiments the packaging is impermeable to bacteria. In some embodiments the packaging is permeable to oxygen and/or carbon dioxide.

### 14. Methods of Treatment with Ammonia Oxidizing Bacteria

The present disclosure provides various methods of treating diseases and conditions using ammonia oxidizing bacteria, e.g., by administering ammonia oxidizing bacteria, e.g., a preparation of ammonia oxidizing bacteria, e.g., a natural product or a fortified natural product (a fortified natural product being fortified with ammonia oxidizing bacteria, e.g., exogenous ammonia oxidizing bacteria), or compositions, preparations, or formulations comprising a natural product or a fortified natural product.

The ammonia oxidizing bacteria that may be used to treat diseases and conditions include all the ammonia oxidizing bacteria compositions described in this application, e.g. a preparation of ammonia oxidizing bacteria, a natural product or a fortified natural product, or compositions, preparations, or formulations comprising a natural product or a fortified natural product.

For instance, the disclosure provides uses, for treating a condition or disease (e.g., inhibiting microbial growth on a subject's skin), a composition of ammonia oxidizing bacteria. In embodiments, the ammonia oxidizing bacteria may be used to treat ulcers or infections in ulcers, e.g., venous ulcer, e.g., leg ulcer, e.g., venous leg ulcer, e.g., diabetic ulcers, e.g., diabetic foot ulcers, chronic wounds, acne, e.g., acne vulgaris, rosacea, eczema, uticaria, or psoriasis.

The ammonia oxidizing bacteria of the present disclosure may provide for, or be useful for treating or preventing a skin disorder, treating or preventing a disease or condition associated with low nitrite levels, a treating or preventing body odor, treating to supply nitric oxide to a subject, or treating to inhibit microbial growth.

The ammonia oxidizing bacteria of the present disclosure may provide for, or be useful in a treatment of at least one of HIV dermatitis, infection in an ulcer, e.g., venous ulcer, e.g., leg ulcer, e.g., venous leg ulcer, e.g. infection in a diabetic foot ulcer, atopic dermatitis, acne, e.g., acne vulgaris, eczema, contact dermatitis, allergic reaction, psoriasis, uticaria, rosacea, skin infections, vascular disease, vaginal yeast infection, bacteria vaginosis, sexually transmitted diseases, heart disease, atherosclerosis, baldness, leg ulcers secondary to diabetes or confinement to bed, angina, particularly chronic, stable angina pectoris, ischemic diseases, congestive heart failure, myocardial infarction, ischemia reperfusion injury, laminitis, hypertension, hypertrophic organ degeneration, Raynaud's phenomenon, fibrosis, fibrotic organ degeneration, allergies, autoimmune sensitization, end stage renal disease, obesity, impotence, pneumonia, primary immunodeficiency, epidermal lysis bulosa, or cancer. In embodiments, the condition is a venous leg ulcer.

In some embodiments, ammonia oxidizing bacteria are used to treat a subject. A subject” may include an animal, a mammal, a human, a non-human animal, a livestock animal, or a companion animal. The term “subject” is intended to include human and non-human animals, for example, vertebrates, large animals, and primates. In certain embodiments, the subject is a mammalian subject, and in particular embodiments, the subject is a human subject. Although applications with humans are clearly foreseen, veterinary applications, for example, with non-human animals, are also envisaged herein. The term “non-human animals” of the disclosure includes all vertebrates, for example, non-mammals (such as birds, for example, chickens; amphibians; reptiles) and mammals, such as non-human primates, domesticated, and agriculturally useful animals, for example, sheep, dog, cat, cow, pig, rat, among others.

In some embodiments, ammonia oxidizing bacteria described herein are used to inhibit the growth of other organisms. For instance, ammonia oxidizing bacteria may be well-adapted for long-term colonization of human skin, and in some embodiments it out-competes other bacteria that are undesirable on the skin. Undesirable skin bacteria include, e.g., those that can infect wounds, raise the risk or severity of a disease, or produce odors. Undesirable bacteria may be referred to as pathogenic bacteria. Certain undesirable skin bacteria, e.g., potentially pathogenic bacteria, e.g., pathogenic bacteria, include Staphylococcus aureus (S. aureus), e.g., methicillin resistant Staphylococcus aureus Psuedomomas aeruginosa (P. aeruginosa), Streptococcus pyogenes (S. pyogenes), Acinetobacter baumannii (A. baumannii), Propionibacteria, and Stenotrophomonas. The ammonia oxidizing bacteria described herein may out-compete other organisms by, e.g., consuming scarce nutrients, or generating byproducts that are harmful to other organisms, e.g., changing the pH of the skin to a level that is not conducive to the undesirable organism's growth.

Accordingly, the present disclosure provides, inter alia, a method of inhibiting microbial growth on a subject's skin, comprising topically administering to a human in need thereof an effective dose of ammonia oxidizing bacteria as described herein. Similarly, the present disclosure provides ammonia oxidizing bacteria as described herein for use in inhibiting microbial growth on a subject's skin. Likewise, the present disclosure provides a use of ammonia oxidizing bacteria in the manufacture of a medicament for inhibiting microbial growth on a subject's skin.

The present disclosure also provides a method of supplying nitric oxide to a subject, comprising positioning an effective dose of ammonia oxidizing bacteria described herein in close proximity to the subject. Similarly, the present disclosure provides ammonia oxidizing bacteria as described herein for use in supplying nitric oxide to a subject. Likewise, the present disclosure provides a use of ammonia oxidizing bacteria in the manufacture of a medicament or composition suitable for position in close proximity to a subject.

The present disclosure also provides a method of reducing body odor, comprising topically administering to a subject in need thereof an effective dose of ammonia oxidizing bacteria described herein. Similarly, the present disclosure provides ammonia oxidizing bacteria as described herein for use in reducing body odor in a subject. Likewise, the present disclosure provides a use of ammonia oxidizing bacteria as described herein in the manufacture of a medicament or composition for reducing body odor.

The present disclosure also provides a method of treating or preventing a disease associated with low nitrite levels, comprising topically administering to a subject in need thereof a therapeutically effective dose of ammonia oxidizing bacteria described herein. Similarly, the present disclosure provides a topical formulation of ammonia oxidizing bacteria as described herein for use in treating a disease associated with low nitrite levels. Likewise, the present disclosure provides a use of ammonia oxidizing bacteria as described herein in the manufacture of a topical medicament for treating a disease associated with low nitrite levels.

The present disclosure also provides a method of treating or preventing a skin disorder or skin infection, comprising topically administering to a subject in need thereof a therapeutically effective dose of ammonia oxidizing bacteria as described herein. Similarly, the present disclosure provides ammonia oxidizing bacteria as described herein for use in treating a skin disorder in a subject. Likewise, the present disclosure provides a use of ammonia oxidizing bacteria as described herein in the manufacture of a medicament for treating skin disorder. In embodiments, the skin disorder is acne, e.g., acne vulgaris, rosacea, eczema, psoriasis, or urticaria; the skin infection is impetigo.

While not wishing to be bound by theory, it is proposed that treatment of acne, e.g., acne vulgaris, with a therapeutically effective dose of ammonia oxidizing bacteria; and/or limiting and/or inhibiting the spread and proliferation of Propionibacterium acnes associated with acne vulgaris through acidified nitrite and NO production.

While not wishing to be bound by theory, it is proposed that treatment of rosacea with a therapeutically effective dose of ammonia oxidizing bacteria as described herein may involve downregulation due to NO generation. This may be due to expression of Kazal-type KLK5/KLK7 inhibitor(s) that may reduce formation of the human cathelicidin peptide LL-37 from its precursor propeptide hCAP18.

While not wishing to be bound by theory, it is proposed that treatment of eczema and/or atopic dermatitis with a therapeutically effective dose of as described herein may involve donwregulation of inflammation due to NO generation; and/or limiting and/or inhibiting the spread and proliferation of S. aureus and other skin pathogens often associated with very high colonization rates and skin loads in atopic dermatitis through acidified nitrite and NO production.

While not wishing to be bound by theory, it is proposed that treatment of psoriasis with a therapeutically effective dose of ammonia oxidizing bacteria described herein may involve downregulation of inflammation due to NO generation and reduction in formation of human cathelicidin peptide LL-37.

While not wishing to be bound by theory, it is proposed that treatment of psoriasis with a therapeutically effective dose of ammonia oxidizing bacteria as described herein may involve downregulation of inflammation due to NO generation.

While not wishing to be bound by theory, it is proposed that treatment of impetigo or other skin and soft tissue infections with a therapeutically effective dose of ammonia oxidizing bacteria as described herein may involve limiting and/or inhibiting the spread and proliferation of Staphylococcus aureus (S. aureus), Psuedomomas aeruginosa (P. aeruginosa), Streptococcus pyogenes (S. pyogenes), Acinetobacter baumannii (A. baumannii), Propionibacteria, and Stenotrophomonas.

The present disclosure also provides a method of promoting wound healing, comprising administering to a wound an effective dose of ammonia oxidizing bacteria as described herein. Similarly, the present disclosure provides ammonia oxidizing bacteria as described herein for use in treating a wound. Likewise, the present disclosure provides a use of ammonia oxidizing bacteria as described herein in the manufacture of a medicament or a composition for treating a wound.

Ammonia oxidizing bacteria as described herein may be used to promote wound healing in a patient that has an impaired healing ability, e.g., a diabetic patient.

In some embodiments, this disclosure provides methods of using ammonia oxidizing bacteria as described herein to prevent a disease or disorder, e.g., a skin disorder. Prevention, in certain embodiments, means reducing the risk of a subject developing a disease, compared to a similar untreated subject. The risk need not be reduced to zero.

In some embodiments, a method of changing a composition of a skin microbiome of a subject is provided. The method may comprise administering, e.g., applying, a preparation comprising ammonia oxidizing bacteria to a surface of the skin. The amount and frequency of administration, e.g., application, is sufficient to reduce the proportion of pathogenic bacteria on the surface of the skin. The subject may be selected on the basis of the subject being in need of a reduction in the proportion of pathogenic bacteria on the surface of the skin.

Individuals having a reduced bathing frequency, such as astronauts, submarine crew members, military personnel during a campaign, civilian workers in remote locations, refugees, bedridden individuals and many others may maintain healthier skin by maintaining ammonia oxidizing bacteria on the skin. With regard to bedridden individuals, the ammonia oxidizing bacteria in some embodiments reduces the frequency or severity of bedsores by augmenting inadequate circulation.

It is appreciated that many modern degenerative diseases may be caused by a lack of NO species, and that ammonia oxidizing bacteria on the external skin can supply those species by diffusion, and that application of ammonia oxidizing bacteria to the skin resolves long standing medical conditions. In certain embodiments, ammonia oxidizing bacteria are applied to a subject to offset modern bathing practices, especially with anionic detergents remove ammonia oxidizing bacteria from the external skin.

One suitable method of topical application to apply sufficient ammonia oxidizing bacteria and then wear sufficient clothing so as to induce sweating. However, many people will want to derive the benefits of ammonia oxidizing bacteria while maintaining their current bathing habits, in which case, a culture of the bacteria can be applied along with sufficient substrate for them to produce NO. A nutrient solution approximating the inorganic composition of human sweat can be used for this purpose. Using bacteria adapted to media approximating human sweat minimizes the time for them to adapt when applied. Since sweat evaporates once excreted onto the skin surface, using a culture media that has a higher ionic strength is desirable. A concentration approximately twice that of human sweat is suitable, but other conditions are also contemplated. This may be applied as a spray, a gel, a film-forming polymer gel, a cream, a foam, or a lotion. Ammonia oxidizing bacteria's nutritional needs are typically met with NH₃ or urea, O₂, CO₂, and minerals. In some embodiments, the substrate comprises trace minerals including iron, copper, zinc, cobalt, molybdenum, manganese, sodium, potassium, calcium, magnesium, chloride, phosphate, sulfate, or any combination thereof. In some embodiments, the nutrient solution will contain a pH buffer system to optimize the delivery of substrate.

In some embodiments, the present disclosure provides a method of treating a wound by applying a bandage comprising ammonia oxidizing bacteria to the wound. Also provided are methods of producing such a bandage. The bandage may comprise, for example, an adhesive portion to affix the bandage to undamaged skin near the wound and a soft, flexible portion to cover or overlay the wound. In some embodiments, the bandage contains no other organisms but ammonia oxidizing bacteria. The bandage may made of a permeable material that allows gasses like oxygen and carbon dioxide to reach the ammonia oxidizing bacteria when the bandage is applied to the wound. In certain embodiments, the bandage comprises nutrients for ammonia oxidizing bacteria such as ammonium, ammonia, urea, or trace minerals. In certain embodiments, the bandage comprises an antibiotic to which the ammonia oxidizing bacteria is resistant. The antibiotic resistance may arise from one or more endogenous resistance gene or from one or more transgenes.

In some embodiments, the ammonia oxidizing bacteria e.g., a preparation of ammonia oxidizing bacteria, is administered at a dose of about 10⁸-10⁹ CFU, 10⁹-10¹⁰ CFU, 10¹⁰-10¹¹ CFU, 10¹¹-10¹² CFU, 10¹²-10¹³ CFU, or 10¹³-10¹⁴ CFU per application. In some embodiments, the ammonia oxidizing bacteria is administered topically at a dose of about 10⁹-10¹⁰ CFU, about 1×10⁹-5×10⁹, 1×10⁹-3×10⁹, or 1×10⁹-10×10⁹ CFU; or about 10¹⁰-10¹¹ CFU, e.g., about 1×10¹⁰-5×10¹⁰, 1×10¹⁰-3×10¹⁰, or 1×10¹⁰-2×10¹⁰ CFU; or about 10¹¹-10¹² CFU, e.g., about 1×10¹¹-5×10¹¹, 1×10¹¹-3×10¹¹, or 1×10¹¹-2×10¹¹ CFU; or about 10¹²-10¹³ CFU, e.g., about 1×10¹²-5×10¹², 1×10¹²-3×10¹², or 1×10¹²-2×10¹² CFU; or about 10¹³-10¹⁴ CFU, e.g., about 1×10¹³-5×10¹³, 1×10¹³-3×10¹³, or 1×10¹³-2×10¹³ CFU.

In some embodiments, the ammonia oxidizing bacteria is administered in a volume of about 1-2, 2-5, 5-10, 10-15, 12-18, 15-20, 20-25, or 25-50 ml per dose. In some embodiments, the solution is at a concentration of about 10⁸-10⁹, 10⁹-10¹⁰, or 10¹⁰40¹¹ CFUs/ml. In some embodiments, the ammonia oxidizing bacteria is administered as two 15 ml doses per day, where each dose is at a concentration of 10⁹ CFU/ml.

In some embodiments, the ammonia oxidizing bacteria is administered once, twice, three, or four times per day. In some embodiments, the ammonia oxidizing bacteria is administered once, twice, three, four, five, or six times per week. In some embodiments, the ammonia oxidizing bacteria is administered shortly after bathing. In some embodiments, the ammonia oxidizing bacteria is administered shortly before sleep.

In certain aspects, the present disclosure provides combination therapies comprising ammonia oxidizing bacteria and a second therapeutic. For instance, the disclosure provides physical admixtures of the two (or more) therapies are physically admixed. In other embodiments, the two (or more) therapies are administered in combination as separate formulation. The second therapy may be, e.g., a pharmaceutical agent, surgery, or any other medical approach that treats the relevant disease or disorder. The following paragraphs describe combination therapies capable of treating an ulcer, e.g., venous ulcer, e.g., leg ulcer, e.g., venous leg ulcer, e.g. diabetic ulcers, chronic wounds, acne, e.g., acne vulgaris, rosacea, eczema, and psoriasis. The combination therapy may be included in the containers or delivery devices as described herein, or may be delivered using a separate delivery device. The combination therapy may be included in the first chamber, the second chamber, or a third chamber of the container or delivery device. The combination therapy may treat a venous leg ulcer.

For instance, in a combination therapy capable of treating ulcers, e.g., venous ulcer, e.g., leg ulcer, e.g., venous leg ulcer, e.g. diabetic ulcers, the second therapy may comprise, e.g., a wound dressing (e.g., absorptive fillers, hydrogel dressings, or hydrocolloids), angiotensin, angiotensin analogues, platelet-rich fibrin therapy, hyperbaric oxygen therapy, negative pressure wound therapy, debridement, drainage, arterial revascularization, hyperbaric oxygen therapy, low level laser therapy, and gastrocnemius recession. The combination therapy may comprise one or more of the above-mentioned treatments.

In a combination therapy capable of treating chronic wounds, the second therapy may comprise, e.g., an antibiotic (e.g., topical or systemic, and bacteriocidal or bacteriostatic) such as Penicillins, cephalosporins, polymyxins, rifamycins, lipiarmycins, quinolones, sulfonamides, macrolides, lincosamides, tetracyclines, cyclic lipopeptides, glycylcyclines, oxazolidinones, and lipiarmycins; angiotensin, angiotensin analogues; debridement; drainage; wound irrigation; negative pressure wound therapy; application of heat; arterial revascularization; hyperbaric oxygen therapy; antioxidants such as ascorbic acid, glutathione, lipoic acid, carotenes, α-tocopherol, or ubiquinol; low level laser therapy; gastrocnemius recession; growth factors such as vascular endothelial growth factor, insulin-like growth factor 1-2, platelet derived growth factor, transforming growth factor-β, or epidermal growth factor; application of autologous platelets such as those that secrete one or more growth factors such as vascular endothelial growth factor, insulin-like growth factor 1-2, platelet derived growth factor, transforming growth factor-β, or epidermal growth factor; implantation of cultured keratinocytes; allograft; collagen, for instance a dressing comprising collagen; or protease inhibitors such as SLPI. The combination therapy may comprise one or more of the above-mentioned treatments.

In a combination therapy capable of treating acne, e.g., acne vulgaris, the second therapy may comprise, e.g., a medication (e.g., systemic or topical) such as Benzoyl peroxide, antibiotics (such as erythromycin, clindamycin, or a tetracycline), Salicylic acid, hormones (e.g., comprising a progestin such as desogestrel, norgestimate or drospirenone), retinoids such as tretinoin, adapalene, tazarotene, or isotretinoin. The second therapy may also be a procedure such as comedo extraction, corticosteroid injection, or surgical lancing. The combination therapy may comprise one or more of the above-mentioned treatments.

In a combination therapy capable of treating rosacea, the second therapy may comprise, e.g., an antibiotic, e.g., an oral tetracycline antibiotic such as tetracycline, doxycycline, or minocycline, or a topical antibiotic such as metronidazole; azelaic acid; alpha-hydroxy acid; isotretinoin can be prescribed; sandalwood oil; clonidine; beta-blockers such as nadolol and propranolol; antihistamines (such as loratadine); mirtazapine; methylsulfonylmethane or silymarin, optionally in combination with each other; lasers such as dermatological vascular laser or CO₂ laser; or light therapies such as intense pulsed light, low-level light therapy or photorejuvenation. The combination therapy may comprise one or more of the above-mentioned treatments.

In a combination therapy capable of treating eczema, the second therapy may comprise, e.g., a corticosteroid such as hydrocortisone or clobetasol propionate, immunosuppressants (topical or systemic) such as pimecrolimus, tacrolimus, cyclosporin, azathioprine or methotrexate, or light therapy such as with ultraviolet light. The combination therapy may comprise one or more of the above-mentioned treatments.

In a combination therapy capable of treating psoriasis, the second therapy may comprise, e.g., a corticosteroid such as desoximetasone; a retinoid; coal tar; Vitamin D or an analogue thereof such as paricalcitol or calcipotriol; moisturizers and emollients such as mineral oil, vaseline, calcipotriol, decubal, or coconut oil; dithranol; or fluocinonide. The combination therapy may comprise one or more of the above-mentioned treatments.

### 15. Experimental Models for Refining Ammonia Oxidizing Bacteria Treatments

Treatments comprising ammonia oxidizing bacteria as described herein (optionally in combination with another therapy) can be refined using a number of model systems. These model systems can be used to determine suitable doses and timing of administration.

For instance, with respect to chronic wounds and ulcers, e.g., venous ulcers, e.g., diabetic ulcers, or other ulcers disclosed herein, one may use the mouse skin puncture model. Other models for these disorders include controlled cutaneous ischemia in a guinea pig model, rabbit ear ulcer model, application of calcium to a wound, or topical application of doxorubicin.

With respect to acne, e.g., acne vulgaris, one may use (for example) the Mexican hairless dog model, the Rhino mouse model, or the rabbit ear assay. With respect to rosacea, one may use (for example) intradermal injection of LL-37 into mouse skin or the Syrian hamster model. With respect to eczema, one may use (for example) application of a crude extract of Dermatophagoides farina, application of dinitrochlorobenzene to the ears of sensitized guinea pigs, or NC/Nga mice. With respect to psoriasis, one may use (for example) xenograft models in which involved and uninvolved psoriatic skin are transplanted onto immunodeficient mice, application of an antibody directed against interleukin 15 to the skin of SCID mice, and the Sharpin^{cpdm}/Sharpin^{cpdm} mouse model.

### 16. Mechanism of Therapeutic Benefit

While not wishing to be bound by theory, it is believed that one or more of the following mechanisms contributes to the beneficial effect of ammonia oxidizing bacteria are found in International Application WO/2005/030147, which is herein incorporated by reference in its entirety.

In order to understand the beneficial aspects of these bacteria, it is helpful to understand angiogenesis. All body cells, except those within a few hundred microns of the external air, receive all metabolic oxygen from the blood supply. The oxygen is absorbed by the blood in the lung, is carried by red blood cells as oxygenated hemoglobin to the peripheral tissues, where it is exchanged for carbon dioxide, which is carried back and exhaled from the lung. Oxygen must diffuse from the erythrocyte, through the plasma, through the endothelium and through the various tissues until it reached the mitochondria in the cell which consumes it. The human body contains about 5 liters of blood, so the volume of the circulatory system is small compared to that of the body. Oxygen is not actively transported. It passively diffuses down a concentration gradient from the air to the erythrocyte, from the erythrocyte to the cell, and from the cell to cytochrome oxidase where it is consumed. The concentration of oxygen at the site of consumption is the lowest in the body, and the O₂ flux is determined by the diffusion resistance and the concentration gradient. Achieving sufficient oxygen supply to all the peripheral tissues requires exquisite control of capillary size and location. If the spacing between capillaries were increased, achieving the same flux of oxygen would require a larger concentration difference and hence a lower O₂ concentration at cytochrome oxidase. With more cells between capillaries, the O₂ demand would be greater. If the spacing between capillaries were decreased, there would be less space available for the cells that perform the metabolic function of the organ.

In certain aspects, it is appreciated that NO from ammonia oxidizing bacteria is readily absorbed by the outer skin and converted into S-nitrosothiols since the outer skin is free from hemoglobin. M. Stucker et al. have shown that the external skin receives all of its oxygen from the external air in “The cutaneous uptake of atmospheric oxygen contributes significantly to the oxygen supply of human dermis and epidermis. (Journal of Physiology (2002), 538.3, pp. 985-994.) This is readily apparent, because the external skin can be seen to be essentially erythrocyte free. There is circulation of plasma through these layers because they are living and do require the other nutrients in blood, just not the oxygen. S-nitrosothiols formed are stable, can diffuse throughout the body, and constitute a volume source of authentic NO and a source of NO to transnitrosate protein thiols.

In some aspects, it is appreciated that capillary rarefaction may be one of the first indications of insufficient levels of NO. F. T. Tarek et al. have shown that sparse capillaries, or capillary rarefaction, is commonly seen in people with essential hypertension. (Structural Skin Capillary Rarefaction in Essential Hypertension. Hypertension. 1999; 33:998-1001

A great many conditions are associated with the capillary density becoming sparser. Hypertension is one, and researchers reported that sparse capillaries are also seen in the children of people with essential hypertension, and also in people with diabetes. Significant complications of diabetes are hypertension, diabetic nephropathy, diabetic retinopathy, and diabetic neuropathy. R. Candido et al. have found that the last two conditions are characterized by a reduction in blood flow to the affected areas prior to observed symptoms. (Haemodynamics in microvascular complications in type 1 diabetes. Diabetes Metab Res Rev 2002; 18: 286-304.) Reduced capillary density is associated with obesity, and simple weight loss increases capillary density as shown by A Philip et al. in “Effect of Weight Loss on Muscle Fiber Type, Fiber Size, Capillarity, and Succinate Dehydrogenase Activity in Humans. The Journal of Clinical Endocrinology & Metabolism Vol. 84, No. 11 4185-4190, 1999.

Researchers have shown that in primary Raynaud's phenomena (PRP), the nailfold capillaries are sparser (slightly) than in normal controls, and more abundant than in patients that have progressed to systemic sclerosis (SSc). M. Bukhari, Increased Nailfold Capillary Dimensions In Primary Raynaud's Phenomenon And Systemic Sclerosis. British Journal of Rheumatology, Vol. 24 No 35: 1127-1131, 1996. They found that the capillary density decreased from 35 loops/mm² (normal controls) to 33 (PRP), to 17 (SSc). The average distance between capillary limbs was 18μ, 18μ, and 30μ for controls, PRP and SSc, respectively.

In certain aspects, it is appreciated that the mechanism that the body normally uses to sense “hypoxia” may affect the body's system that regulates capillary density. According to this aspect of the disclosure, a significant component of “hypoxia” is sensed, not by a decrease in 02 levels, but rather by an increase in NO levels. Lowering of basal NO levels interferes with this “hypoxia” sensing, and so affects many bodily functions regulated through “hypoxia.” For Example, anemia is commonly defined as “not enough hemoglobin,” and one consequence of not enough hemoglobin is “hypoxia”, which is defined as “not enough oxygen.” According to some aspects, these common definitions do not account for the nitric oxide mediated aspects of both conditions.

At rest, acute isovolemic anemia is well tolerated. A ⅔ reduction in hematocrit has minimal effect on venous return PvO2, indicating no reduction in either O₂ tension or delivery throughout the entire body. Weiskopf et al. Human cardiovascular and metabolic response to acute, severe isovolemic anemia. JAMA 1998, vol 279, No. 3, 217-221. At 50% reduction (from 140 to 70 g Hb/L), the average PvO2 (over 32 subjects) declined from about 77% to about 74% (of saturation). The reduction in O₂ capacity of the blood is compensated for by vasodilatation and tachycardia with the heart rate increasing from 63 to 85 bpm. That the compensation is effective is readily apparent, however, the mechanism is not. A typical explanation is that “hypoxia” sensors detected “hypoxia” and compensated with vasodilatation and tachycardia. However, there was no “hypoxia” to detect. There was a slight decrease in blood lactate (a marker for anaerobic respiration) from 0.77 to 0.62 mM/L indicating less anaerobic respiration and less “hypoxia.” The 3% reduction in venous return PvO2 is the same level of “hypoxia” one would get by ascending 300 meters in altitude (which typically does not produce tachycardia). With the O₂ concentration in the venous return staying the same, and the O₂ consumption staying the same, there is no place in the body where there is a reduction in O₂ concentration. Compensation during isovolemic anemia may not occur because of O₂ sensing.

Thus the vasodilatation that is observed in acute isovolemic anemia may be due to the increased NO concentration at the vessel wall. NO mediates dilatation of vessels in response to shear stress and other factors. No change in levels of NO metabolites would be observed, because the production rate of NO is unchanged and continues to equal the destruction rate. The observation of no “hypoxic” compensation with metHb substitution can be understood because metHb binds NO just as Hb does, so there is no NO concentration increase with metHb substitution as there is with Hb withdrawal.

Nitric oxide plays a role in many metabolic pathways. It has been suggested that a basal level of NO exerts a tonal inhibitory response, and that reduction of this basal level leads to a dis-inhibition of those pathways. Zanzinger et al. have reported that NO has been shown to inhibit basal sympathetic tone and attenuate excitatory reflexes. (Inhibition of basal and reflex-mediated sympathetic activity in the RVLM by nitric oxide. Am. J. Physiol. 268 (Regulatory Integrative Comp. Physiol. 37): R958-R962, 1995.)

In some aspects, it is appreciated that one component of a volume source of NO is low molecular weight S-nitrosothiols produced in the erythrocyte free skin from NO produced on the external skin by ammonia oxidizing bacteria. These low molecular weight S-nitrosothiols are stable for long periods, and can diffuse and circulate freely in the plasma. Various enzymes can cleave the NO from various S-nitrosothiols liberating NO at the enzyme site. It is the loss of this volume source of NO from AOB on the skin that leads to disruptions in normal physiology. The advantage to the body of using S-nitrosothiols to generate NO far from a capillary is that O₂ is not required for NO production from S-nitrosothiols. Production of NO from nitric oxide synthase (NOS) does require O₂. With a sufficient background of S-nitrosothiols, NO can be generated even in anoxic regions. Free NO is not needed either since NO only exerts effects when attached to another molecule, such as the thiol of a cysteine residue or the iron in a heme, so the effects of NO can be mediated by transnitrosation reactions even in the absence of free NO provided that S-nitrosothiols and transnitrosation enzymes are present.

Frank et al. have shown that the angiogenesis that accompanies normal wound healing is produced in part by elevated VEGF which is induced by increased nitric oxide. (Nitric oxide triggers enhanced induction of vascular endothelial growth factor expression in cultured keratinocytes (HaCaT) and during cutaneous wound repair. FASEB J. 13, 2002-2014 (1999).)

NO has a role in the development of cancer, indicating that the bacteria described herein may be used in methods of cancer treatment and prevention. According to certain aspects, it is appreciated that the presence of NO during hypoxia may prevent cells from dividing while under hypoxic stress, when cells are at greater risk for errors in copying DNA. One relevant cell function is the regulation of the cell cycle. This is the regulatory program which controls how and when the cell replicates DNA, assembles it into duplicate chromosomes, and divides. The regulation of the cell cycle is extremely complex, and is not fully understood. However, it is known that there are many points along the path of the cell cycle where the cycle can be arrested and division halted until conditions for doing so have improved. The p53 tumor suppressor protein is a key protein in the regulation of the cell cycle, and it serves to initiate both cell arrest and apoptosis from diverse cell stress signals including DNA damage and p53 is mutated in over half of human cancers as reported by Ashcroft et al. in “Stress Signals Utilize Multiple Pathways To Stabilize p53.” (Molecular And Cellular Biology, May 2000, p. 3224-3233.) Hypoxia does initiate accumulation of p53, and while hypoxia is important in regulating the cell cycle, hypoxia alone fails to induce the downstream expression of p53 mRNA effector proteins and so fails to cause arrest of the cell cycle. Goda et al. have reported that hypoxic induction of cell arrest requires hypoxia-inducing factor-1 (HIF-1α). (Hypoxia-Inducible Factor 1α Is Essential for Cell Cycle Arrest during Hypoxia. Molecular And Cellular Biology, January 2003, p. 359-369.) Britta et al. have reported that NO is one of the main stimuli for HIF-1α. (Accumulation of HIF-1a under the influence of nitric oxide. Blood, 15 Feb. 2001, Volume 97, Number 4.) In contrast, NO does cause the accumulation of transcriptionally active p53 and does cause arrest of the cell cycle and does cause apoptosis. Wang et al., P53 Activation By Nitric Oxide Involves Down-Regulation Of Mdm2. THE JOURNAL OF BIOLOGICAL CHEMISTRY Vol. 277, No. 18, Issue Of May 3, Pp. 15697-15702, 2002.

In certain aspect of the disclosure, it is appreciated that preventing the necrotic death of cells by preventing the capillary rarefaction that leads to their hypoxic death may prevent autoimmune disorders. When cells are exposed to chronic hypoxia, the production of reactive oxygen species (ROS) is increased, and there is increased damage to the cells metabolic machinery and ultimately to the cells' DNA. Decreased metabolic capacity will decrease capacity for repair of damage due to ROS and due to exogenous carcinogen exposure. Over time, the damage accumulates and increases the chance of three events: the cell will undergo deletion of cancer-preventing genes and the cell will become cancerous, the cell will die through necrosis, or the cell will die through apoptosis. When cells die, either through necrosis or apoptosis, the cell debris must be cleared from the site. Dead cells are phagocytosed by immune cells, including dendritic cells and macrophages. When these cells phagocytose a body, it is digested by various proteolytic enzymes into antigenic fragments, and then these antigens are attached to the major histocompatibility complex (MHC1, MHC2) and the antigen-MHC complex is moved to the surface of the cell where it can interact with T cells and activate the T cells in various ways. Any cell injury releases adjuvants which stimulate the immune system in various ways. In general, cells that undergo necrosis stimulate a greater immune response than cells that undergo apoptosis. Chronic exposure of immune cells to dead and dying cells is therefore likely to lead to autoimmune disorders.

In certain aspects, it is appreciated that low basal NO leads to fibrotic hypertrophy. Once a dead cell has been cleared, a new cell cannot easily take its place, because there is insufficient O₂ to support it. Any such new cell would suffer the same fate. The space can remain empty, in which case the organ shrinks, the capillaries draw closer together, new cells are now deprived of the VEGF formerly produced by the now-missing cell, so capillaries ablate and the hypoxic zone reforms. This could result in a general shrinkage of the affected tissues. In tissues that support fibrosis, relatively inert collagen fibers can fill the space. Since the metabolic requirements of the body for the particular organ in question are not reduced, the organ may attempt to grow larger, but now with a significant fibrous content. This may result in fibrotic hypertrophy, such as of the heart and liver. Some organs, such as the brain, cannot grow larger or smaller because the three-dimensional connectivity of nerves and blood vessels are important, and cannot be continuously and simultaneously mapped onto an asymmetrically shrinking brain. The space must be filled with something, and β-amyloid might be the (not so inert) space filler. The kidney cannot grow larger because of the renal capsule, so the number of living cells becomes smaller and they are replaced with fibrotic tissue. If the dead cells are cleared, the tissue shrinks, and the ratio of NO/O₂ goes down again, and the capillaries again become sparser. This may set up the vicious circle of end stage renal disease, congestive heart failure/cardiac hypertrophy, primary biliary cirrhosis, Alzheimer's disease, atherosclerosis, inflammatory bowel disease, hypertrophic scar formation, and the multiple connective tissue diseases starting with Raynaud's phenomena and ending with Systemic Sclerosis and primary Sjogren's syndrome where capillary rarefaction is also observed. Ferrini et al, have shown that a reduction in basal NO levels through chronic inhibition of NOS with L-NAME leads to generalized fibrosis of the heart and kidneys. (Antifibrotic Role of Inducible Nitric Oxide Synthase. Nitric Oxide: Biology and Chemistry Vol. 6, No. 3, pp. 283-294 (2002).) It may be that low basal NO leads to fibrotic hypertrophy.

In certain aspects, it is appreciated that capillary rarefaction affects a subject's ability to control their appetite. Capillary rarefaction is observed in the brains of aged humans and animals. Capillary rarefaction is associated with declines in circulating growth factors including insulin like growth factor-1. Neurogenesis in the adult brain is coordinated with angiogenesis. Since the brain regulates many homeostatic functions, increased diffusion lengths between capillaries to control elements of the brain might be “interpreted” as inadequate blood concentrations of those species. The flux of glucose in the brain is quite close to normal metabolic needs, where glucose flux is only 50 to 75% greater than glucose consumption and the glucose transporters across the blood brain barrier are saturable, stereospecific and independent of energy or ion gradients. A large part of the regulation of appetite is mediated through the brain, and capillary rarefaction may cause an adequate blood concentration of “nutrients” (or marker compounds proportional to “nutrients”) to be interpreted as insufficient. This may be one cause of obesity.

According to certain aspects, it is appreciated that capillary rarefaction may be a cause of non-insulin dependent diabetes. Non-insulin dependent diabetes (NIDDM) is also known as the Metabolic Syndrome or Diabetes type 2, and is characterized by insulin resistance. The sensitivity of the body to insulin is reduced, and insulin levels increase People with NIDDM have high blood glucose, high blood triglycerides, are typically obese, hypertensive, and typically have significant visceral fat.

Other symptoms accompany NIDDM, which may point to capillary rarefaction as the cause. In a study of 40 men, with and without NIDDM, obese (BMI 29) and lean (BMI 24) (10 of each), Konrad et al. report that blood lactate levels at rest were 1.78, 2.26, 2.42, and 2.76 (mM/L) for lean men without, obese men without, lean men with NIDDM, obese men with NIDDM respectively. (A-Lipoic acid treatment decreases serum lactate and pyruvate concentrations and improves glucose effectiveness in lean and obese patients with type 2 diabetes. Diabetes Care 22:280-287, 1999.) Lactate is a measure of anaerobic glycolysis. When O₂ is insufficient to generate ATP through oxidative phosphorylation, cells can produce ATP through anaerobic glycolysis. One of the products of anaerobic glycolysis is lactate, which must be exported from the cells, otherwise the pH drops and function is compromised. Blood lactate is commonly measured in exercise studies, where an increase indicates the work load at which maximum oxidative work can be done. Higher levels of lactate at rest would indicate increased anaerobic glycolysis at rest, which is consistent with capillary rarefaction.

Primary biliary cirrhosis is associated with Raynaud's phenomena, pruritus, sicca syndrome, osteoporosis, portal hypertension, neuropathy, and pancreatic insufficiency, and liver abnormalities are associated with rheumatic diseases. Elevated liver enzymes are a symptom of liver inflammation, and elevated liver enzymes are observed as an early symptom of “asymptomatic” primary biliary cirrhosis. Accordingly, the bacteria described herein may be used to treat liver inflammation.

Torre et al have reported that Alzheimer's disease (AD) is a microvascular disorder with neurological degeneration secondary to hypoperfusion, resulting in part from insufficient nitric oxide. (Review: Evidence that Alzheimer's disease is a microvascular disorder: the role of constitutive nitric oxide, Brain Research Reviews 34 (2000) 119-136.) Accordingly, the bacteria described herein may be used to treat AD.

Adverse health effects that are associated with hypertension may also be consequences of low basal NO. The decreased response to vasodilatation is also consistent with low basal NO. NO is a diffusible molecule that diffuses from a source to a sensor site where it has the signaling effect. With low NO levels, every NO source must produce more NO to generate an equivalent NO signal of a certain intensity a certain distance away. NO diffuses in three dimensions and the whole volume within that diffusion range must be raised to the level that will give the proper signal at the sensor location. This may result in higher NO levels at the source and between the source and the sensor. Adverse local effects of elevated NO near a source may then arise from too low a NO background. There is some evidence that this scenario actual occurs. In rat pancreatic islets, Hennings son et al have reported that inhibition of NOS with L-NAME increases total NO production through the induction of iNOS. (Chronic blockade of NO synthase paradoxically increases islet NO production and modulates islet hormone release. Am J Physiol Endocrinol Metab 279: E95-E107, 2000.) Increasing NO by increasing NOS activity will only work up to some limit. When NOS is activated but is not supplied with sufficient tetrahydrobiopterin (BH4) or L-arginine, it becomes “uncoupled” and generates superoxide (02-) instead of NO. This O₂⁻ may then destroy NO. Attempting to produce NO at a rate that exceeds the supply of BH4 or L-arginine may instead decrease NO levels. This may result in positive feedback where low NO levels are made worse by stimulation of NOS, and uncoupled NOS generates significant O₂⁻ which causes local reactive oxygen species (ROS) damage such as is observed in atherosclerosis, end stage renal disease, Alzheimer's, and diabetes.

The bacteria described herein may also be used to delay the signs of aging. Caloric restriction extends lifespan, and Holloszy reported that restricting food intake to 70% of ad lib controls, prolongs life in sedentary rats from 858 to 1,051 days, almost 25%. (Mortality rate and longevity of food restricted exercising male rats: a reevaluation. J. Appl. Physiol. 82(2): 399-403, 1997.) The link between calorie restriction and prolonged life is well established, however, the causal mechanism is not. Lopez-Torres et al. reported that the examination of liver mitochondrial enzymes in rats indicates a reduction in H₂O₂ production due to reduced complex I activity associated with calorie restriction. (Influence Of Aging And Long-Term Caloric Restriction On Oxygen Radical Generation And Oxidative DNA Damage In Rat Liver Mitochondria. Free Radical Biology & Medicine Vol. 32 No 9 pp 882-8899, 2002.) H₂O₂ is produced by dismutation of O₂⁻, which is a major ROS produced by the mitochondria during respiration. The main source of O₂⁻ has been suggested by Kushareva et al. and others to be complex I which catalyzes the NAD/NADH redox couple by reverse flow of electrons from complex III, the site of succinate reduction. The free radical theory, proposed by Beckman, of aging postulates, that free radical damage to cellular DNA, antioxidant systems and DNA repair systems accumulates with age and when critical systems are damaged beyond repair, death ensues. (The Free Radical Theory of Aging Matures. Physiol. Rev. 78: 547-581, 1998.)

As an additional mechanism, NO has been demonstrated by Vasa et al. to activate telomerase and to delay senescence of endothelial cells. (Nitric Oxide Activates Telomerase and Delays Endothelial Cell Senescence. Circ Res. 2000; 87:540-542.) Low basal NO will increase basal metabolic rate by disinhibition of cytochrome oxidase. Increased basal metabolism will also increase cell turn-over and growth rate. Capillary rarefaction, by inducing chronic hypoxia may increase free radical damage and may also increase cell turn-over, and so accelerate aging by both mechanisms.

In some aspects, it is appreciated that autotrophic ammonia-oxidizing bacteria may produce protective aspects for allergies and autoimmune disorders. The best known autoimmune disease is perhaps Diabetes Type 1, which results from the destruction of the insulin producing cells in the pancreas by the immune system. Recurrent pregnancy loss is also associated with autoimmune disorders where the number of positive autoimmune antibodies correlated positively with numbers recurrent pregnancy losses. Systemic Sclerosis, Primary Biliary Cirrhosis, autoimmune hepatitis, and the various rheumatic disorders are other examples of autoimmune disorders. Application of AOB was observed to reduce an allergy, hay fever, as described in WO/2005/030147.

One mechanism by which AOB may exert their protective effect on allergies and autoimmune disorders is through the production of nitric oxide, primarily through the regulatory inhibition of NF- [symbol] B and the prevention of activation of immune cells and the induction of inflammatory reactions. NF- [symbol] B is a transcription factor that up-regulates gene expression and many of these genes are associated with inflammation and the immune response including genes which cause the release of cytokines, chemokines, and various adhesion factors. These various immune factors cause the migration of immune cells to the site of their release resulting in the inflammation response. Constitutive NO production has been shown to inhibit NF- [symbol] B by stabilizing I [symbol] Bα (an inhibitor of NF- [symbol] B) by preventing I [symbol] B a degradation.

Administration of an NO donor has been shown by Xu et al. to prevent the development of experimental allergic encephalomyelitis in rats. (SIN-1, a Nitric Oxide Donor, Ameliorates Experimental Allergic Encephalomyelitis in Lewis Rats in the Incipient Phase: The Importance of the Time Window. The Journal of Immunology, 2001, 166: 5810-5816.) In this study, it was demonstrated that administering an NO donor, reduced the infiltration of macrophages into the central nervous system, reduced the proliferation of blood mononuclear cells, and increased apoptosis of blood mononuclear cells. All of these results are expected to reduce the extent and severity of the induced autoimmune response.

Low basal NO may lead to autism via the mechanism that new connections in the brain are insufficiently formed as a result of insufficient basal nitric oxide. While not wishing to be bound in theory, in some embodiments, formation of neural connections is modulated by NO. In these cases, any condition that lowers the range of NO diffusion may decrease the volume size of brain elements that can undergo connections. A brain which developed under conditions of low basal NO levels may be arranged in smaller volume elements because the reduced effective range of NO.

Additional symptoms exhibited in autistic individuals may also point to low NO as a cause, including increased pitch discrimination, gut disturbances, immune system dysfunction, reduced cerebral blood flow, increased glucose consumption of the brain, increased plasma lactate, attachment disorders, and humming. Each of these symptoms may be attributed to a low basal NO level.

Takashi Ohnishi et al. have reported that autistic individuals show decreased blood flow. Takashi Ohnishi et al., Abnormal regional cerebral blood flow in childhood autism. Brain (2000), 123, 1838-1844. J. M. Rumsey et al. have reported that autistic individuals have increased glucose consumption. Rumsey J M, Duara R, Grady C, Rapoport J L, Margolin R A, Rapoport S I, Cutler N R. Brain metabolism in autism. Resting cerebral glucose utilization rates as measured with positron emission tomography. Arch Gen Psychiatry, 1985 May; 42(5):448-55 (abstract). D. C. Chugani has reported that autistic individuals have an increased plasma lactate levels. Chugani D C, et al., Evidence of altered energy metabolism in autistic children. Prog Neuropsychopharmacol Biol Psychiatry. 1999 May; 23(4):635-41. The occurrence of these effects may be a result of capillary rarefaction in the brain, which may reduce blood flow and O₂ supply, such that some of the metabolic load of the brain may be produced through glycolysis instead of oxidative phosphorylation.

Nitric oxide has been demonstrated by B. A. Klyachko et al. to increase the excitability of neurons by increasing the after hyperpolarization through cGMP modification of ion channels. Vitaly A. Klyachko et al., cGMP-mediated facilitation in nerve terminals by enhancement of the spike after hyperpolarization. Neuron, Vol. 31, 1015-1025, Sep. 27, 2001. C. Sandie et al. have shown that inhibition of NOS reduces startle. Carmen Sandi et al., Decreased spontaneous motor activity and startle response in nitric oxide synthase inhibitor-treated rats. European journal of pharmacology 277 (1995) 89-97. Attention-Deficit Hyperactivity Disorder (ADHD) has been modeled using the spontaneously hypertensive rat (SHR) and the Naples high-excitability (NHE) rat. Both of these models have been shown by Raffaele Aspide et al, to show increased attention deficits during periods of acute NOS inhibition. Raffaele Aspide et al., Non-selective attention and nitric oxide in putative animal models of attention-deficit hyperactivity disorder. Behavioral Brain Research 95 (1998) 123-133. Accordingly, the bacteria herein may be used in the treatment of ADHD.

Inhibition of NOS has also been shown by M. R. Dzoljic to inhibit sleep. M. R. Dzoljic, R. de Vries, R. van Leeuwen. Sleep and nitric oxide: effects of 7-nitro indazole, inhibitor of brain nitric oxide synthase. Brain Research 718 (1996) 145-150. G. Zoccoli has reported that a number of the physiological effects seen during sleep are altered when NOS is inhibited, including rapid eye movement and sleep-wake differences in cerebral circulation. G. Zoccoli, et al., Nitric oxide inhibition abolishes sleep-wake differences in cerebral circulation. Am. J. Physiol. Heart Circ Physiol 280: H2598-2606, 2001. NO donors have been shown by L. Kapas et al. to promote non-REM sleep, however, these increases persisted much longer than the persistence of the NO donor, suggesting perhaps a rebound effect. Levente Kapas et al. Nitric oxide donors SIN-1 and SNAP promote nonrapid-eye-movement sleep in rats. Brain Research Bullitin, vol 41, No 5, pp. 293-298, 1996. M. Rosaria et al., Central NO facilitates both penile erection and yawning. Maria Rosaria Melis and Antonio Argiolas. Role of central nitric oxide in the control of penile erection and yawning. Prog Neuro-Psychopharmacol & Biol. Phychiat. 1997, vol 21, pp 899-922. P. Tani et al, have reported that insomnia is a frequent finding in adults with Asperger's. Pekka Tani et al., Insomnia is a frequent finding in adults with Asperger's syndrome. BMC Psychiatry 2003, 3:12. Y. Hoshino has also observed sleep disturbances in autistic children. Hoshino Y, Watanabe H, Yashima Y, Kaneko M, Kumashiro H. An investigation on sleep disturbance of autistic children. Folia Psychiatr Neurol Jpn. 1984; 38(1):45-51. (abstract) K. A. Schreck et al. has observed that the severity of sleep disturbances correlates with severity of autistic symptoms. Schreck K A, et al., Sleep problems as possible predictors of intensified symptoms of autism. Res Dev Disabil. 2004 January-February; 25(1):57-66. (abstract). Accordingly, the bacteria herein may be used in the treatment of insomnia.

W. D. Ratnasooriya et al reported that inhibition of NOS in male rats reduces pre-coital activity, reduces libido, and reduces fertility. W. D. Ratnasooriya et al., Reduction in libido and fertility of male rats by administration of the nitric oxide (NO) synthase inhibitor N-nitro-L-arginine methyl ester. International journal of andrology, 23: 187-191 (2000).

It may be that a number of seemingly disparate disorders, characterized by ATP depletion and eventual organ failure are actually “caused” by nitropenia, caused by a global deficiency in basal nitric oxide. When this occurs in the heart, the result is dilative cardiomyopathy. When this occurs in the brain, the result is white matter hyperintensity, Alzheimer's, vascular depression, vascular dementia, Parkinson's, and the Lewy body dementias. When this occurs in the kidney, the result is end stage renal disease, when this occurs in the liver, the result is primary biliary cirrhosis. When this occurs in muscle, the consequence is fibromyaligia, Gulf War Syndrome, or chronic fatigue syndrome. When this occurs in the bowel, the consequence is ischemic bowel disease. When this occurs in the pancreas, the consequence is first type 2 diabetes, followed by chronic inflammation of the pancreas, followed by autoimmune attack of the pancreas (or pancreatic cancer), followed by type 1 diabetes. When this occurs in the connective tissue, the consequence is systemic sclerosis.

In the remnant kidney model of end stage renal disease, part of the kidney is removed, (either surgically or with a toxin) which increases the metabolic load on the remainder. Superoxide is generated to decrease NO and increase O₂ diffusion to the kidney mitochondria. Chronic overload results in progressive kidney capillary rarefaction and progressive kidney failure. In acute kidney failure, putting people in dialysis can give the kidney a “rest”, and allows it to recover. In acute renal failure induced by rhabdomyolysis (muscle damage which releases myoglobin into the blood stream) kidney damage is characterized by ischemic damage. Myoglobin scavenges NO, just as hemoglobin does, and would cause vasoconstriction in the kidney leading to ischemia. Myoglobin would also induce local nitropenia and the cascade of events leading to further ATP depletion.

In some aspects, low NO levels lead to reduced mitochondrial biogenesis. Producing the same ATP at a reduced mitochondria density will result in an increase in O₂ consumption, or an accelerated basal metabolic rate. An accelerated basal metabolic rate is observed in a number of conditions, including: Sickle cell anemia, Congestive heart failure, Diabetes, Liver Cirrhosis, Crohn's disease, Amyotrophic lateral sclerosis, Obesity, End stage renal disease, Alzheimer's, and chronic obstructive pulmonary disease.

While some increased O₂ consumption might be productively used, in many of these conditions uncoupling protein is also up-regulated, indicating that at least part of the increased metabolic rate is due to inefficiency. Conditions where uncoupling protein is known to be up-regulated include obesity and diabetes.

With fewer mitochondria consuming O₂ to a lower O₂ concentration, the O₂ gradient driving O₂ diffusion is greater, so the O₂ diffusion path length can increase resulting in capillary rarefaction, which is observed in dilative cardiomyopathy, hypertension, diabetes type 2, and renal hypertension.

Copper, either as Cu2+ or as ceruloplasmin (CP) (the main Cu containing serum protein which is present at 0.38 g/L in adult sera and which is 0.32% Cu and contains 94% of the serum copper) catalyzes the formation of S—NO-thiols from NO and thiol containing groups (RSH). The Cu content of plasma is variable and is increased under conditions of infection. Berger et al. reported that the Cu and Zn content of burn-wound exudates is considerable with patients with ⅓ of their skin burned, losing 20 to 40% of normal body Cu and 5 to 10% of Zn content in 7 days. (Cutaneous copper and zinc losses in burns. Burns. 1992 October; 18(5):373-80.) If the patients skin were colonized by AOB, wound exudates which contains urea and Fe, Cu, and Zn that AOB need, would be converted into NO and nitrite, greatly supplementing the local production of NO by iNOS, without consuming resources (such as O₂ and L-arginine) in the metabolically challenged wound. A high production of NO and nitrite by AOB on the surface of a wound would be expected to inhibit infection, especially by anaerobic bacteria such as the Clostridia which cause tetanus, gas gangrene, and botulism.

The practice of the present disclosure may employ, unless otherwise indicated, conventional methods of immunology, molecular biology, and recombinant DNA techniques within the skill of the art. Such techniques are explained fully in the literature. See, e.g., Sambrook, et al. Molecular Cloning: A Laboratory Manual (Current Edition); and Current Protocols in Molecular Biology (F. M. Ausubel, et al. eds., current edition).

### Examples

### Biome-Friendly Ingredient and Formulae Testing Method

Recovery of N. eutropha D23 after Treatment with Various Excipients

N. eutropha D23 suspensions were obtained from a continuous culture system. The N. eutropha D23 culture was harvested by centrifugation at 10,000×g for 15 minutes at 20° C. The harvested cells were washed in AOB storage solution (50 mM Na₂HPO₄— 2 mM MgCl₂, pH 7.6) and suspended in storage solution at a final optical density (OD₆₀₀) of 5.0 (˜10¹⁰ cells/ml) prior to storing at 4° C. To determine the effect of each excipient, the N. eutropha D23 cell suspension was diluted to a final optical density (OD₆₀₀) of 0.5 (˜10⁹ cells/ml) in 10 ml AOB medium supplemented with ammonium (NH₄⁺) containing various concentrations of the excipient (0 to 100%). Control cultures were supplemented with an equal volume of excipient diluent only. The cultures were incubated at 30° C. At 1 min, 10 min & 60 min time points, 1 ml of the cultures were collected, centrifuged at 17,000×g for 3 minutes. The supernatant was used to measure nitrite by Griess reagents. The bacterial pellet obtained was washed in AOB medium, suspended in 10 ml AOB medium supplemented with NH₄⁺ and incubated at 30° C. shaking at 150 rpm (upright position) on an orbital shaker. Recovery of N. eutropha D23 cells from treatment with excipient was monitored for 24-48 hr by determining OD₆₀₀ values & nitrite accumulation in samples collected at 24 hr intervals.

Depending on the ingredient, excipient, or composition tested, the concentration of nitrite measured may allow for identification of a biome-friendly ingredient, excipient, or composition. In certain embodiments, only nitrite production of greater than 1000 micromolar, measured at the end of a 48 hour period would be indicative of a biome-friendly ingredient, excipient, or composition. In other embodiments, nitrite production of greater than 100 micromolar, measured at the end of a 48 hour period would be indicative of a biome-friendly ingredient, excipient, or composition. In other embodiments, nitrite production of greater than 10 micromolar, measured at the end of a 48 hour period would be indicative of a biome-friendly ingredient, excipient, or composition.

Recovery identified as “+++” is indicative of nitrite production of greater than 1000 micromolar, measured at the end of a 48 hour period of time, and may provide for a biome-friendly ingredient, excipient or composition. Recovery identified as “++” is indicative of nitrite production between about 100 micromolar and 1000 micromolar, at the end of a 48 hour period of time, and may provide for a biome-friendly ingredient, excipient or composition. Recovery identified as “+” is indicative of nitrite production between about 10 and 100 micromolar, at the end of a 48 hour period of time, and may provide for a biome-friendly ingredient, excipient or composition. No recovery (−) is indicative of none or substantially no nitrite production measured, for example, less than 10 micromolar nitrite production in a 48 hour period of time, which would in most instances would not be indicative of a biome-friendly ingredient, excipient or composition.

This procedure may apply to water soluble or miscible ingredients. For oil or immiscible ingredients or formulae, the test substance is may first be adsorbed to a carrier bead or mesh. This may be rinsed in buffer and then the beads or mesh may be placed in the bacterial suspension and incubated for the specified time period. The bare bead or mesh may be incubated as a control. At the end of the test exposure period, the supernatant bacterial suspension may be tested for activity and viability as per the soluble test methodology, discussed above.

For the examples, e.g., shampoo and cleanser, testing was done at 10% and 25% because they are typically diluted with water in approximately these ratios during use.

The compositions tested had the follow composition of ingredients:

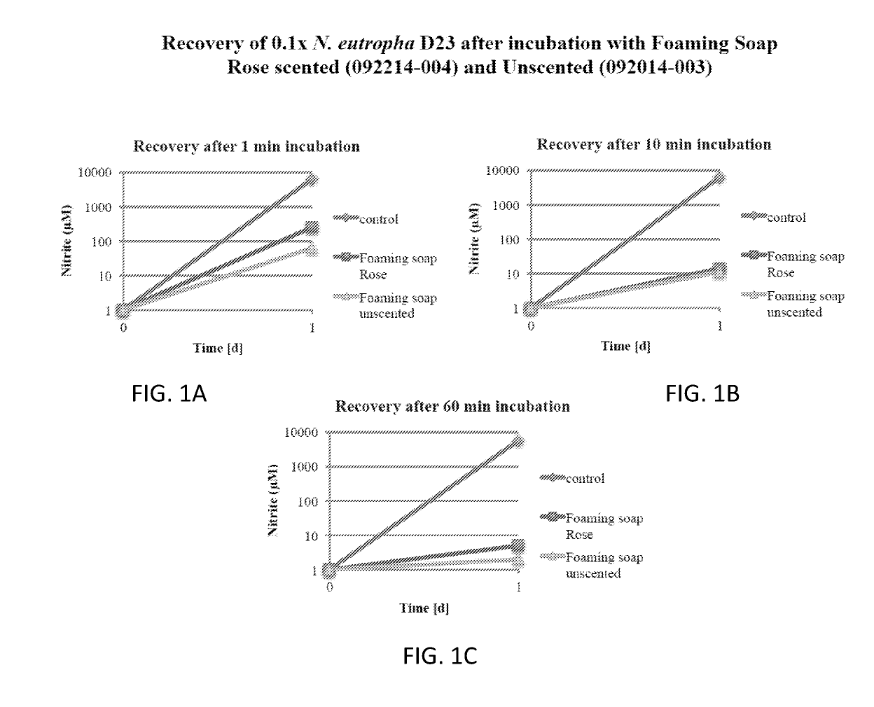

092214-004 rose scented foaming soap (FIG. 1A-1C)

| | | Possible | |
|---|---|---|---|
| Component | Concentration | Concentration | Ingredient |
| Natrasol 300-CS | 0.25% | 0.10-0.5% | Viscosity |
| (Hydroxyethyl | | | modifier |
| Cellulose) | | | |
| Cocamidopropyl | 20.00% | 5.0-12.0% | Cleanser/ |
| Betaine | | | Surfactant |
| Decyl Glucoside | 4.0% | 0.0-8.0% | Cleanser/ |
| | | | Surfactant |
| Botanimoist AMG | 2.0% | 0.0-4.0% | Humectant |
| (Glycerine + | | | |
| Apple ext | | | |
| Natural Rose | 5.0% | 0.0-10.0% | Fragrance |
| Hydrosol | | | |

092014-003 unscented foaming soap (FIG. 1A-1C)

| | | Possible | |
|---|---|---|---|
| Component | Concentration | Concentration | Ingredient |
| Natrasol 300-CS | 0.25% | 0.10-0.5% | Viscosity |
| (Hydroxyethyl | | | modifier |
| Cellulose) | | | |
| Cocamidopropyl | 8.00% | 5.0-12.0% | Cleanser/ |
| Betaine | | | Surfactant |
| Decyl Glucoside | 4.0% | 0.0-8.0% | Cleanser/ |
| | | | Surfactant |
| Botanimoist AMG | 2.0% | 0.0-4.0% | Humectant |
| (Glycerine + | | | |
| Apple ext) | | | |

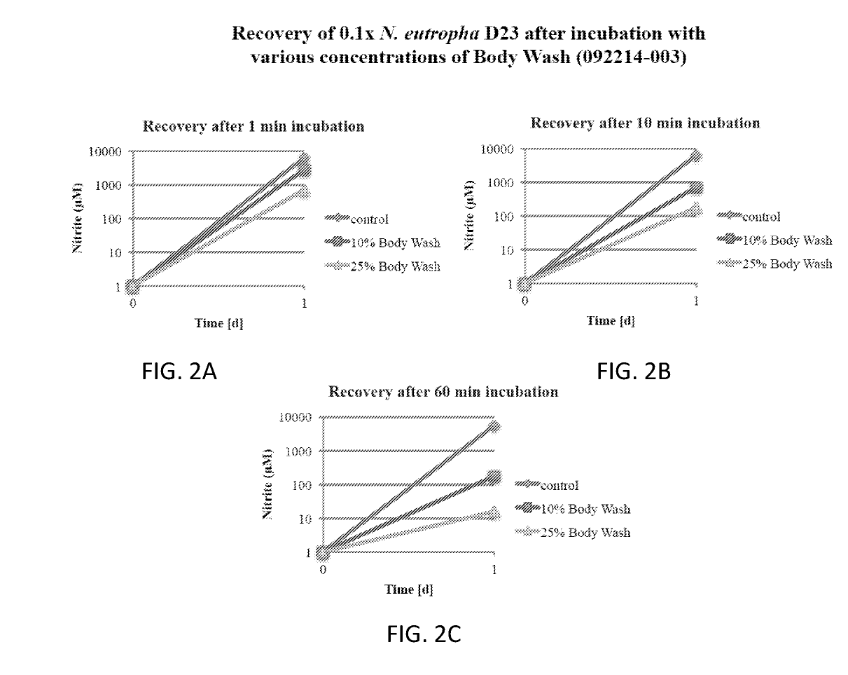

092214-003 body wash (FIG. 2A-2C)

| | | Possible | |
|---|---|---|---|
| Component | Concentration | Concentration | Ingredient |
| Natrasol 300-CS | 2.00% | 1.0-2.5% | Viscosity |
| (Hydroxyethyl | | | modifier |
| Cellulose) | | | |
| Cocamidopropyl | 20.00% | 15.0-30.0% | Cleanser/ |
| Betaine | | | Surfactant |
| Decyl Glucoside | 10.0% | 0.0-15.0% | Cleanser/ |
| | | | Surfactant |
| Botanimoist AMG | 2.0% | 0.0-4.0% | Humectant |
| (Glycerine + | | | |
| Apple ext) | | | |

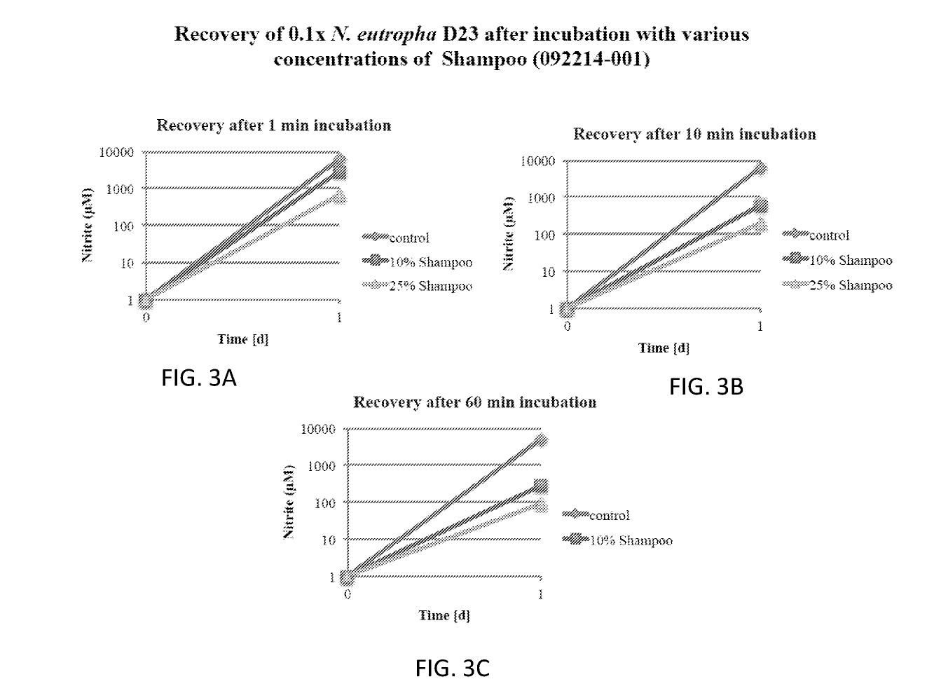

092214-001 shampoo (FIG. 3A-3C)

| | | Possible | |
|---|---|---|---|
| Component | Concentration | Concentration | Ingredient |
| Natrasol 300-CS | 2.00% | 0.5-2.5% | Viscosity |
| (Hydroxyethyl | | | modifier |
| Cellulose) | | | |
| Cocamidopropyl | 20.00% | 10.0-30.0% | Cleanser/ |
| Betaine | | | Surfactant |
| Decyl Glucoside | 5.0% | 0.0-10.0% | Cleanser/ |
| | | | Surfactant |
| Botanimoist AMG | 2.0% | 0.0-4.0% | Humectant |
| (Glycerine + | | | |
| Apple extract) | | | |

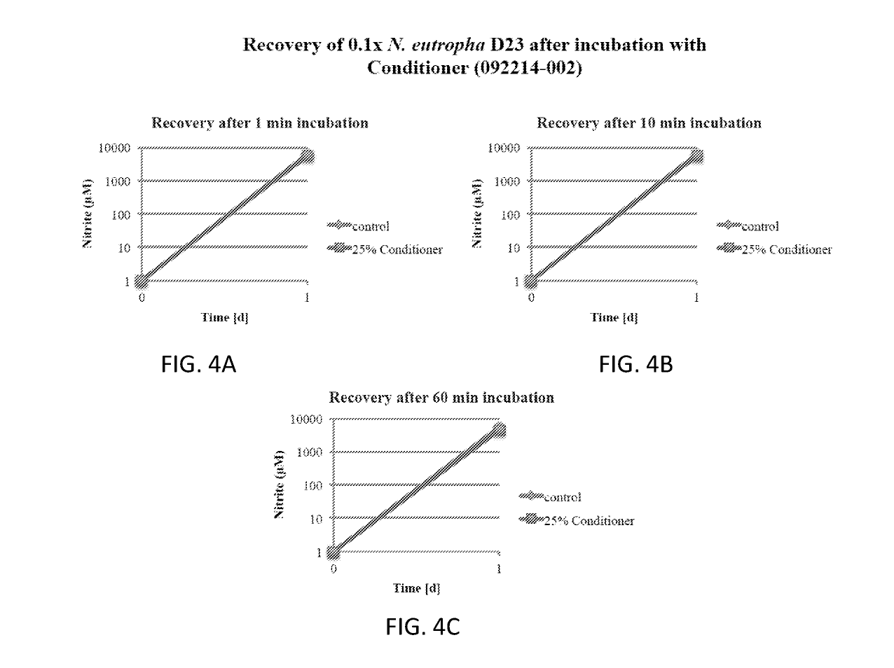

092214-002 conditioner (FIG. 4A-4C)

| | | Possible | |
|---|---|---|---|
| | | Concentration | |
| Component | Concentration | Range | Ingredient |
| Natrasol 300-CS | 2.0% | 0.10-0.5% | Viscosity |
| (Hydroxyethyl | | | modifier |
| Cellulose) | | | |
| Myritol 312 C8-10 | 2.5% | 1.0-5.0% | Conditioner |
| Triglycerides | | | |
| Lamisoft ® PO 65 | 2.5% | 0.0-5.0% | Lipid layer |
| (Coco-Glucoside and | | | enhancer |
| Glyceryl Oleate) | | | |
| Polysorbate-80 | 1.0% | 0.0-3.0% | Emulsifier |
| Natural Rose Water | 5.0% | 0.0-10.0% | Fragrance |

As shown in FIGS. 1A-1C, nitrite production after 1 minute of incubation in the foaming soap, both scented and unscented was achieved. After longer periods of incubation (10 min, and 60 min), nitrite production was achieved, but was less than that after 1 minute of incubation. The nitrite production achieved correlates to the amount of AOB recovered in the test sample.

As shown in FIGS. 2A-2B, nitrite production after 1 minute of incubation in the body wash was achieved and was comparable to the control test sample for both 10% and 25% body wash concentrations (the 25% concentration sample had less nitrite production). With increased incubation time, less nitrite production was achieved. The nitrite production achieved correlates to the amount of AOB recovered in the test sample.

As shown in FIGS. 3A-3B, nitrite production after 1 minute of incubation in the shampoo was achieved and was comparable to the control test sample for both 10% and 25% body wash concentrations (the 25% concentration sample had less nitrite production). With increased incubation time, less nitrite production was achieved. The nitrite production achieved correlates to the amount of AOB recovered in the test sample.

As shown in FIGS. 4A-4B, nitrite production after 1 minute of incubation in the conditioner was achieved and was comparable to the control test sample. With increased incubation time, similar nitrite production was achieved. The nitrite production achieved correlates to the amount of AOB recovered in the test sample.

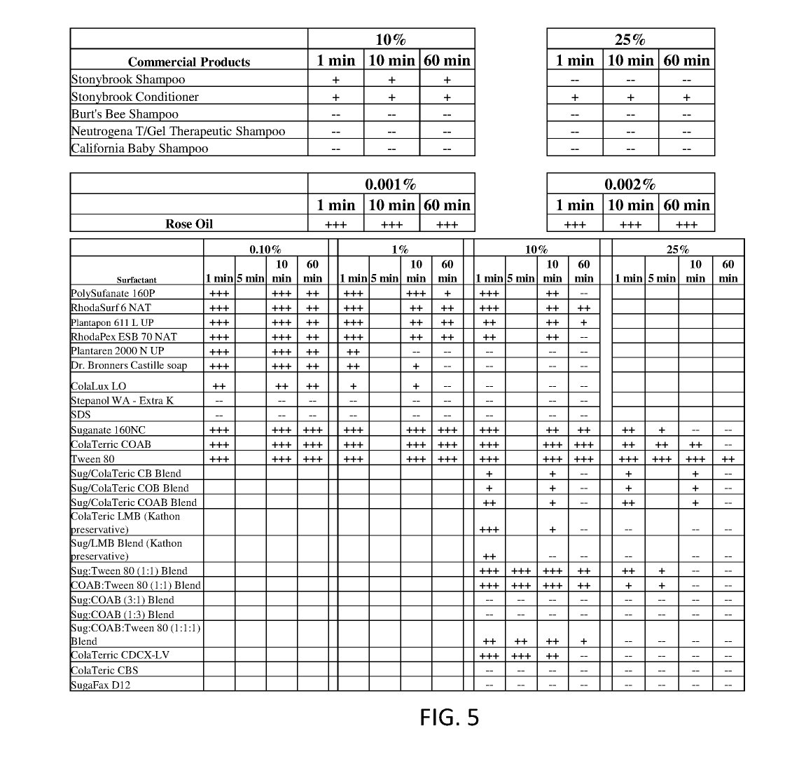

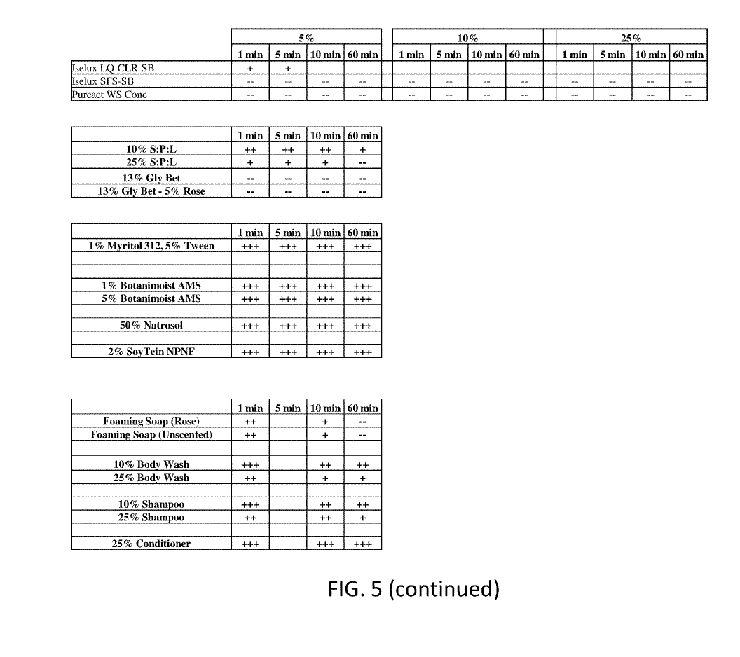

With regard to the Table in FIG. 5, the following tables describe the components of some compositions test:

10% S:P:L and 25% S:P:L (the percentages refer to testing conditions)

| | | Suggested | |
|---|---|---|---|
| | Concentration | Concentration | |
| Component | Tested | Range | Description |
| Suganate 160 | 20.0% | 10.0-30.0% | Biobased |
| Sodium Laurylglucosides | | | surfactant- |
| Hydroxypropylsulfonate | | | cleanser |
| Plantacare 818 (Coco | 2.5% | 1.0-5.0% | Biobased |
| glucoside) | | | surfactant- |
| | | | cleanser |
| Lamisoft ® PO 65 | 3.0% | 0.0-5.0% | Lipid layer |
| (Coco-Glucoside and | | | enhancer |
| Glyceryl Oleate) | | | |

“13% Gly Bet” (in this case the 13% refers to the formula itself)

| | | Suggested | |
|---|---|---|---|
| | Concentration | Concentration | |
| Component | Tested | Range | Description |
| Sodium Cocoyl- | 10.0% | 5.0-20.0% | Biobased |
| Glycinate | | | surfactant- |
| | | | cleanser |
| Cocamidopropyl Betaine | 3.0% | 5.0-12.0% | Cleanser/ |
| | | | surfactant |

### 13% Gly Bet-5% Rose

| | | Suggested | |
|---|---|---|---|
| | Concentration | Concentration | |
| Component | Tested | Range | Description |
| Sodium Cocoyl- | 10.0% | 5.0-20.0% | Biobased |
| Glycinate | | | surfactant- |
| | | | cleanser |
| Cocamidopropyl Betaine | 3.0% | 5.0-12.0% | Cleanser/ |
| | | | surfactant |
| Natural Rose Hydrosol | 5.0% | 0.0-10% | Fragrance |

FIG. 5 shows the degree to which particular surfactants, or blends of surfactants are biome-friendly. The symbols are associated with nitrite production as compared to the control. Recovery identified as “+++” is indicative of nitrite production of greater than 1000 micromolar, measured at the end of a 48 hour period of time. SRecovery identified as “++” is indicative of nitrite production between about 100 micromolar and 1000 micromolar, at the end of a 48 hour period of time. Recovery identified as “+” is indicative of nitrite production between about 10 and 100 micromolar, at the end of a 48 hour period of time. No recovery (−) is indicative of none or substantially no nitrite production measured, for example, less than 10 micromolar nitrite production in a 48 hour period of time.

### Annex VI—ASEAN Cosmetic Documents

### Annex VI—Part 1—List of Preservatives Allowed for Use in Cosmetic Products

ANNEX VI-PART 1
LIST OF PRESERVATIVES ALLOWED

| | | | | Conditions of use and |
|---|---|---|---|---|
| Reference | | Maximum authorized | Limitations and | warnings which must be |
| Number | Substance | concentration | requirements | printed on the label |
| a | b | c | d | e |
| 1 | Benzoic acid (CAS No. 65-85-0), and its | Rinse off products, except | | |
| | sodium salt (CAS no 532-32-1) | oral care products: 2.5% (acid) | | |
| | | Oral care products: 1.7% (acid) | | |
| | | Leave-on producs: 0.5% (acid) | | |
| 1a | Salts of benzoic acid other than that listed | 0.6% (acid) | | |
| | under reference number 1 and esters of | | | |
| | benzoic acid | | | |
| 2 | Propionic acid and its salts | 2% (acid) | | |
| 3 | Salicylic acid and its salts (+)⁽¹⁾ | 0.5% (acid) | Not to be used in | Not to be used for |
| | | | preparations for children | children under three |
| | | | under three years of age, | years of age⁽²⁾ |
| | | | except for shampoos | |
| 4 | Sorbic acid (hexa-2,4-diencic acid) and its salts | 0.6% (acid) | | |
| 5 | Formaldehyde and paraformaldehyde (+)⁽⁷⁾ | 0.2% (except for products | Prohibited in aerosol | |
| | | for oral hygiene) | dispensers (sprays) | |
| | | 0.1% (for oral hygiiene) | | |
| | | expressed as free | | |
| | | formaldehyde | | |
| 6 | Entry moved or deleted | | | |
| 7 | Biphenyl-2-ol (α-phenylphenol) and its salts | 0.2% expressed as phenol | | |
| 8 | Zinc pyrithione (+)⁽³⁾ (CAS No 13453-41-7) | Hair products: 1.0% | Rinse off products only. | |
| | | Other products: 0.5% | Not for use in products for | |
| | | | oral hygiene | |
| 9 | Inorganice sulphites and | 0.2% expressed as free SO₂ | | |
| | hydrogensulphites (+) ⁽⁴⁾ | | | |
| 10 | Entry moved or deleted | | | |
| 11 | Chlorobutanol (INN) | 0.5% | Prohibited in aerosol | Contains chlorobutanol |
| | | | dispensers (sprays) | |
| 12 | 4-Hydroxybenzoic acid and its salts and esters | 0.4% (acid) for 1 ester: | | |
| | | 0.8% (acid) for mixtures of esters | | |
| 13 | 3-Acetyl-6-methylpyran-2,4 (3H)-dione | 0.6% (acid) | Prohibited in aerosol | |
| | (Dehydroacetic acid) and its salts | | dispensers (sprays) | |
| 14 | Formic acid and its sodium salt | 0.5% (express as acid) | | |
| 15 | 3,3′-Dibromo-4,4′-hexamethylenedioxydl- | 0.1% | | |
| | benzamidine (Dibromohexamidine) and its salts | | | |
| | (including isethionate) | | | |
| 16 | Thiomersal (INN) | 0.007% (of Hg) | For eye make-up and eye | Contains thiomersal |
| | | if mixed with other mercurial | make-up remover only | |
| | | compunds authorized by this | | |
| | | Directive, the maximum | | |
| | | concentration of Hg remains | | |
| | | fixed at 0.007% | | |
| 17 | Phenylmercuric salts (including borate) | 0.007% (of Hg) | For eye make-up and eye | Contains phenylmercuric |
| | | if mixed with other mercurial | make-up remover only | compounds |
| | | compunds authorized by this | | |
| | | Directive, the maximum | | |
| | | concentration of Hg remains | | |
| | | fixed at 0.007% | | |
| 18 | Undec-10-enoic acid and its salts (+) | 0.2% (acid) | | |
| 19 | Hexetidine (INN) | 0.1% | | |
| 20 | 5-Bromo-5-nitro-1,3 dioxane | 0.1% | Rinse-off products only | |
| | | | Avoid formation of | |
| | | | nitrosamines | |
| 21 | Bronopol (INN) | 0.1% | Avoid formation of | |
| | | | nitrosamines | |
| 22 | 2,4-Dichlorobenzyl alchohol | 0.15% | | |
| 23 | Tricicarbon (INN) (+)⁽⁵⁾ | 0.2% | Purity criteria: 3,3′,4,4′- | |
| | | | Tetrachlorozabenzene <1 | |
| | | | ppm: 3,3′,4,4′-Tetra- | |
| | | | chloroazoxybenzene <1 | |
| | | | ppm | |
| 24 | 4-Chloro-m-cresol | 0.2% | Prohibited in products | |
| | | | intended to come into | |
| | | | contact with mucous | |
| | | | membranes | |
| 25 | Triclosan (INN) (+) | 0.3% | | |
| 26 | 4-Chloro-3,5-xylenol | 0.5% | | |
| 27 | 3,3′-Bis(1-hydroxymethyl-2,5-dioxolmidazolidin- | | | |
| | 4-yl)-1,1′-methylenediurea (“imidazolidinyl | | | |
| | urea”) | | | |
| 28 | Poly(1-hexamethylenbiguanide hydrochloride) | 0.3% | | |
| 29 | 2-Phenoxyethanol | 1.0% | | |
| 30 | Hexamethylenetramine (methenamine) (INN) | 0.15% | | |
| 31 | Methenamine-3-chloroallylochloride (INNM) | 0.2% | | |
| 32 | 1-(4-Chlorophenoxy)-1-(imidazol-1-yl)-3,3- | 0.6% | | |
| | dimethylbutan-2-one) (+) | | | |
| 33 | 1,3-Bis(hydroxymethyl)-5,5- | 0.6% | | |
| | dimethylimidazolidine-2,4-dione) | | | |
| 34 | Benzyl alchohol (+)⁽⁸⁾ | 1% | | |
| 35 | 1-Hydroxy-4-methyl-6-(2,4,4-trimethylpentyl)-2 | 1% | Rinse-off products | |
| | pyridon and its monoethanolamine salt (+) | 0.5% | For other products | |
| 36 | Entry deleted | | | |
| 37 | 6,6-Dibromo-4,4-dichloro-2,2′-methylene- | 0.1% | | |
| | diphenol (Bromochlorophen) | | | |
| 38 | 4-Isopropyl-m-cresol | 0.1% | | |
| 39 | Mixture of 5-Chloro-2-methyl-isothiazol-3(2H)- | 0.0015% (of a mixture in the | | |
| | one and 2-Methylisothiazol-3(2H)-one with | ratio 3:1 of 5-Chloro-2- | | |
| | magnesium chloride and magnesium nitrate) | methyl-isothiazol-3(2H)-one | | |
| | | and 2-methylisothiazol- | | |
| | | 3(2H)-one) | | |
| 40 | 2-Benzyl-4-chlorphenol (chlorophene) | 0.2% | | |
| 41 | 2-Chloroacetamide | 0.3% | | Contains |
| | | | | chloroacetamide |
| 42 | Chlorhexidine (INN) and its digluconate, | 0.3% expressed as | | |
| | diacetate and dihydrochloride (+) | chlorhexidine | | |
| 43 | 1-Phenoxypropan-2-ol (+)⁽⁹⁾ | 1.0% | Only for rinse-off products | |
| 44 | Alkyl (C12-C22) trimethyl ammonium, bromide | 0.1% | | |
| | and chloride) (+) | | | |
| 45 | 4,4-Dimethyl-1,3-oxazolidine | 0.1% | The pH of the finished | |
| | | | product must not be lower | |
| | | | than 8 | |
| 46 | N-(Hydroxymethyl)-N-(dihydroxymethyl-1,3- | 0.5% | | |
| | dioxo-2,5-imidazolinidyl-4)-N′-(hydroxymethyl) | | | |
| | urea) | | | |
| 47 | 1,6-Di(4-amidinophenoxy)-n-hexane | 0.1% | | |
| | (hexamidine) and its salts (including | | | |
| | isethionate and p-hydroxybenzoate (+) | | | |
| 48 | Gluatraldehyde (Pentane-1,5-diol) | 0.1% | Prohibited in aerosols | Contains glutaraldehyde |
| | | | (sprays) | (where glutaraldehyde |
| | | | | concentration in the |
| | | | | finished product exceeds |
| | | | | 0.05%) |
| 49 | 5-Ethyl-3,7-dioxa-1-azabicyclo [3.3.0] octane | 0.3% | Prohibited in oral hygiene | |
| | | | products and in products | |
| | | | intended to come into | |
| | | | contact with mucous | |
| | | | membranes | |
| 50 | 3-(p-Chlorophenoxy)-propane-1,2-diol | 0.3% | | |
| | (chlorphenesin) | | | |
| 51 | Sodium hydroxymethylamino acetate (Sodium | 0.5% | | |
| | hydroxymethylglycinate | | | |
| 52 | Silver chloride deposited on titanium dioxide | 0.004% calculated as AgCl | 20% AgCl (w/w) on TiO₂ | |
| | | | Prohibited in products for | |
| | | | children under three years of | |
| | | | age, in oral hygiene | |
| | | | products and in products | |
| | | | intended for application | |
| | | | around the eyes and on the | |
| | | | lips | |
| 53 | BenzethoniumChloride (INCI) | 0.1% | (a) Rinse-off products | |
| | | | (b) Leave on products other | |
| | | | than for oral care use. | |
| 54 | Benzalkonium chloride, bromide and | 0.1% calculated as | | Avoid contact with the |
| | saccharinate (+)⁽¹⁰⁾ | Benzalkonium chloride | | eyes |
| 55 | Benzythemiformal | 0.15% | Only for products to be | |
| | | | removed by rinsing | |
| 56 | Iodopropynylbutylcarbamate (IPBC): | (a) rinse-off | Not to be used in oral | (a) Not to be used for |
| | 3-Iodo-2-propynylbutylcarbamate CAS No: | products: 0.02% | hygiene and lip care | children under three |
| | 55406-53-6 | (b) leave-on | products | years of age (**) |
| | | products: 0.01% | (a) Not to be used in | (b) Not to be used for |
| | | except in | preparations for children | children under three |
| | | deodorants & | under three years of age, | years of age (***) |
| | | antiperspirants: 0.0075% | except in bath | |
| | | | products/shower gels and | |
| | | | shampoos. | |
| | | | (b) Not to be used in body | |
| | | | lotion and body cream | |
| | | | (*) | |
| | | | Not to be used in | |
| | | | preparations for children | |
| | | | under three years of age. | |
| 57 | Methylisothiazolinone (INCI) | 0.01% | | |
| 58 | Ethyl LauroylArginateHCl (INCI) (+)⁽⁵⁾ Ethyl-N | 0.4% | Not to be used in lip | |
| | α-dodecanoyl-L-arginate hydrochloride CAS | | products, oral products and | |
| | No. 60372-77-2 | | spray products. | |
| | EC No 434-630-6 | | | |

With regard to Annex VI:
1. Preservatives are substances which may be added to cosmetic products for the primary purpose of inhibiting the development of micro-organisms in such products.
2. The substances marked with the symbol (+) may also be added to cosmetic products in concentration other than those laid down in this ANNEX for other purposes apparent from the presentation of the products, e.g. as deodorants in soaps or as anti-dandruff agents in shampoos.
3. Otter substances used in the formulation of cosmetic products may also have anti-microbial properties and thus help in the preservation of the products, as, for instance, many essential oils and some alcohols. These substances are not included in the ANNEX.
4. For the purposes of this list
   “Salts” is taken to mean: salts of the cations sodium, potassium, calcium, magnesium, ammonium, aid ethanolamines, salts of the anions chloride, bromide, sulphate, acetate.
“Esters” is taken to mean: esters of methyl, ethyl, propyl, isopropyl, butyl, isobutyl, phenyl.
5. All finished products containing formaldehyde or substances in this ANNEX and which release formaldehyde must be labeled with the warning “contains formaldehyde” where the concentration of formaldehyde in the finished product exceeds 0.05%.

[symbol] 1211 [symbol] Sterilization and Sterility Assurance of Compendial Articles

This informational chapter provides a general description of the concepts and principles involved in the quality control of articles that must be sterile. Any modifications of or variations in sterility test procedures from those described under Sterility Tests [symbol] 71 [symbol] should be validated in the context of the entire sterility assurance program and are not intended to be methods alternative to those described in that chapter.

Within the strictest definition of sterility, a specimen would be deemed sterile only when there is complete absence of viable microorganisms from it. However, this absolute definition cannot currently be applied to an entire lot of finished compendial articles because of limitations in testing. Absolute sterility cannot be practically demonstrated without complete destruction of every finished article. The sterility of a lot purported to be sterile is therefore defined in probabilistic terms, where the likelihood of a contaminated unit or article is acceptably remote. Such a state of sterility assurance can be established only through the use of adequate sterilization cycles and subsequent aseptic processing, if any, under appropriate current good manufacturing practice, and not by reliance solely on sterility testing. The basic principles for validation and certification of a sterilizing process are enumerated as follows:
1. Establish that the process equipment has capability of operating within the required parameters.
2. Demonstrate that the critical control equipment and instrumentation are capable of operating within the prescribed parameters for the process equipment.
3. Perform replicate cycles representing the required operational range of the equipment and employing actual or simulated product. Demonstrate that the processes have been carried out within the prescribed protocol limits and finally that the probability of microbial survival in the replicate processes completed is not greater than the prescribed limits.
4. Monitor the validated process during routine operation. Periodically as needed, requalify and recertify the equipment.
5. Complete the protocols, and document steps (1) through (4) above.

The principles and implementation of a program to validate an aseptic processing procedure are similar to the validation of a sterilization process. In aseptic processing, the components of the final dosage form are sterilized separately and the finished article is assembled in an aseptic manner.

Proper validation of the sterilization process or the aseptic process requires a high level of knowledge of the field of sterilization and clean room technology. In order to comply with currently acceptable and achievable limits in sterilization parameters, it is necessary to employ appropriate instrumentation and equipment to control the critical parameters such as temperature and time, humidity, and sterilizing gas concentration, or absorbed radiation. An important aspect of the validation program in many sterilization procedures involves the employment of biological indicators (see Biological Indicators [symbol] 1035 [symbol] ). The validated and certified process should be revalidated periodically; however, the revalidation program need not necessarily be as extensive as the original program.

A typical validation program, as outlined below, is one designed for the steam autoclave, but the principles are applicable to the other sterilization procedures discussed in this informational chapter. The program comprises several stages.

The installation qualification stage is intended to establish that controls and other instrumentation are properly designed and calibrated. Documentation should be on file demonstrating the quality of the required utilities such as steam, water, and air. The operational qualification stage is intended to confirm that the empty chamber functions within the parameters of temperature at all of the key chamber locations prescribed in the protocol. It is usually appropriate to develop heat profile records, i.e., simultaneous temperatures in the chamber employing multiple temperature-sensing devices. A typical acceptable range of temperature in the empty chamber is ±1° when the chamber temperature is not less than 121°. The confirmatory stage of the validation program is the actual sterilization of materials or articles. This determination requires the employment of temperature-sensing devices inserted into samples of the articles, as well as either samples of the articles to which appropriate concentrations of suitable test microorganisms have been added, or separate BIs in operationally fully loaded autoclave configurations. The effectiveness of heat delivery or penetration into the actual articles and the time of the exposure are the two main factors that determine the lethality of the sterilization process. The final stage of the validation program requires the documentation of the supporting data developed in executing the program.

It is generally accepted that terminally sterilized injectable articles or critical devices purporting to be sterile, when processed in the autoclave, attain a 10⁻⁶ microbial survivor probability, i.e., assurance of less than 1 chance in 1 million that viable microorganisms are present in the sterilized article or dosage form. With heat-stable articles, the approach often is to considerably exceed the critical time necessary to achieve the 10⁻⁶ microbial survivor probability (overkill). However, with an article where extensive heat exposure may have a damaging effect, it may not be feasible to employ this overkill approach. In this latter instance, the development of the sterilization cycle depends heavily on knowledge of the microbial burden of the product, based on examination, over a suitable time period, of a substantial number of lots of the presterilized product.

The D value is the time (in minutes) required to reduce the microbial population by 90% or 1 log cycle (i.e., to a surviving fraction of 1/10), at a specific temperature. Therefore, where the D value of a BI preparation of, for example, Bacillus stearothermophilus spores is 1.5 minutes under the total process parameters, e.g., at 121°, if it is treated for 12 minutes under the same conditions, it can be stated that the lethality input is 8D. The effect of applying this input to the product would depend on the initial microbial burden. Assuming that its resistance to sterilization is equivalent to that of the BI, if the microbial burden of the product in question is 10² microorganisms, a lethality input of 2D yields a microbial burden of 1 (10° theoretical), and a further 6D yields a calculated microbial survivor probability of 10⁻⁶. (Under the same conditions, a lethality input of 12D may be used in a typical “overkill” approach.) Generally, the survivor probability achieved for the article under the validated sterilization cycle is not completely correlated with what may occur with the BI. For valid use, therefore, it is essential that the resistance of the BI be greater than that of the natural microbial burden of the article sterilized. It is then appropriate to make a worst-case assumption and treat the microbial burden as though its heat resistance were equivalent to that of the BI, although it is not likely that the most resistant of a typical microbial burden isolates will demonstrate a heat resistance of the magnitude shown by this species, frequently employed as a BI for steam sterilization. In the above example, a 12-minute cycle is considered adequate for sterilization if the product had a microbial burden of 10² microorganisms. However, if the indicator originally had 10⁶ microorganisms content, actually a 10⁻² probability of survival could be expected; i.e., 1 in 100 BIs may yield positive results. This type of situation may be avoided by selection of the appropriate BI. Alternatively, high content indicators may be used on the basis of a predetermined acceptable count reduction.

The D value for the Bacillus stearothermophilus preparation determined or verified for these conditions should be reestablished when a specific program of validation is changed.

Determination of survival curves (see Biological Indicators [symbol] 1035 [symbol] ), or what has been called the fractional cycle approach, may be employed to determine the D value of the biological indicator preferred for the specific sterilization procedure. The fractional cycle approach, may also be used to evaluate the resistance of the microbial burden. Fractional cycles are studied either for microbial count-reduction or for fraction negative achievement. These numbers may be used to determine the lethality of the process under production conditions. The data can be used in qualified production equipment to establish appropriate sterilization cycles. A suitable biological indicator such as the Bacillus stearothermophilus preparation may be employed also during routine sterilization. Any microbial burden method for sterility assurance requires adequate surveillance of the microbial resistance of the article to detect any changes, in addition to periodic surveillance of other attributes.

### Methods of Sterilization

In this informational chapter, five methods of terminal sterilization, including removal of microorganisms by filtration and guidelines for aseptic processing, are described. Modern technological developments, however, have led to the use of additional procedures. These include blow-molding (at high temperatures), forms of moist heat other than saturated steam and UV irradiation, as well as on-line continuous filling in aseptic processing. The choice of the appropriate process for a given dosage form or component requires a high level of knowledge of sterilization techniques and information concerning any effects of the process on the material being sterilized.¹ ¹ A number of guidelines dealing particularly with the development and validation of sterilization cycles and related topics have been published. These include, by the Parenteral Drug Association, Inc. (PDA), Validation of Steam Sterilization Cycles (Technical Monograph No. 1), Validation of Aseptic Filling for Solution Drug Products (Technical Monograph No. 2), and Validation of Dry Heat Processes Used for Sterilization and Depyrogenation (Technical Monograph No. 3); and by the Pharmaceutical Manufacturers Association (PMA), Validation of Sterilization of Large-Volume Parenterals—Current Concepts (Science and Technology Publication No. 25). Other series of technical publications on these subjects by the Health Industry Manufacturers Association (HIMA) include Validation of Sterilization Systems (Report No. 78-4.1), Sterilization Cycle Development (Report No. 78-4.2), Industrial Sterility: Medical Device Standards and Guidelines (Document #9, Vol. 1), and Operator Training . . . for Ethylene Oxide Sterilization, for Steam Sterilization Equipment, for Dry Heat Sterilization Equipment, and for Radiation Sterilization Equipment (Report Nos. 78-4.5 through 4.8). Recommended practice guidelines published by the Association for the Advancement of Medical Instrumentation (AAMI) include Guideline for Industrial Ethylene Oxide Sterilization of Medical Devices—Process Design, Validation, Routine Sterilization (No. OPEO-12/81) and Process Control Guidelines for the Radiation Sterilization of Medical Devices (No. RS-P 10/82). These detailed publications should be consulted for more extensive treatment of the principles and procedures described in this chapter.

### Steam Sterilization

The process of thermal sterilization employing saturated steam under pressure is carried out in a chamber called an autoclave. It is probably the most widely employed sterilization process.² The basic principle of operation is that the air in the sterilizing chamber is displaced by the saturated steam, achieved by employing vents or traps. In order to displace air more effectively from the chamber and from within articles, the sterilization cycle may include air and steam evacuation stages. The design or choice of a cycle for given products or components depends on a number of factors, including the heat lability of the material, knowledge of heat penetration into the articles, and other factors described under the validation program (see above). Apart from that description of sterilization cycle parameters, using a temperature of 121°, the F₀ concept may be appropriate. The F₀, at a particular temperature other than 121°, is the time (in minutes) required to provide the lethality equivalent to that provided at 121° for a stated time. Modern autoclaves generally operate with a control system that is significantly more responsive than the steam reduction valve of older units that have been in service for many years. In order for these older units to achieve the precision and level of control of the cycle discussed in this chapter, it may be necessary to upgrade or modify the control equipment and instrumentation on these units. This modification is warranted only if the chamber and steam jacket are intact for continued safe use and if deposits that interfere with heat distribution can be removed. ² An autoclave cycle, where specified in the compendia for media or reagents, is a period of 15 minutes at 121°, unless otherwise indicated.

### Dry-Heat Sterilization

The process of thermal sterilization of Pharmacopeial articles by dry heat is usually carried out by a batch process in an oven designed expressly for that purpose. A modern oven is supplied with heated, filtered air, distributed uniformly throughout the chamber by convection or radiation and employing a blower system with devices for sensing, monitoring, and controlling the critical parameters. The validation of a dry-heat sterilization facility is carried out in a manner similar to that for a steam sterilizer described earlier. Where the unit is employed for sterilizing components such as containers intended for intravenous solutions, care should be taken to avoid accumulation of particulate matter in the chamber. A typical acceptable range in temperature in the empty chamber is ±15° when the unit is operating at not less than 250°.

In addition to the batch process described above, a continuous process is frequently employed to sterilize and depyrogenate glassware as part of an integrated continuous aseptic filling and sealing system. Heat distribution may be by convection or by direct transfer of heat from an open flame. The continuous system usually requires a much higher temperature than cited above for the batch process because of a much shorter dwell time. However, the total temperature input during the passage of the product should be equivalent to that achieved during the chamber process. The continuous process also usually necessitates a rapid cooling stage prior to the aseptic filling operation. In the qualification and validation program, in view of the short dwell time, parameters for uniformity of the temperature, and particularly the dwell time, should be established.

A microbial survival probability of 10⁻¹² is considered achievable for heat-stable articles or components. An example of a biological indicator for validating and monitoring dry-heat sterilization is a preparation of Bacillus subtilis spores. Since dry heat is frequently employed to render glassware or containers free from pyrogens as well as viable microbes, a pyrogen challenge, where necessary, should be an integral part of the validation program, e.g., by inoculating one or more of the articles to be treated with 1000 or more USP Units of bacterial endotoxin. The test with Limulus lysate could be used to demonstrate that the endotoxic substance has been inactivated to not more than 1/1000 of the original amount (3 log cycle reduction). For the test to be valid, both the original amount and, after acceptable inactivation, the remaining amount of endotoxin should be measured. For additional information on the endotoxin assay, see Bacterial Endotoxins Test [symbol] 85 [symbol] .

### Gas Sterilization

The choice of gas sterilization as an alternative to heat is frequently made when the material to be sterilized cannot withstand the high temperatures obtained in the steam sterilization or dry-heat sterilization processes. The active agent generally employed in gaseous sterilization is ethylene oxide of acceptable sterilizing quality. Among the disadvantages of this sterilizing agent are its highly flammable nature unless mixed with suitable inert gases, its mutagenic properties, and the possibility of toxic residues in treated materials, particularly those containing chloride ions. The sterilization process is generally carried out in a pressurized chamber designed similarly to a steam autoclave but with the additional features (see below) unique to sterilizers employing this gas. Facilities employing this sterilizing agent should be designed to provide adequate post sterilization degassing, to enable microbial survivor monitoring, and to minimize exposure of operators to the potentially harmful gas.³ ³ See Ethylene Oxide, Encyclopedia of Industrial Chemical Analysis, 1971, 12, 317-340, John Wiley & Sons, Inc., and Use of Ethylene Oxide as a Sterilant in Medical Facilities, NIOSH Special Occupational Hazard Review with Control Recommendations, August 1977, U. S. Department of Health and Human Services, Public Health Service, Centers for Disease Control and Prevention, National Institute for Occupational Safety and Health, Division of Criteria Documentation and Standards Development, Priorities and Research Analysis Branch, Rockville, Md.

Qualification of a sterilizing process employing ethylene oxide gas is accomplished along the lines discussed earlier. However, the program is more comprehensive than for the other sterilization procedures, since in addition to temperature, the humidity, vacuum/positive pressure, and ethylene oxide concentration also require rigid control. An important determination is to demonstrate that all critical process parameters in the chamber are adequate during the entire cycle. Since the sterilization parameters applied to the articles to be sterilized are critical variables, it is frequently advisable to precondition the load to achieve the required moisture content in order to minimize the time of holding at the required temperature before placement of the load in the ethylene oxide chamber. The validation process is generally made employing product inoculated with appropriate (BIs) such as spore preparations of Bacillus subtilis. For validation they may be used in full chamber loads of product, or simulated product.

The monitoring of moisture and gas concentration requires the utilization of sophisticated instrumentation that only knowledgeable and experienced individuals can calibrate, operate, and maintain. The BI may be employed also in monitoring routine runs.

As is indicated elsewhere in this chapter, the BI may be employed in a fraction negative mode to establish the ultimate microbiological survivor probability in designing an ethylene oxide sterilization cycle using inoculated product or inoculated simulated product.

One of the principal limitations of the ethylene oxide sterilization process is the limited ability of the gas to diffuse to the innermost product areas that require sterilization. Package design and chamber loading patterns therefore must be determined so that there is minimal resistance to gas diffusion.

### Sterilization by Ionizing Radiation

The rapid proliferation of medical devices unable to withstand heat sterilization and the concerns about the safety of ethylene oxide have resulted in increasing applications of radiation sterilization. It is applicable also to drug substances and final dosage forms. The advantages of sterilization by irradiation include low chemical reactivity, low measurable residues, and the fact that there are fewer variables to control. In fact, radiation sterilization is unique in that the basis of control is essentially that of the absorbed radiation dose, which can be precisely measured. Because of this characteristic, new procedures have been developed to determine the sterilizing dose. These, however, are still under review and appraisal, particularly with regard to the need, or otherwise, for additional controls and safety measures. Irradiation causes only a minimal temperature rise but can affect certain grades and types of plastics and glass.

The two types of ionizing radiation in use are radioisotope decay (gamma radiation) and electron-beam radiation. In either case the radiation dose established to yield the required degree of sterility assurance should be such that, within the range of minimum and maximum doses set, the properties of the article being sterilized are acceptable.

For gamma irradiation, the validation of a procedure includes the establishment of article materials compatibility, establishment of product loading pattern and completion of dose mapping in the sterilization container (including identification of the minimum and maximum dose zones), establishment of timer setting, and demonstration of the delivery of the required sterilization dose. For electron-beam irradiation, in addition, the on-line control of voltage, current, conveyor speed, and electron beam scan dimension must be validated.

For gamma radiation sterilization, an effective sterilizing dose that is tolerated without damaging effect should be selected. Although 2.5 megarads (Mrad) of absorbed radiation was historically selected, it is desirable and acceptable in some cases to employ lower doses for devices, drug substances, and finished dosage forms. In other cases, however, higher doses are essential. In order to validate the efficacy particularly of the lower exposure levels, it is necessary to determine the magnitude (number, degree, or both) of the natural radiation resistance of the microbial population of the product. Specific product loading patterns must be established, and absorbed minimum and maximum dosage distribution must be determined by use of chemical dosimeters. (These dosimeters are usually dyed plastic cylinders, slides, or squares that show color intensification based directly on the amount of absorbed radiation energy; they require careful calibration.)

The setting of the preferred absorbed dose has been carried out on the basis of pure cultures of resistant microorganisms and employing inoculated product, e.g., with spores of Bacillus pumilus as biological indicators. A fractional experimental cycle approach provides the data to be utilized to determine the D₁₀ value of the biological indicator. This information is then applied in extrapolating the amount of absorbed radiation to establish an appropriate microbial survivor probability. The most recent procedures for gamma radiation sterilization base the dose upon the radiation resistance of the natural heterogeneous microbial burden contained on the product to be sterilized. Such procedures are currently being refined but may provide a more representative assessment of radiation resistance, especially where significant numbers of radiation-resistant organisms are present.⁴ These range from inoculation with standard resistant organisms such as Bacillus pumilus to subprocess (sublethal) dose exposure of finished product samples taken from production lines. Certain hypotheses are common to all these methods. Although the total microbial population present on an article generally consists of a mixture of microorganisms of differing sensitivity to radiation, the step of subjecting the article to a less than totally lethal sterilization dose eliminates the less resistant microbial fraction. This results in a residual relatively homogeneous population with respect to radiation resistance and yields consistent and reproducible results of determinations with the residual population. The amount of laboratory manipulation required is dependent upon the particular procedure used. ⁴ Detailed descriptions of these procedures have been published by the Association for the Advancement of Medical Instrumentation (AAMI) in the document entitled Process Control Guidelines for Radiation Sterilization of Medical Devices (No. RS-P 10/82).

One such procedure requires the enumeration of the microbial population on representative samples of independently manufactured lots of the article. The resistance of the microbial population is not determined, and dose setting is based on a standard arbitrary radiation resistance assigned to the microbial population, derived from data obtained from manufacturers and from the literature. The assumption is made that the distribution of resistances chosen represents a more severe challenge than the natural microbial population on the product to be sterilized. This assumption, however, is verified by experiment. After verification, the appropriate radiation sterilization dose is read from a table.

Another and, more elaborate method does not require the enumeration of the microbial population but uses a series of incremental dose exposures to allow a dose established to be such that approximately one out of 100 samples irradiated at that dose will be nonsterile. This is not the ultimate sterilization dose, but it provides the basis on which to determine the sterilization dose by extrapolation from the dose yielding one out of 100 nonsterile samples, using an appropriate resistance factor that characterizes the remaining microorganism-resistant population. A periodic audit is conducted to check that the findings continue to be operative.

More elaborate procedures, requiring more experimentation and including the isolation of microbial cultures, include one in which, after determining the substerilization dose (yielding one out of 100 nonsterile samples), the resistance of the surviving microorganisms is used to determine the sterilizing dose. Another is based on different determinations, starting with a substerilization incremental dose that results in not more than 50% of the samples being nonsterile. After irradiation of sufficient samples at this dose, a number of microbial isolates are obtained. The radiation resistance of each of these is determined. The sterilization dose is then calculated using the resistance determinations and the 50% sterilizing dose initially determined. Audit procedures are required for these methods, as for the others described.

Where the required minimum radiation dose has been determined and delivery of that dose has been confirmed (by chemical or physical dosimeters), release of the article being sterilized could be effected within the overall validation of sterility assurance (which may include such confirmation of applied dosage, the use of biological indicators, and other means).

### Sterilization by Filtration

Filtration through microbial retentive materials is frequently employed for the sterilization of heat-labile solutions by physical removal of the contained microorganisms. A filter assembly generally consists of a porous matrix sealed or clamped into an impermeable housing. The effectiveness of a filter medium or substrate depends upon the pore size of the porous material and may depend upon adsorption of bacteria on or in the filter matrix or upon a sieving mechanism. There is some evidence to indicate that sieving is the more important component of the mechanism. Fiber-shedding filters, particularly those containing asbestos, are to be avoided unless no alternative filtration procedures are possible. Where a fiber-shedding filter is required, it is obligatory that the process include a nonfiber-shedding filter introduced downstream or subsequent to the initial filtration step.

### Filter Rating—

The pore sizes of filter membranes are rated by a nominal rating that reflects the capability of the filter membrane to retain microorganisms of size represented by specified strains, not by determination of an average pore size and statement of distribution of sizes. Sterilizing filter membranes (those used for removing a majority of contaminating microorganisms) are membranes capable of retaining 100% of a culture of 10⁷ microorganisms of a strain of Pseudomonas diminuta (ATCC 19146) per square centimeter of membrane surface under a pressure of not less than 30 psi (2.0 bar). Such filter membranes are nominally rated 0.22 μm or 0.2 μm, depending on the manufacturer's practice.⁵ This rating of filter membranes is also specified for reagents or media that have to be sterilized by filtration (see treatment of Isopropyl Myristate under Oils and Oily Solutions or Ointments and Creams in the chapter Sterility Tests [symbol] 71 [symbol] ). Bacterial filter membranes (also known as analytical filter membranes), which are capable of retaining only larger microorganisms, are labeled with a nominal rating of 0.45 μm. No single authoritative method for rating 0.45-μm filters has been specified, and this rating depends on conventional practice among manufacturers; 0.45-μm filters are capable of retaining particular cultures of Serratia marcescens (ATCC 14756) or Ps. diminuta. Test pressures used vary from low (5 psi, 0.33 bar for Serratia, or 0.5 psi, 0.34 bar for Ps. diminuta) to high (50 psi, 3.4 bar). They are specified for sterility testing (see Membrane Filtration in the section Test for Sterility of the Product to be Examined under Sterility Tests [symbol] 71 [symbol] ) where less exhaustive microbial retention is required. There is a small probability of testing specimens contaminated solely with small microorganisms). Filter membranes with a very low nominal rating may be tested with a culture of Acholeplasma laidlawii or other strain of Mycoplasma, at a pressure of 7 psi (0.7 bar) and be nominally rated 0.1 μm. The nominal ratings based on microbial retention properties differ when rating is done by other means, e.g., by retention of latex spheres of various diameters. It is the user's responsibility to select a filter of correct rating for the particular purpose, depending on the nature of the product to be filtered. It is generally not feasible to repeat the tests of filtration capacity in the user's establishment. Microbial challenge tests are preferably performed under a manufacturer's conditions on each lot of manufactured filter membranes. ⁵ Consult “Microbiological Evaluation of Filters for Sterilizing Liquids,” Health Industry Manufacturers Association, Document No. 3, Vol. 4, 1982.

The user must determine whether filtration parameters employed in manufacturing will significantly influence microbial retention efficiency. Some of the other important concerns in the validation of the filtration process include product compatibility, sorption of drug, preservative or other additives, and initial effluent endotoxin content.

Since the effectiveness of the filtration process is also influenced by the microbial burden of the solution to be filtered, determining the microbiological quality of solutions prior to filtration is an important aspect of the validation of the filtration process, in addition to establishing the other parameters of the filtration procedure, such as pressures, flow rates, and filter unit characteristics. Hence, another method of describing filter-retaining capability is the use of the log reduction value (LRV). For instance, a 0.2-μm filter that can retain 10⁷ microorganisms of a specified strain will have an LRV of not less than 7 under the stated conditions.

The process of sterilization of solutions by filtration has recently achieved new levels of proficiency, largely as a result of the development and proliferation of membrane filter technology. This class of filter media lends itself to more effective standardization and quality control and also gives the user greater opportunity to confirm the characteristics or properties of the filter assembly before and after use. The fact that membrane filters are thin polymeric films offers many advantages but also some disadvantages when compared to depth filters such as porcelain or sintered material. Since much of the membrane surface is a void or open space, the properly assembled and sterilized filter offers the advantage of a high flow rate. A disadvantage is that since the membrane is usually fragile, it is essential to determine that the assembly was properly made and that the membrane was not ruptured during assembly, sterilization, or use. The housings and filter assemblies that are chosen should first be validated for compatibility and integrity by the user. While it may be possible to mix assemblies and filter membranes produced by different manufacturers, the compatibility of these hybrid assemblies should first be validated. Additionally, there are other tests to be made by the manufacturer of the membrane filter, which are not usually repeated by the user. These include microbiological challenge tests. Results of these tests on each lot of manufactured filter membranes should be obtained from the manufacturer by users for their records.

Filtration for sterilization purposes is usually carried out with assemblies having membranes of nominal pore size rating of 0.2 μm or less, based on the validated challenge of not less than 10⁷ Pseudomonas diminuta (ATCC No. 19146) suspension per square centimeter of filter surface area. Membrane filter media now available include cellulose acetate, cellulose nitrate, fluorocarbonate, acrylic polymers, polycarbonate, polyester, polyvinyl chloride, vinyl, nylon, polytef, and even metal membranes, and they may be reinforced or supported by an internal fabric. A membrane filter assembly should be tested for initial integrity prior to use, provided that such test does not impair the validity of the system, and should be tested after the filtration process is completed to demonstrate that the filter assembly maintained its integrity throughout the entire filtration procedure. Typical use tests are the bubble point test, the diffusive airflow test, the pressure hold test, and the forward flow test. These tests should be correlated with microorganism retention.

### Aseptic Processing

Although there is general agreement that sterilization of the final filled container as a dosage form or final packaged device is the preferred process for ensuring the minimal risk of microbial contamination in a lot, there is a substantial class of products that are not terminally sterilized but are prepared by a series of aseptic steps. These are designed to prevent the introduction of viable microorganisms into components, where sterile, or once an intermediate process has rendered the bulk product or its components free from viable microorganisms. This section provides a review of the principles involved in producing aseptically processed products with a minimal risk of microbial contamination in the finished lot of final dosage forms.

A product defined as aseptically processed is likely to consist of components that have been sterilized by one of the processes described earlier in this chapter. For example, the bulk product, if a filterable liquid, may have been sterilized by filtration. The final empty container components would probably be sterilized by heat, dry heat being employed for glass vials and an autoclave being employed for rubber closures. The areas of critical concern are the immediate microbial environment where these presterilized components are exposed during assembly to produce the finished dosage form and the aseptic filling operation.

The requirements for a properly designed, validated, and maintained filling or other aseptic processing facility are mainly directed to (1) an air environment free from viable microorganisms, of a proper design to permit effective maintenance of air supply units, and (2) the provision of trained operating personnel who are adequately equipped and gowned. The desired environment may be achieved through the high level of air filtration technology now available, which contributes to the delivery of air of the requisite microbiological quality.⁶ The facilities include both primary (in the vicinity of the exposed article) and secondary (where the aseptic processing is carried out) barrier systems. ⁶ Available published standards for such controlled work areas include the following: (1) Federal Standard No. 209B, Clean Room and Work Station Requirements for a Controlled Environment, Apr. 24, 1973. (2) NASA Standard for Clean Room and Work Stations for Microbially Controlled Environment, publication NHB5340.2, August 1967. (3) Contamination Control of Aerospace Facilities, U.S. Air Force, T.O. 00-25-203, 1 Dec. 1972, change 1-1, October 1974.

For a properly designed aseptic processing facility or aseptic filling area, consideration should be given to such features as nonporous and smooth surfaces, including walls and ceilings that can be sanitized frequently; gowning rooms with adequate space for personnel and storage of sterile garments; adequate separation of preparatory rooms for personnel from final aseptic processing rooms, with the availability if necessary of devices such as airlocks and air showers; proper pressure differentials between rooms, the most positive pressure being in the aseptic processing rooms or areas; the employment of laminar (unidirectional) airflow in the immediate vicinity of exposed product or components, and filtered air exposure thereto, with adequate air change frequency; appropriate humidity and temperature environmental controls; and a documented sanitization program. Proper training of personnel in hygienic and gowning techniques should be undertaken so that, for example, gowns, gloves, and other body coverings substantially cover exposed skin surfaces.

Certification and validation of the aseptic process and facility are achieved by establishing the efficiency of the filtration systems, by employing microbiological environmental monitoring procedures, and by processing of sterile culture medium as simulated product.

Monitoring of the aseptic facility should include periodic environmental filter examination as well as routine particulate and microbiological environmental monitoring and may include periodic sterile culture medium processing.

### Sterility Testing of Lots

It should be recognized that the referee sterility test might not detect microbial contamination if present in only a small percentage of the finished articles in the lot because the specified number of units to be taken imposes a significant statistical limitation on the utility of the test results. This inherent limitation, however, has to be accepted, because current knowledge offers no nondestructive alternatives for ascertaining the microbiological quality of every finished article in the lot, and it is not a feasible option to increase the number of specimens significantly.

The primary means of supporting the claim that a lot of finished articles purporting to be sterile meets the specifications consists of the documentation of the actual production and sterilization record of the lot and of the additional validation records that the sterilization process has the capability of totally inactivating the established product microbial burden or a more resistant challenge. Further, it should be demonstrated that any processing steps involving exposed product following the sterilization procedure are performed in an aseptic manner to prevent contamination. If data derived from the manufacturing process sterility assurance validation studies and from in-process controls are judged to provide greater assurance that the lot meets the required low probability of containing a contaminated unit (compared to sterility testing results from finished units drawn from that lot), any sterility test procedures adopted may be minimal, or dispensed with on a routine basis. However, assuming that all the above production criteria have been met, it may still be desirable to perform sterility testing on samples of the lot of finished articles. Such sterility testing is usually carried out directly after the lot is manufactured as a final product quality control test.⁷ Sterility tests employed in this way in manufacturing control should not be confused with those described under Sterility Tests [symbol] 71 [symbol] . The procedural details may be the same with regard to media, inocula and handling of specimens, but the number of units and/or incubation time(s) selected for testing may differ. The number should be chosen relative to the purpose to be served, i.e., according to whether greater or lesser reliance is placed on sterility testing in the context of all the measures for sterility assurance in manufacture. Also, longer times of incubation would make the test more sensitive to slow-growing microorganisms. In the growth promotion tests for media, such slow growers, particularly if isolated from the product microbial burden, should be included with the other test stains. Negative or satisfactory sterility test results serve only as further support of the existing evidence concerning the quality of the lot if all the pertinent production records of the lot are in order and the sterilizing or aseptic process is known to be effective. Unsatisfactory test results, however, in manufacturing quality control indicate a need for further action (see Performance, Observation, and Interpretation). ⁷ Radioactive Pharmaceutical Products—Because of rapid radioactive decay, it is not feasible to delay the release of some radioactive pharmaceutical products in order to complete sterility tests on them. In such cases, results of sterility tests provide only retrospective confirmatory evidence for sterility assurance, which therefore depends on the primary means thereto established in the manufacturing and validation/certification procedures.

### Definition of a Lot and Selection of Specimens for Sterility Test Purposes

Articles may be terminally sterilized either in a chamber or by a continuous process. In the chamber process, a number of articles are sterilized simultaneously under controlled conditions—for example, in a steam autoclave—so that for the purpose of sterility testing, the lot is considered to be the contents of a single chamber. In the continuous process, the articles are sterilized individually and consecutively (for example, by exposure to electron-beam radiation), so that the lot is considered to be not larger than the total number of similar items subjected to uniform sterilization for a period of not more than 24 hours.

For aseptic fills, the term “filling operation” describes a group of final containers, identical in all respects, that have been aseptically filled with the same product from the same bulk within a period not longer than 24 consecutive hours without an interruption or a change that would affect the integrity of the filling assembly. The items tested should be representative of each filling assembly and should be selected at appropriate intervals throughout the entire filling operation. If more than three filling machines, each with either single or multiple filling stations, are used for filling a single lot, a minimum of 20 filled containers (not less than 10 per medium) should be tested for each filling machine, but the total number generally need not exceed 100 containers.

For small lots, in the case of either aseptic filling or terminal sterilization, if the number of final containers in the lot is between 20 and 200, about 10% of the containers should usually be tested. If the number of final containers in the lot is 20 or less, not fewer than 2 final containers should be tested.

### Performance, Observation, and Interpretation

The facility for sterility testing should be such as to offer no greater a microbial challenge to the articles being tested than that of an aseptic processing production facility. The sterility testing procedure should be performed by individuals having a high level of aseptic technique proficiency. The test performance records of these individuals should be documented.

The extensive aseptic manipulations required to perform sterility testing may result in a probability of non-product-related contamination of the order of 10⁻³, a level similar to the overall efficiency of an aseptic operation and comparable to the microbial survivor probability of aseptically processed articles. This level of probability is significantly greater than that usually attributed to a terminal sterilization process, namely, 1 in 1 million or 10⁻⁶ microbial survivor probability. Appropriate, known-to-be-sterile finished articles should be employed periodically as negative controls as a check on the reliability of the test procedure. Preferably, the technicians performing the test should be unaware that they are testing negative controls. Of these tests, a false-positive frequency not exceeding 2% is desirable.

For aseptically processed articles, these facts support the routine use of the test set forth under Sterility Tests [symbol] 71 [symbol] or a more elaborate one. The production and validation documentation should be acceptable and complete. For effectively terminally sterilized products, however, the lower microbial survivor probability may direct the use of a less extensive test than the compendial procedure specified under Sterility Tests [symbol] 71 [symbol] , or even preclude altogether the necessity for performing one. This added reliability of sterility assurance of terminal sterilization depends upon a properly validated and documented sterilization process. Sterility testing alone is no substitute.

Interpretation of Quality Control Tests—The overall responsibility for the operation of the test unit and the interpretation of test results in relation to acceptance or rejection of a lot should be in the hands of those who have appropriate formal training in microbiology and have knowledge of industrial sterilization, aseptic processing, and the statistical concepts involved in sampling. These individuals should be knowledgeable also concerning the environmental control program in the test facility to ensure that the microbiological quality of the air and critical work surfaces are consistently acceptable.

Quality control sterility tests (either according to the official referee test or modified tests) may be carried out in two separate stages in order to rule out false positive results. First Stage. Regardless of the sampling plan used, if no evidence of microbial growth is found, the results of the test may be taken as indicative of absence of intrinsic contamination of the lot.

If microbial growth is found, proceed to the Second Stage (unless the First Stage test can be invalidated). Evidence for invalidating a First Stage test in order to repeat it as a First Stage test may be obtained from a review of the testing environment and the relevant records thereto. Finding of microbial growth in negative controls need not be considered the sole grounds for invalidating a First Stage test. When proceeding to the Second Stage, particularly when depending on the results of the test for lot release, concurrently, initiate and document a complete review of all applicable production and control records. In this review, consideration should be paid to the following: (1) a check on monitoring records of the validated sterilization cycle applicable to the product, (2) sterility test history relating to the particular product for both finished and in-process samples, as well as sterilization records of supporting equipment, containers/closures, and sterile components, if any, and (3) environmental control data, including those obtained from media fills, exposure plates, filtering records, any sanitization records and microbial monitoring records of operators, gowns, gloves, and garbing practices.

Failing any lead from the above review, the current microbial profile of the product should be checked against the known historical profile for possible change. Records should be checked concomitantly for any changes in source of product components or in-processing procedures that might be contributory. Depending on the findings, and in extreme cases, consideration may have to be given to revalidation of the total manufacturing process. For the Second Stage, it is not possible to specify a particular number of specimens to be taken for testing. It is usual to select double the number specified for the First Stage under Sterility Tests [symbol] 71 [symbol] , or other reasonable number. The minimum volumes tested from each specimen, the media, and the incubation periods are the same as those indicated for the First Stage.

If no microbial growth is found in the Second Stage, and the documented review of appropriate records and the indicated product investigation does not support the possibility of intrinsic contamination, the lot may meet the requirements of a test for sterility. If growth is found, the lot fails to meet the requirements of the test. As was indicated for the First Stage test, the Second Stage test may similarly be invalidated with appropriate evidence, and, if so done, repeated as a Second Stage test.

### Auxiliary Information—Staff Liaison: Radhakrishna S Tirumalai, Ph.D., Scientist

### Expert Committee: (MSA05) Microbiology and Sterility Assurance

### USP31-NF26 Page 670

### Pharmacopeial Forum: Volume No. 30(5) Page 1729

### Phone Number: 1-301-816-8339

### 71 Sterility Tests

[symbol] Portions of this general chapter have been harmonized with the corresponding texts of the European Pharmacopeia and/or the Japanese Pharmacopeia. Those portions that are not harmonized are marked with symbols ( [symbol] [symbol] ) to specify this fact.

[symbol]

The following procedures are applicable for determining whether a Pharmacopeial article purporting to be sterile complies with the requirements set forth in the individual monograph with respect to the test for sterility. Pharmacopeial articles are to be tested by the Membrane Filtration method under Test for Sterility of the Product to be Examined where the nature of the product permits. If the membrane filtration technique is unsuitable, use the Direct Inoculation of the Culture Medium method under Test for Sterility of the Product to be Examined. All devices, with the exception of Devices with Pathways Labeled Sterile, are tested using the Direct Inoculation of the Culture Medium method. Provisions for retesting are included under Observation and Interpretation of Results.

Because sterility testing is a very exacting procedure, where asepsis of the procedure must be ensured for a correct interpretation of results, it is important that personnel be properly trained and qualified. The test for sterility is carried out under aseptic conditions. In order to achieve such conditions, the test environment has to be adapted to the way in which the sterility test is performed. The precautions taken to avoid contamination are such that they do not affect any microorganisms that are to be revealed in the test. The working conditions in which the tests are performed are monitored regularly by appropriate sampling of the working area and by carrying out appropriate controls.

These Pharmacopeial procedures are not by themselves designed to ensure that a batch of product is sterile or has been sterilized. This is accomplished primarily by validation of the sterilization process or of the aseptic processing procedures.

When evidence of microbial contamination in the article is obtained by the appropriate Pharmacopeial method, the result so obtained is conclusive evidence of failure of the article to meet the requirements of the test for sterility, even if a different result is obtained by an alternative procedure. [symbol] For additional information on sterility testing, see Sterilization and Sterility Assurance of Compendial Articles [symbol] 1211 [symbol] .

[symbol]

### Media

Prepare media for the tests as described below, or dehydrated formulations may be used provided that, when reconstituted as directed by the manufacturer or distributor, they meet the requirements of the Growth Promotion Test of Aerobes, Anaerobes, and Fungi. Media are sterilized using a validated process.

The following culture media have been found to be suitable for the test for sterility. Fluid Thioglycollate Medium is primarily intended for the culture of anaerobic bacteria. However, it will also detect aerobic bacteria. Soybean-Casein Digest Medium is suitable for the culture of both fungi and aerobic bacteria.

Fluid Thioglycollate Medium

| | | |
|---|---|---|
| L-Cystine | 0.5 | g |
| Sodium Chloride | 2.5 | g |
| Dextrose (C₆H₁₂O₆•H₂O) | 5.5/5.0 | g |
| Agar, granulated (moisture content not | 0.75 | g |
| exceeding 15%) | | |
| Yeast Extract (water-soluble) | 5.0 | g |
| Pancreatic Digest of Casein | 15.0 | g |
| Sodium Thioglycollate | 0.5 | g |
| or Thioglycolic Acid | 0.3 | mL |
| Resazurin Sodium Solution (1 in 1000), | 1.0 | mL |
| freshly prepared | | |
| Purified Water | 1000 | mL |

Mix the L-cystine, sodium chloride, dextrose, yeast extract, and pancreatic digest of casein with the purified water, and heat until solution is effected. Dissolve the sodium thioglycollate or thioglycolic acid in the solution and, if necessary, add 1 N sodium hydroxide so that, after sterilization, the solution will have a pH of 7.1±0.2. If filtration is necessary, heat the solution again without boiling, and filter while hot through moistened filter paper. Add the resazurin sodium solution, mix, and place the medium in suitable vessels that provide a ratio of surface to depth of medium such that not more than the upper half of the medium has undergone a color change indicative of oxygen uptake at the end of the incubation period. Sterilize using a validated process. If the medium is stored, store at a temperature between 2° and 25° in a sterile, airtight container. If more than the upper one-third of the medium has acquired a pink color, the medium may be restored once by heating the containers in a water-bath or in free-flowing steam until the pink color disappears and by cooling quickly, taking care to prevent the introduction of nonsterile air into the container.

Fluid Thioglycollate Medium is to be incubated at 32.5±2.5°.

### Alternative Thioglycollate Medium

Prepare a mixture having the same composition as that of the Fluid Thioglycollate Medium, but omitting the agar and the resazurin sodium solution, sterilize as directed above, and allow to cool prior to use. The pH after sterilization is 7.1±0.2. Incubate under anaerobic conditions for the duration of the incubation period.

Alternative Fluid Thioglycollate Medium is to be incubated at 32.5±2.5°.

[symbol]

Soybean-Casein Digest Medium

| | | |
|---|---|---|
| Pancreatic Digest of Casein | 17.0 | g |
| Papaic Digest of Soybean Meal | 3.0 | g |
| Sodium Chloride | 5.0 | g |
| Dibasic Potassium Phosphate | 2.5 | g |
| Dextrose (C₆H₁₂O₆•H₂O) | 2.5/2.3 | g |
| Purified Water | 1000 | mL |

Dissolve the solids in the Purified Water, heating slightly to effect a solution. Cool the solution to room temperature, and adjust the pH with 1 N sodium hydroxide so that, after sterilization, it will have a pH of 7.3±0.2. Filter, if necessary to clarify, dispense into suitable containers, and sterilize using a validated procedure. Store at a temperature between 2° and 25° in a sterile well-closed container, unless it is intended for immediate use.

Soybean-Casein Digest Medium is to be incubated at 22.5±2.5°.

### Media for Penicillins or Cephalosporins

Where sterility test media are to be used in the Direct Inoculation of the Culture Medium method under Test for Sterility of the Product to be Examined, modify the preparation of Fluid Thioglycollate Medium and the Soybean-Casein Digest Medium as follows. To the containers of each medium, transfer aseptically a quantity of β-lactamase sufficient to inactivate the amount of antibiotic in the specimen under test. Determine the quantity of β-lactamase required to inactivate the antibiotic by using a β-lactamase preparation that has been assayed previously for its penicillin- or cephalosporin-inactivating power. [NOTE—Supplemented β-lactamase media can also be used in the membrane filtration test.]

Alternatively (in an area completely separate from that used for sterility testing), confirm that an appropriate amount of β-lactamase is incorporated into the medium, following either method under Validation Test, using less than 100 colony-forming units (cfu) of Staphylococcus aureus (see Table 1) as the challenge. Typical microbial growth of the inoculated culture must be observed as a confirmation that the β-lactamase concentration is appropriate.

[symbol]

TABLE 1
Strains of the Test Microorganisms Suitable for Use
in the Growth Promotion Test and the Validation Test

| | |
|---|---|
| Aerobic bacteria | |
| Staphylococcus aureus [symbol] ¹ [symbol] | ATCC 6538, CIP 4.83, NCTC 10788, |
| | NCIMB 9518 |
| Bacillus subtilis | ATCC 6633, CIP 52.62, NCIMB 8054 |
| Pseudomonas aeruginosa [symbol] ² [symbol] | ATCC 9027, NCIMB 8626, CIP 82.118 |
| Anaerobic bacterium | |
| Clostridium sporogenes [symbol] ³ [symbol] | ATCC 19404, CIP 79.3, NCTC 532 or |
| | ATCC 11437 |
| Fungi | |
| Candida albicans | ATCC 10231, IP 48.72, NCPF 3179 |
| Aspergillus niger | ATCC 16404, IP 1431.83, IMI 149007 |

[symbol]

¹An alternative to Staphylococcus aureus is Bacillus subtilis (ATCC 6633). [symbol] [symbol]
²An alternative microorganism is Micrococcus luteus (Kocuria rhizophila), ATCC 9341. [symbol] [symbol]
³An alternative to Clostridium sporogenes, when a nonspore-forming microorganism is desired, is Bacetroides vulgatus (ATCC 8482). [symbol]
[NOTE-Seed-lot culture maintenance techniques (seed-lot systems) are used so that the viable microorganisms used for inoculation are not more than five passages removed from the original master seed lot.]

### Suitability Tests

The media used comply with the following tests, carried out before, or in parallel, with the test on the product to be examined.

### Sterility

Confirm the sterility of each sterilized batch of medium by incubating a portion of the media at the specified incubation temperature for 14 days. No growth of microorganisms occurs.

### Growth Promotion Test of Aerobes, Anaerobes, and Fungi

Test each lot of ready-prepared medium and each batch of medium prepared either from dehydrated medium or from ingredients [symbol] 1 [symbol] . Suitable strains of microorganisms are indicated in Table 1. [symbol] ¹ In appropriate cases, periodic testing of the different batches prepared from the same lot of dehydrated medium is acceptable.

[symbol]

Inoculate portions of Fluid Thioglycollate Medium with a small number (not more than 100 cfu) of the following microorganisms, using a separate portion of medium for each of the following species of microorganism: Clostridium sporogenes, Pseudomonas aeruginosa, and Staphylococcus aureus. [symbol] Inoculate portions of Alternative Fluid Thioglycollate Medium with a small number (not more than 100 cfu) of Clostridium sporogenes. [symbol] Inoculate portions of Soybean-Casein Digest Medium with a small number (not more than 100 cfu) of the following microorganisms, using a separate portion of medium for each of the following species of microorganism: Aspergillus niger, Bacillus subtilis, and Candida albicans. Incubate for not more than 3 days in the case of bacteria and not more than 5 days in the case of fungi.

The media are suitable if a clearly visible growth of the microorganisms occurs.

### Storage

If prepared media are stored in unsealed containers, they can be used for 1 month, provided that they are tested for growth promotion within 2 weeks of the time of use and that color indicator requirements are met. If stored in tight containers, the media can be used for 1 year, provided that they are tested for growth promotion within 3 months of the time of use and that the color indicator requirements are met.

[symbol]

### Diluting and Rinsing Fluids for Membrane Filtration

### Fluid A

### Preparation

Dissolve 1 g of peptic digest of animal tissue in water to make 1 L, filter or centrifuge to clarify, if necessary, and adjust to a pH of 7.1±0.2. Dispense into containers, and sterilize using a validated process.

### Preparation for Penicillins or Cephalosporins

Aseptically add to the above Preparation, if necessary, a quantity of sterile β-lactamase sufficient to inactivate any residual antibiotic activity on the membranes after the solution of the test specimen has been filtered (see Media for Penicillins or Cephalosporins).

### Fluid D

To each L of Fluid A add 1 mL of polysorbate 80, adjust to a pH of 7.1±0.2, dispense into containers, and sterilize using a validated process. Use this fluid for articles containing lecithin or oil, or for devices labeled as “sterile pathway.”

### Fluid K

Dissolve 5.0 g of peptic digest of animal tissue, 3.0 g of beef extract, and 10.0 g of polysorbate 80 in water to make 1 L. Adjust the pH to obtain, after sterilization, a pH of 6.9±0.2. Dispense into containers, and sterilize using a validated process.

[symbol]

### Validation Test

Carry out a test as described below under Test for Sterility of the Product to be Examined using exactly the same methods, except for the following modifications.

### Membrane Filtration

After transferring the content of the container or containers to be tested to the membrane, add an inoculum of a small number of viable microorganisms (not more than 100 cfu) to the final portion of sterile diluent used to rinse the filter.

### Direct Inoculation

After transferring the contents of the container or containers to be tested (for catgut and other surgical sutures for veterinary use: strands) to the culture medium, add an inoculum of a small number of viable microorganisms (not more than 100 cfu) to the medium.

In both cases use the same microorganisms as those described above under Growth Promotion Test of Aerobes, Anaerobes, and Fungi. Perform a growth promotion test as a positive control. Incubate all the containers containing medium for not more than 5 days.

If clearly visible growth of microorganisms is obtained after the incubation, visually comparable to that in the control vessel without product, either the product possesses no antimicrobial activity under the conditions of the test or such activity has been satisfactorily eliminated. The test for sterility may then be carried out without further modification.

If clearly visible growth is not obtained in the presence of the product to be tested, visually comparable to that in the control vessels without product, the product possesses antimicrobial activity that has not been satisfactorily eliminated under the conditions of the test. Modify the conditions in order to eliminate the antimicrobial activity, and repeat the validation test.

This validation is performed (a) when the test for sterility has to be carried out on a new product; and (b) whenever there is a change in the experimental conditions of the test. The validation may be performed simultaneously with the Test for Sterility of the Product to be Examined.

### Test for Sterility of the Product to be Examined

### Number of Articles to be Tested

Unless otherwise specified elsewhere in this chapter or in the individual monograph, test the number of articles specified in Table 3. If the contents of each article are of sufficient quantity (see Table 2), they may be divided so that equal appropriate portions are added to each of the specified media. [NOTE—Perform sterility testing employing two or more of the specified media.] If each article does not contain sufficient quantities for each medium, use twice the number of articles indicated in Table 3.

[symbol]

TABLE 2
Minimum Quantity to be Used for Each Medium

| | Minimum Quantity to be Used |
|---|---|
| | (unless otherwise justified |
| Quantity per Container | and authorized) |
| Liquids (other than anitbiotics) | |
| Less than 1 mL | The whole contents of each container |
| 1-40 mL | Half the contents of each container, |
| | but not less than 1 mL |
| Greater than 40 mL, and not | 20 mL |
| greater than 100 mL | |
| Greater than 100 mL | 10% of the contents of the container, |
| | but not less than 20 mL |
| Antibiotic liquids | 1 mL |
| Other preparations soluble in | The whole contents of each container to |
| water or in isopropyl myristate | provide not less than 200 mg |
| Insoluble preparations, | Use the contents of each container to |
| creams, and ointments to be | provide not less than 200 mg |
| suspended or emulsified | |
| Solids | |
| Less than 50 mg | The whole contents of each container |
| 50 mg or more, but less | Half the contents of each container, |
| than 300 mg | but not less than 50 mg |
| 300 mg-5 g | 150 mg |
| Greater than 5 g | 500 mg |
| Devices | |
| Catgut and other surgical | 3 sections of a strand (each 30-cm long) |
| sutures for veterinary use | |
| [symbol] Surgical dressing/cotton/ | 100 mg per package |
| gauze (in packages) | |
| Sutures and other individually | The whole device |
| packaged single-use material | |
| Other medical devices | The whole device, cut into pieces or |
| | disassembled [symbol] |

TABLE 3
Minimum Number of Articles to be Tested in Relation
to the Number of Articles in the Batch

| | Minimum Number of Items to be |
|---|---|
| | Tested for Each Medium |
| | (unless otherwise justified |
| Number of Items in the Batch | and authorized)* |
| Parenteral preparations | |
| Not more than 100 containers | 10% or 4 containers, whichever is the |
| | greater |
| More than 100 but not more | 10 containers |
| than 500 containers | |
| More than 500 containers | 2% or 20 containers, whichever is less |
| [symbol] For large-volume parenterals | 2% or 10 containers, whichever is less |
| Antibiotic solids | |
| Pharmacy bulk packages (<5 g) | 20 containers |
| Pharmacy bulk packages (≥5 g) | 6 containers |
| Bulks and blends | See Bulk solid products [symbol] |
| Ophthalmic and other | |
| noninjectable preparations | |
| Not more than 200 containers | 5% or 2 containers, whichever is the |
| | greater |
| More than 200 containers | 10 containers |
| If the product is presented in | |
| the form of single-dose | |
| containers, apply the scheme | |
| shown above for preparations | |
| for parenteral use. | |
| Devices | |
| Catgut and other surgical sutures | 2% or 5 packages, whichever is the |
| for veterinary use | greater, up to a maximum total |
| | of 20 packages |
| [symbol] Not more than 100 articles | 10% or 4 articles, whichever is |
| | greater |
| More than 100, but not more | 10 articles |
| than 500 articles | |
| More than 500 articles | 2% or 20 articles, whichever is less [symbol] |
| Bulk solid products | |
| Up to 4 containers | Each container |
| More than 4 containers, but not | 20% or 4 containers, whichever is |
| more than 50 containers | greater |
| More than 50 containers | 2% or 10 containers, whichever is |
| | greater |

*If the contents of one container are enough to inoculate the two media, this column gives the number of containers needed for both the media together.

The test may be carried out using the technique of Membrane Filtration or by Direct Inoculation of the Culture Medium with the product to be examined. Appropriate negative controls are included. The technique of membrane filtration is used whenever the nature of the product permits; that is, for filterable aqueous preparations, for alcoholic or oily preparations, and for preparations miscible with, or soluble in, aqueous or oily solvents, provided these solvents do not have an antimicrobial effect in the conditions of the test.

### Membrane Filtration

Use membrane filters having a nominal pore size not greater than 0.45 μm whose effectiveness to retain microorganisms has been established. Cellulose nitrate filters, for example, are used for aqueous, oily, and weakly alcoholic solutions; and cellulose acetate filters, for example, are used for strongly alcoholic solutions. Specially adapted filters may be needed for certain products (e.g., for antibiotics).

The technique described below assumes that membranes about 50 mm in diameter will be used. If filters of a different diameter are used, the volumes of the dilutions and the washings should be adjusted accordingly. The filtration apparatus and membrane are sterilized by appropriate means. The apparatus is designed so that the solution to be examined can be introduced and filtered under aseptic conditions: it permits the aseptic removal of the membrane for transfer to the medium, or it is suitable for carrying out the incubation after adding the medium to the apparatus itself.

### Aqueous Solutions

If appropriate, transfer a small quantity of a suitable, sterile diluent such as [symbol] Fluid A (see Diluting and Rinsing Fluids for Membrane Filtration)γ onto the membrane in the apparatus and filter. The diluent may contain suitable neutralizing substances and/or appropriate inactivating substances, for example, in the case of antibiotics.

Transfer the contents of the container or containers to be tested to the membrane or membranes, if necessary, after diluting to the volume used in the Validation Test with the chosen sterile diluent, but using not less than the quantities of the product to be examined prescribed in Tables 2 and 3. Filter immediately. If the product has antimicrobial properties, wash the membrane not less than three times by filtering through it each time the volume of the chosen sterile diluent used in the Validation Test. Do not exceed a washing cycle of 5 times 200 mL, even if during validation it has been demonstrated that such a cycle does not fully eliminate the antimicrobial activity. Transfer the whole membrane to the culture medium or cut it aseptically into two equal parts, and transfer one half to each of two suitable media. Use the same volume of each medium as in the Validation Test. Alternatively, transfer the medium onto the membrane in the apparatus. Incubate the media for not less than 14 days.

### Soluble Solids (Other than Antibiotics)

Use for each medium not less than the quantity prescribed in Tables 2 and 3 of the product dissolved in a suitable solvent, such as [symbol] Fluid A (Diluting and Rinsing Fluids for Membrane Filtration), [symbol] and proceed with the test as described above for Aqueous Solutions using a membrane appropriate to the chosen solvent.

### Oils and Oily Solutions

Use for each medium not less than the quantity of the product prescribed in Tables 2 and 3. Oils and oily solutions of sufficiently low viscosity may be filtered without dilution through a dry membrane. Viscous oils may be diluted as necessary with a suitable sterile diluent such as isopropyl myristate shown not to have antimicrobial activity in the conditions of the test. Allow the oil to penetrate the membrane by its own weight, and then filter, applying the pressure or suction gradually. Wash the membrane at least three times by filtering through it each time about 100 mL of a suitable sterile solution such as [symbol] Fluid A (see Diluting and Rinsing Fluids for Membrane Filtration) [symbol] containing a suitable emulsifying agent at a concentration shown to be appropriate in the validation of the test, for example polysorbate 80 at a concentration of 10 g per L [symbol] (Fluid K) [symbol] . Transfer the membrane or membranes to the culture medium or media, or vice versa, as described above for Aqueous Solutions, and incubate at the same temperatures and for the same times.

### Ointments and Creams

Use for each medium not less than the quantities of the product prescribed in Tables 2 and 3. Ointments in a fatty base and emulsions of the water-in-oil type may be diluted to 1% in isopropyl myristate as described above, by heating, if necessary, to not more than 40°. In exceptional cases it may be necessary to heat to not more than 44°. Filter as rapidly as possible, and proceed as described above for Oils and Oily Solutions.

### Prefilled Syringes

For prefilled syringes without attached sterile needles, expel the contents of each syringe into one or two separate membrane filter funnels or into separate pooling vessels prior to transfer. If a separate sterile needle is attached, directly expel the syringe contents as indicated above, and proceed as directed for Aqueous Solutions. Test the sterility of the needle, using Direct Inoculation under Validation Test.

### Solids for Injection Other than Antibiotics

Constitute the test articles as directed on the label, and proceed as directed for Aqueous Solutions or Oils and Oily Solutions, whichever applies. [NOTE—If necessary, excess diluent can be added to aid in the constitution and filtration of the constituted test article.]

### Antibiotic Solids for Injection

Pharmacy Bulk Packages, <5 g—From each of 20 containers, aseptically transfer about 300 mg of solids, into a sterile 500-mL conical flask, dissolve in about 200 mL of Fluid A (see Diluting and Rinsing Fluids for Membrane Filtration), and mix; or constitute, as directed in the labeling, each of 20 containers and transfer a quantity of liquid or suspension, equivalent to about 300 mg of solids, into a sterile 500-mL conical flask, dissolve in about 200 mL of Fluid A, and mix. Proceed as directed for Aqueous Solutions or Oils and Oily Solutions, whichever applies.

Pharmacy Bulk Packages, ≥5 g—From each of 6 containers, aseptically transfer about 1 g of solids into a sterile 500-mL conical flask, dissolve in about 200 mL of Fluid A, and mix; or constitute, as directed in the labeling, each of 6 containers and transfer a quantity of liquid, equivalent to about 1 g of solids, into a sterile 500-mL conical flask, dissolve in about 200 mL of Fluid A, and mix. Proceed as directed for Aqueous Solutions.

### Antibiotic Solids, Bulks, and Blends

Aseptically remove a sufficient quantity of solids from the appropriate amount of containers (see Table 2), mix to obtain a composite, equivalent to about 6 g of solids, and transfer to a sterile 500-mL conical flask. Dissolve in about 200 mL of Fluid A, and mix. Proceed as directed for Aqueous Solutions.

### Sterile Aerosol Products

For fluid products in pressurized aerosol form, freeze the containers in an alcohol-dry ice mixture at least at −20° for about 1 hour. If feasible, allow the propellant to escape before aseptically opening the container, and transfer the contents to a sterile pooling vessel. Add 100 mL of Fluid D to the pooling vessel, and mix gently. Proceed as directed for Aqueous Solutions or Oils and Oily Solutions, whichever applies.

### Devices with Pathways Labeled Sterile

Aseptically pass not less than 10 pathway volumes of Fluid D through each device tested. Collect the fluids in an appropriate sterile vessel, and proceed as directed for Aqueous Solutions or Oils and Oily Solutions, whichever applies.

In the case of sterile, empty syringes, draw sterile diluent into the barrel through the sterile needle, if attached, or through a sterile needle attached for the purpose of the test, and express the contents into a sterile pooling vessel. Proceed as directed above.

[symbol]

### Direct Inoculation of the Culture Medium

Transfer the quantity of the preparation to be examined prescribed in Tables 2 and 3 directly into the culture medium so that the volume of the product is not more than 10% of the volume of the medium, unless otherwise prescribed.

If the product to be examined has antimicrobial activity, carry out the test after neutralizing this with a suitable neutralizing substance or by dilution in a sufficient quantity of culture medium. When it is necessary to use a large volume of the product, it may be preferable to use a concentrated culture medium prepared in such a way that it takes into account the subsequent dilution. Where appropriate, the concentrated medium may be added directly to the product in its container.

### Oily Liquids

Use media to which have been added a suitable emulsifying agent at a concentration shown to be appropriate in the validation of the test, for example polysorbate 80 at a concentration of 10 g per L.

### Ointments and Creams

Prepare by diluting to about 1 in 10 by emulsifying with the chosen emulsifying agent in a suitable sterile diluent such as [symbol] Fluid A (see Diluting and Rinsing Fluids for Membrane Filtration). [symbol] Transfer the diluted product to a medium not containing an emulsifying agent. Incubate the inoculated media for not less than 14 days. Observe the cultures several times during the incubation period. Shake cultures containing oily products gently each day. However, when thioglycollate medium or other similar medium is used for the detection of anaerobic microorganisms, keep shaking or mixing to a minimum in order to maintain anaerobic conditions.

### Catgut and Other Surgical Sutures for Veterinarian Use

Use for each medium not less than the quantities of the product prescribed in Tables 2 and 3. Open the sealed package using aseptic precautions, and remove three sections of the strand for each culture medium. Carry out the test on three sections, each 30-cm long, which have been cut off from the beginning, the center, and the end of the strand. Use whole strands from freshly opened cassette packs. Transfer each section of the strand to the selected medium. Use sufficient medium to cover adequately the material to be tested (20 mL to 150 mL).

### Solids

Transfer a quantity of the product in the form of a dry solid (or prepare a suspension of the product by adding sterile diluent to the immediate container), corresponding to not less than the quantity indicated in Tables 2 and 3. Transfer the material so obtained to 200 mL of Fluid Thioglycollate Medium, and mix. Similarly, transfer the same quantity to 200 mL of Soybean-Casein Digest Medium, and mix. Proceed as directed above.

### Purified Cotton, Gauze, Surgical Dressings, and Related Articles

From each package of cotton, rolled gauze bandage, or large surgical dressings being tested, aseptically remove two or more portions of 100- to 500-mg each from the innermost part of the sample. From individually packaged, single-use materials, aseptically remove the entire article. Immerse the portions or article in each medium, and proceed as directed above.

### Sterile Devices

Articles can be immersed intact or disassembled. To ensure that device pathways are also in contact with the media, immerse the appropriate number of units per medium in a volume of medium sufficient to immerse the device completely, and proceed as directed above. For extremely large devices, immerse those portions of the device that are to come into contact with the patient in a volume of medium sufficient to achieve complete immersion of those portions.

For catheters where the inside lumen and outside are required to be sterile, either cut them into pieces such that the medium is in contact with the entire lumen or fill the lumen with medium, and then immerse the intact unit.

[symbol]

### Observation and Interpretation of Results

At intervals during the incubation period and at its conclusion, examine the media for macroscopic evidence of microbial growth. If the material being tested renders the medium turbid so that the presence or absence of microbial growth cannot be readily determined by visual examination, 14 days after the beginning of incubation transfer portions (each not less than 1 mL) of the medium to fresh vessels of the same medium, and then incubate the original and transfer vessels for not less than 4 days.

If no evidence of microbial growth is found, the product to be examined complies with the test for sterility. If evidence of microbial growth is found, the product to be examined does not comply with the test for sterility, unless it can be clearly demonstrated that the test was invalid for causes unrelated to the product to be examined. The test may be considered invalid only if one or more of the following conditions are fulfilled:
a. The data of the microbiological monitoring of the sterility testing facility show a fault.
b. A review of the testing procedure used during the test in question reveals a fault.
c. Microbial growth is found in the negative controls.
d. After determination of the identity of the microorganisms isolated from the test, the growth of this species (or these species) may be ascribed unequivocally to faults with respect to the material and or the technique used in conducting the sterility test procedure.

If the test is declared to be invalid, it is repeated with the same number of units as in the original test. If no evidence of microbial growth is found in the repeat test, the product examined complies with the test for sterility. If microbial growth is found in the repeat test, the product examined does not comply with the test for sterility.

### Application of the Test to Parenteral Preparations, Ophthalmic, and Other Noninjectable Preparations Required to Comply with the Test for Sterility

When using the technique of membrane filtration, use, whenever possible, the whole contents of the container, but not less than the quantities indicated in Tables 2 and 3, diluting where necessary to about 100 mL with a suitable sterile solution, such as [symbol] Fluid A (see Diluting and Rinsing Fluids for Membrane Filtration).

[symbol]

When using the technique of direct inoculation of media, use the quantities shown in Tables 2 and 3, unless otherwise justified and authorized. The tests for bacterial and fungal sterility are carried out on the same sample of the product to be examined. When the volume or the quantity in a single container is insufficient to carry out the tests, the contents of two or more containers are used to inoculate the different media.

### Auxiliary Information—Staff Liaison: Radhakrishna S Tirumalai, Ph.D., Scientist

### Expert Committee: (MSA05) Microbiology and Sterility Assurance

### USP31-NF26 Page 85

### Phone Number: 1-301-816-8339

### <51> Antimicrobial Effectiveness Testing

Antimicrobial preservatives are substances added to nonsterile dosage forms to protect them from microbiological growth or from microorganisms that are introduced inadvertently during or subsequent to the manufacturing process. In the case of sterile articles packaged in multiple-dose containers, antimicrobial preservatives are added to inhibit the growth of microorganisms that may be introduced from repeatedly withdrawing individual doses.

Antimicrobial preservatives should not be used as a substitute for good manufacturing practices or solely to reduce the viable microbial population of a nonsterile product or control the presterilization bioburden of multidose formulations during manufacturing. Antimicrobial preservatives in compendial dosage forms meet the requirements for Added Substances under Ingredients and Processes in the General Notices.

All useful antimicrobial agents are toxic substances. For maximum protection of patients, the concentration of the preservative shown to be effective in the final packaged product should be below a level that may be toxic to human beings.

The concentration of an added antimicrobial preservative can be kept at a minimum if the active ingredients of the formulation possess an intrinsic antimicrobial activity. Antimicrobial effectiveness, whether inherent in the product or whether produced because of the addition of an antimicrobial preservative, must be demonstrated for all injections packaged in multiple-dose containers or for other products containing antimicrobial preservatives. Antimicrobial effectiveness must be demonstrated for multiple-dose topical and oral dosage forms and for other dosage forms such as ophthalmic, otic, nasal, irrigation, and dialysis fluids (see

Pharmaceutical Dosage Forms [symbol] 1151 [symbol] ).

This chapter provides tests to demonstrate the effectiveness of antimicrobial protection. Added antimicrobial preservatives must be declared on the label. The tests and criteria for effectiveness apply to a product in the original, unopened container in which it was distributed by the manufacturer.

### Product Categories

For the purpose of testing, compendial articles have been divided into four categories (see Table 1). The criteria of antimicrobial effectiveness for these products are a function of the route of administration.

TABLE 1
Compendial Product Categories

| Category | Product Description |
|---|---|
| 1 | Injections, other parenterals including |
| | emulsions, otic products, sterile nasal |
| | products, and ophthalmic products made |
| | with aqueous bases or vehicles. |
| 2 | Topically used products made with aqueous |
| | bases or vehicles, nonsterile nasal products, |
| | and emulsions, including those applied |
| | to mucous membranes. |
| 3 | Oral products other than antacids, made with |
| | aqueous bases or vehicles. |
| 4 | Antacids made with an aqueous base. |

### Test Organisms

Use cultures of the following microorganisms¹: Candida albicans (ATCC No. 10231), Aspergillus niger (ATCC No. 16404), Escherichia coli (ATCC No. 8739), Pseudomonas aeruginosa (ATCC No. 9027), and Staphylococcus aureus (ATCC No. 6538). The viable microorganisms used in the test must not be more than five passages removed from the original ATCC culture. For purposes of the test, one passage is defined as the transfer of organisms from an established culture to fresh medium. All transfers are counted. In the case of organisms maintained by seed-lot techniques, each cycle of freezing, thawing, and revival in fresh medium is taken as one transfer. A seed-stock technique should be used for long-term storage of cultures. Cultures received from the ATCC should be resuscitated according to directions. If grown in broth, the cells are pelleted by centrifugation. Resuspend in 1/20th the volume of fresh maintenance broth, and add an equal volume of 20% (v/v in water) sterile glycerol. Cells grown on agar may be scraped from the surface into the 10% glycerol broth. Dispense small aliquots of the suspension into sterile vials. Store the vials in liquid nitrogen or in a mechanical freezer at no more than −50°. When a fresh seed-stock vial is required, it may be removed and used to inoculate a series of working cultures. These working cultures may then be used periodically (each day in the case of bacteria and yeast) to start the inoculum culture.

### Media

All media used in the test must be tested for growth promotion. Use the microorganisms indicated above under Test Organisms.

### Preparation of Inoculum

Preparatory to the test, inoculate the surface of a suitable volume of solid agar medium from a recently revived stock culture of each of the specified microorganisms. The culture conditions for the inoculum culture are described in Table 2 in which the suitable media are Soybean-Casein Digest or Sabouraud Dextrose Agar Medium (see Microbial Limit Tests [symbol] 61 [symbol] ).

To harvest the bacterial and C. albicans cultures, use sterile saline TS, washing the surface growth, collecting it in a suitable vessel, and adding sufficient sterile saline TS to obtain a microbial count of about 1×10⁸ colony-forming units (cfu) per mL. To harvest the cells of A. niger, use sterile saline TS containing 0.05% of polysorbate 80, and add sufficient sterile saline TS to obtain a count of about 1×10⁸ cfu per mL.

Alternatively, the stock culture organisms may be grown in a suitable liquid medium (i.e., Soybean-Casein Digest Broth or Sabouraud Dextrose Broth) and the cells harvested by centrifugation, then washed and resuspended in sterile saline TS to obtain a microbial count of about 1×10⁸ cfu per mL. [NOTE—The estimate of inoculum concentration may be performed by turbidimetric measurements for the challenge microorganisms. Refrigerate the suspension if it is not used within 2 hours.]

Determine the number of cfu per mL in each suspension, using the conditions of media and microbial recovery incubation times listed in Table 2 to confirm the initial cfu per mL estimate. This value serves to calibrate the size of inoculum used in the test. The bacterial and yeast suspensions are to be used within 24 hours of harvest, but the fungal preparation may be stored under refrigeration for up to 7 days.

### Procedure

The test can be conducted either in five original containers if sufficient volume of product is available in each container and the product container can be entered aseptically (i.e., needle and syringe through an elastomeric rubber stopper), or in five sterile, capped bacteriological containers of suitable size into which a sufficient volume of product has been transferred. Inoculate each container with one of the prepared and standardized inoculum, and mix. The volume of the suspension inoculum used is between 0.5% and 1.0% of the volume of the product. The concentration of test microorganisms that is added to the product (Categories 1, 2, and 3) are such that the final concentration of the test preparation after inoculation is between 1×10⁵ and 1×10⁶ cfu per mL of the product. For Category 4 products (antacids) the final concentration of the test preparation after inoculation is between 1×10³ and 1×10⁴ cfu per mL of the product.

The initial concentration of viable microorganisms in each test preparation is estimated based on the concentration of microorganisms in each of the standardized inoculum as determined by the plate-count method.

Incubate the inoculated containers at 22.5±2.5°. Sample each container at the appropriate intervals specified in Table 3. Record any changes observed in appearance at these intervals. Determine by the plate-count procedure the number of cfu present in each test preparation for the applicable intervals (see Procedure under Microbial Limit Tests (61)). Incorporate an inactivator (neutralizer) of the specific antimicrobial in the plate count or in the appropriate dilution prepared for plating. These conditions are determined in the validation study for that sample based upon the conditions of media and microbial recovery incubation times listed in Table 2. Using the calculated concentrations of cfu per mL present at the start of the test, calculate the change in log₁₀ values of the concentration of cfu per mL for each microorganism at the applicable test intervals, and express the changes in terms of log reductions.

TABLE 2
Culture Conditions for Inoculum Preparation

| | | | | Microbial |
|---|---|---|---|---|
| | | | Inoculum | Recovery |
| | Suitable | Incubation | Incubation | Incubation |
| Organism | Medium | Temperature | Time | Time |
| Escherichia | Soybean-Casein | 32.5 ± 2.5° | 18 to 24 | 3 to 5 |
| coli | Digest Broth; | | hours | days |
| (ATCC No. | Soybean-Casein | | | |
| 8739) | Digest Agar | | | |
| Pseudomonas | Soybean-Casein | 32.5 ± 2.5° | 18 to 24 | 3 to 5 |
| aeruginosa | Digest Broth; | | hours | days |
| (ATCC No. | Soybean-Casein | | | |
| 9027) | Digest Agar | | | |
| Staphylococcus | Soybean-Casein | 32.5 ± 2.5° | 18 to 24 | 3 to 5 |
| aureus | Digest Broth; | | hours | days |
| (ATCC No. | Soybean-Casein | | | |
| 6538) | Digest Agar | | | |
| Candida | Sabouraud | 22.5 ± 2.5° | 44 to 52 | 3 to 5 |
| albicans | Dextrose Agar; | | hours | days |
| (ATCC No. | Sabouraud | | | |
| 10231) | Dextrose Broth | | | |
| Aspergillus | Sabouraud | 22.5 ± 2.5° | 6 to 10 | 3 to 7 |
| niger | Dextrose Agar; | | days | days |
| (ATCC No. | Sabouraud | | | |
| 16404) | Dextrose Broth | | | |

### Criteria for Antimicrobial Effectiveness

The requirements for antimicrobial effectiveness are met if the criteria specified under Table 3 are met (see Significant Figures and Tolerances under General Notices). No increase is defined as not more than 0.5 log₁₀ unit higher than the previous value measured.

TABLE 3
Criteria for Tested Microorganisms
For Category 1 Products

| | |
|---|---|
| Bacteria: | Not less than 1.0 log reduction from the initial |
| | calculated count at 7 days, not less than 3.0 log |
| | reduction from the initial count at 14 days, and |
| | no increase from the 14 days' count at 28 days. |
| Yeast and | No increase from the initial calculated count at 7, |
| Molds: | 14, and 28 days. |

For Category 2 Products

| | |
|---|---|
| Bacteria: | Not less than 2.0 log reduction from the initial |
| | count at 14 days, and no increase from the 14 |
| | days' count at 28 days. |
| Yeast and | No increase from the initial calculated count at |
| Molds: | 14 and 28 days. |

For Category 3 Products

| | |
|---|---|
| Bacteria: | Not less than 1.0 log reduction from the initial |
| | count at 14 days, and no increase from the 14 |
| | days' count at 28 days. |
| Yeast and | No increase from the initial calculated count at |
| Molds: | 14 and 28 days. |

For Category 4 Products

| | |
|---|---|
| Bacteria, | No increase from the initial calculated count |
| Yeast, | at 14 and 28 days. |
| and Molds: | |

¹ Available from American Type Culture Collection, 10801 University Boulevard, Manassas, VA 20110-2209 (http://www.atcc.org).

### Auxiliary Information—Staff Liaison: Radhakrishna S Tirumalai, Ph.D., Scientist

### Expert Committee: (MSA05) Microbiology and Sterility Assurance

### USP31-NF26 Page 67

### Phone Number: 1-301-816-8339

### INCORPORATION BY REFERENCE

All publications and patents mentioned herein are hereby incorporated by reference in their entirety as if each individual publication or patent was specifically and individually indicated to be incorporated by reference.

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

Certain embodiments are within the following claims.

1. A finished cosmetic product comprising a cosmetic product, e.g., shampoo, e.g., body cleanser, disposed in an end-use container, wherein the finished cosmetic product has one or more of the following properties:
a) said cosmetic product, or said finished cosmetic product, is substantially free of a preservative, e.g., a paraben;
b) said end-use container is configured to reduce retrograde flow;
c) said cosmetic product, or finished cosmetic product, is irradiated; or
d) said cosmetic product consists essentially of the composition of one of (i) or (ii):
(i) water, cocamidopropyl betaine, rosa damascena flower water, decyl glucoside, pyrus malus (apple) fruit extract, glycerin, hydrolyzed adansonia digitata (baobab) seed protein, hydroxypropylcellulose;
(ii) water, cocamidopropyl betaine, rosa damascena flower water, decyl glucoside, pyrus malus (apple) fruit extract, glycerin, hydrolyzed quinoa, hydroxypropylcellulose, and citric acid;

2. The finished cosmetic product of claim 1, having 1, 2, 3, 4, or all of the properties of a, b, c, and wherein the cosmetic product comprises a shampoo.

3. The finished cosmetic product of claim 1, having 1, 2, 3, 4, or all of the properties of a, b, c, and wherein the cosmetic product comprises a cleanser, e.g., body cleanser

4. The finished cosmetic product of any one of claims 1-3, having the property of a.

5. The finished cosmetic product of any one of claims 1-4, having the property of b.

6. The finished cosmetic product of any one of claims 1-5, having the property of c.

7. The finished cosmetic product of any one of claims 1-6, having the property of d(i).

8. The finished cosmetic product of any one of claims 1-6, having the property of d(ii).

9. The finished cosmetic product of any one of claims 1-8, having the properties of a and b.

10. The finished cosmetic product of any one of claims 1-9, having the properties of a and c.

11. The finished cosmetic product of any one of claims 1-10, having the properties of a and d(i).

12. The finished cosmetic product of any one of claims 1-10, having the properties of a and d(ii).

13. The finished cosmetic product of any one of claims 1-12, having the properties of b and c.

14. The finished cosmetic product of any one of claims 1-13, having the properties of b and d(i).

15. The finished cosmetic product of any one of claims 1-13, having the properties of b and d(ii).

16. The finished cosmetic product of any one of claims 1-13, having the properties of c and d(i).

17. The finished cosmetic product of any one of claims 1-13, having the properties of c and d(ii).

18. The finished cosmetic product of any one of claims 1-17, having the properties of a, b, and c.

19. The finished cosmetic product of any one of claims 1-18, having the properties of a, b, and d(i).

20. The finished cosmetic product of any one of claims 1-18, having the properties of a, b, and d(ii).

21. The finished cosmetic product of any one of claims 1-20, having the properties of b, c, and d(i).

22. The finished cosmetic product of any one of claims 1-20, having the properties of b, c, and d (ii).

23. The finished cosmetic product of any one of claims 1-22, wherein the cosmetic product is an irradiated finished cosmetic product.

24. The cosmetic product of any one of claims 1-23, wherein the cosmetic product is a gamma-irradiated finished cosmetic product.

25. The finished cosmetic product of any one of claims 1-24, wherein said cosmetic product or said finished cosmetic product is substantially free of a preservative listed in Annex VI, e.g., substantially free of at least 1, 2, 5, 10, 15, 20, 30, 40, 50, or all of the preservatives listed in Annex VI.

26. The finished cosmetic product of any one of claims 1-25, wherein said cosmetic product or said finished cosmetic product has less than about 500 ppb of a preservative, e.g., less than 500 ppb.

27. The finished cosmetic product of any one of claims 1-26, wherein said cosmetic product or said finished cosmetic product has less than about 500 ppb of a preservative listed in Annex VI, e.g., less than 500 ppb of a preservative listed in Annex VI.

28. The finished cosmetic product of any one of claims 1-26, wherein said cosmetic product or said finished cosmetic product does not include a preservative.

29. The finished cosmetic product of any one of claims 1-26, wherein said cosmetic product or said finished cosmetic product does not include a preservative disclosed in Annex VI.

30. The finished cosmetic product of any one of claims 1-29, wherein said cosmetic product or said finished cosmetic product, if exposed to challenge with a microbe, e.g., a bacterium or fungus, will support growth of said microbe, e.g., as determined by U.S.P. 51, Antimicrobial Effectiveness Testing.

31. The finished cosmetic product of any one of claims 1-30, wherein said cosmetic product or said finished cosmetic product, in the absence of a treatment, e.g., sterilization treatment or the addition of a preservative, supports microbe growth, e.g., bacterial or fungal growth, e.g., as measured by U.S.P. 51, Antimicrobial Effectiveness Testing.

32. A cosmetic product, e.g., shampoo, e.g., body cleanser, disposed in an end-use container, wherein a finished cosmetic product comprises:
water, cocamidopropyl betaine, rosa damascena flower water, decyl glucoside, pyrus malus (apple) fruit extract, glycerin, hydrolyzed adansonia digitata (baobab) seed protein, hydroxypropylcellulose.

33. A cosmetic product, e.g., shampoo, e.g., body cleanser, disposed in an end-use container, wherein a finished cosmetic product comprises:
water, cocamidopropyl betaine, rosa damascena flower water, decyl glucoside, pyrus malus (apple) fruit extract, glycerin, hydrolyzed quinoa, hydroxypropylcellulose, and citric acid.

34. A cosmetic product, e.g., shampoo, e.g., body cleanser, disposed in an end-use container, wherein a finished cosmetic product consisting essentially of:
water, cocamidopropyl betaine, rosa damascena flower water, decyl glucoside, pyrus malus (apple) fruit extract, glycerin, hydrolyzed adansonia digitata (baobab) seed protein, hydroxypropylcellulose.

35. A cosmetic product, e.g., shampoo, e.g., body cleanser, disposed in an end-use container, wherein a finished cosmetic product consisting essentially of:
water, cocamidopropyl betaine, rosa damascena flower water, decyl glucoside, pyrus malus (apple) fruit extract, glycerin, hydrolyzed quinoa, hydroxypropylcellulose, and citric acid.

36. A cosmetic product, e.g., shampoo, e.g., body cleanser, disposed in an end-use container, wherein a finished cosmetic product consisting of:
water, cocamidopropyl betaine, rosa damascena flower water, decyl glucoside, pyrus malus (apple) fruit extract, glycerin, hydrolyzed adansonia digitata (baobab) seed protein, hydroxypropylcellulose.

37. A cosmetic product, e.g., shampoo, e.g., body cleanser, disposed in an end-use container, wherein a finished cosmetic product consisting of:
water, cocamidopropyl betaine, rosa damascena flower water, decyl glucoside, pyrus malus (apple) fruit extract, glycerin, hydrolyzed quinoa, hydroxypropylcellulose, and citric acid.

38. The finished cosmetic product of any one of claims 1-37, wherein said cosmetic product, or said finished cosmetic product, comprises a component added to provide one or more of the following: a fragrance, a color, viscosity, foam forming and foam stability, adhesion, moisture retention, moisture binding, pH stabilization, cleansing, thickening, softening, conditioning, e.g., hair or skin conditioning, lipid layer enhancing, barrier-forming, or film-forming.

39. The finished cosmetic product of any one of claims 1-38, wherein said cosmetic product, or said finished cosmetic product, comprises one or more of an antioxidant, fatty substance/oil, thickener, softener, emulsifier, light-screening agent, foam forming and foam stability, antifoaming agent, moisturizer, fragrance, surfactant, filler, sequestering agent, polymers, acidifying or basifying agent, dyes, colorant, pigment, pearlizer, opacifier, organic or inorganic particle, viscosity modifier, cleanser, adherent, moisture binder, pH stabilizer, conditioner, de-tangler, biobased surfactant cleanser, lipid layer enhancer, skin conditioner, and natural hair nutrient such as botanicals, fruit extracts, sugar derivatives and/or amino acids, hydrolyzed proteins, or vitamins.

40. The finished cosmetic product of any one of claims 1-39, wherein said cosmetic product or said finished cosmetic product is one of the following:
a baby product, e.g., a baby shampoo, a baby lotion, a baby oil, a baby powder, a baby cream; a bath preparation, e.g., a bath oil, a tablet, a salt, a bubble bath, a bath capsule; an eye makeup preparation, e.g., an eyebrow pencil, an eyeliner, an eye shadow, an eye lotion, an eye makeup remover, a mascara; a fragrance preparation, e.g., a colognes, a toilet water, a perfume, a powder (dusting and talcum), a sachet; hair preparations, e.g., hair conditioners, hair sprays, hair straighteners, permanent waves, rinses, shampoos, tonics, dressings, hair grooming aids, wave sets; hair coloring preparations, e.g., hair dyes and colors, hair tints, coloring hair rinses, coloring hair shampoos, hair lighteners with color, hair bleaches; makeup preparations, e.g., face powders, foundations, leg and body paints, lipstick, makeup bases, rouges, makeup fixatives; manicuring preparations, e.g., basecoats and undercoats, cuticle softeners, nail creams and lotions, nail extenders, nail polish and enamel, nail polish and enamel removers; oral hygiene products, e.g., dentrifices, mouthwashes and breath fresheners; bath soaps, e.g., foaming body cleansers, and detergents, deodorants, douches, feminine hygiene deodorants; shaving preparations, e.g., aftershave lotions, beard softeners, talcum, preshave lotions, shaving cream, shaving soap; skin care preparations, e.g., cleansing, depilatories, face and neck, body and hand, foot powders and sprays, moisturizing, night preparations, paste masks, skin fresheners; and suntan preparations, e.g., gels, creams, and liquids, and indoor tanning preparations.

41. The finished cosmetic product of any one of claims 1-40, comprising a shampoo.

42. The finished cosmetic product of any one of claims 1-41, the shampoo comprising water, cocamidopropyl betaine, rosa damascena flower water, decyl glucoside, pyrus malus (apple) fruit extract, glycerin, hydrolyzed adansonia digitata (baobab) seed protein, hydroxypropylcellulose.

43. The finished cosmetic product of any one of claims 1-42, the shampoo consisting essentially of water, cocamidopropyl betaine, rosa damascena flower water, decyl glucoside, pyrus malus (apple) fruit extract, glycerin, hydrolyzed adansonia digitata (baobab) seed protein, hydroxypropylcellulose.

44. The finished cosmetic product of any one of claims 1-43, the shampoo consisting of water, cocamidopropyl betaine, rosa damascena flower water, decyl glucoside, pyrus malus (apple) fruit extract, glycerin, hydrolyzed adansonia digitata (baobab) seed protein, hydroxypropylcellulose.

45. The finished cosmetic product of any one of claims 1-40, comprising a foaming body cleanser.

46. The finished cosmetic product of any one of claims 1-40, 45 the foaming body cleanser comprising water, cocamidopropyl betaine, rosa damascena flower water, decyl glucoside, pyrus malus (apple) fruit extract, glycerin, hydrolyzed quinoa, hydroxypropylcellulose, and citric acid.

47. The finished cosmetic product of any one of claims 1-40, 45, 46, the foaming body cleanser consisting essentially of water, cocamidopropyl betaine, rosa damascena flower water, decyl glucoside, pyrus malus (apple) fruit extract, glycerin, hydrolyzed quinoa, hydroxypropylcellulose, and citric acid.

48. The finished cosmetic product of any one of claims 1-40, 45-47, the foaming body cleanser consisting of water, cocamidopropyl betaine, rosa damascena flower water, decyl glucoside, pyrus malus (apple) fruit extract, glycerin, hydrolyzed quinoa, hydroxypropylcellulose, and citric acid.

49. The finished cosmetic product of any one of claims 1-40, comprising a conditioner.

50. The finished cosmetic product of any one of claims 1-40, 49, the conditioner comprising hydroxypropyl cellulose, cationic guar, coconut oil, and a fragrance.

51. The finished cosmetic product of any one of claims 1-40, 49, 50, the conditioner consisting essentially of hydroxypropyl cellulose, cationic guar, coconut oil, and a fragrance.

52. The finished cosmetic product of any one of claims 1-40 49-51, the conditioner consisting of hydroxypropyl cellulose, cationic guar, coconut oil, and a fragrance.

53. The finished cosmetic product of any one of claims 1-52, wherein said finished cosmetic product, or cosmetic product, is treated by sterilization.

54. The finished cosmetic product of any one of claims 1-53, wherein sterilization comprises irradiation.

55. The finished cosmetic product of any one of claims 1-54, wherein sterilization comprises heating.

56. The finished cosmetic product of any one of claims 1-55, wherein said finished cosmetic product, or cosmetic product, is sterile, e.g., as determined by sterility assurance level testing.

57. The finished cosmetic product of any one of claims 1-56, wherein at least 10, 20, 30, 40, 50, 60, 70, 80, 90, 95, 99, or 99.9% of the bacteria, are dead or incapable of cell division.

58. The finished cosmetic product of any one of claims 1-57, wherein at least 10, 20, 30, 40, 50, 60, 70, 80, 90, 95, 99, or 99.9% of the bacteria, have radiation induced DNA damage sufficient to inhibit cell division.

59. The finished cosmetic product of any one of claims 1-58, wherein the end-use container is unopened, e.g., the factory seal has not been broken.

60. The finished cosmetic product of any one of claims 1-59, wherein said cosmetic product, or said finished cosmetic product, is irradiated, e.g., with ionizing radiation, e.g., with gamma rays, e.g., with x-rays, e.g., from an isotope, e.g., cobalt 60, or with ultraviolet, e.g., ultraviolet C (UVC).

61. The finished cosmetic product of any one of claims 1-60, wherein said irradiation is sufficient to provide a sterile product.

62. The finished cosmetic product of any one of claims 1-61, wherein the sterile product is characterized in that it is substantially free from microorganisms, e.g., bacteria, e.g., fungi, e.g, spores, capable of growth, e.g., consistent with U.S.P. Chapter 1211, as determined by U.S.P. 71 Sterility Testing Methods and Standards.

63. The finished cosmetic product of any one of claims 1-62, wherein, when challenged for microorganisms capable of growth, said cosmetic product shows no growth, e.g., when said microorganisms are measured by U.S.P. 71 Sterility Testing Methods and Standards.

64. The finished cosmetic product of any one of claims 1-63, wherein said cosmetic product, or said finished cosmetic product, comprises an exogenously added additive selected from an oxidant, e.g., a naturally occurring oxidant, a free radical scavenger, or a free radical quencher.

65. The finished cosmetic product of any one of claims 1-64, wherein said cosmetic product, or said finished cosmetic product, contains a plurality of components, and is irradiated after mixture of the plurality of components.

66. The finished cosmetic product of any one of claims 1-65, wherein said finished cosmetic product is irradiated after said cosmetic product is disposed in the end-use container.

67. The finished cosmetic product of any one of claims 1-66, wherein said finished cosmetic product is irradiated after closure of the end-use container.

68. The finished cosmetic product of any one of claims 1-67, wherein said finished cosmetic product is irradiated after sealing of the end-use container.

69. The finished cosmetic product of any one of claims 1-68, wherein said finished cosmetic product is irradiated prior to closure of the end-use container.

70. The finished cosmetic product of any one of claims 1-69, comprising an indicator that indicates if said cosmetic product, or said finished cosmetic product, has been irradiated.

71. The finished cosmetic product of any one of claims 1-70, wherein said cosmetic product, or finished cosmetic product, is heated, e.g., by microwave oven or autoclave.

72. The finished cosmetic product of any one of claims 1-71, wherein said heating is sufficient to provide a sterile product.

73. The finished cosmetic product of any one of claims 1-72, wherein the sterile product is characterized in that it is substantially free from microorganisms, e.g., bacteria, e.g., fungi capable of growth, e.g., as determined by U.S.P. 71 Sterility Testing Methods and Standards.

74. The finished cosmetic product of any one of claims 1-73, wherein, when challenged for microorganisms capable of growth, said cosmetic product shows no growth, e.g., when said microorganisms are measured by when said microorganisms are measured by U.S.P. 71 Sterility Testing Methods and Standards.

75. The finished cosmetic product of any one of claims 1-74, wherein said cosmetic product, or said finished cosmetic product, contains a plurality of components, and is heated after mixture of the plurality of components.

76. The finished cosmetic product of any one of claims 1-75, wherein the finished cosmetic product is heated after said cosmetic product is disposed in the end-use container.

77. The finished cosmetic product of any one of claims 1-76, wherein said finished cosmetic product is heated after closure of the end-use container.

78. The finished cosmetic product of any one of claims 1-77, wherein said finished cosmetic product is heated after sealing of the end-use container.

79. The finished cosmetic product of any one of claims 1-78, wherein said finished cosmetic product is heated prior to closure of the end-use container.

80. The finished cosmetic product of any one of claims 1-79, comprising an indicator that indicates if said cosmetic product, or said finished cosmetic product, has been heated.

81. The finished cosmetic product of any one of claims 1-80, wherein said cosmetic product, or said finished product, is heated at or above 121 degrees Celsius for at least 15 minutes during or after formulation, e.g., after mixing, or filling, e.g., after disposing.

82. The finished cosmetic product of any one of claims 1-81, wherein the end-use container is configured to inhibit retrograde flow, e.g., backflow, e.g., reverse flow, e.g., rearward movement, of material into said end-use container.

83. The finished cosmetic product of any one of claims 1-82, wherein the end-use container is configured to inhibit retrograde flow, e.g., backflow, e.g., reverse flow, e.g., rearward movement, of material, e.g., said cosmetic product, into said end-use container.

84. The finished cosmetic product of any one of claims 1-83, wherein the end-use container is configured to inhibit retrograde flow, e.g., backflow, e.g., reverse flow, e.g., rearward movement, of material, e.g., a contaminant, into said end-use container.

85. The finished cosmetic product of any one of claims 1-84, wherein the contaminant is atmospheric, e.g., an aerosol, or a liquid, e.g., water, or solid, or a gas.

86. The finished cosmetic product of any one of claims 1-85, wherein the end-use container comprises a reservoir in which said cosmetic product is disposed, and a dispenser through which said cosmetic product from said reservoir can be dispensed, wherein said dispenser inhibits retrograde flow of material into said reservoir.

87. The finished cosmetic product of any one of claims 1-86, wherein the end-use container comprises a reservoir in which said cosmetic product is disposed, and a dispenser through which said cosmetic product from said reservoir can be dispensed, wherein said dispenser inhibits retrograde flow of dispensed cosmetic product, or atmospheric aerosols, into said reservoir.

88. The finished cosmetic product of any one of claims 1-87, wherein the end-use container is an anti-retrograde flow dispenser comprising a first pressure activated valve disposed in said dispenser and proximal to said reservoir and a second pressure activated valve disposed in said dispenser and distal to said reservoir, wherein the activation pressure of said first valve is higher than the activation pressure of said second valve.

89. The finished cosmetic product of any one of claims 1-88, wherein the end-use container comprises an anti-retrograde mechanism configured to prevent movement of the cosmetic product in a direction opposite the operational direction associated with dispensing the finished cosmetic product.

90. The finished cosmetic product of any one of claims 1-89, wherein the amount of cosmetic product in said end-use container is sufficient for no more than X applications, wherein X is between about 1 and about 180.

91. The finished cosmetic product of any one of claims 1-90, wherein the amount of cosmetic product in said finished cosmetic product is selected such that the finished cosmetic product is sufficient for no more than X applications, wherein X is about 1 and about 180.

92. The finished cosmetic product of any one of claims 1-91, wherein the amount of cosmetic product in said finished cosmetic product is selected such that the finished cosmetic product is sufficient for no more than about 28 days of use.

93. The finished cosmetic product of any one of claims 1-92, wherein the amount of cosmetic product in said finished cosmetic product is selected such that the finished cosmetic product is sufficient for not more than X days of Y/day use, wherein X is about two weeks to about 6 weeks and Y is about zero per day use to about six times per day use.

94. The finished cosmetic product of any one of claims 1-93, having a deterioration-based expiration date.

95. The finished cosmetic product of any one of claims 1-94, having an indication of deterioration-based expiration date.

96. The finished cosmetic product of any one of claims 1-95, having a biome-compatible-based expiration date.

97. The finished cosmetic product of any one of claims 1-96, having an indication of a biome-compatible-based expiration date.

98. The finished cosmetic product of any one of claims 1-97, wherein said finished product comprises an indication of expiration, or lifetime, e.g., recommended lifetime.

99. The finished cosmetic product of any one of claims 1-98, wherein said finished product comprises an indication of expiration, or lifetime, e.g., recommended lifetime, after a preselected period of time, e.g., expressed in days or weeks.

100. The finished cosmetic product of any one of claims 1-99, wherein said finished product comprises an indication of expiration, or lifetime, e.g., recommended lifetime, after the preselected period of time, e.g., expressed in days, that is less than X days from the date of one of manufacturing, filling, sealing, shipping, releasing into commerce, or selling, wherein X is about 5-7 days, about 5-10 days, about 7-14 days, about 14-21 days, about 21-28 days, about 28-35 days, about 35-42 days, about 42-49 days, about 49-56 days, about 56-63 days, about 63-70 days, about 70-77 days, about 75-100 days, about 100-150 days, about 150-200 days, about 200-300 days, about 300-400 days, about 400-750 days.

101. The finished cosmetic product of any one of claims 1-100, wherein said finished product comprises an indication of expiration, or lifetime, e.g., recommended lifetime, after the preselected period of time, e.g., expressed in days, that is less than X days from the date of one of manufacturing, filling, sealing, shipping, releasing into commerce, or selling, wherein X is about 28 days, e.g., 28 days.

102. The finished cosmetic product of any one of claims 1-101, wherein said finished product comprises an indication of expiration, or lifetime, e.g., recommended lifetime, expressed as a preselected period of time, e.g., days from the opening or unsealing of said finished cosmetic product.

103. The finished cosmetic product of any one of claims 1-102, wherein said finished product comprises an indication of expiration, or lifetime, e.g., recommended lifetime, expressed as a preselected period of time, e.g., days from the opening or unsealing of said finished cosmetic product that is less than X days from the date of opening or unsealing, wherein X is about 5-7 days, about 5-10 days, about 7-14 days, about 14-21 days, about 21-28 days, about 28-35 days, about 35-42 days, about 42-49 days, about 49-56 days, about 56-63 days, about 63-70 days, about 70-77 days, about 75-100 days, about 100-150 days, about 150-200 days, about 200-300 days, about 300-400 days.

104. The finished cosmetic product of any one of claims 1-103, wherein said finished product comprises an indication of expiration, or lifetime, e.g., recommended lifetime, expressed as a preselected period of time, e.g., days from the opening or unsealing of said finished cosmetic product that is less than X days from the date of opening or unsealing, wherein X is about 28 days, e.g., 28 days.

105. The finished cosmetic product of any one of claims 1-104, wherein the indication of expiration, or lifetime, e.g., recommended lifetime, is expressed as a preselected number of uses or applications.

106. The finished cosmetic product of any one of claims 1-105, wherein the indication of expiration, or lifetime, e.g., recommended lifetime, is expressed as a preselected number of uses or applications, wherein the preselected number is between about 5-7, about 5-10, about 7-14, about 14-21, about 21-28, about 28-35, about 35-42, about 42-49, about 49-56, about 56-63, about 63-70, about 70-77, about 75-100, about 100-150, about 150-200, about 200-300, about 300-400.

107. The finished cosmetic product of any one of claims 1-106, having an expiration date, or lifetime, e.g., recommended lifetime.

108. The finished cosmetic product of any one of claims 1-107, wherein said expiration date, or lifetime, e.g., recommended lifetime, is expressed:
a) in units of time, e.g., days, from a preselected event, e.g., unsealing of said finished cosmetic product or the first use of said finished cosmetic product; or
b) as the number of uses or applications.

109. The finished cosmetic product of claim 108, wherein said expiration date, or lifetime, e.g., recommended lifetime, is expressed as a.

110. The finished cosmetic product of claim 108, wherein said expiration date, or lifetime, e.g., recommended lifetime, is expressed as b.

111. The finished cosmetic product of claim 108, wherein said expiration date, or lifetime, e.g., recommended lifetime, is expressed as a and b.

112. The finished cosmetic product of claim 108, wherein said expiration date, or lifetime, e.g., recommended lifetime, is expressed as a or b.

113. The finished cosmetic product of any one of claims 1-112, wherein the end-use container comprises a polymer, e.g., polyethylene terephthalate (PET), high density polyethylene (HDPE), polypropylene, polycarbonate, polytetrafluoroethylene (Teflon®), polyviylidene fluoride (PVDF), or a cellulosic.

114. The finished cosmetic product of any one of claims 1-112, wherein the end-use container comprises glass.

115. The finished cosmetic product of any one of claims 1-114, comprising a sensor, e.g., an oxygen sensor, that indicates a presence of viable bacterial.

116. The finished cosmetic product of any one of claims 1-115, wherein the end-use container allows passage of at least about 5, 10, 20, 30, 40, 50, 60, 70, 80, 90, 95, 99, or 100 percent of transmission of ionizing radiation, e.g., with gamma rays, e.g., with x-rays, e.g., from an isotope, e.g., cobalt 60, or with ultraviolet, e.g., ultraviolet C (UVC) through the end-use container.

117. A method of distributing a finished cosmetic product, comprising:
a) supplying (or causing a designee to supply) an end-user with a first unit of a finished cosmetic product;
b) providing (or causing a designee to provide) said end-user, or an entity designated by said end user, e.g., a second end-user, with:
i) a subsequent unit of said finished cosmetic product, or a unit of a second finished cosmetic product; or
ii) a notification that said first unit has reached the end of its recommended life;
c) optionally, providing (or causing a designee to provide) said end-user or an entity designated by said end user, e.g., a second end-user, with information regarding disposal, e.g., recycling, said first unit of finished cosmetic product, e.g., a finished cosmetic product on which the recommended life has elapsed.
thereby distributing a finished cosmetic product.

118. The method of claim 117, comprising:
a) supplying an end-user with a first unit of a finished cosmetic product;
b) providing said end-user, with:
i) a subsequent unit of said finished cosmetic product; or
ii) a notification that said first unit has reached the end of its recommended life; and
c) providing said end-user, with information regarding disposal, e.g., recycling, said first unit of finished cosmetic product, e.g., a finished cosmetic product on which the recommended life has elapsed.
thereby distributing a finished cosmetic product.

119. The method of any one of claims 117-118, wherein the finished cosmetic product comprises the finished cosmetic product of any one of claims 1-116.

120. The method of any one of claims 117-119, wherein said first unit of finished cosmetic product comprises a cosmetic product described herein.

121. The method of any one of claims 117-120, wherein said first unit of finished cosmetic product comprises a cosmetic product that is free, or substantially free, of bacteria or fungi.

122. The method of any one of claims 117-121, wherein said first unit of finished cosmetic product comprises a cosmetic product that is free, or substantially free, of preservative.

123. The method of any one of claims 117-122, wherein said first unit of finished cosmetic product comprises a cosmetic product selected from:
a baby product, e.g., a baby shampoo, a baby lotion, a baby oil, a baby powder, a baby cream; a bath preparation, e.g., a bath oil, a tablet, a salt, a bubble bath, a bath capsule; an eye makeup preparation, e.g., an eyebrow pencil, an eyeliner, an eye shadow, an eye lotion, an eye makeup remover, a mascara; a fragrance preparation, e.g., a colognes, a toilet water, a perfume, a powder (dusting and talcum), a sachet; hair preparations, e.g., hair conditioners, hair sprays, hair straighteners, permanent waves, rinses, shampoos, tonics, dressings, hair grooming aids, wave sets; hair coloring preparations, e.g., hair dyes and colors, hair tints, coloring hair rinses, coloring hair shampoos, hair lighteners with color, hair bleaches; makeup preparations, e.g., face powders, foundations, leg and body paints, lipstick, makeup bases, rouges, makeup fixatives; manicuring preparations, e.g., basecoats and undercoats, cuticle softeners, nail creams and lotions, nail extenders, nail polish and enamel, nail polish and enamel removers; oral hygiene products, e.g., dentrifices, mouthwashes and breath fresheners; bath soaps, e.g., foaming body cleansers, and detergents, deodorants, douches, feminine hygiene deodorants; shaving preparations, e.g., aftershave lotions, beard softeners, talcum, preshave lotions, shaving cream, shaving soap; skin care preparations, e.g., cleansing, depilatories, face and neck, body and hand, foot powders and sprays, moisturizing, night preparations, paste masks, skin fresheners; and suntan preparations, e.g., gels, creams, and liquids, and indoor tanning preparations.

124. The method of claim 123, wherein said first unit of finished cosmetic product comprises a shampoo.

125. The method of claim 123, wherein said first unit of finished cosmetic product comprises a foaming body cleanser.

126. The method of claim 123, wherein said first unit of finished cosmetic product comprises a conditioner.

127. The method of any one of claims 117-126, wherein said end-user is supplied with, e.g., by sale, or gifting, said first unit of a finished cosmetic product from an internet-based outlet.

128. The method of any one of claims 117-127, wherein said end-user is supplied with, e.g., by sale, or gifting, said first unit of a finished cosmetic product from a non-internet-based outlet, e.g., a store.

129. The method of any one of claims 117-128, wherein providing said end-user comprises providing said end-user with i.

130. The method of any one of claims 117-129, wherein providing said end-user comprises providing said end-user with ii.

131. The method of any one of claims 117-130, wherein providing said end-user comprises providing said end-user with i and ii.

132. The method of any one of claims 117-131, wherein said provision is made within a preselected number of days, e.g., 1-28 days, e.g., 3-7 days prior to the end of the recommended life of the first unit of a finished cosmetic product.

133. The method of any one of claims 117-132, wherein said provision is made within a preselected number of days, e.g., 1 day to about 28 days, e.g., 21-25 days, after supply of said first unit of a finished cosmetic product.

134. The method of any one of claims 117-133, wherein said provision is made within a preselected number of days, e.g., 1 day to about 28 days, prior to or subsequent to a first expected use of the finished cosmetic product.

135. The method of any one of claims 117-134, wherein said subsequent unit of finished cosmetic product comprises a cosmetic product described herein.

136. The method of any one of claims 117-135, wherein said subsequent unit of finished cosmetic product comprises a cosmetic product that is free, or substantially free, of bacteria or fungi.

137. The method of any one of claims 117-136, wherein said subsequent unit of finished cosmetic product comprises a cosmetic product that is free, or substantially free, of preservative.

138. The method of any one of claims 117-137, wherein said subsequent unit of finished cosmetic product comprises a cosmetic product selected from one of the following:
a baby product, e.g., a baby shampoo, a baby lotion, a baby oil, a baby powder, a baby cream; a bath preparation, e.g., a bath oil, a tablet, a salt, a bubble bath, a bath capsule; an eye makeup preparation, e.g., an eyebrow pencil, an eyeliner, an eye shadow, an eye lotion, an eye makeup remover, a mascara; a fragrance preparation, e.g., a colognes, a toilet water, a perfume, a powder (dusting and talcum), a sachet; hair preparations, e.g., hair conditioners, hair sprays, hair straighteners, permanent waves, rinses, shampoos, tonics, dressings, hair grooming aids, wave sets; hair coloring preparations, e.g., hair dyes and colors, hair tints, coloring hair rinses, coloring hair shampoos, hair lighteners with color, hair bleaches; makeup preparations, e.g., face powders, foundations, leg and body paints, lipstick, makeup bases, rouges, makeup fixatives; manicuring preparations, e.g., basecoats and undercoats, cuticle softeners, nail creams and lotions, nail extenders, nail polish and enamel, nail polish and enamel removers; oral hygiene products, e.g., dentrifices, mouthwashes and breath fresheners; bath soaps, e.g., foaming body cleansers, and detergents, deodorants, douches, feminine hygiene deodorants; shaving preparations, e.g., aftershave lotions, beard softeners, talcum, preshave lotions, shaving cream, shaving soap; skin care preparations, e.g., cleansing, depilatories, face and neck, body and hand, foot powders and sprays, moisturizing, night preparations, paste masks, skin fresheners; and suntan preparations, e.g., gels, creams, and liquids, and indoor tanning preparations.

139. The method of claim 138, wherein said subsequent unit of finished cosmetic product comprises a shampoo.

140. The method of claim 138, wherein said subsequent unit of finished cosmetic product comprises a foaming body cleanser.

141. The method of claim 138, wherein said subsequent unit of finished cosmetic product comprises a conditioner.

142. The method of any one of claims 117-141, wherein said subsequent unit of said finished cosmetic product, or a unit of a second finished cosmetic product, is delivered, e.g., by mail or a commercial delivery entity, to said end-user.

143. The method of any one of claims 117-142, wherein said subsequent unit of said finished cosmetic product, or a unit of a second finished cosmetic product, is delivered, e.g., by mail or a commercial delivery entity, to an entity, e.g., a second end user, designated by said end-user.

144. The method of any one of claims 117-143, wherein said subsequent unit of said finished cosmetic product, or a unit of a second finished cosmetic product, is delivered, e.g., by mail or a commercial delivery entity, to a location designated by said end-user.

145. The method of any one of claims 117-144, wherein said notification, is delivered, e.g., by mail or a commercial delivery entity, or electronically, e.g., by internet or telephone, e.g., by telephone call, e.g., by recorded message, or text message to said end-user.

146. The method of any one of claims 117-145, wherein said notification, is delivered, e.g., by mail or a commercial delivery entity, or electronically, e.g., by internet or telephone, e.g., by telephone call, e.g., by recorded message, or text message, to an entity, e.g., a second end user, designated by said end-user.

147. The method of any one of claims 117-146, wherein said notification, is delivered, e.g., by mail or a commercial delivery entity, or electronically, e.g., by internet or telephone, to a location designated by said end-user.

148. The method of any of one of claims 117-147, comprising:
c) providing (or causing a designee to provide) said end-user or an entity designated by said end user, e.g., a second end-user, with information regarding disposal, e.g., recycling, of said first unit of finished cosmetic product, e.g., a finished cosmetic product on which the recommended life has elapsed.

149. The method of any one of claims 117-148, wherein said information comprises the name or location, e.g., address, of an entity (a collection entity) which will accept said first unit of finished cosmetic product, e.g., after its recommended life.

150. The method of any one of claims 117-149, comprising providing said end-user, or designee, with a container configured to receive said first unit of finished cosmetic product, e.g., after its recommended life.

151. The method of claim 150, wherein said container is provided with the first unit of finished cosmetic product.

152. The method of any one of claims 150-151, wherein said container is provided with said notification.

153. The method of any one of claims 150-152, wherein said container comprises a mailing label addressed to said collection entity.

154. The method of any one of claim 153, wherein said collection entity is a recycler.

155. The method of any one of claims 117-154, comprising:
d) providing (or causing a designee to provide) said end-user or an entity designated by said end user, e.g., a second end-user, with information regarding disposal, e.g., recycling, of said subsequent unit of finished cosmetic product, e.g., a finished cosmetic product, or cosmetic product, on which the recommended lifetime has elapsed.

156. The method of claim 155, wherein said information comprises the name or location, e.g., address, of an entity (a collection entity) which will accept said subsequent unit of finished cosmetic product, e.g., after its recommended life.

157. The method of any one of claims 155-156, comprising providing said end-user, or designee, with a container configured to receive said subsequent unit of finished cosmetic product, e.g., after its recommended life.

158. The method of claim 157, wherein said container is provided with the subsequent unit of finished cosmetic product.

159. The method of any one of claims 157-158, wherein said container is provided with said notification.

160. The method of any one of claims 157-159, wherein said container comprises a mailing label addressed to said collection entity.

161. The method of claim 160, wherein said collection entity is a recycler.

162. A method of obtaining a finished cosmetic product, comprising:
a) receiving a first unit of a finished cosmetic product;
b) receiving:
i) a subsequent unit of said finished cosmetic product, or a unit of a second finished cosmetic product; or
ii) a notification that said first unit has reached the end of its recommended life;
c) optionally, receiving information regarding disposal, e.g., recycling, said first unit of finished cosmetic product, e.g., a finished cosmetic product on which the recommended life has elapsed.
thereby obtaining a finished cosmetic product.

163. A method of distributing a finished cosmetic product comprising:
obtaining, e.g., manufacturing, a finished cosmetic product of any of the above claims, said finished cosmetic product comprising a cosmetic product, e.g., shampoo, e.g., body wash, disposed in an end use container;
communicating to an end user of said finished product, one or more of the following:
the finished cosmetic product, or cosmetic product, is biome-friendly, e.g., biome-compatible;
the finished cosmetic product, or cosmetic product, should not be used after an indicated expiration date, or lifetime, e.g., recommended lifetime, based on, e.g., deterioration, e.g., biome-compatibility,
wherein said indicated expiration date or lifetime, e.g., recommended lifetime is expressed:
(a) in units of time, e.g., days, from a preselected event, e.g, unsealing of said finished cosmetic product or a first use of said finished cosmetic product; or
(b) as the number of uses or applications;
the finished cosmetic product, or cosmetic product, should not be used after X applications, for example, wherein X is between about 1 and about 180; and
the finished cosmetic product, or cosmetic product, should not be used after X days of use, e.g., X days of Y/day use, wherein X is between about (7 days) one week and about 42 days (6 weeks), and Y is between about zero uses per day and about ten uses per day. For example X days may be about 7-10, 10-13, 14-17, 18-21, 22-25, 26-29, 30-33, 34-37, 38-42 days; and Y may be about 0-1, 2-4, 5-7, 8-10 uses per day.

164. The method of claim 163, further comprising shipping said finished cosmetic product.

165. A method of making a finished cosmetic product, comprising:
disposing a cosmetic product in an end-use container to form a filled end-use container; and
treating said filled end-use container to kill or inactivate bacteria,
thereby providing a finished cosmetic product.

166. A method of making a biome-friendly cosmetic product comprising:
selecting a first component, e.g., a surfactant, from a list of biome-friendly components;
selecting a second component, e.g., a humectant, from a list of biome-friendly components;
providing a mixture of said first and second component, thereby making a biome-friendly cosmetic product.

167. The method of claim 166, wherein said first, second, or first and second components are selected from Table 3.

168. The method of any one of claims 166-167, further comprising, determining the biome-friendliness expiration date or recommend life of said finished cosmetic product.

169. The method of claim 168, wherein said determination comprises evaluating the viability of an AOB or of the ability of a AOB to produce nitrite after contact with the mixture of the first and second component.

170. A method of making a finished cosmetic product comprising:
providing a first and second component, wherein each has been shows to be biome-friendly;
combining said first and second component to form a mixture;
determining if said mixture is biome-friendly, thereby making a finished cosmetic product.

171. The method of claim 170, wherein determining comprises evaluating the viability of an AOB or of the ability of a AOB to produce nitrite after contact with the mixture of the first and second component.

172. A method of manufacturing (evaluating) a cosmetic product or finished cosmetic product comprising:
providing a product having at least 2 components selected from Table 3;
acquiring an evaluation of whether the cosmetic product is safe for bacteria, e.g., beneficial bacteria, e.g., AOB, on the skin of the user,
thereby manufacturing the cosmetic product or finished cosmetic product.

173. The method of claim 172, wherein evaluation comprises evaluating the viability of an AOB or of the ability of a AOB to produce nitrite after contact with the product.

174. A method of evaluating a cosmetic product or a finished cosmetic product, comprising:
contacting an aliquot of cosmetic product with a test organism, e.g., an ammonia oxidizing bacteria; and
evaluating an effect of the cosmetic product on the test organism,
wherein evaluating comprises evaluating the effect of the cosmetic product on an ability of the test organism, e.g., ammonia oxidizing bacteria, to produce nitrite.

175. The method of claim 174, wherein evaluating comprises determining if the ability of the ammonia oxidizing bacteria to produce nitrite meets a preselected criteria, e.g., at least has the ability to recover so as to produce nitrite over a given period of time as shown in the Figures of the Examples disclosed herein.

176. The method of any one of claims 174-175, wherein the evaluating the effect of the cosmetic product on the ability of the test organism provides for the finished cosmetic product to be identified as “tested and confirmed to have the live test organisms.”

177. The method of any one of claims 174-176, wherein evaluating the effect of the cosmetic product on the ability of ammonia oxidizing bacteria to produce nitrite provides for the finished cosmetic product to be identified as “tested and confirmed to have live ammonia oxidizing bacteria.”

178. A method of selecting an ammonia oxidizing bacteria-friendly excipient comprising:
obtaining an ammonia oxidizing bacteria (AOB) cell suspension, e.g., from a continuous culture system;
harvesting AOB cells from the cell suspension;
washing the AOB cells in a storage solution, e.g., a storage solution comprising 50 mM Na₂HPO₄— 2 mM MgCl₂, pH 7.6;
suspending the AOB cells in the storage solution at a final optical density (OD₆₀₀) of 5.0 (˜10¹⁰ cells/ml);
storing the AOB cells at about 4° C.;
diluting the AOB cells to a final optical density (OD₆₀₀) of 0.5 (˜10⁹ cells/ml) in 10 ml AOB medium supplemented with ammonium (NH₄⁺), e.g., 50 mM ammonium, containing an excipient at a pre-determined final concentration;
incubating at 30° C. for a first pre-determined time period to provide an incubated culture;
collecting an aliquot of the incubated culture; and
measuring a concentration of nitrite in a supernatant of the incubated culture.

179. The method of claim 178, wherein the supernatant is provided by centrifuging the aliquot of the incubated culture to provide the supernatant and a bacterial pellet.

180. The method of any one of claims 178-179, further comprising, identifying the excipient as an ammonia oxidizing bacteria-friendly ingredient based on the concentration of nitrite in the supernatant of the incubated culture.

181. The method of any one of claims 178-180, further comprising:
washing the bacterial pellet in the AOB medium;
suspending the bacterial pellet in the AOB medium supplemented with NH₄⁺;
incubating the bacterial pellet in the AOB medium supplemented with NH₄⁺;
recovering AOB cells at a second pre-determined time periods to provide recovered AOB cell samples; and
measuring the recovered AOB cell sample for at least one of an OD₆₀₀ value and nitrite accumulation.

182. The method of any one of claims 178-181, further comprising, identifying the excipient as an ammonia oxidizing bacteria-friendly ingredient based on at least one of the OD₆₀₀ value and nitrite accumulation in the recovered AOB cell sample.

183. The method of any one of claims 178-182, wherein harvesting AOB cells from the cell suspension comprises centrifuging the cell suspension.

184. The method of any one of claims 178-183, wherein the pre-determined final concentration of excipient is between about 0% and about 100%.

185. The method of any one of claims 178-184, wherein the first pre-determined time period is at least one of about 1 minute, about 10 minutes, about 60 minutes, about 2 hours, about 4 hours, about 6 hours, about 12 hours, and about 24 hours.

186. The method of any one of claims 178-185, wherein the second pre-determined time period is at least one of about 1 minute, about 10 minutes, about 60 minutes, about 2 hours, about 4 hours, about 6 hours, about 12 hours, about 24 hours, about 36 hours, about 48 hours, about 72 hours, about 96 hours, about

187. A method of selecting an ammonia oxidizing bacteria-friendly excipient comprising:
obtaining a sample of ammonia oxidizing bacteria (AOB);
contacting the AOB with an excipient for a first pre-determined time period to provide an incubated culture;
collecting an aliquot of the incubated culture;
measuring a concentration of nitrite in a supernatant of the incubated culture.

188. The method of claim 187, further comprising, identifying the excipient as an ammonia oxidizing bacteria-friendly excipient based on the concentration of nitrite in the supernatant of the incubated culture.

189. The method of claim 188, wherein the concentration of nitrite in the supernatant of the incubated culture is at least as shown in the Figures of the Examples disclosed herein to be identified as an ammonia-oxidizing bacteria-friendly excipient.

190. The method of any one of claims 187-189, further comprising:
contacting the AOB from the incubated culture with NH₄⁺; and
measuring the recovered AOB cell sample for at least one of an OD₆₀₀ value and nitrite accumulation after a second pre-determined time period.

191. The method of claim 190, further comprising, identifying the excipient as an ammonia oxidizing bacteria-friendly ingredient based on at least one of the OD₆₀₀ value and nitrite concentration in the recovered AOB cell sample.

192. The method of claim 191, wherein the OD₆₀₀ value in the recovered AOB cell sample is greater than about 0.01 to be identified as an ammonia-oxidizing bacteria-friendly excipient.

193. The method of claim 191, wherein the nitrite concentration in the recovered AOB cell sample is at least as shown in the Figures of the Examples and as disclosed herein to be identified as an ammonia-oxidizing bacteria-friendly excipient.

194. A method of producing a biome-friendly composition comprising:
acquiring knowledge that a compound is friendly to ammonia oxidizing bacteria; and
combining the compound with ammonia oxidizing bacteria to provide a cosmetic product.

195. A method of maintaining ammonia oxidizing bacteria (AOB) on a subject comprising:
applying a cosmetic product or a finished cosmetic product of any one of claims 1 to 116.

196. The method of claim 195, further comprising, applying the preparation comprising AOB to the subject prior to applying the cosmetic product or the finished cosmetic product.

197. The method of any one of claims 195-196, further comprising, applying the preparation comprising AOB to the subject subsequent to applying the cosmetic product or the finished cosmetic product.

198. The method of any one of claims 195-197, further comprising applying the preparation comprising AOB to the subject prior to applying the cosmetic product or the finished cosmetic product, wherein the preparation comprising AOB is applied between about one of the following ranges: about 1-5, 5-10, 10-20, 20-30. 30-40, 40-50, 50-60 minutes, 2-5, 5-10, 10-15, 15-20, 20-25 hours, 2-5, 5-10, 10-15, days, 3-4, 5-10 weeks prior to applying the cosmetic product or the finished cosmetic product.

199. The method of any one of claims 195-198, further comprising applying the preparation comprising AOB to the subject subsequent to applying the cosmetic product or the finished cosmetic product, wherein the preparation comprising AOB is applied between about one of the following ranges: about 1-5, 5-10, 10-20, 20-30. 30-40, 40-50, 50-60 minutes, 2-5, 5-10, 10-15, 15-20, 20-25 hours, 2-5, 5-10, 10-15, days, 3-4, 5-10 weeks subsequent to applying the cosmetic product or the finished cosmetic product.

200. The method of any one of claims 195-199, further comprising not applying a non biome-friendly cosmetic product or finished cosmetic product prior to or subsequent.

201. The method of any one of claims 195-200, wherein at least one of the preparation of AOB and the cosmetic product, e.g., finished cosmetic product is applied to a pre-defined area of the subject.

202. The method of claim 201, wherein the pre-defined area of the subject is at least one of a portion of a head, e.g., a face, cheek, chin, eyelid, lip, nose, scalp, hair, forehead; neck; underarm; arm; hand; leg; foot; chest; abdomen region; buttocks; genital area; and back.

203. The method of any one of claims 195-202, wherein the AOB is N. eutropha D23.

204. The finished cosmetic product of claim 1, having 1, 2, 3, 4, or all of the properties of a, b, c, and d(i).

205. The finished cosmetic product of claim 1, having 1, 2, 3, 4, or all of the properties of a, b, c, and d(ii).

206. The finished product of any of claims 1-30, wherein said cosmetic product or said finished cosmetic product, is free of preservatives.

207. The finished cosmetic product of any one of claims 1-56, wherein all bacteria, spores, mold, and fungal species are dead or incapable of cell division.

208. The finished cosmetic product of any one of claims 1-57, wherein all bacteria, spores, mold, and fungal species, have radiation induced DNA damage sufficient to inhibit cell division.

209. The finished cosmetic product of any one of claims 1-61, wherein the sterile product is characterized in that it is substantially free from microorganisms, e.g., bacteria, e.g., fungi, e.g., spores, capable of growth, e.g., as determined by standard culture methods as described in USP 71.

210. The finished cosmetic product of any one of claims 1-62, wherein, when challenged for microorganisms capable of growth, said cosmetic product shows no growth e.g., when said microorganisms are measured by standard culture methods as described in USP 71.

211. The finished cosmetic product of any one of claims 1-72, wherein the sterile product is characterized in that it is substantially free from microorganisms, e.g., bacteria, e.g., fungi capable of growth, e.g., as determined by standard culture methods as described in USP 71. 74A. The finished cosmetic product of any one of claims 1-73, wherein, when challenged for microorganisms capable of growth, said cosmetic product shows no growth, e.g., when said microorganisms are measured by standard culture methods as described in USP 71.

212. The finished cosmetic product of any one of claims 1-80, wherein said cosmetic product, or finished cosmetic product was not heated above 140 degrees F. during or after formulation, e.g., after mixing, or filling, e.g., after disposing, or after sealing the end-use container.

213. The finished cosmetic product of any one of claims 1-91, wherein the amount of cosmetic product in said finished cosmetic product is equal to or less than an amount sufficient for 10, 20, 30, 40 or 50 uses or applications.

214. The finished cosmetic product of any one of claims 1-100, wherein said finished product comprises an indication of expiration, or lifetime, e.g., recommended lifetime, after the preselected period of time, e.g., expressed in days, that is less than X days from the date of one of manufacturing, filling, sealing, shipping, releasing into commerce, or selling, wherein X is about 180 days, e.g., 180 days.

215. The method of any one of claims 117-133, wherein said provision is made within a preselected number of days, e.g., 1-28 days, e.g., 3-7 days, of an expiration date.

216. The method of claim 174, wherein evaluating comprises determining if the ability of the ammonia oxidizing bacteria to produce nitrite meets a preselected criteria, e.g., is at least 100 micromolar, or is in the range of 100-1000 micromolar, after 48 hours of incubation.

217. The method of claim 174, wherein evaluating comprises determining if the ability of the ammonia oxidizing bacteria to produce nitrite meets a preselected criteria, e.g., is at least 1000 micromolar nitrite after 48 hours of incubation.

218. The method of claim 188, wherein the concentration of nitrite in the supernatant of the incubated culture is between about 100 and 1000 micromolar, measured at 48 hours after incubation, to be identified as an ammonia-oxidizing bacteria-friendly excipient.

219. The method of claim 188, wherein the concentration of nitrite in the supernatant of the incubated culture is greater than 1000 micromolar, measured at 48 hours after incubation, to be identified as an ammonia-oxidizing bacteria-friendly excipient.
